SEQUENCE LISTING (100 sequences)

SEQ ID NO: 1 - DNA, 25 bp, Artificial Sequence
  Description of Artificial Sequence Synthetic oligonucleotide Pair ID 265003; MM probe is SEQ ID NO2
aatggaatttgatagaacctctgga

SEQ ID NO: 2 - DNA, 25 bp, Artificial Sequence
  Description of Artificial Sequence Synthetic oligonucleotide Pair ID 265003; PM probe is SEQ ID NO1
aatggaatttgaaagaacctctgga

SEQ ID NO: 3 - DNA, 25 bp, Artificial Sequence
  Description of Artificial Sequence Synthetic oligonucleotide Pair ID 231625; MM probe is SEQ ID NO4
cagtaaggcatgagtaacttaccgt

SEQ ID NO: 4 - DNA, 25 bp, Artificial Sequence
  Description of Artificial Sequence Synthetic oligonucleotide Pair ID 231625; PM probe is SEQ ID NO3
cagtaaggcatgggtaacttaccgt

SEQ ID NO: 5 - DNA, 25 bp, Artificial Sequence
  Description of Artificial Sequence Synthetic oligonucleotide Pair ID 186992; MM probe is SEQ ID NO6
aaacgatgaccactagacgttgggg

SEQ ID NO: 6 - DNA, 25 bp, Artificial Sequence
  Description of Artificial Sequence Synthetic oligonucleotide Pair ID 186992; PM probe is SEQ ID NO5
aaacgatgaccattagacgttgggg

SEQ ID NO: 7 - DNA, 25 bp, Artificial Sequence
  Description of Artificial Sequence Synthetic oligonucleotide Pair ID 327194; MM probe is SEQ ID NO8
ataaagttacttgcctgcatttggt

SEQ ID NO: 8 - DNA, 25 bp, Artificial Sequence
  Description of Artificial Sequence Synthetic oligonucleotide Pair ID 327194; PM probe is SEQ ID NO7
ataaagttacttccctgcatttggt

SEQ ID NO: 9 - DNA, 25 bp, Artificial Sequence
  Description of Artificial Sequence Synthetic oligonucleotide Pair ID 151981; MM probe is SEQ ID NO10
ccgtagcgtacgtgatctacgatta

SEQ ID NO: 10 - DNA, 25 bp, Artificial Sequence
  Description of Artificial Sequence Synthetic oligonucleotide Pair ID 151981; PM probe is SEQ ID NO9
ccgtagcgtacgggatctacgatta

SEQ ID NO: 11 - DNA, 25 bp, Artificial Sequence
  Description of Artificial Sequence Synthetic oligonucleotide Pair ID 191212; MM probe is SEQ ID NO12
taaaatttcgatggatacggattgg

SEQ ID NO: 12 - DNA, 25 bp, Artificial Sequence
  Description of Artificial Sequence Synthetic oligonucleotide Pair ID 191212; PM probe is SEQ ID NO11
taaaatttcgatcgatacggattgg

SEQ ID NO: 13 - DNA, 25 bp, Artificial Sequence
  Description of Artificial Sequence Synthetic oligonucleotide Pair ID 276480; MM probe is SEQ ID NO14
caggcacgaaaacgtggggatccaa

SEQ ID NO: 14 - DNA, 25 bp, Artificial Sequence
  Description of Artificial Sequence Synthetic oligonucleotide Pair ID 276480; PM probe is SEQ ID NO13
caggcacgaaaaggtggggatccaa

SEQ ID NO: 15 - DNA, 25 bp, Artificial Sequence
  Description of Artificial Sequence Synthetic oligonucleotide Pair ID 300668; MM probe is SEQ ID NO16
cggttctttttgatggcgaccggcg

SEQ ID NO: 16 - DNA, 25 bp, Artificial Sequence
  Description of Artificial Sequence Synthetic oligonucleotide Pair ID 300668; PM probe is SEQ ID NO15
cggttctttttgttggcgaccggcg

SEQ ID NO: 17 - DNA, 25 bp, Artificial Sequence
  Description of Artificial Sequence Synthetic oligonucleotide Pair ID 25579; MM probe is SEQ ID NO18
ggccttagcccgtgctgccgtacga

SEQ ID NO: 18 - DNA, 25 bp, Artificial Sequence
  Description of Artificial Sequence Synthetic oligonucleotide Pair ID 25579; PM probe is SEQ ID NO17
ggccttagcccgagctgccgtacga

SEQ ID NO: 19 - DNA, 25 bp, Artificial Sequence
  Description of Artificial Sequence Synthetic oligonucleotide Pair ID 176744; MM probe is SEQ ID NO20
cgcatgtcgagtatgttaaatatcc

SEQ ID NO: 20 - DNA, 25 bp, Artificial Sequence
  Description of Artificial Sequence Synthetic oligonucleotide Pair ID 176744; PM probe is SEQ ID NO19
cgcatgtcgagtttgttaaatatcc

SEQ ID NO: 21 - DNA, 25 bp, Artificial Sequence
  Description of Artificial Sequence Synthetic oligonucleotide Pair ID 263709; MM probe is SEQ ID NO22
cccaggttcgatagtaccaattagg

SEQ ID NO: 22 - DNA, 25 bp, Artificial Sequence
  Description of Artificial Sequence Synthetic oligonucleotide Pair ID 263709; PM probe is SEQ ID NO21
cccaggttcgatggtaccaattagg

SEQ ID NO: 23 - DNA, 25 bp, Artificial Sequence
  Description of Artificial Sequence Synthetic oligonucleotide Pair ID 289612; MM probe is SEQ ID NO24
ccaaggacgttcgtgagccgttcga

SEQ ID NO: 24 - DNA, 25 bp, Artificial Sequence
  Description of Artificial Sequence Synthetic oligonucleotide Pair ID 289612; PM probe is SEQ ID NO23
ccaaggacgttcttgagccgttcga

SEQ ID NO: 25 - DNA, 25 bp, Artificial Sequence
  Description of Artificial Sequence Synthetic oligonucleotide Pair ID 4284; MM probe is SEQ ID NO26
aaacgacccactgggactgtcctga

SEQ ID NO: 26 - DNA, 25 bp, Artificial Sequence
  Description of Artificial Sequence Synthetic oligonucleotide Pair ID 4284; PM probe is SEQ ID NO25
aaacgacccactaggactgtcctga

SEQ ID NO: 27 - DNA, 25 bp, Artificial Sequence
  Description of Artificial Sequence Synthetic oligonucleotide Pair ID 111799; MM probe is SEQ ID NO28
aaaattgcccgaaggaggatctatt

SEQ ID NO: 28 - DNA, 25 bp, Artificial Sequence
  Description of Artificial Sequence Synthetic oligonucleotide Pair ID 111799; PM probe is SEQ ID NO27
aaaattgcccgatggaggatctatt

SEQ ID NO: 29 - DNA, 25 bp, Artificial Sequence
  Description of Artificial Sequence Synthetic oligonucleotide Pair ID 57769; MM probe is SEQ ID NO30
atattactccccataacattcgtcc

SEQ ID NO: 30 - DNA, 25 bp, Artificial Sequence
  Description of Artificial Sequence Synthetic oligonucleotide Pair ID 57769; PM probe is SEQ ID NO29
atattactccccctaacattcgtcc

SEQ ID NO: 31 - DNA, 25 bp, Artificial Sequence
  Description of Artificial Sequence Synthetic oligonucleotide Pair ID 277222; MM probe is SEQ ID NO32
aacttccttagctccaaatacattc

SEQ ID NO: 32 - DNA, 25 bp, Artificial Sequence
  Description of Artificial Sequence Synthetic oligonucleotide Pair ID 277222; PM probe is SEQ ID NO31
aacttccttagcaccaaatacattc

SEQ ID NO: 33 - DNA, 25 bp, Artificial Sequence
  Description of Artificial Sequence Synthetic oligonucleotide Pair ID 39458; MM probe is SEQ ID NO34
aagtcccgtacgagcgcaacccttg

SEQ ID NO: 34 - DNA, 25 bp, Artificial Sequence
  Description of Artificial Sequence Synthetic oligonucleotide Pair ID 39458; PM probe is SEQ ID NO33
aagtcccgtacgcgcgcaacccttg

SEQ ID NO: 35 - DNA, 25 bp, Artificial Sequence
  Description of Artificial Sequence Synthetic oligonucleotide Pair ID 298577; MM probe is SEQ ID NO36
aaggcccggcactaactatgaccgg

SEQ ID NO: 36 - DNA, 25 bp, Artificial Sequence
  Description of Artificial Sequence Synthetic oligonucleotide Pair ID 298577; PM probe is SEQ ID NO35
aaggcccggcacgaactatgaccgg

SEQ ID NO: 37 - DNA, 25 bp, Artificial Sequence
  Description of Artificial Sequence Synthetic oligonucleotide Pair ID 126538; MM probe is SEQ ID NO38
acagtacttaaagaataagcaccgg

SEQ ID NO: 38 - DNA, 25 bp, Artificial Sequence
  Description of Artificial Sequence Synthetic oligonucleotide Pair ID 126538; PM probe is SEQ ID NO37
acagtacttaaaaaataagcaccgg

SEQ ID NO: 39 - DNA, 25 bp, Artificial Sequence
  Description of Artificial Sequence Synthetic oligonucleotide Pair ID 252982; MM probe is SEQ ID NO40
aaatattggggctcaaccttaagca

SEQ ID NO: 40 - DNA, 25 bp, Artificial Sequence
  Description of Artificial Sequence Synthetic oligonucleotide Pair ID 252982; PM probe is SEQ ID NO39
aaatattggggcgcaaccttaagca

SEQ ID NO: 41 - DNA, 25 bp, Artificial Sequence
  Description of Artificial Sequence Synthetic oligonucleotide Pair ID 197225; MM probe is SEQ ID NO42
cgtatagtacggtcagttttctaca

SEQ ID NO: 42 - DNA, 25 bp, Artificial Sequence
  Description of Artificial Sequence Synthetic oligonucleotide Pair ID 197225; PM probe is SEQ ID NO41
cgtatagtacgggcagttttctaca

SEQ ID NO: 43 - DNA, 25 bp, Artificial Sequence
  Description of Artificial Sequence Synthetic oligonucleotide Pair ID 162084; MM probe is SEQ ID NO44
ccgaaggaccggttctcgctcgact

SEQ ID NO: 44 - DNA, 25 bp, Artificial Sequence
  Description of Artificial Sequence Synthetic oligonucleotide Pair ID 162084; PM probe is SEQ ID NO43
ccgaaggaccggctctcgctcgact

SEQ ID NO: 45 - DNA, 25 bp, Artificial Sequence
  Description of Artificial Sequence Synthetic oligonucleotide Pair ID 228980; MM probe is SEQ ID NO46
cctagttaccgatcttgccctaggc

SEQ ID NO: 46 - DNA, 25 bp, Artificial Sequence
  Description of Artificial Sequence Synthetic oligonucleotide Pair ID 228980; PM probe is SEQ ID NO45
cctagttaccgagcttgccctaggc

SEQ ID NO: 47 - DNA, 25 bp, Artificial Sequence
  Description of Artificial Sequence Synthetic oligonucleotide Pair ID 123763; MM probe is SEQ ID NO48
cacagatttccctgtgttattcccc

SEQ ID NO: 48 - DNA, 25 bp, Artificial Sequence
  Description of Artificial Sequence Synthetic oligonucleotide Pair ID 123763; PM probe is SEQ ID NO47
cacagatttcccggtgttattcccc

SEQ ID NO: 49 - DNA, 25 bp, Artificial Sequence
  Description of Artificial Sequence Synthetic oligonucleotide Pair ID 240310; MM probe is SEQ ID NO50
tagcaatacatcaggtagttagtgg

SEQ ID NO: 50 - DNA, 25 bp, Artificial Sequence
  Description of Artificial Sequence Synthetic oligonucleotide Pair ID 240310; PM probe is SEQ ID NO49
tagcaatacatccggtagttagtgg

SEQ ID NO: 51 - DNA, 25 bp, Artificial Sequence
  Description of Artificial Sequence Synthetic oligonucleotide Pair ID 16702; MM probe is SEQ ID NO52
gactcaacaggcatcacatcagata

SEQ ID NO: 52 - DNA, 25 bp, Artificial Sequence
  Description of Artificial Sequence Synthetic oligonucleotide Pair ID 16702; PM probe is SEQ ID NO51
gactcaacaggcttcacatcagata

SEQ ID NO: 53 - DNA, 25 bp, Artificial Sequence
  Description of Artificial Sequence Synthetic oligonucleotide Pair ID 16703; MM probe is SEQ ID NO54
tatctgatgtgatgcctgttgagtc

SEQ ID NO: 54 - DNA, 25 bp, Artificial Sequence
  Description of Artificial Sequence Synthetic oligonucleotide Pair ID 16703; PM probe is SEQ ID NO53
tatctgatgtgaagcctgttgagtc

SEQ ID NO: 55 - DNA, 25 bp, Artificial Sequence
  Description of Artificial Sequence Synthetic oligonucleotide Pair ID 24979; MM probe is SEQ ID NO56
tcgggcgttgttaaatcaatcaaga

SEQ ID NO: 56 - DNA, 25 bp, Artificial Sequence
  Description of Artificial Sequence Synthetic oligonucleotide Pair ID 24979; PM probe is SEQ ID NO55
tcgggcgttgtttaatcaatcaaga

SEQ ID NO: 57 - DNA, 25 bp, Artificial Sequence
  Description of Artificial Sequence Synthetic oligonucleotide Pair ID 24980; MM probe is SEQ ID NO58
tcttgattgatttaacaacgcccga

SEQ ID NO: 58 - DNA, 25 bp, Artificial Sequence
  Description of Artificial Sequence Synthetic oligonucleotide Pair ID 24980; PM probe is SEQ ID NO57
tcttgattgattaaacaacgcccga

SEQ ID NO: 59 - DNA, 25 bp, Artificial Sequence
  Description of Artificial Sequence Synthetic oligonucleotide Pair ID 41927; MM probe is SEQ ID NO60
aagcatctgactcaacaggcatcac

SEQ ID NO: 60 - DNA, 25 bp, Artificial Sequence
  Description of Artificial Sequence Synthetic oligonucleotide Pair ID 41927; PM probe is SEQ ID NO59
aagcatctgactgaacaggcatcac

SEQ ID NO: 61 - DNA, 25 bp, Artificial Sequence
  Description of Artificial Sequence Synthetic oligonucleotide Pair ID 41928; MM probe is SEQ ID NO62
gtgatgcctgttgagtcagatgctt

SEQ ID NO: 62 - DNA, 25 bp, Artificial Sequence
  Description of Artificial Sequence Synthetic oligonucleotide Pair ID 41928; PM probe is SEQ ID NO61
gtgatgcctgttcagtcagatgctt

SEQ ID NO: 63 - DNA, 25 bp, Artificial Sequence
  Description of Artificial Sequence Synthetic oligonucleotide Pair ID 44977; MM probe is SEQ ID NO64
ctgatcgtatgtatggtctatggcc

SEQ ID NO: 64 - DNA, 25 bp, Artificial Sequence
  Description of Artificial Sequence Synthetic oligonucleotide Pair ID 44977; PM probe is SEQ ID NO63
ctgatcgtatgtttggtctatggcc

SEQ ID NO: 65 - DNA, 25 bp, Artificial Sequence
  Description of Artificial Sequence Synthetic oligonucleotide Pair ID 44978; MM probe is SEQ ID NO66
ggccatagaccatacatacgatcag

SEQ ID NO: 66 - DNA, 25 bp, Artificial Sequence
  Description of Artificial Sequence Synthetic oligonucleotide Pair ID 44978; PM probe is SEQ ID NO65
ggccatagaccaaacatacgatcag

SEQ ID NO: 67 - DNA, 25 bp, Artificial Sequence
  Description of Artificial Sequence Synthetic oligonucleotide Pair ID 46837; MM probe is SEQ ID NO68
taaatcaatcaagacatccggttgt

SEQ ID NO: 68 - DNA, 25 bp, Artificial Sequence
  Description of Artificial Sequence Synthetic oligonucleotide Pair ID 46837; PM probe is SEQ ID NO67
taaatcaatcaacacatccggttgt

SEQ ID NO: 69 - DNA, 25 bp, Artificial Sequence
  Description of Artificial Sequence Synthetic oligonucleotide Pair ID 46838; MM probe is SEQ ID NO70
acaaccggatgtcttgattgattta

SEQ ID NO: 70 - DNA, 25 bp, Artificial Sequence
  Description of Artificial Sequence Synthetic oligonucleotide Pair ID 46838; PM probe is SEQ ID NO69
acaaccggatgtgttgattgattta

SEQ ID NO: 71 - DNA, 25 bp, Artificial Sequence
  Description of Artificial Sequence Synthetic oligonucleotide Pair ID 48919; MM probe is SEQ ID NO72
cgatgatggctccgatccctttttc

SEQ ID NO: 72 - DNA, 25 bp, Artificial Sequence
  Description of Artificial Sequence Synthetic oligonucleotide Pair ID 48919; PM probe is SEQ ID NO71
cgatgatggctcggatccctttttc

SEQ ID NO: 73 - DNA, 25 bp, Artificial Sequence
  Description of Artificial Sequence Synthetic oligonucleotide Pair ID 48920; MM probe is SEQ ID NO74
gaaaaagggatcggagccatcatcg

SEQ ID NO: 74 - DNA, 25 bp, Artificial Sequence
  Description of Artificial Sequence Synthetic oligonucleotide Pair ID 48920; PM probe is SEQ ID NO73
gaaaaagggatccgagccatcatcg

SEQ ID NO: 75 - DNA, 25 bp, Artificial Sequence
  Description of Artificial Sequence Synthetic oligonucleotide Pair ID 74275; MM probe is SEQ ID NO76
cagtatcgcaccgagcgcaaaattt

SEQ ID NO: 76 - DNA, 25 bp, Artificial Sequence
  Description of Artificial Sequence Synthetic oligonucleotide Pair ID 74275; PM probe is SEQ ID NO75
cagtatcgcacccagcgcaaaattt

SEQ ID NO: 77 - DNA, 25 bp, Artificial Sequence
  Description of Artificial Sequence Synthetic oligonucleotide Pair ID 74276; MM probe is SEQ ID NO78
aaattttgcgctcggtgcgatactg

SEQ ID NO: 78 - DNA, 25 bp, Artificial Sequence
  Description of Artificial Sequence Synthetic oligonucleotide Pair ID 74276; PM probe is SEQ ID NO77
aaattttgcgctgggtgcgatactg

SEQ ID NO: 79 - DNA, 25 bp, Artificial Sequence
  Description of Artificial Sequence Synthetic oligonucleotide Pair ID 80839; MM probe is SEQ ID NO80
aatcagcttctgagaaaccggttgt

SEQ ID NO: 80 - DNA, 25 bp, Artificial Sequence
  Description of Artificial Sequence Synthetic oligonucleotide Pair ID 80839; PM probe is SEQ ID NO79
aatcagcttctgtgaaaccggttgt

SEQ ID NO: 81 - DNA, 25 bp, Artificial Sequence
  Description of Artificial Sequence Synthetic oligonucleotide Pair ID 80840; MM probe is SEQ ID NO82
acaaccggtttctcagaagctgatt

SEQ ID NO: 82 - DNA, 25 bp, Artificial Sequence
  Description of Artificial Sequence Synthetic oligonucleotide Pair ID 80840; PM probe is SEQ ID NO81
acaaccggtttcacagaagctgatt

SEQ ID NO: 83 - DNA, 25 bp, Artificial Sequence
  Description of Artificial Sequence Synthetic oligonucleotide Pair ID 83957; MM probe is SEQ ID NO84
ctcaacaggcatcacatcagataat

SEQ ID NO: 84 - DNA, 25 bp, Artificial Sequence
  Description of Artificial Sequence Synthetic oligonucleotide Pair ID 83957; PM probe is SEQ ID NO83
ctcaacaggcatgacatcagataat

SEQ ID NO: 85 - DNA, 25 bp, Artificial Sequence
  Description of Artificial Sequence Synthetic oligonucleotide Pair ID 83958; MM probe is SEQ ID NO86
attatctgatgtgatgcctgttgag

SEQ ID NO: 86 - DNA, 25 bp, Artificial Sequence
  Description of Artificial Sequence Synthetic oligonucleotide Pair ID 83958; PM probe is SEQ ID NO85
attatctgatgtcatgcctgttgag

SEQ ID NO: 87 - DNA, 25 bp, Artificial Sequence
  Description of Artificial Sequence Synthetic oligonucleotide Pair ID 93455; MM probe is SEQ ID NO88
tttctgtaaaacaggcatggatatc

SEQ ID NO: 88 - DNA, 25 bp, Artificial Sequence
  Description of Artificial Sequence Synthetic oligonucleotide Pair ID 93455; PM probe is SEQ ID NO87
tttctgtaaaactggcatggatatc

SEQ ID NO: 89 - DNA, 25 bp, Artificial Sequence
  Description of Artificial Sequence Synthetic oligonucleotide Pair ID 93456; MM probe is SEQ ID NO90
gatatccatgcctgttttacagaaa

SEQ ID NO: 90 - DNA, 25 bp, Artificial Sequence
  Description of Artificial Sequence Synthetic oligonucleotide Pair ID 93456; PM probe is SEQ ID NO89
gatatccatgccagttttacagaaa

SEQ ID NO: 91 - DNA, 25 bp, Artificial Sequence
  Description of Artificial Sequence Synthetic oligonucleotide Pair ID 140612; MM probe is SEQ ID NO92
ctcaacatcctcaaaatagttggca

SEQ ID NO: 92 - DNA, 25 bp, Artificial Sequence
  Description of Artificial Sequence Synthetic oligonucleotide Pair ID 140612; PM probe is SEQ ID NO91
ctcaacatcctctaaatagttggca

SEQ ID NO: 93 - DNA, 25 bp, Artificial Sequence
  Description of Artificial Sequence Synthetic oligonucleotide Pair ID 140613; MM probe is SEQ ID NO94
tgccaactattttgaggatgttgag

SEQ ID NO: 94 - DNA, 25 bp, Artificial Sequence
  Description of Artificial Sequence Synthetic oligonucleotide Pair ID 140613; PM probe is SEQ ID NO93
tgccaactatttagaggatgttgag

SEQ ID NO: 95 - DNA, 25 bp, Artificial Sequence
  Description of Artificial Sequence Synthetic oligonucleotide Pair ID 193149; MM probe is SEQ ID NO96
atttcatcatcagtatcgcaccgag

SEQ ID NO: 96 - DNA, 25 bp, Artificial Sequence
  Description of Artificial Sequence Synthetic oligonucleotide Pair ID 193149; PM probe is SEQ ID NO95
atttcatcatcactatcgcaccgag

SEQ ID NO: 97 - DNA, 25 bp, Artificial Sequence
  Description of Artificial Sequence Synthetic oligonucleotide Pair ID 193150; MM probe is SEQ ID NO98
ctcggtgcgatactgatgatgaaat

SEQ ID NO: 98 - DNA, 25 bp, Artificial Sequence
  Description of Artificial Sequence Synthetic oligonucleotide Pair ID 193150; PM probe is SEQ ID NO97
ctcggtgcgatagtgatgatgaaat

SEQ ID NO: 99 - DNA, 25 bp, Artificial Sequence
  Description of Artificial Sequence Synthetic oligonucleotide Pair ID 195043; MM probe is SEQ ID NO100
taaaacaggcatggatatctgtgta

SEQ ID NO: 100 - DNA, 25 bp, Artificial Sequence
  Description of Artificial Sequence Synthetic oligonucleotide Pair ID 195043; PM probe is SEQ ID NO99
taaaacaggcatcgatatctgtgta

(19) United States
(12) Patent Application Publication
(10) Pub. No.: US 2018/0223339 A1
(43) Pub. Date: Aug. 9, 2018

(54) METHODS AND SYSTEMS FOR PHYLOGENETIC ANALYSIS

(71) Applicant: The Regents of the University of California, Oakland, CA (US)

(72) Inventors: Gary L. Andersen, Berkeley, CA (US); Todd Z. DeSantis, Livermore, CA (US); Eoin Brodie, San Francisco, CA (US)

(21) Appl. No.: 15/910,905

(22) Filed: Mar. 2, 2018

Related U.S. Application Data
(62) Division of application No. 13/379,839, filed on Mar. 12, 2012, now abandoned.
(60) Provisional application No. 61/220,937, filed on Jun. 26, 2009.
(60) Provisional application No. 61/259,565, filed on Nov. 9, 2009.
(60) Provisional application No. 61/317,644, filed on Mar. 25, 2010.
(60) Provisional application No. 61/347,817, filed on May 24, 2010.

(51) Int. Cl. C12Q 1/689; C12Q 1/6837

(52) U.S. Cl. CPC C12Q 1/689; C12Q 2600/158; C12Q 1/6837

(57) ABSTRACT

The present invention discloses methods and systems for designing and using organism-specific and/or operational taxon unit (OTU)-specific probes. The methods and systems allow for detecting, identifying and quantitating a plurality of biomolecules or microorganisms in a sample based on the hybridization or binding of target molecules in the sample with the probes. Some embodiments provide methods of selecting an oligonucleotide probe specific for a node on a clustering tree. Other embodiments provide methods of selecting organism-specific or OTU-specific oligonucleotide probes for use in accurately detecting a plurality of organisms in a sample with high confidence. Some embodiments provide methods and systems to detect the presence of a rare OTU in a sample.

### CROSS-REFERENCE

This application is related to and claims priority to the following co-pending U.S. provisional patent applications: U.S. Application Ser. No. 61/220,937 [Attorney Docket No. IB-2733P], filed on Jun. 26, 2009; U.S. Application Ser. No. 61/259,565 [Attorney Docket No. IB-2733P1], filed on Nov. 9, 2009; U.S. Application Ser. No. 61/317,644 [Attorney Docket No. IB-2733P2], filed on Mar. 25, 2010; U.S. Application Ser. No. 61/347,817 [Attorney Docket No. IB-2733P3], filed on May 24, 2010; each of which are incorporated herein by reference.

### STATEMENT AS TO FEDERALLY SPONSORED RESEARCH

This invention was made with Government support under Contract No. DE-AC02-05CH11231 awarded by the Department of Energy; a grant from the Department of Homeland Security and Agreement Number 07-576-550-0 from State of California Water Quality Board. The government has certain rights in this invention.

### BACKGROUND OF THE INVENTION

With as many as 10³⁰ microbial genomes globally, across multiple different environmental and host conditions, variety both within and between microbiomes is well recognized (Huse et al. (2008), PLoS Genetics 4(11): e1000255). As a result of this variety, characterizing the contents of a microbiome is a challenge for current approaches. Firstly, standard culturing techniques are successful in maintaining only a small fraction of the microorganisms in nature. Means of more direct profiling, such as sequencing, face two additional challenges. Both the sheer number of different genomes in a given sample and the degree of homology between members present a complex problem for already laborious procedures.

Biopolymers such as nucleic acids and proteins are often identified in the search for useful genes, to diagnose diseases or to identify organisms. Frequently, hybridization or another binding reaction is used as part of the identification step. As the number of possible targets increases in a sample, the design of systems to detect the different hybridization reactions increases in difficulty along with the analysis of the binding or hybridization data. The design and analysis problems become acute when there are many similar targets in a sample as is the case when the individual species or groups that comprise a microbiome are detected or quantified in a single assay based on a highly conserved polynucleotide. For example, while approximately 98% of bacteria found in the human gut belong to only four bacterial divisions, this includes approximately 36,000 different phylotypes at the strain level, having ≥99% sequence identity (Hattori et al. (2009), DNA Res. 16: 1-12). While possibly containing certain overlapping taxa, the different environments presented by the guts of other hosts are expected to support different microbiomes. In situations where contributions from multiple sub-enviroments are combined, such as a water source potentially contaminated by a variety of sources, just identifying the thousands of taxa is a significant challenge to current methods of detection.

Since the study of microbiomes can offer new insight into origins of environmental change, disease, immunological functions, and physiological functions, improved methods for designing nucleic acids, proteins, or other probes that can recognize specific organisms, or taxa are needed. Similarly, improved methods for data analysis that allow detection and quantification of the members of a microbial community at high confidence levels are also needed.

### SUMMARY OF THE INVENTION

Some embodiments provide a system comprising a plurality of probes capable of determining the presence, absence, relative abundance, and/or quantity of at least 10,000 different OTUs in a single assay. In some embodiments, the system is configured to produce a biosignature that is indicative of fecal contamination. In some embodiments, the probes selectively hybridize to one or more highly conserved polynucleotides, which can include 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, cox1 gene, nif13 gene, RNA molecules derived therefrom, or a combination thereof. In some embodiments, the conserved polynucleotides are amplicons. In some embodiments, the probes can be attached to a substrate. In some embodiments, the probes can form an array. In some embodiments, the substrate comprises a bead, microsphere, glass, plastic, or silicon. In some embodiments, the system is capable of performing sequencing reactions on the same highly conserved region of each of the OTUs. In some embodiments, the system further comprises one or more species-specific probes. In some embodiments, each of the OTUs is bacterial, archaeal, or fungal.

In some embodiments, the system further comprises a plurality of positive control probes. In some embodiments, the system further comprises a plurality of negative control probes. In some embodiments, the negative control probes comprise sequences that are not complementary to sequence found in the highly conserved polynucleotide. In some embodiments, the positive control probes comprise sequences that are complementary to a polynucleotide selected from SEQ ID NOs:51-100. In some embodiments, the positive control probes comprise one or more sequences selected from SEQ ID NOs: 51-100.

In some embodiments, the system removes data from at least a subset of said interrogation probes before making a final call on the presence, absence, relative abundance, and/or quantity of said OTUs. In some embodiments, the data is removed based on interrogation probe cross-hybridization potential.

Some embodiments provide a system capable of detecting one or more first nucleic acid sequences comprising 1×10⁻³ or less of the total nucleic acids present in a single assay with a confidence level greater than 95% and sensitivity level greater than 95%, wherein the one or more first nucleic acid sequences and set of remaining target nucleic acids are at least 95% homologous. In some embodiments, the system is configured to produce a biosignature that is indicative of fecal contamination. In some embodiments, one or more of the nucleic acid sequences are 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, cox1 gene, nif13 gene, RNA molecules derived therefrom, or a combination thereof. In some embodiments, the nucleic acids comprise amplicons.

Some embodiments provide a system for determining the presence, absence, relative abundance, and/or quantity of a plurality of different OTUs in a single assay, said system comprising a plurality of polynucleotide interrogation probes, a plurality of polynucleotide positive control probes, and a plurality of polynucleotide negative control probes. In some embodiments, the system is configured to produce a biosignature that is indicative of fecal contamination. In some embodiments, the system removes data from at least a subset of said interrogation probes before making a final call on the presence, absence, relative abundance, and/or quantity of said microorganisms. In some embodiments, data is removed based on interrogation probe cross hybridization potential.

Some embodiments provide a system capable of detecting the presence, absence, relative abundance, and/or quantity of more than 10,000 different OTUs of a single domain (e.g. bacterial, archaeal, or fungal) in a single assay with confidence greater than 95%. In some embodiments, the system is configured to produce a biosignature that is indicative of fecal contamination. In some embodiments, the system comprises a plurality of probes that selectively hybridize to the same highly conserved region in each of said OTUs. In some embodiments, the system is capable of performing sequencing reactions on the same highly conserved region of each of said OTUs. In some embodiments, the system further comprises species-specific probes, wherein the probes do not hybridize to said highly conserved sequence. In some embodiments, the system comprises 100 species-specific probes.

Some embodiments provide a system for determining the presence, absence, relative abundance, and/or quantity of one or more microorganisms from a sample, said system comprising a plurality of OTUs, wherein the median number of probes per OTU is less than 26. Some embodiments provide a system for determining the presence, absence, relative abundance, and/or quantity of one or more microorganisms from a sample, said system comprising a plurality of OTUs, wherein the median number of cross-hybridizations per probe is less than 20. In some embodiments, the system is configured to produce a biosignature that is indicative of fecal contamination.

Some embodiments provide a method for determining a condition of a sample comprising: a) contacting said sample with a plurality of different probes; b) determining hybridization signal strength for each of said probes, wherein said determination establishes a biosignature for said sample; and c) comparing the biosignature of said sample to a biosignature for fecal contamination.

In one aspect of the invention, a method is provided for determining the probability of the presence, relative abundance, and/or quantity of a microorganism in a sample comprising a) determining hybridization signal strength distributions of negative control probes that do not specifically hybridize to a highly conserved polynucleotide in the microorganism; b) determining hybridization signal strength distributions of positive control probes; c) determining hybridization signal strengths for a plurality of different interrogation probes, each of which is complementary to a section within the highly conserved polynucleotide; and d) using the hybridization signal strengths of the negative and positive probes to determine the probability that the hybridization signal for the different interrogation probes represents the presence, relative abundance, and/or quantity of the microorganism. In some embodiments, the hybridization signal strengths of the negative and positive probes are used to normalize or fit the interrogation probes hybridization data. In further embodiments, the normalization or fitting of interrogation probes hybridization data utilizes A+T content or normal and gamma distributions of the negative and positive control probes. In other embodiments, the negative control probes and/or the positive control probes comprise perfect match and mismatch probes. In further embodiments, the normal and gamma distribution of the negative and positive control probes involves calculating a pair difference score for said probes. In other embodiments, the hybridization signal strengths for the plurality of different interrogation probes are attenuated based on the G+C content of each probe.

In one aspect, a method is provided for determining the probability of the presence or quantity of a unique polynucleotide or microorganism in a sample comprising a) contacting the sample with a plurality of different probes; b) determining hybridization signal strength for sample polynucleotides to each of the probes; c) removing or attenuating from analysis an OTU/taxa from the possible list based on hybridization signal strength data, thereby increasing the confidence level of the remaining hybridization signal strength data. In some embodiments, the removing or attenuating is performed only on OTUs having a percentage of probes that pass a certain threshold intensity within such OTU. In some embodiments, only OTUs that pass a certain threshold are further analyzed. In still further embodiments, the removing or attenuating is performed by penalizing OTUs present in the sample based on potential cross hybridization of probes from the OTU with polynucleotides from other OTUs. In some embodiments, the penalization positively correlates with potential for cross hybridization with other OTUs. In other embodiments, penalization based on cross hybridization is performed at each level of a phylogenic tree starting with the lowest level. In further embodiments, only penalized OTUs scoring above a hybridization signal strength threshold are further analyzed. In still other embodiments, only parts of phylogenic tree that include an OTU are analyzed.

In a further aspect of the invention, a method is provided for determining presence or quantity of a plurality of different organisms in a sample comprising determining GC content of each probe and comparing each probe intensity to a positive control probe intensity and negative probe intensity to determine quantity of said probes.

In another aspect of the invention, computer executable logic is provided for determining a probability that one or more organisms from a set of different organisms are present in a sample said logic comprising: a) an algorithm for determining likelihood that individual interrogation probe intensities are accurate based on comparison with intensities of negative control probes and positive control probes; b) an algorithm for determining likelihood that an individual OTU is present based on intensities of interrogation probes from said OTU passing a first quantile threshold; and c) an algorithm for penalizing one or more OTUs that have passed the first quantile threshold based on potential for cross-hybridization of probes analyzing said OTUs sequences with sequences from other OTUs.

In a further aspect, computer executable logic is provided for determining the presence and optionally quantity of one or more microorganisms in a sample comprising: logic for analyzing intensities from a set of probes that selectively binds each of at least 10,000, 20,000, 30,000, 40,000, 50,000, 60,000, 70,000, 80,000, 90,000 or 100,000 highly conserved polynucleotides, and determining the presence of at least 90%, 95%, 97%, or more of all species present in said sample. The determination can be made with at least a 90%, 95%, 98%, 99%, or 99.5% confidence level.

In another aspect, computer executable logic is provided for determining the presence of one or more microorganisms in a sample comprising: logic for analyzing a set of at least 1000 different interrogation perfect probes, and logic for discarding information from at least 10% of said interrogation perfect probes in the process of making said determination.

In one aspect, a method is provided for probe selection comprising: a) selecting a set of highly conserved polynucleotides; b) comparing said plurality of polynucleotides against a plurality of standard polynucleotides to identify chimeric sequences; c) removing chimeric sequences identified in the comparison step; and d) selecting probes that are complementary to the remaining polynucleotides. In some embodiments, at least 500,000 highly conserved polynucleotides are selected. In other embodiments, a member of the plurality of polynucleotides is considered not a chimeric sequence if it shares greater than 95% similarity with a member of the plurality of standard polynucleotides. In still other embodiments, the plurality of polynucleotides are compared against themselves to identify chimeric sequences. In other embodiments, highly conserved polynucleotides comprise sequences from a 16S RNA gene, 23S RNA gene, 5S RNA gene, 5.8S rRNA gene, 12S rRNA gene, 18S rRNA gene, 28S rRNA gene, gyrB gene, rpoB gene, fusA gene, recA gene, cox1 gene, nifD gene, or combinations thereof.

In another aspect, a method of probe selection is provided comprising: a) selecting a plurality of nucleic acid sequences; b) aligning the plurality of nucleic acid sequences with a plurality of standard nucleic acid sequences to identify insertion points in each of the plurality of nucleic acid sequences; c) removing sequences with at least 10, 20, 30, 40, 50, or more insertion points or with insertions that are at least 100 nucleic acids in length; and d) selecting probes that are complementary to the remaining nucleic acids.

In a further aspect, a method of probe selection is provided comprising: a) selecting a plurality of nucleic acid sequences; b) filtering the plurality of nucleic acid sequences; c) performing hierarchical clustering on remaining nucleic acid sequences to generate a guide tree; and d) selecting probes that are complementary to each node in said guide tree. In some embodiments, filtering the plurality of nucleic acid sequences comprises removing sequences that are identified to comprise PCR primer artifacts, removing sequences that are identified to comprise insertions, removing sequences that are identified as chimeric, or any combination thereof.

In one aspect, a method is provided for identifying a microbiome signature indicative of a condition, the method comprising a) comparing the presence and optionally abundance of at least 1,000 different OTUs in a control sample without said condition and a reference sample with said condition; and b) identifying one or more OTUs that associate with said condition. In some embodiments, the condition is an oil spill. In some embodiments, an increase in the similarity in the presence and optionally abundance of said OTUs in said reference sample with respect to said control sample is indicative of remediation of said condition. In some embodiments, changes in the degree of similarity in the presence and optionally abundance of said OTUs in said reference sample with respect to said control sample are provided as a measure of remediation of said condition. In some embodiments, the method further comprises projecting a time to reaching a predetermined level of remediation of said condition.

In one aspect, a method is provided for selecting probes for assaying a condition in a sample comprising: a) applying one or more test samples having said condition to a detection system that simultaneously assays for the probability of the presence or absence of at least 10,000 OTUs of a single domain, such as bacteria, archea, fungus, or each known OTU of a single domain; b) applying one or more control samples not having said condition to said detection system to determine the probability of the presence or absence of said OTUs in said control samples; c) determining a pattern of OTUs associated with the test samples that is not associated with the control samples; and d) identifying probes that selectively detect the OTUs associated with the test sample. In some embodiments, one or more of the identified probes are selected for use in a low-density probe system. In some embodiments, the pattern consists of up to 200 different OTUs. In other embodiments, the sample is a water sample and the condition is fecal contamination, toxic alga-bloom contamination, presence of fish pathogens, a point source contamination, a non-point source contamination, or a combination thereof. In some embodiments, a unique biosignature of a type of contamination is used to determine the source of the contamination. In some embodiments, the sample is a human or animal sample. In some embodiments, the sample is obtained from the gut, respiratory system, oral cavity, sinuses, nares, urogenital tract, skin, feces, udders, or a combination thereof. In some embodiments, the condition being characterized (e.g., diagnosed or prognosed) in that sample is Crohn's Disease, irritable bowel syndrome, cancer, rhinitis, stomach ulcers, colitis, atopy, asthma, neonatal necrotizing enterocolitis, acne, food allergy, Gastroesophageal reflux disease, obesity or periodontal disease. In some embodiments, the sample is a food, water, soil, or air sample. In some embodiments, the sample is from a forest, industrial crop, or other plant.

In one aspect, a method is provided to identify at least one new indicator species for a condition comprising: a) assaying in a single experiment a control sample without said condition to determine the presence or absence of each OTU of all known bacteria, archaea, or fungi; b) assaying in a single experiment a test sample with said condition to determine the presence or absence of each OTU of all known bacteria, archaea, or fungi; c) comparing results from (a) and (b) to identify at least one microorganism whose abundance changes by a predetermined measure in response to the change in the condition, wherein the identified microorganism species represents said new indicator species for said condition. In some embodiments, the identified microorganism decreases in abundance in the presence of the condition while in others the identified microorganism increases in abundance. In some embodiments, the predetermined measure is at least a 2-fold change in abundance. In some embodiments, the predetermined measure is a statistically significant change in abundance.

In another aspect, a system is provided for determining the probability that a microorganism or a select group of microorganisms are present in a sample, the system comprising two or more probes identified by the disclosed algorithms. In some embodiments, the system determines the probability with a confidence level greater than 95%, 99% or 99.5%. In other embodiments, the determination is performed simultaneously or using a single assay.

In one aspect, a system is provided that is capable in a single assay of distinguishing between two OTUs on a phylogenetic tree with an accuracy/confidence of greater than or equal to 95%, 99% or 99.5% based on the selective hybridization of a plurality of probes to highly conserved nucleic acids isolated from each organism to be distinguished.

In another aspect, a system is provided that is capable of generating a microbiome signature comprising at least 10,000 OTUs from an environment in a single assay with an accuracy and/or confidence level greater than 95%. In some embodiments the probes selectively hybridize to nucleic acids from each organism being detected.

In one aspect a method is provided for detecting a source of microorganism contamination, the method comprising in a single assay, determining the present and quantity of at least 20, 50, 100, or more microorganism OTUs not naturally occurring in said sample and identifying the source of the contamination using a pattern of the presence and quantity of the OTUs.

In another aspect, a system is provided that is capable of detecting the presence and quantity of at least 50 different fecal taxa in a single assay. In some embodiments, the detection is based on the selective hybridization of a plurality of probes to highly conserved nucleic acids isolated from each organism to be detected. In some embodiments, detection is based on the selective hybridization of a plurality of probes that identify the organisms or taxa listed in Table 4. In some embodiments, detection comprises detecting hybridization of one or more probes that selectively hybridize to nucleic acids indicative of clean water taxa, wherein said probes are selected from that a plurality of probes that identify the organisms or taxa listed in Table 11.

In a further aspect, a method is provided for detecting fecal contamination in water comprising: detecting the presence or absence in the water sample of one or more polynucleotides which detect the taxa listed in Table 4. In some embodiments, the method further comprises detecting hybridization of one or more probes that selectively hybridize to polynucleotides indicative of clean water taxa listed in Table 11.

In another aspect, a method is provided for testing a water sample, the method comprising calculating a ratio of Bacilli, Bacteroidetes and Clostridia (BBC) species and α-proteobacteria (A) species in said water, wherein a value greater than 1.0 is indicative of fecal contamination. In some embodiments, calculating the ratio does not rely on culturing, directly counting, PCR cloning, sequencing or use of a gene expression array. In some embodiments, the Bacilli, Bacteroidetes, Clostridia and α-proteobacteria species comprise the species listed in Table 4. In some embodiments, calculating the ratio of BBC species to A species comprises contacting the water sample with a plurality of probes. In some embodiments, the plurality of probes are complimentary to a highly conserved gene.

In a further aspect, a method is provided for predicting the likelihood of a toxic alga bloom, the method comprising: a) contacting a water sample with a plurality of probes that selectively bind to nucleic acids derived from cyanobacteria selected from Table 6; b) using hybridization data derived to determine the quantity and composition of cyanobacteria in the water sample; c) measuring environmental conditions; and d) predicting the likelihood of a toxic alga bloom based on cyanobacteria quantity and composition and environmental conditions. In some embodiments, the probes to cyanobacteria nucleic acids are selected using the present methods and detect the genera listed in Table 6. In some embodiments, the environmental conditions comprise water temperature, turbidity, nitrogen concentration, oxygen concentration, carbon concentration, phosphate concentration and/or sunlight level. In further embodiments, a water management decision is made based on the likelihood of a toxic alga bloom.

In one aspect, a method is provided for determining a condition of a subject or a therapy for a subject, the method comprising performing a single nucleic acid assay on a sample from said subject to determine the presence and/or amount of at least 1000 OTUs.

In another aspect, a method is provided for predicting a condition of a sample, the method comprising a) determining microorganism population data as the probability of the presence or absence of at least 1,000 OTUs of microorganisms in the sample; b) determining gene expression data of one or more genes of said microorganisms in the sample; and c) using the expression data and population data to predict the condition of the sample. In some embodiments, the sample is a water or soil sample.

In another aspect, the invention provides a method for assessing damage caused by an oil spill. In some embodiments, the method comprises (a) determining the presence, absence, and/or abundance of at least 1,000 OTUs in one or more samples from one or more locations unaffected by said oil spill, thereby establishing an unaffected biosignature; (b) determining the presence, absence, and/or abundance of at least 1,000 OTUs in one or more samples from a location affected by said oil spill, thereby establishing an oil-spill-affected biosignature; and (c) comparing said unaffected biosignature to said oil-spill-affected biosignature, wherein differences in said biosignatures are indicative of affects on the microbiome of said location affected by said oil spill. In some embodiments, step (b) is performed at a first time and a second time. In some embodiments, a change in said differences in said biosignatures between said first time and said second time are used to track the progress of remediation of oil spill damage.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an example of a suitable computer system environment.

FIG. 2 illustrates a networked system for the remote acquisition or analysis of data obtained through a method of the invention.

FIG. 3 illustrates a flow chart of the probe selection process.

FIGS. 4A-B demonstrate the distribution of observed pair difference score, d, from quantitative standards (QS) probes and negative controls (NC) probes.

FIG. 5 is a graph showing variations of gamma scale across 79 arrays.

FIG. 6 illustrates the pre-partition process for computational load balancing.

FIG. 7 is a vector plot comparing the microbial community composition in polluted water samples compared to three potential pollution sources: sewage, septage and cattle waste to determine the source of the pollution.

FIG. 8 is a logarithmic bar graph showing the number of OTUs detected (y-axis) by the PhyloChip for each pooled clean room sample (x-axis). The number of spores detected by the spore count are shown.

FIG. 9 is a graphical representation showing the network of common and unique families detected in each pooled clean room sample.

FIG. 10A shows a graph of the pair diffusion score frequencies of probes on the PhyloChip for the pooled clean room samples.

FIG. 10B is a graphical representation showing the commonly detected phyla detected by the PhyloChip in PCR negative pooled clean room samples as a relationship network.

FIG. 11 is a graphical representation comparing the probe responses to Faecalibacterium OTU 36742 observed on two different PhyloChip experiments.

FIG. 12 is a graphical representation comparing the probe responses to Ruminococcus OTU 38712 observed on two different PhyloChip experiments.

FIG. 13 is a density plots demonstrating the d observation of the Negative Control probes.

FIG. 14 is a chart showing the concentration of 16S amplicon versus PhyloChip response.

FIG. 15 is boxplot comparison of the detection algorithm based on pair “response score”,r, distribution (novel) versus the positive fraction calculation (previously used with the G2 PhyloChip.

FIG. 16 is two graphs that show the comparison of the r score metric versus the pf by receiver operator characteristic (R.O.C) plots.

FIG. 17 is a chart showing PhyloChip results from similar biological communities form ordination clusters.

FIG. 18 is a chart showing PhyloChip results from similar biological communities form ordination clusters.

FIG. 19 shows an NMS analysis demonstrating that the four sampling sites are quite distinct, and that the biological replicates show quite high levels of similarity.

FIG. 20 is a heatplot summary of an analysis called the Method of Shrunken Centroids to identify the ˜50 or so microbial OTUs that most significantly define the observed differences in overall community structure between sampling locations.

FIG. 21 is a representation of differing degrees of change in community composition in response to a change in climate.

FIG. 22 is two charts showing NMS ordinations of PhyloChip bacteria OTUs of: a) Fresh samples collected from the North, Mid and South-lat. sites in August 2005 and b) fresh samples and transplant-control samples from the same sited at the same time (1 year after transplanting). The fresh samples depicted in both graphs are the same samples. The bars represent 1 s.d. of 3 replicates.

FIG. 23 is four charts showing NMS ordinations of PhyloChip bacteria OTUs of PhyloChip bacteria OTUs of reciprocally transplanted samples and transplanted controls collected 1 year after they were transplanted. Arrows show the trajectory of the change in composition of transplanted samples away from that of their site-of-origin controls.

FIG. 24 shows 2 charts showing the NMS ordinations of PhyloChip bacteria OTUs of: a) Fresh samples collected from the North, Mid and South-lat. sites in September 2007 and b) fresh samples and transplant-control samples from the same sites at the same time (3 years after transplanting). The fresh samples depicted in both graphs are the same samples. The bars represent 1 s.d. of 3 replicates.

FIG. 25 is four charts showing NMS ordinations of PhyloChip bacteria OTUs of reciprocally transplanted samples and transplanted controls collected 3 years after they were transplanted. Arrows show the trajectory of the change in composition of transplanted samples away from that of their site-of-origin controls.

FIG. 26 is a schematic showing cluster analysis of detected bacterial taxa in fecal samples by species and type of animal (ruminants and grazers, pinnipeds, birds).

FIG. 27 is a bar chart showing the number of indicator OTUs for each type of species.

FIG. 28 is an ordination chart showing indicator communities were compared to polluted water samples for source identification.

FIG. 29 is a bar chart showing sewage taxa with strong correlations to FIB.

FIG. 30 is schematic showing results of cluster analysis which showed the comparison of community composition. Communities can be clustered according to the time in the receiving waters, source, and type of receiving waters.

FIG. 31 is a bar chart showing the effect of time in receiving waters on fecal microbial communities.

FIG. 32 is a bar chart showing the effect of creek versus bay water on waste microbial communities.

FIG. 33 illustrates enrichment of bacterial taxa by an oil plume.

### DETAILED DESCRIPTION OF THE INVENTION

### Definitions

As used herein, the term “oligonucleotide” refers to a polynucleotide, usually single stranded, that is either a synthetic polynucleotide or a naturally occurring polynucleotide. The length of an oligonucleotide is generally governed by the particular role thereof, such as, for example, probe, primer and the like. Various techniques can be employed for preparing an oligonucleotide, for instance, biological synthesis or chemical synthesis. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)); phosphorothioate (Mag, et al, Nucleic Acids Res., 19:1437 (1991); and U.S. Pat. No. 5,644,048); phosphorodithioate (Briu, et al., J. Am. Chem. Soc., 111:2321 (1989)); O-methylphophoroamidite linkages (see Eckstein, Oligonucleotides and Analogues: A Practical Approach, Oxford University Press); and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and U.S. Pat. No. 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleosides & Nucleotides, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)); and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference.

The nucleic acid may be DNA, RNA, or a hybrid and may contain any combination of deoxyribo- and ribo-nucleotides, and any combination of bases, including uracil, adenine, thymine, cytosine, guanine, inosine, xanthanine, hypoxanthanine, isocytosine, isoguanine, and base analogs such as nitropyrrole and nitroindole, etc. Oligonucleotides can be synthesized by standard methods such as those used in commercial automated nucleic acid synthesizers and later attached to an array, bead or other suitable surface. Alternatively, the oligonucleotides can be synthesized directly on the assay surface using photolithographic or other techniques. In some embodiments, linkers are used to attach the oligonucleotides to an array surface or to beads.

As used herein, the term “nucleic acid molecule” or “polynucleotide” refers to a compound or composition that is a polymeric nucleotide or nucleic acid polymer. The nucleic acid molecule may be a natural compound or a synthetic compound. The nucleic acid molecule can have from about 2 to 5,000,000 or more nucleotides. The larger nucleic acid molecules are generally found in the natural state. In an isolated state, the nucleic acid molecule can have about 10 to 50,000 or more nucleotides, usually about 100 to 20,000 nucleotides. It is thus obvious that isolation of a nucleic acid molecule from the natural state often results in fragmentation. It may be useful to fragment longer target nucleic acid molecules, particularly RNA, prior to hybridization to reduce competing intramolecular structures. Fragmentation can be achieved chemically or enzymatically. Typically, when the sample contains DNA, a nuclease such as deoxyribonuclease (DNase) is employed cleave the phosphodiester linkages. Nucleic acid molecules, and fragments thereof, include, but are not limited to, purified or unpurified forms of DNA (dsDNA and ssDNA) and RNA, including tRNA, mRNA, rRNA, mitochondrial DNA and RNA, chloroplast DNA and RNA, DNA/RNA hybrids, biological material or mixtures thereof, genes, chromosomes, plasmids, cosmids, the genomes of microorganisms, e.g., bacteria, yeasts, phage, chromosomes, viruses, viroids, molds, fungi, or other higher organisms such as plants, fish, birds, animals, humans, and the like. The polynucleotide can be only a minor fraction of a complex mixture such as a biological sample.

As used herein, the term “hybridize” refers to the process by which single strands of polynucleotides form a double-stranded structure through hydrogen bonding between the constituent bases. The ability of two polynucleotides to hybridize with each other is based on the degree of complementarity of the two polynucleotides, which in turn is based on the fraction of matched complementary nucleotide pairs. The more nucleotides in a given polynucleotide that are complementary to another polynucleotide, the more stringent the conditions can be for hybridization and the more specific will be the binding between the two polynucleotides. Increased stringency may be achieved by elevating the temperature, increasing the ratio of co-solvents, lowering the salt concentration, and combinations thereof.

As used herein, the terms “complementary,” “complement,” and “complementary nucleic acid sequence” refer to the nucleic acid strand that is related to the base sequence in another nucleic acid strand by the Watson-Crick base-pairing rules. In general, two polynucleotides are complementary when one polynucleotide can bind another polynucleotide in an anti-parallel sense wherein the 3′-end of each polynucleotide binds to the 5′-end of the other polynucleotide and each A, T(U), G, and C of one polynucleotide is then aligned with a T(U), A, C, and G, respectively, of the other polynucleotide. Polynucleotides that comprise RNA bases can also include complementary G/U or U/G basepairs.

As used herein, the term “clustering tree” refers to a hierarchical tree structure in which observations, such as organisms, genes, and polynucleotides, are separated into one or more clusters. The root node of a clustering tree consists of a single cluster containing all observations, and the leaf nodes correspond to individual observations. A clustering tree can be constructed on the basis of a variety of characteristics of the observations, such as sequences of the genes and morphological traits of the organisms. Many techniques known in the art, e.g. hierarchical clustering analysis, can be used to construct a clustering tree. A non-limiting example of the clustering tree is a phylogenetic, taxonomic or evolutionary tree.

As used herein, the terms “operational taxon unit,” “OTU,” “taxon,” “hierarchical cluster,” and “cluster” are used interchangeably. An operational taxon unit (OTU) refers to a group of one or more organisms that comprises a node in a clustering tree. The level of a cluster is determined by its hierarchical order. In one embodiment, an OTU is a group tentatively assumed to be a valid taxon for purposes of phylogenetic analysis. In another embodiment, an OTU is any of the extant taxonomic units under study. In yet another embodiment, an OTU is given a name and a rank. For example, an OTU can represent a domain, a sub-domain, a kingdom, a sub-kingdom, a phylum, a sub-phylum, a class, a subclass, an order, a sub-order, a family, a subfamily, a genus, a subgenus, or a species. In some embodiments, OTUs can represent one or more organisms from the kingdoms eubacteria, protista, or fungi at any level of a hierarchal order. In some embodiments, an OTU represents a prokaryotic or fungal order.

As used herein, the term “kmer” refers to a polynucleotide of length k. In some embodiments, k is an integer from 1 to 1000. In some embodiments, k is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 250, 300, 400, 500, 600, 700, 800, 900, or 1000.

As used herein, the term “perfect match probe” (PM probe) refers to a kmer which is 100% complementary to at least a portion of a highly conserved target gene or polynucleotide. The perfect complementarity usually exists throughout the length of the probe. Perfect probes, however, may have a segment or segments of perfect complementarity that is/are flanked by leading or trailing sequences lacking complementarity to the target gene or polynucleotide.

As used herein, the term “mismatch probe” (MM probe) refers a control probe that is identical to a corresponding PM probe at all positions except for one, 2, 3, 4, 5, 6, 7, 8, 9 or 10 nucleotides of the PM probe. Typically, the non-identical position or positions are located at or near the center of the PM probe. In some embodiments, the mismatch probes are universal mismatch probes, e.g., a collection of mismatch probes that have no more than a set number of nucleotide variations or substitutions compared to positive probes. For example, the universal mismatch probes may differ in nucleotide sequence by no more than five nucleotides compared to any one PM probe in the PM probe set. In some embodiments, a MM probe is used adjacent to each test probe, e.g., a PM probe targeting a bacterial 16S rRNA sequence, in the array.

As used herein, the term “probe pair” refers to a PM probe and its corresponding MM probe. In some embodiments, the PM probes and the MM probes are scored in relation to each other during data processing and statistic analysis. As used herein, the term “a probe pair associated with an OTU” is defined as a pair of probes consisting of an OTU-specific PM probe and its corresponding MM probe.

As used herein, a “sample” is from any source, including, but not limited to, a gas sample, a fluid sample, a solid sample, or any mixture thereof

As used herein, a “microorganism” or “organism” includes, but is not limited to, a virus, viroids, bacteria, archaea, fungi, protozoa and the like.

The term “sensitivity” refers to a measure of the proportion of actual positives which are correctly identified as such.

The term “specificity” refers to a measure of the proportion of actual negatives which are correctly identified as such

The term “confidence level” refers to the likelihood, expressed as a percentage, that the results of a test are real and repeatable, and not random. Confidence levels are used to indicate the reliability of an estimate and can be calculated by a variety of methods.

The present invention relates to systems and methods for detecting contamination broadly, and more specifically in water. “Contamination,” as used herein, refers to the presence of any undesirable element or substance (a “contaminant”) in an analyzed composition. In some embodiments, the analyzed composition is water. In further embodiments, the contaminant is a microorganism. Contamination may result from the presence of one or more contaminants above a threshold level.

In one aspect, the invention utilizes a biosignature of OTUs. As used herein, the term “biosignature” refers to an association of the level of one or more members of one or more OTUs with a particular condition. In one embodiment, the biosignature comprises a determination of the presence, absence, and/or quantity of at least 5, 10, 20, 50, 100, 250, 500, 1000, 5000, 10,000, 20,000, 30,000, 40,000, 50,000, 60,000, 70,000, 80,000, 90,000, 100,000, 250,000, 500,000 or 1,000,000 OTUs in a sample using a single assay. In some embodiments, the biosignature comprises the presence of or changes in the level of at least 1, 2, 3, 4, 5, 6, 7, 8, 9, 10, 15, 20, 30, 40, 50, 75, 100, 125, 150, 175, 200, 250, 300, or more OTUs.

In one embodiment, the biosignature is associated with a single condition, for example contamination by a single source. In another embodiment, the biosignature is associated with a combination of conditions, for example contamination by two or more sources, such as contamination by 2, 3, 4, 5, 6, 7, 8, 9, 10 or more sources. A biosignature can be obtained for any sample, including but not limited to, fresh water, drinking water, marine water, reclaimed water, treated water, desalinated water, sewage, lakes, rivers, streams, oceans, surface water, groundwater, runoff, waste water, aquifers, other natural or non-natural bodies of water, and known contaminants. A biosignature can be determined for a pure sample, a known contaminant, or a combination thereof. In some embodiments, a biosignature of a test sample is compared to a known biosignature, and a determination is made as to likelihood that the signatures are the same. In further embodiments, a biosignature of a sample is compared to a biosignature from a contamination source. The biosignature to which the biosignature of the test sample is compared can be determined before, after, or at substantially the same time as that of the test sample. Biosignatures can be the result of one or more analyses of one or more samples from a particular source. Examples of contamination sources whose signatures can be analyzed include, but are not limited to, fecal matter from humans; fecal matter from avian sources, including migratory and non-migratory birds; fecal matter from cattle and livestock, including elk, cows, deer, sheep, horses, pigs, and goats; and fecal matter from aquatic animals, including sea lions, seals, and otters. Water contamination detected herein can also be from decaying matter (e.g. plant or animal decay), oil spills, industrial waste or byproducts, and any other contaminant to which an OTU biosignature can be correlated.

In some embodiments, the biosignature of a test sample is a combination of two or more independent signatures, such as 2, 3, 4, 5, 6, 7, 8, 9, 10 or more independent signatures. In a preferred embodiment, each of the two or more biosignatures contained in a sample are assayed simultaneously. In a further embodiment, a subset of biosignatures can be evaluated through the use of low-density detection systems, comprising the determination of the presence, absence, and/or level of no more than 10, 25, 50, 100, 250, 500, 1000, 2000, or 5000 OTUs.

In one aspect, the invention provides methods, systems, and compositions for detecting and identifying a plurality of biomolecules and organisms in a sample. The invention utilizes the ability to differentiate between individual organisms or OTUs. In one aspect, the individual organisms or OTUs are identified using organism-specific and/or OTU-specific probes, e.g., oligonucleotide probes. More specifically, some embodiments relate to selecting organism-specific and/or OTU-specific oligonucleotide probes useful in detecting and identifying biomolecules and organisms in a sample. In some embodiments, an oligonucleotide probe is selected on the basis of the cross-hybridization pattern of the oligonucleotide probe to regions within a target oligonucleotide and its homologs in a plurality of organisms. The homologs can have nucleotide sequences that are at least 80%, 85%, 90%, 91%, 92%, 93%, 94%, 95%, 96%, 97%, 98%, 99% or 99.5% identical. Such oligonucleotides can be gene, or intergenetic sequences, in whole or a portion thereof. The oligonucleotides can range from 10 to over 10,000 nucleotides in length. In some other embodiments, a method is provided for detecting the presence of an OTU in a sample based at least partly on the cross-hybridization of the OTU-specific oligonucleotide probes to probes specific for other organisms or OTUs. In some embodiments, the biosignature to which a sample biosignature is compared comprises a positive result for the presence of the targets for one or more probes.

In one aspect, the invention provides a diagnostic system for the determination or evaluation of a biosignature of a sample. In one embodiment, the diagnostic system comprises at least 1, 2, 3, 4, 5, 6, 7, 8, 9, 10, 15, 20, 30, 40, 50, 75, 100, 125, 150, 175, 200, 250, 300, or more probes. In another embodiment, the diagnostic system comprises up to 1, 2, 3, 4, 5, 6, 7, 8, 9, 10, 15, 20, 30, 40, 50, 75, 100, 125, 150, 175, 200, 250, 300, or more probes.

### High Capacity Systems

In one aspect of the invention, a high capacity system is provided for determining a biosignature of a sample by assessing the total microorganism population of a sample in terms of the microorganisms present and their percent composition of the total population. The system comprises of a plurality of probes that are capable of determining the presence or quantity of at least 10,000, 20,000, 30,000, 40,000, 50,000, 60,000, or more different OTUs in a single assay. Typically, the probes selectively hybridize to a highly conserved polynucleotide. Usually, the probes hybridize to the same highly conserved polynucleotide or within a portion thereof. Generally, the highly conserved polynucleotide or fragment thereof comprises a gene or fragment thereof. Exemplary highly conserved polynucleotides comprise nucleotide sequences found in the 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, cox1 gene and nifD gene. In other embodiments, two or more, three or more, four or more, five or more, six or more, seven or more, eight or more, nine or more, ten or more, 15 or more, 20 or more, 25 or more, or 50 or more collections of probes are employed, each of which specifically hybridizes to a different highly conserved polynucleotides. For example, one collection of probes binds to the same region of the 16S rRNA gene, while a second collection of probes binds to the same region of the 23S rRNA gene. The use of two or more collections of probes where each collection recognizes distinct and separate highly conserved polynucleotides allows for the generation and testing of more probes the use of which can provide greater discrimination between species or OTUs.

Highly conserved polynucleotides usually show at least 80%, 85%, 90%, 92%, 94%, 95%, or 97% homology across a domain, kingdom, phylum, class, order, family or genus, respectively. The sequences of these polynucleotides can be used for determining evolutionary lineage or making a phylogenetic determination and are also known as phylogenetic markers. In some embodiments, a biosignature comprises the presence, absence, and/or abundance of a combination of phylogenetice markers. The OTUs detected by the probes disclosed herein can be bacterial, archeal, fungal, or eukaryotic in origin. Additionally, the methodologies disclosed herein can be used to quantify OTUs that are bacterial, archaeal, fungal, or eukaryotic. By combining the various probes sets, a system for the detection of bacteria, archaea, fungi, eukaryotes, or combinations thereof can be designed. Such a universal microorganism test that is conducted as a single assay can provide great benefit for assessing and understanding the composition and ecology of numerous environments, including characterization of biosignatures for various samples, environments, conditions, and contaminants.

In another aspect of the invention, a system is provided that is capable of determining the probability of presence and optionally quantity of at least 10,000, 20,000, 30,000, 40,000, 50,000 or 60,000 different OTUs of a single domain in a single assay. Such a system makes a probability determination with a confidence level greater than 90%, 91%, 92%, 93%, 94%, 95%, 99% or 99.5%. In some embodiments, a biosignature can comprise the combined result of each probability determination.

Some embodiments provide a method of selecting an oligonucleotide probe that is specific for a node in a clustering tree. In some embodiments, the method comprises selecting a highly conserved target polynucleotide and its homologs for a plurality of organisms; clustering the polynucleotides and homologs of the plurality of organisms into a clustering tree; and determining a cross-hybridization pattern of a candidate oligonucleotide probe that hybridizes to a first polynucleotide to each node on the clustering tree. This determination is performed (e.g., in silico) to determine the likelihood that the probe would cross hybridize with homologs of its target complementary sequence. The candidate oligonucleotide probe can be complementary to a highly conserved target polynucleotide, a fragment of the highly conserved target or one of its homologs in one of the plurality of organisms. In some embodiments, a method is provided for the determination of the cross-hybridization pattern of a variant of the candidate oligonucleotide probe to each node on the clustering tree, wherein the variant corresponds to the candidate oligonucleotide probe but comprises at least 1 nucleotide mismatch; and selecting or rejecting the candidate oligonucleotide probe on the basis of the cross-hybridization pattern of the candidate oligonucleotide probe and the cross-hybridization pattern of the variant. In some embodiments, the node is an operational taxon unit (OTU). In some embodiments, the node is a single organism.

Some embodiments provide a method of selecting an OTU-specific oligonucleotide probe for use in detecting a plurality of organisms in a sample. In some embodiments, the method comprises: selecting a highly conserved target polynucleotide and its homologs from the plurality of organisms; clustering the polynucleotides of the target gene and its homologs from the plurality of organisms into one or more operational taxonomic units (OTUs), wherein each OTU comprises one or more groups of similar nucleotide sequence; determining the cross-hybridization pattern of a candidate OTU-specific oligonucleotide probe to the OTUs, wherein the candidate OTU-specific oligonucleotide probe corresponds to a fragment of the target gene or its homolog from one of the plurality of organisms; determining the cross-hybridization pattern of a variant of the candidate OTU-specific oligonucleotide probe to the OTUs, wherein the variant comprises at least 1 nucleotide mismatch from the candidate OTU-specific oligonucleotide probe; and selecting or rejecting the candidate OTU-specific oligonucleotide probe on the basis of the cross-hybridization pattern of the candidate OTU-specific oligonucleotide probe and the cross-hybridization pattern of the variant. In some embodiments, the candidate OTU-specific oligonucleotide probe is selected if the candidate OTU-specific oligonucleotide probe does not cross-hybridize with any polynucleotide that is complementary to probes from other OTUs. In further embodiments, the candidate OTU-specific oligonucleotide probe is selected if the candidate OTU-specific oligonucleotide probe cross-hybridizes with the polynucleotide in no more than 1, 2, 3, 4, 5, 6, 7, 8, 9, 10, 11, 12, 13, 14, 15, 20, 30, 40, 50, 100, 200, 500, or 1000 other OTU groups.

Some embodiments provide a method of selecting a set of organism-specific oligonucleotide probes for use in detecting a plurality of organisms in a sample. In some embodiments, the method comprises: identifying a highly conserved target polynucleotide and its homologs in the plurality of organisms; determining the cross-hybridization pattern of a candidate organism-specific oligonucleotide probe to the sequences of the highly conserved target polynucleotide and its homologs in the plurality of organisms, wherein the candidate oligonucleotide probe corresponds to a fragment of the target sequence or its homolog from one of the plurality of organisms; determining the cross-hybridization pattern of a variant of the candidate organism-specific oligonucleotide probe to the sequences of the highly conserved target sequence and its homologs in the plurality of organisms, wherein the variant comprises at least 1 nucleotide mismatch from the candidate organism-specific oligonucleotide probe; and selecting or rejecting the candidate organism-specific oligonucleotide probe on the basis of the cross-hybridization pattern of the candidate organism-specific oligonucleotide probe and the cross-hybridization pattern of the variant of the candidate organism-specific oligonucleotide probe.

In some embodiments, an OTU-specific oligonucleotide probe does not cross-hybridize with any polynucleotide that is complementary to probes from other OTUs. In other embodiments, an OTU-specific oligonucleotide probe cross-hybridizes with the polynucleotide in no more than 1, 2, 3, 4, 5, 6, 7, 8, 9, 10, 11, 12, 13, 14, 15, 20, 30, 40, 50, 100, 200, 500, or 1000 other OTU groups. Some embodiments utilize a set of organism-specific oligonucleotide probes for use in detecting a plurality of organisms in a sample. In further embodiments, the candidate organism-specific oligonucleotide probe is selected if the candidate organism-specific oligonucleotide probe only hybridizes with the target nucleic acid molecule of no more than 1, 2, 3, 4, 5, 6, 7, 8, 9, 10, 11, 12, 13, 14, 15, 20, 30, 40, 50 unique organisms in the plurality of organisms. In other embodiments, the process is iterative with multiple candidate specific-specific oligonucleotide probes selected. Frequently, the selected organism-specific oligonucleotide probes are clustered and aligned into groups of similar sequences that allow for the detection of an organism with high confidence based on no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 50, or 60 organism-specific oligonucleotide probe matches per OTU. Generally, the candidate organism that the organism-specific oligonucleotide probes detect corresponds to a leaf or node of at least one phylogenetic, genealogic, evolutionary, or taxonomic tree. Knowledge of the position that a candidate organism detected by the organism-specific oligonucleotide probe occupies on a tree provides relational information of the organism to other members of its domain, phylum, class, subclass, order, family, subfamily, or genus.

In some embodiments, the method disclosed herein selects and/or utilizes a set of organism-specific oligonucleotide probes that are a hierarchical set of oligonucleotide probes that can be used to detect and differentiate a plurality of organisms. In some embodiments, the method selects and/or utilizes organism-specific or OTU-specific oligonucleotide probes that allow a comprehensive screen for at least 80%, 85%, 90%, 95%, 99% or 100% of all known bacterial or archaeal taxa in a single analysis, and thus provides an enhanced detection of different desired taxonomic groups. In some embodiments, the identity of all known bacterial or archaeal taxa comprises taxa that were previously identified by the use of oligonucleotide specific probes, PCR cloning, and sequencing methods. Some embodiments provide methods of selecting and/or utilizing a set of oligonucleotide probes capable of correctly categorizing mixed target nucleic acid molecules into their proper operational taxonomic unit (OTU) designations. Such methods can provide comprehensive prokaryotic or eukaryotic identification, and thus comprehensive biosignature characterization.

In some embodiments, the selected OTU-specific oligonucleotide probe is used to calculate the relative abundance of one or more organisms that belong to a specific OTU at differing levels of taxonomic identification. In some embodiments, an array or collection of microparticles comprising at least one organism-specific or OTU-specific oligonucleotide probe selected by the method disclosed herein is provided to infer specific microbial community activities. For example, the identity of individual taxa in a microbial consortium from an anaerobic environment for instance, a marsh, can be determined along with their relative abundance. If the consortium is suspected of harboring microorganisms capable of butanol fermentation, then after providing a suitable feedstock in an anaerobic environment if the production of butanol is noted, then those taxa responsible for butanol fermentation can be inferred by the microorganisms that have abundant quantities of 16S rRNA. The invention provides methods to measure taxa abundance based on the detection of directly labeled 16S rRNA capable of the anaerobic fermentation of butanol can be identified from a sample obtained from a marsh or other anaerobic environment.

Some embodiments select multiple probes for increasing the confidence level and/or sensitivity level of identification of a particular organism or OTU. The use of multiple probes can greatly increase the confidence level of a match to a particular organism. In some embodiments, the selected organism-specific oligonucleotide probes are clustered and aligned into groups of similar sequence such that detection of an organism is based on 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35 or more oligonucleotide probe matches. In some embodiments, the oligonucleotide probes are specific for a species. In other embodiments, the oligonucleotide probe recognizes related organisms such as organisms in the same subgenus, genus, subfamily, family, sub-order, order, sub-class, class, sub-phylum, phylum, sub-kingdom, or kingdom.

Perfect match (PM) probes are perfectly complementary to the target polynucleotide, e.g., a sequence that identifies a particular organism. In some embodiments, a system of the invention comprises mismatch (MM) control probes. Usually, MM probes are otherwise identical to PM probes, but differ by one or more nucleotides. Probes with one or more mismatch can be used to indicate non-specific binding and a possible non-match to the target sequence. In some embodiments, the MM probes have one mismatch located in the center of the probe, e.g., in position 13 for a 25mer probe. The MM probe is scored in relation to its corresponding PM probe as a “probe pair.” MM probes can be used to estimate the background hybridization, thereby reducing the occurrence of false positive results due to non-specific hybridization, a significant problem with many current detection systems. If an array is used, such as an Affymetrix high density probe array or Illumina bead array, ideally, the MM probe is positioned adjacent or close to its corresponding PM probe on the array.

Some embodiments relate to a method of selecting and/or utilizing a set of oligonucleotide probes that enable simultaneous identification of multiple prokaryotic taxa with a relatively high confidence level. Typically, the confidence level of identification is at least 90%, 91%, 92%, 93%, 94%, 95%, 96%, 97%, 98%, 99% or 99.5%. An OTU refers to an individual species or group of highly related species that share an average of at least 85%, 90%, 91%, 92%, 93%, 94%, 95%, 96%, 97%, 98%, 99% or 99.5% sequence homology in a highly conserved region. Multiple MM probes may be utilized to enhance the quantification and confidence of the measure. In some embodiments, each interrogation probe of a plurality of interrogation probes has from about 1 to about 20 corresponding mismatch control probes. In further embodiments, each interrogation probe has from about 1 to about 10, about 1 to about 5, about 1 to 4, 1 to 3, 2 or 1 corresponding mismatch probes. These interrogation probes target unique regions within a target nucleic acid sequence, e.g., a 16S rRNA gene, and provide the means for identifying at least about 10, 20, 50, 100, 500, 1,000, 2,000, 5,000, 10,000, 20,000, 30,000, 40,000, 50,000, 60,000, 70,000, 80,000, 90,000, 100,000, 250,000, 500,000 or 1,000,000 taxa. In some embodiments, multiple targets can be simultaneously assayed or detected in a single assay through a high-density oligonucleotide probe system. The sum of all target hybridizations is used to identify specific prokaryotic taxa. The result is a more efficient and less time consuming method of identifying unculturable or unknown organisms. The invention can also provide results that could not previously be achieved, e.g., providing results in hours where other methods would require days. In some embodiments, a microbiome (i.e., sample) can be assayed to determine the identity and abundance of its constituent microorganisms in less than 20, 19, 18, 17, 16, 15, 14, 13, 12, 11, 10, 9, 8, 7, 6, 5, 4, 3, 2, or 1 hour.

In some embodiments, the set of OTU-specific oligonucleotide probes comprises from about 1 to about 500 probes for each taxonomic group. In some embodiments, the probes are proteins including antibodies, or nucleic acid molecules including oligonucleotides or fragments thereof. In some embodiments, an oligonucleotide probe corresponds to a nucleotide fragment of the target nucleic acid molecule. In some embodiments, from about 1 to about 500, about 2 to about 200, about 5 to about 150, about 8 to about 100, about 10 to about 35, or about 12 to about 30 oligonucleotide probes can be designed for each taxonomic grouping. In other embodiments, a taxonomic group can have at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, or more probes. In some embodiments, various taxonomic groups can have different numbers of probes, while in other embodiments, all taxonomic groups have a fixed number of probes per group. Multiple probes in a taxonomic group can provide additional data that can be used to make a determination, also known as “making a call” as to whether an OTU is present or not. Multiple probes also allow for the removal of one or more probes from the analysis based on insufficient signal strength, cross hybridization or other anomalies Removing probes can increase the confidence level of results and further allow for the detection of low abundant microorganisms. The oligonucleotide probes can each be from about 5 to about 100 nucleotides, from about 10 to about 50 nucleotides, from about 15 to about 35 nucleotides, or from about 20 to about 30 nucleotides. In some embodiments, the probes are at least 5-mers, 6-mers, 7-mers, 8-mers, 9-mers, 10-mers, 11-mers, 12-mers, 13-mers, 14-mers, 15-mers, 16-mers, 17-mers, 18-mers, 19-mers, 20-mers, 21-mers, 22-mers, 23-mers, 24-mers, 25-mers, 26-mers, 27-mers, 28-mers, 29-mers, 30-mers, 31-mers, 32-mers, 33-mers, 34-mers, 35-mers, 36-mers, 37-mers, 38-mers, 39-mers, 40-mers, 41-mers, 42-mers, 43-mers, 44-mers, 45-mers, 46-mers, 47-mers, 48-mers, 49-mers, 50-mers, 51-mers, 52-mers, 53-mers, 54-mers, 55-mers, 56-mers, 57-mers, 58-mers, 59-mers, 60-mers, 61-mers, 62-mers, 63-mers, 64-mers, 65-mers, 66-mers, 67-mers, 68-mers, 69-mers, 70-mers, 71-mers, 72-mers, 73-mers, 74-mers, 75-mers, 76-mers, 77-mers, 78-mers, 79-mers, 80-mers, 81-mers, 82-mers, 83-mers, 84-mers, 85-mers, 86-mers, 87-mers, 88-mers, 89-mers, 90-mers, 91-mers, 92-mers, 93-mers, 94-mers, 95-mers, 96-mers, 97-mers, 98-mers, 99-mers, 100-mers or combinations thereof

Some embodiments provide methods of selecting multiple, confirmatory, organism-specific or OTU-specific probes to increase the confidence of detection. In some embodiments, the methods also select one or more mismatch (MM) probes for every perfect match (PM) probe to minimize the effect of cross-hybridization by non-target regions. The organism-specific and OTU-specific oligonucleotide probes selected by the methods disclosed herein can simultaneously identify thousands of taxa present in an environmental sample and allow accurate identification of microorganisms and their phylogenetic relationships in a community of interest. Systems that use the organism-specific and OTU-specific oligonucleotide probes selected by the methods disclosed herein and the computational analysis disclosed herein have numerous advantages over rRNA gene sequencing techniques. Such advantages include reduced cost per microbiome analysis, and increased processing speed per sample or microbiome from both the physical analysis and the computational analysis point of view the analysis procedures are not adversely affected by chimeras, are not subject to creating artificial phylotypes and are not subject to barcode PCR bias. Additionally, quantitative standards can be run with a microbiome sample of the invention, something that is not possible with pyrosequencing.

Some embodiments provide a method for selecting and/or utilizing a set of OTU- or organism-specific oligonucleotide probes for use in an analysis system or bead multiplex system for simultaneously detecting a plurality of organisms in a sample. The method targets known diversity within target nucleic acid molecules to determine microbial community composition and establish a biosignature. The target nucleic acid molecule is typically a highly conserved polynucleotide. In some embodiments, the highly conserved polynucleotide is from a highly conserved gene, whereas in other embodiments the polynucleotide is from a highly conserved region of a gene with moderate or large sequence variation. In further embodiments, the highly conserved region may be an intron, exon, or a linking section of nucleic acid that separates two genes. In some embodiments, the highly conserved polynucleotide is from a “phylogenetic” gene. Phylogenetic genes include, but are not limited to, the 5.8S rRNA gene, 12S rRNA gene, 16S rRNA gene-prokaryotic, 16S rRNA gene-mitochondrial, 18S rRNA gene, 23S rRNA gene, 28S rRNA gene, gyrB gene, rpoB gene, fusA gene, recA gene, cox1 gene, and the nifD gene. With eukaryotes, the rRNA gene can be nuclear, mitochondrial, or both. In some embodiments, the 16S-23S rRNA gene internal transcribed spacer (ITS) can be used for differentiation of closely related taxa with or without the use of other rRNA genes. For example, rRNA, e.g., 16S or 23S rRNA, acts directly in the protein assembly machinery as a functional molecule rather than having its genetic code translated into protein. Due to structural constraints of 16S rRNA, specific regions throughout the gene have a highly conserved polynucleotide sequence although non-structural segments may have a high degree of variability. Probing the regions of high variability can be used to identify OTUs that represent a single species level, while regions of less variability can be used to identify OTUs that represent a subgenus, a genus, a subfamily, a family, a sub-order, an order, a sub-class, a class, a sub-phylum, a phylum, a sub-kingdom, or a kingdom. The methods disclosed herein can be used to select organism-specific and OTU-specific oligonucleotide probes that offer high level of specificity for the identification of specific organisms, OTUs representing specific organisms, or OTUs representing specific taxonomic group of organisms. The systems and methods disclosed herein are particularly useful in identifying closely related microorganisms and OTUs from a background or pool of closely related organisms.

The probes selected and/or utilized by the methodologies of the invention can be organized into OTUs that provide an assay with a sensitivity and/or specificity of more than 80%, 85%, 90%, 91%, 92%, 93%, 94%, 95%, 96%, 97%, 98% or 99%. In some embodiments, sensitivity and specificity depends on the hybridization signal strength, number of probes in the OTU, the number of potential cross hybridization reactions, the signal strength of the mismatch probes, if present, background noise, or combinations thereof. In some embodiments, an OTU containing one probe may provide an assay with a sensitivity and specificity of at least 90%, while another OTU may require at least 20 probes to provide an assay with sensitivity and specificity of at least 90%.

Some embodiments relate to methods for phylogenetic analysis system design and signal processing and interpretation for use in detecting and identifying a plurality of biomolecules and organisms in a sample. More specifically, some embodiments relate to a method of selecting a set of organism-specific oligonucleotide probes for use in detecting a plurality of organisms in a sample with a high confidence level. Some embodiments relate to a method of selecting a set of OTU-specific oligonucleotide probes for use in detecting a plurality of organisms in a sample with a high confidence level.

In the case of highly conserved polynucleotides like 16S rRNA that may have only one to a few nucleotides of sequence variability over any 15- to 30-bp region targeted by probes for discrimination between related microbial species, it is advantageous to maximize the probe-target sequence specificity in an assay system. Some embodiments of the present invention provide methods of selecting organism-specific oligonucleotide probes that effectively minimize the influence of cross-hybridization. In one embodiment, the method comprises: (a) identifying sequences of a target nucleic acid molecule corresponding to the plurality of organisms; (b) determining the cross-hybridization pattern of a candidate organism-specific oligonucleotide probe to the target nucleic acid molecule from the plurality of organisms, wherein the candidate oligonucleotide probe corresponds to a sequence fragment of the target nucleic acid molecule from the plurality of organisms; (c) determining the cross-hybridization pattern of a variant of the candidate organism-specific oligonucleotide probe to the target nucleic acid molecule from the plurality of organisms, wherein the variant of the candidate organism-specific oligonucleotide probe comprises at least 1 nucleotide mismatch compared to the candidate organism-specific oligonucleotide probe; and (d) selecting or rejecting the candidate organism-specific oligonucleotide probe on the basis of the cross-hybridization pattern of the candidate organism-specific oligonucleotide probe and the cross-hybridization pattern of the variant of the candidate organism-specific oligonucleotide probe. In some embodiments, a method of selecting a set of OTU-specific oligonucleotide probes for use in detecting a plurality of organisms in a sample is provided. In some embodiments, the method comprises: (a) identifying sequences of a target nucleic acid molecule corresponding to the plurality of organisms; (b) clustering the sequences of the target nucleic acid molecule from the plurality of organisms into one or more Operational Taxonomic Units (OTUs), wherein each OTU comprises one or more groups of similar sequences; (c) determining the cross-hybridization pattern of a candidate OTU-specific oligonucleotide probe to the OTUs, wherein the candidate OTU-specific oligonucleotide probe corresponds to a sequence fragment of the target nucleic acid molecule from one of the plurality of organisms; (d) determining the cross-hybridization pattern of a variant of the candidate OTU-specific oligonucleotide probe to the OTUs, wherein the variant of the candidate OTU-specific oligonucleotide probe comprises at least 1 nucleotide mismatch compared to the candidate OTU-specific oligonucleotide probe; and (e) selecting or rejecting the candidate OTU-specific oligonucleotide probe on the basis of the cross-hybridization pattern of the candidate OTU-specific oligonucleotide probe to the OTUs and the cross-hybridization pattern of the variant of the candidate OTU-specific oligonucleotide probe to the OTUs. In some embodiments, candidate OTU-specific oligonucleotide probe are rejected when the candidate OTU-specific oligonucleotide probe or its variant are predicted to cross-hybridize with other target sequences. In some embodiments, a predetermined amount of predicted cross-hybridization is allowed.

In some embodiments, selected oligonucleotide probes are synthesized by any relevant method known in the art. Some examples of suitable methods include printing with fine-pointed pins onto glass slides, photolithography using pre-made masks, photolithography using dynamic micromirror devices, ink-jet printing, or electrochemistry. In one example, a photolithographic method can be used to directly synthesize the chosen oligonucleotide probes onto a surface. Suitable examples for the surface include glass, plastic, silicon and any other surface available in the art. In certain examples, the oligonucleotide probes can be synthesized on a glass surface at an approximate density from about 1,000 probes per μm² to about 100,000 probes per μm², preferably from about 2000 probes per μm² to about 50,000 probes per μm², more preferably from about 5000 probes per μm² to about 20,000 probes per μm². In one example, the density of the probes is about 10,000 probes per μm². The number of probes on the array can be quite large e.g., at least 10⁵, 10⁶, 10⁷, 10⁸ or 10⁹ probes per array. Usually, for large arrays only a relatively small proportion (i.e., less than about 1%, 0.1% 0.01%, 0.001%, 0.00001%, 0.000001% or 0.0000001%) of the total number of probes of a given length target an individual OTU. Frequently, lower limit arrays have no more than 10, 25, 50, 100, 500, 1,000, 5,000, or 10,000, 25,000, 50,000, 100,000 or 250,000 probes.

Typically, the arrays or microparticles have probes to one or more highly conserved polynucleotides. The arrays or microparticles may have further probes (e.g. confirmatory probes) that hybridize to functionally expressed genes, thereby providing an alternate or confirmatory signal upon which to base the identification of a taxon. For example, an array may contain probes to 16S rRNA gene sequences from Yersinia pestis and Vibrio cholerae and also confirmatory probes to Y. pestis cafl virulence gene or V. cholerae zonula occludens toxin (zot) gene. The detection of hybridization signals based on probes binding to 16S rRNA polynucleotides associated with a particular OTU coupled with the detection of a hybridization signal based on a confirmatory probe can provide a higher level of confidence that the OTU is present. For instance, if hybridization signals are detected for the probes associated Y. pestis OTU and the confirmatory probe also displays a hybridization signal for the expression of Y. pestis cafl then the confidence level subscribed to the presence or quantity of Y. pestis will be higher than the confidence level obtained from the use of OTU probes alone.

A range of lengths of probes can be employed on the arrays or microparticles. As noted above, a probe may consist exclusively of a complementary segments, or may have one or more complementary segments juxtaposed by flanking, trailing and/or intervening segments. In the latter situation, the total length of complementary segment(s) can be more important that the length of the probe. In functional terms, the complementary segment(s) of the PM probes should be sufficiently long to allow the PM probes to hybridize more strongly to a target polynucleotide e.g., 16S rRNA, compared with a MM probe. A PM probe usually has a single complementary segment having a length of at least 15 nucleotides, and more usually at least 16, 17, 18, 19, 20, 21, 22, 23, 24, 25 or 30 bases exhibiting perfect complementarity.

In some arrays or lots of microparticles, all probes are the same length. In other arrays or lots of microparticles, probe length varies between quantification standard (QS) probes, negative control (NC) probes, probe pairs, probe sets (OTUs) and combinations thereof. For example, some arrays may have groups of OTUs that comprise probe pairs that are all 23 mers, together with other groups of OTUs or probe sets that comprise probe pairs that are all 25 mers. Additional groups of probes pairs of other lengths can be added. Thus, some arrays may contain probe pairs having sizes of 15 mers, 16mers, 17mers, 18mers, 19mers, 20mers, 21mers, 22mers, 23mers, 24mers, 25 mers, 26mers, 27 mers, 28mers, 29 mers, 30mers, 31mers, 32mers, 33mers, 34mers, 35mers, 36mers, 37mers, 38mers, 39mers, 40mers or combinations thereof. Other arrays may have different size probes within the same group, OTU, or probe set. In these arrays, the probes in a given OTU or probe set can vary in length independently of each other. Having different length probes can be used to equalize hybridization signals from probes depending on the hybridization stability of the oligonucleotide probe at the pH, temperature, and ionic conditions of the reaction.

In another aspect of the invention, a system is provided for determining the presence or quantity of a plurality of different OTUs in a single assay where the system comprises a plurality of polynucleotide interrogation probes, a plurality of polynucleotide positive control probes, and a plurality of polynucleotide negative control probes. In some embodiments, the system is capable of detecting the presence, absence, relative abundance, and/or quantity of at least 5, 10, 20, 50, 100, 250, 500, 1000, 5000, 10,000, 20,000, 30,000, 40,000, 50,000, 60,000, 70,000, 80,000, 90,000, 100,000, 250,000, 500,000 or 1,000,000 OTUs in a sample using a single assay. In some embodiments, the polynucleotide positive control probes include 1) probes that target sequences of prokaryotic or eukaryotic metabolic genes spiked into the target nucleic acid sequences in defined quantities prior to fragmentation, or 2) probes complimentary to a pre-labeled oligonucleotide added into the hybridization mix after fragmentation and labeling. The control added prior to fragmentation collectively tests the fragmentation, biotinylation, hybridization, staining and scanning efficiency of the system. It also allows the overall fluorescent intensity to be normalized across multiple analysis components used in a single or combined experiment, such as when two or more arrays are used in a single experiment or when data from two separate experiments is combined. The second control directly assays the hybridization, staining and scanning of the system. Both types of control can be used in a single experiment.

In some embodiments, the QS standards (positive controls) are PM probes. In other embodiments, the QS standards are PM and MM probe pairs. In further embodiments, the QS standards comprise a combination of PM and MM probe pairs and PM probes without corresponding MM probes. In another embodiment, the QS standards comprise at least one, two, three, four, five, six, seven, eight, nine, ten or more MM probes for each corresponding PM probe. In a further embodiment, the QS standards comprise at least one, two, three, four, five, six, seven, eight, nine, ten or more PM probes for each corresponding MM probe. A system can comprise at least 1 positive control probe for each 1, 10, 100, or 1000 different interrogation probes.

In some cases, the spiked-in oligonucleotides that are complementary to the positive control probes vary in G+C content, uracil content, concentration, or combinations thereof. In some embodiments, the G+C % ranges from about 30% to about 70%, about 35% to about 65% or about 40% to about 60%. QS standards can also be chosen based on the uracil incorporation frequency. The QS standards may incorporate uracil in a range from about 1 in 100 to about 60 in 100, about 4 in 100 to about 50 in 100, or about 10 in 100 to about 50 in 100. In some cases, the concentration of these added oligonucleotides will range over 1, 2, 3, 4, 5, 6, or 7 orders of magnitude. Concentration ranges of about 10⁵ to 10¹⁴, 10⁶ to 10¹³, 10⁷ to 10¹², 10⁷ to 10¹¹, 10⁸ to 10¹¹, and 10⁸ to 10¹⁰ can be employed and generally feature a linear hybridization signal response across the range. In some embodiments, positive control probes for the conduction of the methods disclosed herein comprise polynucleotides that are complementary to the positive control sequences shown in Table 1. Other genes that can be used as targets for positive controls include genes encoding structural proteins, proteins that control growth, cell cycle or reproductive regulation, and house keeping genes. Additionally, synthetic genes based on highly conserved genes or other highly conserved polynucleotides can be added to the sample. Useful highly conserved genes from which synthetic genes can be designed include 16S rRNA genes, 18S rRNA genes, 23SrRNA genes. Exemplary control probes are provided as SEQ ID NOs:51-100.

TABLE 1
Positive Control Sequences

| | Description |
|---|---|
| Positive | |
| Control ID | |
| AFFX-BioB-5_at | E. coli biotin synthetase |
| AFFX-BioB-M_at | E. coli biotin synthetase |
| AFFX-BioC-5_at | E. coli bioC protein |
| AFFX-BioC-3_at | E. coli bioC protein |
| AFFX-BioDn-3_at | E. coli dethiobiotin synthetase |
| AFFX-CreX-5_at | Bacteriophage P1 cre recombinase protein |
| AFFX-DapX-5_at | B. subtilis dapB, dihydrodipicolinate reductase |
| AFFX-DapX-M_at | B. subtilis dapB, dihydrodipicolinate reductase |
| YFL039C | Saccharomyces, Gene for actin (Act 1p) protein |
| YER022W | Saccharomyces, RNA polymerase II mediator |
| | complex subunit (SRB4p) |
| YER 148 W | Saccharomyces, TATA-binding protein, general |
| | transcription factor (SPT15) |
| YEL002C | Saccharomyces, Beta subunit of the oligosaccharyl |
| | transferase (OST) glycoprotein complex (WBP1) |
| YEL024W | Saccharomyces, Ubiquinol-cytochrome-c |
| | reductase (RIP1) |
| Synthetic 16S | |
| rRNA controls | |
| SYNM neurolyt_st | Synthetic derivative of Mycoplasma neurolyticum |
| | 16S rRNA gene |
| SYNLc.oenos_st | Synthetic derivative of Leuconostoc oenos 16S |
| | rRNA gene |
| SYNCau.cres8_st | Synthetic derivative of Caulobacter crescenius |
| | 16S rRNA gene |
| SYNFer.nodosm_st | Synthetic derivative of Fervidobacterium nodosum |
| | 16S rRNA gene |
| SYNSap.grandi_st | Synthetic derivative of Saprospira grandis 16S |
| | rRNA gene |

In some embodiments, the negative controls comprise PM and MM probe pairs. In further embodiments, the negative controls comprise a combination of PM and MM probe pairs and PM probes without corresponding MM probes. In other embodiments, the negative control probes comprise at least one, two, three, four, five, six, seven, eight, nine, ten or more MM probes for each corresponding negative control PM probe. A system can comprise at least 1 negative control probe for each 1, 10, 100, or 1000 different interrogation probes (PMs).

Generally, the negative control probes hybridize weakly, if at all, to 16S rRNA gene or other highly conserved gene targets. The negative control probes can be complementary to metabolic genes of prokaryotic or eukaryotic origin. Generally, with negative control probes, no target material is spiked into the sample. In some embodiments, negative control probes are from the same collection of probes that are also used for positive controls, but no material complementary to the negative control probes are spiked into the sample, in contrast to the positive control probe methodology. In essence, the control probes are universal control probes and play the role of a positive or negative control probes depending on the system's design. One of skill in the art will appreciate that the universal control probes are not limited to highly conserved sequence analysis systems and have applications beyond the present embodiments disclosed herein.

In a further embodiment, probes to non-highly conserved polynucleotides are added to a system to provide species-specific identification or confirmation of results achieved with the probes to the highly conserved polynucleotides. Usually, these “confirmatory” probes cross hybridize very weakly, if at all, to highly conserved polynucleotides recognized by the perfect match probes. Useful species-specific genes include metabolic genes, genes encoding structural proteins, proteins that control growth, cell cycle or reproductive regulation, housekeeping genes or genes that encode virulence, toxins, or other pathogenic factors. In some embodiments, the system comprises at least 1, 5, 10, 20, 30, 40, 50 60, 70, 80, 90 100, 150, 200, 250, 300, 400, 500, 600, 700, 800, 900, 1000, 5,000 or 10,000 species-specific probes.

In some embodiments, a system of the invention comprises an array. Non-limiting examples of arrays include microarrays, bead arrays, through-hole arrays, well arrays, and other arrays known in the art suitable for use in hybridizing probes to targets. Arrays can be arranged in any appropriate configuration, such as, for example, a grid of rows and columns. Some areas of an array comprise the OTU detection probes whereas other areas can be used for image orientation, normalization controls, signal scaling, noise reduction processing, or other analyses. Control probes can be placed in any location in the array, including along the perimeter of the array, diagonally across the array, in alternating sections or randomly. In some embodiments, the control probes on the array comprise probe pairs of PM and MM probes. The number of control probes can vary, but typically the number of control probes on the array range from 1 to about 500,000. In some embodiments, at least 10, 100, 500, 1,000, 5,000, 10,000, 25,000, 50,000, 100,000, 250,000 or 500,000 control probes are present. When control probe pairs are used, the probe pairs will range from 1 to about 250,000 pairs. In some embodiments, at least 5, 50, 250, 500, 2,500, 5,000, 12,500, 25,000, 50,000, 125,000 or 250,000 control probe pairs are present. The arrays can have other components besides the probes, such as linkers attaching the probes to a support. In some embodiments, materials for fabricating the array can be obtained from Affymetrix (Santa Clara, Calif.), GE Healthcare (Little Chalfont, Buckinghamshire, United Kingdom) or Agilent Technologies (Palo Alto, Calif.)

Besides arrays where probes are attached to the array substrate, numerous other technologies may be employed in the disclosed system for the practice of the methods of the invention. In one embodiment, the probes are attached to beads that are then placed on an array as disclosed by Ng et al. (Ng et al. A spatially addressable bead-based biosensor for simple and rapid DNA detection. Biosensors & Bioelectronics, 23:803-810, 2008).

In another embodiment, probes are attached to beads or microspheres, the hybridization reactions are performed in solution, and then the beads are analyzed by flow cytometry, as exemplified by the Luminex multiplexed assay system. In this analysis system, homogeneous bead subsets, each with beads that are tagged or labeled with a plurality of identical probes, are combined to produce a pooled bead set that is hybridized with a sample and then analyzed in real time with flow cytometry, as disclosed in U.S. Pat. No. 6,524,793. Bead subsets can be distinguished from each other by variations in the tags or labels, e.g., using variable in laser excitable dye content.

In a further embodiment, probes are attached to cylindrical glass microbeads as exemplified by the Illumina Veracode multiplexed assay system. Here, subsets of microbeads embedded with identical digital holographic elements are used to create unique subsets of probe-labeled microbeads. After hybridization, the microbeads are excited by laser light and the microbead code and probe label are read in real time multiplex assay.

In another embodiment, a solution based assay system is employed as exemplified by the NanoString nCounter Analysis System (Geiss G et al. Direct multiplexed measurement of gene expression with color-coded probe pairs. Nature Biotech. 26:317-325, 2008). With this methodology, a sample is mixed with a solution of reporter probes that recognize unique sequences and capture probes that allow the complexes formed between the nucleic acids in the sample and the reporter probes to be immobilized on a solid surface for data collection. Each reporter probe is color-coded and is detected through fluorescence.

In a further embodiment, branched DNA technology, as exemplified by Panomics QuantiGene Plex 2.0 assay system, is used. Branched DNA technology comprises a sandwich nucleic acid hybridization assay for RNA detection and quantification that amplifies the reporter signal rather than the sequence. By measuring the RNA at the sample source, the assay avoids variations or errors inherent to extraction and amplification of target polynucleotides. The QuantiGene Plex technology can be combined with multiplex bead based assay system such as the Luminex system described above to enable simultaneous quantification of multiple RNA targets directly from whole cells or purified RNA preparations.

### Probes and the Selection Thereof

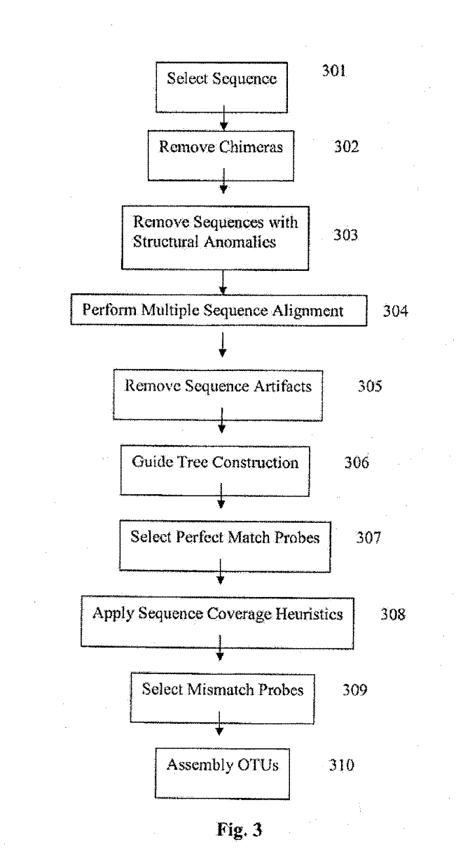

An exemplary process 300 for the design of target probes for use in the simultaneous detection of a plurality of microorganisms is illustrated in FIG. 3. Briefly, sequences are extracted from a database at a state 301. Typically, the database contains phylogenetic sequences or other highly conserved or homologous sequences. The sequences are analyzed for chimeras at a state 302 that are removed from further consideration. Chimeric sequences result from the union of two or more unrelated sequences, typically from different genes. Optionally, sequences can be further analyzed for structural anomalies, such as propensity for hairpin loop formation, at a state 303 with the identified sequences subsequently removed from further consideration. Next, multiple sequence alignments are performed on the remaining sequences in the dataset at a state 304. The aligned sequences are then checked for laboratory artifacts, such as PCR primer sequences, at a state 305, with identified sequences removed from further consideration. The remaining sequences are clustered at a state 306 and perfect match (PM) probes are selected at a state 307 that have perfect complementarity to sections of the clustered sequences. Optionally, sequence coverage heuristics are performed at a state 308 prior to selecting the mismatch (MM) probes at a state 309 for the corresponding PM probes to create probe pairs. Finally, OTUs represented by probe sets comprising a plurality of probe pairs are assembled at a state 310 to construct a hierarchal taxonomy.

Generally, a database for extraction of sequences to be used for probe selection is chosen based on the particular conserved gene or highly homologous sequence of interest, the total number of sequences within the database, the length of the overall sequences or the length of highly conserved regions within the sequences listed in the database, and the quality of the sequences therein. Typically, between two databases of equal sequence number but of different sequence length, the database with longer target regions of highly conserved sequence will generally contain a larger total number of possible sequences that can be compared. In some embodiments, the sequences are at least 300, 400, 500, 600, 700, 800, 900, 1,000, 1,200, 1,400, 1,600, 1,800, 2,000, 4,000, 8,000, 16,000 or 24,000 nucleotides long. Generally, databases with larger number of total sequences provide more material to compare. In a further embodiment, the database contains at least 10,000, 20,000, 30,000, 40,000, 50,000, 60,000, 70,000, 80,000, 100,000, 200,000, 500,000, 1,000,000 or 2,000,000 sequence listings. A gene of particular interest for probe construction is 16S rDNA (16S rRNA gene). Other conserved genes include 18S rDNA, 23S rDNA, gyrA, gyrB gene, groEL, rpoB gene, fusA gene, recA gene, sodA, cox1 gene, and nifD gene. In a further embodiment, the spacer region between highly conserved segments of two genes can be used. For example, the spacer region between 16S and 23S rDNA genes can be used in conjunction with conserved sections of the 16S and 23S rDNA.

In some embodiments, the detection of a biosignature comprises the use of probes designed to hybridize with known or discovered targets within one or more OTUs. In some embodiments, targets are selected from a collection of known targets, such as in a database. In some embodiments of the invention, a database used for the selection of probes comprises at least 50%, 60%, 70%, 80%, 90%, 95%, 96%, 97%, 98%, 99% or up to 100% of the known sequences of the organisms of interest, e.g., of the bacteria, archaea, fungi, eukaryotes, microorganisms, or prokaryotes of interest. The sequences for each individual organism in the database can include more than 20%, 30%, 40%, 50%, 60%, 70%, 80%, 90%, or more than 95% of the genome of the organism, or of the non-redundant regions thereof. In some embodiments, the database includes up to 100% of the genome of the organisms whose sequenced are contained therein, or of the non-redundant sequences thereof. A listing of almost 40,000 aligned 16S rDNA sequences greater than 1250 nucleotides in length can be found on the Greengenes web application, a publicly accessible database run by Lawrence Berkeley National Laboratory. Other publicly accessible databases include GenBank, Michigan State University's ribosomal database project, the Max Planck Institute for Marine Microbiology's Silva database, and the National Institute of Health's NCBI. Proprietary sequence databases or combinations created by amalgamating the contents of two or more private and/or public databases can also be used to practice the methods of this invention. In some embodiments, a sample is assayed for all targets in one or more chosen databases simultaneously. In other embodiments, a sample is assayed for subsets of targets identified in one or more databases simultaneously. In some embodiments, a biosignature comprises the results of assaying a sample for some or all targets in one or more chosen databases. In other embodiments, a biosignature comprises a subset of the results of assaying a sample for some or all targets in one or more chosen databases.

The analysis of the selected sequences from the database for the detection and removal of chimeras at state 302 is typically performed by generating overlapping fragments and comparing these fragments against each other. Fragments may be retained if they have at least 60%, 70%, 80%, 90%, 95% or 99% sequence identity. It was realized that the above process potentially missed chimeras because the sequence diversity of the selected sequences may be low. By comparing the fragments against a core set of diverse chimera-free sequences, more chimeras can be identified and removed from the sequence set. In cases where one or more sequences are identified that as an ambiguous chimera, e.g., a chimera with a chimeric parent, the chimera is removed and the parent chimera is fragmented and a second comparison cycle is performed. Sequences from a dataset can also be screened for chimeras using a proprietary software program such as Bellerophon3 available from the Greengenes website at greengenes.lbl.gov.

The dataset of retained non-chimeric sequences can then be screened for structural anomalies at state 303 by aligning the retained sequences against the core set of known sequences. Sequences in the retained dataset that have at least 25, 30, 35, 40, 45, 50, 60, 70 or 80 gaps in their alignment when compared against a core set or have insertions of greater than 50, 60, 70, 80, 90, 100, 110, 120, 130, 140, 150, 160, 170, 180, 190, 200, 250, 300 or 400 basepairs when compared against the core set are tagged as having a sequence anomaly and are removed from the dataset.

The screened sequences are then aligned into a multiple sequence alignment (MSA) at state 304 for comparison against the known, chimeric free core set. One alignment tool for performing intensive alignment computations is NAST (Nearest Alignment Space Termination) web tool (DeSantis et al., Nucleic Acids Res. (2006) 34:W394-399). Any appropriate alignment tool can be used to compile the MSAs, for example, clustalw (Thompson et al., Nucleic Acids Res (1994) 22:4673-4680) and MUSCLE (Edgar, Nucleic Acids Res. (2004) 32:1792-1797).

The aligned sequences are searched for sequences harboring PCR primer sequences at state 305 and any so-identified sequences are removed from the dataset.

The aligned sequences can then be clustered at the state 306 to create what is termed a “guide tree.” First, the sequences are converted to a list of kmers. A pair-wise comparison of the lists of kmers is performed and the percent of kmers in common is recorded in a sparse matrix only if a threshold similarity is found. The sparse matrix is clustered e.g., using complete linkage. Clustering includes agglomerative “bottom-up” or divisive “top-down” hierarchical clustering, distance “partition” clustering and alignment clustering. From each cluster, the sequence with the most information content is chosen as a representative. Usually, sequences derived from genome sequencing projects are given priority in cluster creation because they are less likely to be chimeras or have other sequence anomalies. The cyclic process is repeated using only the representatives from the previous cycle. For each new cycle, the threshold for recording in the sparse matrix is reduced. At the final stage, a root node is linked to the final representative sequences in a multifurcated tree. The representative sequences found in each cycle represent a node in the resulting guide tree. All nodes are linked based on their clustering results via a self-referential table allowing rapid access to any hierarchical point in the guide tree. In some embodiments, the results are stored in a database format, e.g., in a Structured Query Language (SQL) compliant format. In the resulting guide tree, each leaf node represents an individual organism and each node above the lowest level of the guide tree represents a candidate OTU.

Typical distance matrixes built from approximately 2×10⁵ sequences can require 40 billion intersections that would require about 40 gigabytes of data space if encoded to disk. Doubling the amount of sequences to 4×10⁵ requires a quadrupling of the file size (approximately 160 GB). The clustering methodology illustrated here using a sparse matrix avoids the need for large files and the expected increase in computing time. Therefore the methodology can be performed more efficiently than conventional sequence clustering methods. Moreover, with distance matrices created from sequence alignments (e.g., DNA alignments), one misalignment can affect many distance values. In contrast, the clustering method illustrated herein is based on the alignment of kmers, and thus the effect of a misalignment on clustering values is significantly reduced.

Following guide tree construction, the dataset of remaining sequences, now termed the “filtered sequence dataset” is used to select candidate probes, e.g., PM probes. First, unsupported sequence polymorphisms are identified and removed from the filtered sequence dataset using a pre-clustering process that uses the guide tree generated above to create clusters over a minimum similarity and under a maximum size. Typically, clustered sequences are at least 80%, 85%, 90%, 95%, 97% or 99% similar. Usually, clusters have no more than 1,000, 500, 200, 100, 80, 60, 50, 40, 30, 20 or 10 sequences. This process allows sequence data outliers to be detected by comparison within near-neighbors and removed from the filtered sequence dataset.

Next, the remaining sequences are fragmented to the desired size to generate candidate target probes. Typically, the fragments range from about 10mer to 100mer, 15mer to about 50mer, about 20mer to about 40mer, about 20mer to about 30mer. Usually, the fragments are at least 15mer, 20mer, 25mer, 30mer, 40mer, 50mer or 100mer in size. Each candidate target probe is required to be found within a threshold fraction of at least one pre-cluster. Generally, threshold fractions of at least 80%, 90% or 95% are used.

All candidate PM probes that are within a threshold fraction of at least one pre-cluster are then evaluated for various biophysical parameters, such as melting temperature (61-80° C.), G+C content (35-70%), hairpin energy over −4 kcal/mol, potential for self-dimerization (>35° C.). Candidate PM probes that fall outside of the setting boundaries of the biophysical parameters are eliminated from the dataset. Optionally, probes can be further filtered for ease of photolithographic synthesis.

The likelihood of cross-hybridization of each PM candidate probe to each non-target input 16s rRNA gene sequence is determined. The cross-hybridization pattern for each PM candidate probe is recorded.

Sequence coverage heuristics are performed at the state 308 are then applied to candidate PM probes with acceptable biophysical parameters.

For each candidate PM probe, corresponding MM probes can be generated at the state 309. Each MM probe differs from its corresponding PM probe by at least one nucleotide. In some embodiments, the MM probe differs from its corresponding PM probe by 1, 2, 3, 4, 5, 6, 7, 8, 9 or 10 nucleotides. Within a MM probe, the mismatched nucleotide or nucleotides can include any of the 3 central bases that are not found in the same position or positions in the PM probe. For example, with a 25mer PM probe that has a guanine at the 13^{th} position, i.e., the central nucleotide, the MM probes comprise probes with adenine, thymine, uracil or cytosine at the 13^{th} position. Similarly, with a 25mer PM probe with an adenine at the 12^{th} nucleotide position and a guanine at the 13^{th} nucleotide position when read from the 3′ direction, the possible MM probes comprise probes with guanine at the 12^{th} nucleotide and adenine, thymine or cytosine at the 13^{th} nucleotide position; cytosine at the 12^{th} nucleotide position and adenine, thymine or cytosine at the 13^{th} nucleotide position; and thymine at the 12^{th} nucleotide position and adenine, thymine or cytosine at the 13^{th} nucleotide position. In some embodiments, the mismatched nucleotide or nucleotides include any one or more of the nucleotides in a corresponding PM probe. Increasing the number of MM probes and/or the mis-match positions represented may be used to enhance quantification, accuracy, and confidence.

As describe above for the PM probes, each candidate MM probe is required to meet the set boundaries of one or more biophysical parameters, such as melting temperature, G+C content, hairpin energy, self-dimers and photolithography synthesis steps. Generally, these parameters are identical or substantially similar to the PM probe biophysical parameters.

Candidate MM probes that meet the biophysical parameters and optionally, photolithographic parameters above are then screened for the likelihood of cross-hybridization to a target sequence. Usually, a central kmer length is evaluated. For a 25mer candidate MM, a central kmer from the candidate MM, generally a 15mer, 16mer, 17mer, 18mer, or 19mer is compared against the target sequences. A candidate MM probe that contains a central kmer that is identical to a target sequence is eliminated. Next, candidate PM probes for which no suitable candidate MM probes can be identified are also eliminated.

Each candidate OTU may be evaluated to determine the number of PM probes that are incapable of hybridization to sequences outside the OTU.

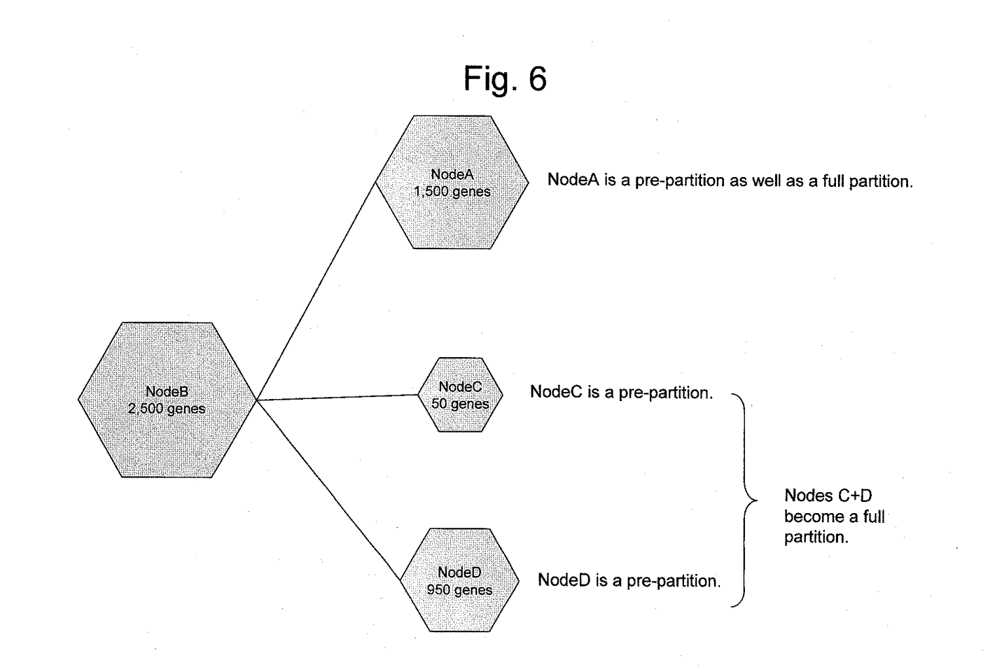

In one embodiment, a pre-partition process is performed. A pre-partition is the largest possible clade (node_id) that does not exceed the max partition size. See FIG. 6. Typically, useful partition sizes range from about 1,000 to about 8,000 nodes. Any pre-partition that is in a predetermined size range becomes a full-partition. Pre-partitions that are below the minimum partition size are combined into partitions by assembling sister nodes where possible. For example, assume that partitions are allowed to range in size from 1000 to 2000 members. If node A represents 1500 genes and its parent, node B, represents 2500 genes, then node A is considered a pre-partition. If node C is a sibling of node A, and node C represents only 50 genes, then node C is also a pre-partition because moving node C to its parent, node B, would encapsulate more than the maximum partition size of 2000 members.

To create candidate sequence clusters, transitive sequence clusters are identified using a sliding threshold of two distance matrixes based on either the count of pairwise unique candidate targets or the count of pairwise common candidate targets. Probes prevalent in a large fraction of the sequences in a candidate sequence cluster, e.g., >=90% of the sequence in the cluster, are identified using the count of sequences containing the PM and the count of sequences with unambiguous data for given PM's locus. For each prevalent probe, a cross-hybridization potential outside the cluster is also tested. All information regarding cluster-PM sets is recorded. Futile clusters are defined as clusters for which only cross-hybridizing probes are identified are removed from the dataset.

Where necessary, probes that are expected to display some degree of cross-hybridization can be selected. Potentially hybridization-prone probes are constrained to reduce the probability that sequences outside the cluster could hybridize to many of the cluster-specific PM probes. A distribution algorithm can be used to examine a graph of probe-sequence interconnections (edges) and to favor sets of probes that minimize overlapping edges.

After solutions from all partitions are completed, a global reconciliation of set solutions across partitions is performed. The sequence clusters are locked as OTUs and each cluster's PM probe set is tested for global cross-hybridization against the other remaining PM probe sets. Probes are ranked for utility based on global cross-hybridization patterns.

The OTUs are assembled and annotated. Typically, each OTU is taxonomically annotated using one term for each rank from domain, kingdom, phylum, sub-phylum, class, sub-class, order, and family. As a result, all the 16S rRNA sequences presented without taxonomic nomenclature and annotated as “environmental samples” or “unclassified” are assigned with taxonomic annotation.

Each genus-level name recognized by NCBI is read and recorded. For each lineage of taxonomic terms, duplicate adjacent terms are removed; domain-level terms are found by direct pattern match; and phylum-level terms are found as rank immediately subordinate to domain Order-level terms are found by -ales suffix and family-level terms are found by -eae suffix. If a family level-term is unavailable but a genus is identified (e.g., by match to an accepted list), the genus-level term is used to derive a family level-term. All unrecognized terms found between recognized terms are fit into available ranks (new ranks are not created for extra terms) Empty ranks are filled by deriving root terms from subordinate terms and adding pre-determined suffixes. Finally, the family of an OTU is determined by vote from the family assignment of the sequences. Ties are broken by priority sequences (e.g., sequences derived from genome sequencing projects can be given highest priority). All OTUs within a subfamily are compared by kmer distance among the sequences and OTUs are linked into a subfamily whenever a threshold similarity is observed. Each candidate OTU is evaluated to determine the count of targets which are prevalent across the sequences of the candidate OTU and are not expected to hybridize to sequences outside the OTU.

Exemplary PM and MM 25mer probes generated using the disclosed algorithms are provided as SEQ ID Nos. 1-50. It should be noted that the above process is applicable to the selection of probes ranging in size from at least 15 nucleotides to at least 200 nucleotides in length and includes probes that are flanked on one or both sides by common or irrelevant sequences, including linking sequences. Furthermore, probes selected by this process can be further processed to yield probes that are smaller than or larger than the original selected probes. For example, probes listed as SEQ ID Nos. 1-50 can be further processed by removing sequences from the 3′end, 5′end or both to produce smaller sequences that are identical to at least a portion of the sequence of the 25mers. In other embodiments, larger probes can be generated by incorporating the sequences of probes identified by the disclosed algorithms, i.e., a 25mer probe can be incorporated into a 30mer or larger, 35mer or larger, 40mer or larger, 45mer or larger, 50mer or larger, 55mer or larger, 60mer or larger, 65mer or larger, 70mer or larger, 75mer or larger, 80mer or larger, 85mer or larger or 90mer or larger probe. Additionally, probes listed as SEQ ID Nos. 1-50 can be shortened on one end and lengthened on the other end to yield probes that range from 10mer to 200mer.

Probes selected by the above process also include probes that comprise one or more base substitutions, for example uracil in the place of thymine; incorporate one or more base analogs such as nitropyrrole and nitroindole; comprise of one or more sugar substitutions, e.g., ribose in the place of deoxyribose, or any combination thereof. Similarly, probes selected by the process of the invention, may further comprise alternate backbone chemistry, for example, comprising of phosphoramide.

The size of the collection of putative probes generated by the methodologies of the invention is partially dependent on the length of the particular highly conserved sequence with longer sequences like that of 23S rRNA gene allowing for a greater number of homologous sequences than a smaller highly conserved sequence such as 16S rRNA gene. In some embodiments, the length of the highly conserved sequence is at least 100 bp, 250 bp, 500 bp, 1,000 bp, 2,000 bp, 4,000 bp, 8,000 bp, 10,000 bp, or 20,000 bp. Additionally, the size of the collection of putative probes generated by the methodologies of the invention is also dependent on the size of the collection of homologous sequences in one or more databases from which sequences are selected for the analysis and generation of probes. Larger collections of homologous sequences, by providing a larger pool of sequences that can be analyzed, allow for the generation of more putative probes. In some embodiments, the starting collection of homologous sequences in one or more databases contains at least 100,000, 250,000, 500,000, 1,000,000, 2,000,000, 5,000,000 or 10,000,000 sequences. The size of the collection of putative probes is further dependent on the length of the desired probe, because the probe length decreases, as the number of probes that bind to unique sequences increases. Depending on the particular highly conserved sequence, the size of the database and the length of the desired probe, collections of putative probes of at least 100, 1,000, 10,000, 25,000, 50,000, 100,000, 250,000, 500,000, 1,000,000, 2,000,000, 5,000,000 or 10,000,000 probes can be generated.

Detection systems can be constructed from the putative probes generated by the above methods. The detection system can have any number of probes and range from 1 probe to all the probes selected by the methodology. In some embodiments, the detection system comprises at least 1, 2, 3, 4, 5, 6, 7, 8, 9, 10, 15, 20, 25, 30, 35, 36, 40, 45, 50, 55, 60, 65, 70, 80, 90, 100, 125, 150, 200, 300, 400, 500, 1000, 2,000, 5,000, 10,000, 20,000, 40,000, 50,000, 100,000, 200,000, 500,000, 1,000,000 or 2,000,000 probes. Systems with large number of probes can be used to identify relevant microorganisms in a sample, e.g., an environment or clinical sample, and/or to generate a biosignature. In another embodiment, once relevant microorganisms are known, detection systems with low (e.g., 1-10,000) to medium (e.g., 10,000-100,000) numbers of probes can be designed for special purpose applications, such as determining one or more specific biosignatures. In some embodiments, knowledge of the identity of relevant microorganisms can be used to select further probes to these microorganisms. If, for instance, five 25mer probes in a first set of probes hybridize to a relevant microorganism, then variants of these five probes can be generated and tested (e.g. in silico) for their binding and biophysical characteristics. Alternately, identification of relevant microorganisms can lead to the generation of new probes that are unlike the probes first used to identify the microorganisms. For example, once novel microorganisms are identified, antibodies can be generated for specific applications.

To select OTU-specific probes, e.g., oligonucleotide probes specific for organisms that are included within a hierarchical node, additional PM probes can be chosen for each hierarchical node that has more than one child node. To qualify targets for selection to a certain node, a threshold fraction of sequences within a node matching a PM set are enforced. Examples of the threshold fractions included 0.2%, 0.5%, 1%, 2%, 3%, 4%, 5%, 6%, 7%, 8%, 9%, and 10%. Coverage of direct sub-nodes (children) is also enforced. For example, each target should be representative of at least 25% of at least one sub-node.

The specificity of the probes selected by the methods disclosed herein can be validated experimentally in a number of ways. For example, the hybridization signal of a probe in the presence of the target sequence can be measured and compared to the background signal. Target sequences can be derived from one or more pure cultures or from environmental or clinical samples that are known to contain the target sequence. A specific taxa can be identified as present in a sample if a majority (about 70% to about 100%, about 80% to about 100% or about 90% to about 100%) of the probes on the array have a hybridization signal at least about 50 times, 100 times, 150 times, 200 times, 250 times, 300 times, 350 times, 400 times, 450 times, 500 times, or 1,000 times greater than that of the background. Also, the hybridization signal of the probe can be compared to the hybridization signal of one or more of its mismatch probes. A PM:MM ratio of at least 1.05, 1.10, 1.15, 1.20, 1.25, 1.30, 1.40, 1.45, or 1.50 can indicate that the PM probe can selectively hybridize to its target sequence. An additional way to test the ability of a probe to selectively hybridize to its target is to calculate a pair difference score (d), further explained below. A pair difference score above 1.0 indicates that the probe can selectively hybridize to the target compared to one of its mismatch probes.

The methods disclosed herein can be used to select and/or utilize organism-specific and/or OTU-specific oligonucleotide probes for biomolecules, such as proteins, DNA, RNA, DNA or RNA amplicons, and native rRNA from a target nucleic acid molecule. In some embodiments, probes are designed to be antisense to the native rRNA so that rRNA from samples can be placed on the array to identify actively metabolizing organisms in a sample with no bias from PCR amplification. Actively metabolizing organisms have significantly higher numbers of ribosomes used for the production of proteins, compared to quiescent or dead organisms. Therefore, in some embodiments, the capacity of one or more organisms to make proteins at a particular point in time can be measured. In this way, the array system of the present embodiments can be used to directly identify the metabolizing organisms within diverse communities.

### Sample Preparation

In some embodiments, the sample used can be an environmental sample from any source, for example, naturally occurring or artificial atmosphere, water systems and sources, soil or any other sample of interest. In some embodiments, the environmental sample may be obtained from, for example, indoor or outdoor air or atmospheric particle collection systems; indoor surfaces and surfaces of machines, devices or instruments. In some embodiments, ecosystems are sampled. Ecosystems can be terrestrial and include all known terrestrial environments including, but not limited to soil, surface and above surface environments. Ecosystems include those classified in the Land Cover Classification System (LCCS) of the Food and Agriculture Organization and the Forest-Range Environmental Study Ecosystems (FRES) developed by the United States Forest Service. Exemplary ecosystems include forests such as tropical rainforests, temperate rainforest, temperate hardwood forests, boreal forests, taiga and montane coniferous forests; grasslands including savannas and steppes; deserts; wetlands including marshes, swamps, bogs, estuaries, and sloughs; riparian ecosystems, alpine and tundra ecosystems. Ecosystems further include those associated with aquatic environments such as lakes, streams, springs, coral reefs, beaches, estuaries, sea mounts, trenches, and intertidal zones. Ecosystems also comprise soils, humus, mineral soils and aquifiers. Ecosystems further encompass underground environments, such as mines, oil fields, caves, faults and fracture zones, geothermal zones and aquifers. Ecosystems additionally include the microbiomes associated with plants, animals, and humans. Exemplary plant associated microbiomes include those found in or near roots, bark, trunks, leaves, and flowers. Animal and human associated microbiomes include those found in the gastrointestinal tract, respiratory system, nares, urogenital tract, mammary glands, oral cavity, auditory canal, feces, urine, and skin.

In other embodiments, the sample can be any kind of clinical or medical sample. For example, samples from blood, urine, feces, nares, the lungs or the gut of mammals may be assayed using the array system. Also, the probes selected by the methods disclosed herein and the array system of the present embodiments can be used to identify an infection in the blood of an animal. The probes selected by the methods disclosed herein and the array system of the present embodiments can also be used to assay medical samples that are directly or indirectly exposed to the outside of the body, such as the lungs, ear, nose, throat, the entirety of the digestive system or the skin of an animal Hospitals currently lack the resources to identify the complex microbial communities that reside in these areas.

Techniques and systems to obtain genetic sequences from multiple organisms in a sample, such as an environmental or clinical sample, are well known by persons skilled in the art. For example, Zhou et al. (Appl. Environ. Microbiol. (1996) 62:316-322) provides a robust nucleic acid extraction and purification. This protocol may also be modified depending on the experimental goals and environmental sample type, such as soils, sediments, and groundwater. Many commercially available DNA extraction and purification kits can also be used. Samples with lower than 2 pg purified DNA may require amplification, which can be performed using conventional techniques known in the art, such as a whole community genome amplification (WCGA) method (Wu et al., Appl. Environ. Microbiol. (2006) 72, 4931-4941). In some embodiments, highly conserved sequences such as those found in the 16S RNA gene, 23S RNA gene, 5S RNA gene, 5.8S rRNA gene, 12S rRNA gene, 18S rRNA gene, 28S rRNA gene, gyrB gene, rpoB gene, fusA gene, recA gene, cox1 gene and nifD gene are amplified. Usually, amplification is performed using PCR, but other types of nucleic acid amplification can be employed. Generally, amplification is performed using a single pair of universal primers specific to a highly conserved sequence. For redundancy or for increased amount of total amplicon concentration, two or more universal probe pairs each specific to a different highly conserved sequence can be used. Representative PCR primers include: bacterial primers 27F and 1492R.

Techniques and systems for obtaining purified RNA from environmental samples are also well known by persons skilled in the art. For example, the approach described by Hurt et al. (Appl. Environ. Microbiol. (2001) 67:4495-4503) can be used. This method can isolate DNA and RNA simultaneously within the same sample. A gel electrophoresis method can also be used to isolate community RNA (McGrath et al., J. Microbiol. Methods (2008) 75:172-176). Samples with lower than 5 pg purified RNA may require amplification, which can be performed using conventional techniques known in the art, such as a whole community RNA amplification approach (WCRA) (Gao et al., Appl. Environ. Microbiol. (2007) 73:563-571) to obtain cDNA. In some embodiments, environmental sampling and DNA extraction are conducted as previously described (DeSantis et al., Microbial Ecology, 53(3):371-383, 2007). In other embodiments, 16S rRNA or 23S rRNA is directly labeled and used without any amplification.

### Probe Preparation

Techniques and means for generating oligonucleotide probes to be used on analysis systems, beads or in other systems are well-known by persons skilled in the art. For example, the oligonucleotide probes can be generated by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 15 and about 100 bases, and most preferably between about 20 and about 40 bases in length. In some embodiments, synthetic nucleic acids include non-natural bases, such as, but by no means limited to, inosine. An example of a suitable nucleic acid analogue is peptide nucleic acid (see, e.g., Egholm et al., Nature 363:566-568 (1993); U.S. Pat. No. 5,539,083). In some embodiments, at least 10, 25, 50, 100, 500, 1,000, 5,000, 10,000, 20,000, 40,000, 50,000, 60,000, 70,000, 80,000, 90,000 100,000, 200,000, 500,000, 1,000,000 or 2,000,000 probes are included on the array. In further embodiments, each PM probe has a corresponding MM probe present on the array. Typically, each probe pair is associated with an OTU. In some embodiments, at least 10, 25, 50, 100, 500, 1,000, 5,000, 10,000, 20,000, 40,000, 50,000, 60,000, 70,000, 80,000, 90,000 100,000, 200,000 or 500,000 probe pairs are placed on the array. Generally, sets of probe pairs have at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 probe pairs present.

In some embodiments, positive control probes that are complementary to particular sequences in the target sequences (e.g., 16S rRNA gene) are used as internal quantification standards (QS) and included in the system. In other embodiments, positive control probes, also known as internal DNA quantification standards (QS) probes are probes that hybridize to spiked-in nucleic acid sequence targets. Usually, the sequences are from metabolic genes. In some embodiments, negative control (NC) probes, e.g., probes that are not complementary or do not appreciably hybridize to sequences in the target sequences (e.g., 16S rRNA gene) are included on the array. Unlike the QS probes, no target material is spiked into the sample mix for the NC probes, prior to sample processing.

### Hybridization Platform Fabrication

In some embodiments, the probes are synthesized separately and then attached to a solid support or surface, which may be made, e.g., from glass, latex, plastic (e.g., polypropylene, nylon, polystyrene), polyacrylamide, nitrocellulose, gel, silicon, or other porous or nonporous material. In some embodiments, the surface is spherical or cylindrical as in the case of microbeads or rods. In other embodiments, the surface is planar, as in an array or microarray. For example, the method described generally by Schena et al, Science 270:467¬470 (1995) can be used for attaching the nucleic acids to a surface by printing on glass plates. In other embodiments, typically used for making high-density oligonucleotide arrays, thousands of oligonucleotides complementary to defined sequences are synthesized in situ at defined locations on a surface by photolithographic techniques (see e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (e.g., Blanchard et al., Biosensors & Bioelectronics 11:687-690). In some of these methods, oligonucleotides (e.g., 25-mers) of known sequence are synthesized directly on a surface such as a derivatized glass slide. Other methods for making analysis systems are also available, e.g., by masking (Maskos and Southern, 1992, Nuc. Acids. Res. 20:1679¬1684). Embodiments of the present invention are applicable to any type of array, for example, bead-based arrays, arrays on glass plates or derivatized glass slides as discussed above, and dot blots on nylon hybridization membranes.

Embodiments of the invention are applicable for use in any analysis system, including but not limited to bead or solution multiplex reaction platforms, or across multiple platforms, for example, Affymetrix GeneChip® Arrays, Illumina BeadChip® Arrays, Luminex xMAP® Technology, Agilent Two-Channel Arrays, MAGIChips (Analysis systems of Gel-immobilized Compounds) or the NanoString nCounter Analysis System. The Affymetrix (Santa Clara, Calif., USA) platform DNA arrays can have the oligonucleotide probes (approximately 25mer) synthesized directly on the glass surface by a photolithography method at an approximate density of 10,000 molecules per μm² (Chee et al., Science (1996) 274:610-614). Spotted DNA arrays use oligonucleotides that are synthesized individually at a predefined concentration and are applied to a chemically activated glass surface. In general, oligonucleotide lengths can range from a few nucleotides to hundreds of bases in length, but are typically from about 10mer to 50mer, about 15mer to 40mer, or about 20mer to about 30mer in length.

### Microparticle Systems

Oligonucleotides produced using techniques known in the art can be built on and/or coupled to microspheres, beads, microbeads, rods, or other microscopic particles for use in arrays, flow cytometry and other multiplex assay systems. Numerous microparticles are commercially available from about 0.01 to 100 micrometers in diameter. Generally, microparticles from about 0.1-50 μm, about 1-20 μm, or about 3-10 μm are preferred. The size and shapes of microparticles can be uniform or they can vary. In some embodiments, sublots of different sizes, shapes or both are conjugated to probes before combining the sublots to make a final mixed lot of labeled microparticles. The individual sublots can therefore be distinguished and classified based on their size and shape. The size of the microparticles can be measured in practically any flow cytometry apparatus by so-called forward or small-angle scatter light. The shape of the particle can be also discriminated by flow cytometry, e.g., by high-resolution slit-scanning method.

Microparticles can be made out of any solid or semisolid material including glass, glass composites, metals, ceramics, or polymers. Frequently, the microparticles are polystyrene or latex material, but any type of polymeric material is acceptable including but not limited to brominated polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, or combinations thereof. Microparticles, can be magnetic or non-magnetic and may also have a fluorescent dye, quantum dot, or other indicator material incorporated into the microparticle structure or attached to the surface of the microparticles. Frequently, microparticles may also contain 1 to 30% of a cross-linking agent, such as divinyl benzene, ethylene glycol dimethacrylate, trimethylol propane trimethacrylate, or N,N′methylene-bis-acrylamide or other functionally equivalent agents known in the art.

### Target Labeling

In one embodiment, the nucleic acid targets are labeled so that a laser scanner tuned to a specific wavelength of light can measure the number of fluorescent molecules that hybridized to a specific DNA probe. For arrays, the nucleic acid targets are typically fragmented to between 15 and 100 nucleotides in length and a biotinylated nucleotide is added to the end of the fragment by terminal DNA transferase. At a later stage, the biotinylated fragments that hybridize to the oligonucleotide probes are used as a substrate for the addition of multiple phycoerythrin fluorophores by a sandwich (Streptavidin) method. For some arrays, such as those made by AGILENT or NIMBLEGEN, the purified community DNA can be fluorescently labeled by random priming using the Klenow fragment of DNA polymerase and more than one fluorescent moiety can be used (e.g. controls could be labeled with Cy3, and experimental samples labeled with Cy5 for direct comparison by hybridization to a single analysis system). Some labeling methods incorporate the molecular label into the target during an amplification or enzymatic step to produce multiple labeled copies of the target.

In some embodiments, the detection system is able to measure the microbial diversity of complex communities without PCR amplification, and consequently, without the inherent biases associated with PCR amplification. Actively metabolizing cells typically have about 20,000 or more ribosomal copies within their cell for protein assembly compared to quiescent or dead cells that have few. In some embodiments, rRNA can be purified directly from environmental samples and processed with no amplification step, thereby avoiding any of the biases caused by the preferential amplification of some sequences over others. Thus, in some embodiments, the signal from the analysis system can reflect the true number of rRNA molecules that are present in the samples. This can be expressed as the number of cells multiplied by the number of rRNA copies within each cell. The number of cells in a sample can then be inferred by several different methods, such as, for example, quantitative real-time PCR, or FISH (fluorescence in situ hybridization.). Then the average number of ribosomes within each cell may be calculated.

### Hybridization

Hybridizations can be carried out under conditions well-known by persons skilled in the art. See Rhee et al. (Appl. Environ. Microbiol. (2004) 70:4303-4317) and Wu et al. (Appl. Environ. Microbiol. (2006) 72:4931-4941). The temperature can be varied to reduce or increase stringency and allow the detection of more or less divergent sequences. Robotic hybridization and stringency wash stations can be used to give more consistent results and reduce processing time. In some embodiments, the hybridization and washing process can be accomplished in less than about half an hour, 1 hour, 2 hours, 3 hours, 4 hours, 5 hours, 6 hours, 7 hours, 8 hours, 9 hours, 10 hours, 11 hours, 12 hours, 14 hours, 16 hours, 18 hours, 20 hours or 24 hours. Generally, hybridization and washing times are reduced for microparticle based detection systems owing to the greater accessibility of the probes to the target molecules. Generally, hybridization times may be reduced for low complexity assays and/or assays for which there is an excess of target analytes.

### Signal Quantification

After hybridization, arrays can be scanned using any suitable scanning device. Non-limiting examples of conventional microarray scanners include GeneChip Scanner 3000 or GeneArray Scanner, (Affymetrix, Santa Clara, Calif.); and ProScan Array (Perkin Elmer, Boston, Mass.); and can be equipped with lasers having resolutions of 10 pm or finer. The scanned image displays can be captured as a pixel image, saved, and analyzed by quantifying the pixel density (intensity) of each spot on the array using image quantification software (e.g., GeneChip Analysis system Analysis Suite, version 5.1 Affymetrix, Santa Clara, Calif.; and ImaGene 6.0, Biodiscovery Inc. Los Angeles, Calif., USA). For each probe, an individual signal value can be obtained through imaging parsing and conversion to xy-coordinates. Intensity summaries for each feature can be created and variance estimations among the pixels comprising a feature can be calculated.

With flow cytometry based detection systems, a representative fraction of microparticles in each sublot of microparticles can be examined. The individual sublots, also known as subsets, can be prepared so that microparticles within a sublot are relatively homogeneous, but differ in at least one distinguishing characteristic from microparticles in any other sublot. Therefore, the sublot to which a microparticle belongs can readily be determined from different sublots using conventional flow cytometry techniques as described in U.S. Pat. No. 6,449,562. Typically, a laser is shined on individual microparticles and at least three known classification parameter values measured: forward light scatter (C₁) which generally correlates with size and refractive index; side light scatter (C₂) which generally correlates with size; and fluorescent emission in at least one wavelength (C₃) which generally results from the presence of fluorochrome incorporated into the labeled target sequence. Because microparticles from different subsets differ in at least one of the above listed classification parameters, and the classification parameters for each subset are known, a microparticle's sublot identity can be verified during flow cytometric analysis of the pool of microparticles in a single assay step and in real-time. For each sublot of microparticles representing a particular probe, the intensity of the hybridization signal can be calculated along with signal variance estimations after performing background subtraction.

### Data Processing and Statistical Analysis

Simultaneous detection of at least 500, 1,000, 5,000, 10,000, 20,000, 30,000, 40,000, 50,000, 60,000, or more taxa with a high level of confidence can incorporate techniques to de-convolute the signal intensity of numerous probe sets into probability estimates. In some embodiments, the methods, compositions, and systems of the invention enable detection in one assay the presence or absence of a microorganism in a community of microorganisms, such as an environmental or clinical sample when the microorganism comprises less than 0.05% of the total population of microorganisms. In some embodiments, detection includes determining the quantity of the microorganism, e.g., the percentage of the microorganism in the total microorganism population. De-convolution techniques can include the incorporation of NC probe pairs into the analysis system and the use of the data to fit the hybridization signals from the QS probe pairs to the hybridization distribution of the NC probe pairs.

De-convolution techniques can allow the detection and quantification of nucleic acids in a sample and by inference, the detection and quantification of microorganisms in a sample. In one aspect of the invention, a system is provided for determining the presence or quantity of a microorganism in a sample comprising contacting a sample with a plurality of probes, detecting the hybridization signals of the sample nucleic acids with the probes and de-convoluting the signals to determine the presence, absence and/or quantity of a particular nucleic acid present in a population of nucleic acids where the particular nucleic acid is present at less than 0.01% of the total nucleic acid population. In some embodiments, the particular nucleic acid is at least 80%, 85%, 90%, 91%, 92%, 93%, 94%, 95%, 96% or 97% homologous to other nucleic acids in the population.

In some embodiments, the data output from an imaged or scanned sample is de-convoluted and analyzed using the following methods. Using an array as an illustrative example, the hybridization signals are converted to xy-coordinates with intensity summaries and variance estimates generated for the pixels using commercial software. The data is outputted using a standard data format like a CEL file (Affymetrix), or a Feature Report file (NimbleGen).

The hybridization signals undergo background subtraction. Typically, the background intensity is computed independently for each quadrant as the average signal intensity of the least intense 2% of the probes in the quadrant. Other threshold values may also be used, e.g., 0.5%, 1%, 3%, 4%, 5% or 10%. Background intensity is then subtracted from all probes in a quadrant before further computation is performed. This noise removal procedure can be done on a quadrant-by-quadrant basis or across a whole array.

In some embodiment, array signals are normalized to allow for the comparison of results achieved in different experiments or for the comparison of replicate experiments. Normalization can be achieved by a number of methods. In one embodiment, reproducibility between different probes for the same target are evaluated using a Position Dependent Nearest Neighbor (PDNN) model as described in Zhang L. et al., A model of molecular interactions on short oligonucleotide analysis systems, Nat. Biotechnol. 2003, 21(7):818-821. The PDNN model allows estimation of the sequence specific noise signal and a non-specific background signal, and thus enables estimation of the true intensity for the probes.

In other embodiments, per-array models of signal and background distributions using responses observed from comparison of the PM and MM probe pairs and the internal DNA quantification standards (QS) probe pairs are created. In one embodiment, the probability that each probe pair is “positive” is determined by calculating a difference score, d, for each probe pair. d may be defined as:

$$d = 1 - ( \frac{PM - MM}{PM + MM} ) \qquad Eqn . 1$$

wherein:
PM=scaled intensity of the perfect match probe;
MM=scaled intensity of the mismatch probe; and,
d=pair difference score.
   The value of d can range from 0 to 2. When PM>>MM, the value of d approaches 0; when PM=MM, d=1; and when PM<<MM, the value of d approaches 2.

In some embodiments, the internal DNA quantification standards (QS) and negative control (NC) probe pairs are binned and sorted by attributes of the probes. Examples of the attributes of the probes that can be used in the embodiments of the present invention include, but are not limited to binding energy; base composition, including A+T count, G+C count, and T count; sequence complexity; cross-hybridization binding energy; secondary structure; hair-pin forming potential; melting temperature; and length of the probe. These attributes of the probes may affect hybridization properties of the probes, for example, A+T count may affect hydrogen bonding of the probe, and T count may affect the length and base composition of the fragments produced by the use of DNase. Fragmentation with other enzyme systems may be influenced by the composition of other bases.

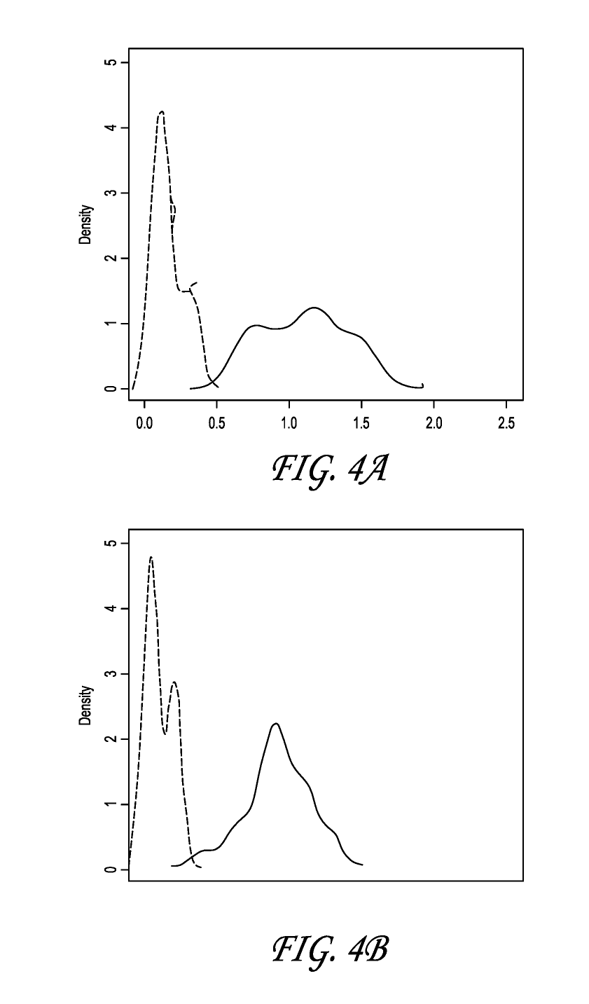

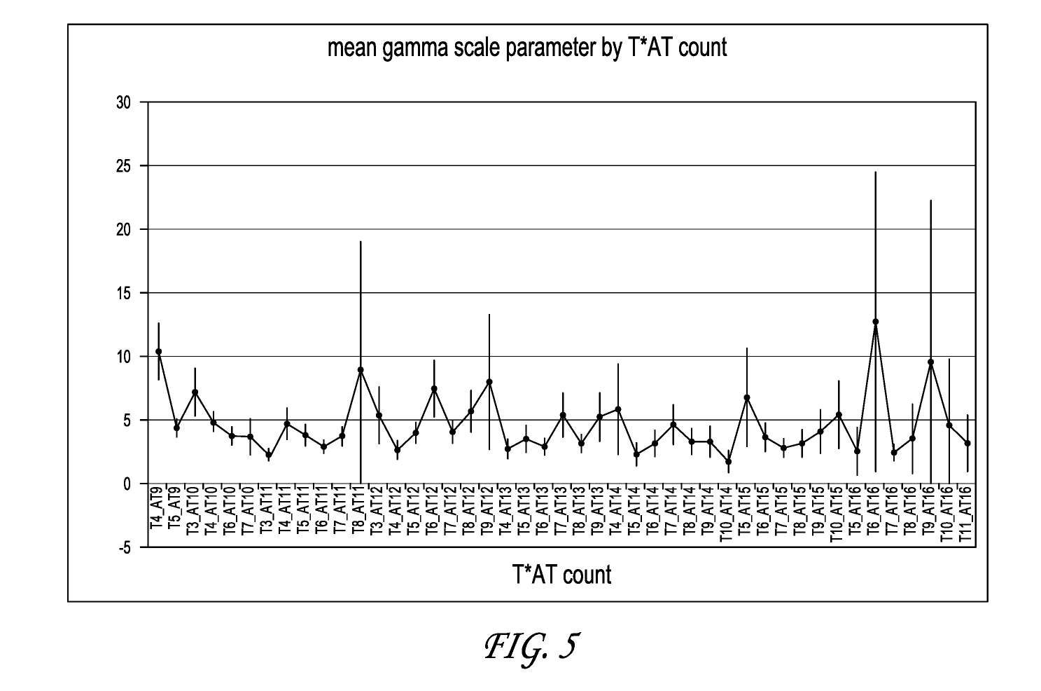

In one embodiment, QS and NC probe pairs are binned and sorted based on the individual probe's A+T count and T count. For each bin (A+T count by T count), the d values from the negative control probes are fit to a normal distribution to derive the scale (mean) and shape (standard deviation). Then, the d values from QS are fit to a gamma distribution to derive scale and shape. For each array, multiple density plots are produced by this process. Two examples of density plots generated from two different probe bins within the same array are shown in FIG. 4A-B. The AT count is 14 for the probes represented both figures. The T count is 9 for the probes in FIG. 4A, while the T count is 10 for the probes represented in FIG. 4B. As these graphs demonstrate, even one extra T, as shown in FIG. 4B, can result in appreciable difference in the probe gamma scale parameter. Variations of gamma scale across 79 arrays are shown in FIG. 5.

The parameters derived from gamma and normal distributions are used to derive a pair response score, r, for each probe pair. r is an indicator of the probability that a probe pair is positive, i.e., the probability for a probe pair to be responsive to the target sequence. r may be defined as:

$$r = ( \frac{{pdf}_{γ} ( X = d )}{{pdf}_{γ} ( X = d ) + {pdf}_{norm} ( X = d )} ) \qquad Eqn . 2$$

where:

r=response score to measure the potential that a specific probe pair is binding a target sequence and not a background signal, i.e. the probability of the probe pair being positive for the specific target sequence;
pdf_{γ} (X=d)=probability that d could be drawn from the gamma distribution estimated for the target class ATx Ty;
Pdfₙₒᵣₘ (X=d)=probability that d could be drawn from the normal distribution estimated for the target class ATx Ty.
r can range from 0 to 1. r approaches 1 when PM>>MM, and r approaches 0 when PM<<MM.

Each set of interrogation probe pairs, e.g., an OTU, can be scored based on pair response scores, cross-hybridization relationships or both. In some embodiments, the system removes data from at least a subset of probe pair sets before making a final call on the presence or quantity of said microorganisms. In one embodiment, the data is removed based on interrogation probe cross hybridization potential. In one embodiment, the scoring of probe pairs is performed by a two-stage process as discussed below.

For example, a two stage analysis can be performed wherein only probe pairs that pass a first stage are analyzed in the next stage. In the first stage, the distribution of r across each set of probe pairs, R, is determined. For each set of probe pairs that is associated with an OTU, the r values of all probe pairs are ranked within the set, and percentage of probe pairs that meet one or more threshold r values are determined. Frequently, three threshold determinations are made at 25% increments across the total range of ranked probe pairs (interquartile Q1, Q2, and Q3); however, any number of threshold determinations or percentage increments can be used. For example, a determination may use one increment at 70% in which probe pairs must pass a threshold value of 80%.

Typically, to differentiate signal from noise, an OTU is considered to pass Stage 1 if Q1, Q2, and Q3 of the set of probe pairs that is associated with this OTU surpass the threshold of Q1ₘᵢₙ, Q2ₘᵢₙ, and Q3ₘᵢₙ, respectively. That is, for an OTU to pass Stage 1, the r value of 75% of the probe pairs in the set of probe pairs that is associated with that OTU has to be at least Q1ₘᵢₙ, the r value of 50% of the probe pairs in that set of probe pairs have to be at least Q2ₘᵢₙ, and the r value of 25% of the probe pairs in that set of probe pairs have to be at least Q3ₘᵢₙ. Q1ₘᵢₙ is at least about 0.5, about 0.55, about 0.6, about 0.65, about 0.7, about 0.75, about 0.8, about 0.82, about 0.84, about 0.86, about 0.88, about 0.90, about 0.91, about 0.92, about 0.93, about 0.94, about 0.95, about 0.96, about 0.97, about 0.98, or about 0.99. Q2ₘᵢₙ is at least about 0.5, about 0.55, about 0.6, about 0.65, about 0.7, about 0.75, about 0.8, about 0.82, about 0.84, about 0.86, about 0.88, about 0.90, about 0.91, about 0.92, about 0.93, about 0.94, about 0.95, about 0.96, about 0.97, about 0.98, or about 0.99. Q3ₘᵢₙ is at least about 0.5, about 0.55, about 0.6, about 0.65, about 0.7, about 0.75, about 0.8, about 0.82, about 0.84, about 0.86, about 0.88, about 0.90, about 0.91, about 0.92, about 0.93, about 0.94, about 0.95, about 0.96, about 0.97, about 0.98, about 0.99, about 0.992, about 0.994, about 0.996, about 0.998, or about 0.999. In some embodiments, Q₁_{ₘᵢₙ}_{, Q}2ₘᵢₙ, and Q3ₘᵢₙ are determined empirically from spike-in experiments. For example, Q1ₘᵢₙ, Q2ₘᵢₙ, and Q_{3 min} are chosen to allow 2 pM amplicon concentration to pass. In one embodiment, Q1ₘᵢₙ, Q2ₘᵢₙ, and Q3ₘᵢₙ are 0.98, 0.97, and 0.82, respectively. These threshold numbers were empirically derived using DNase to fragment the sample sequences. Since DNase has a T-bias, the use of other enzymes may require a shift in the threshold numbers and can be empirically derived.

In the second stage only the OTUs passing the first are considered as potential sources of cross-hybridization. In some embodiments, for each OTU, only probe-pairs with r>0.5 (these are the probe pairs considered as to be likely responsive to the target sequence) are further analyzed. In other instances, only probe pairs with r>0.6, 0.7, 0.8, or 0.9 are considered responsive and are further analyzed. Probe pairs that are unlikely to be responsive (i.e., r<0.5) are not analyzed further even if their set R, was responsive overall. R_{0.5} represents the subset of probe pairs in which all probe pairs have r>0.5. Typically, based on the interquartile Q1, Q2 and Q3 values chosen at Stage 1, most of the probe pairs in the OTUs passing Stage 1 are analyzed. In other embodiments, only the probe-pairs with r>0.55, 0.60, 0.65, 0.70, 0.75, 0.80, 0.85, or 0.90 are further analyzed.

For each probe pair in the R_{0.5} subset, the count of putatively cross-hybridizing OTUs (i.e., the number of OTUs with which the probe pair can cross-hybridize) is determined. In this process, only the OTUs that have passed Stage 1 are considered as potential sources of cross-hybridization. Each probe pair in the R_{0.5} subset is penalized by dividing its r value by the count of putatively cross-hybridizing OTUs to determine its modified possibility of being positive. The modified possibility of being positive for a probe pair may be represented by a rₓ value. rₓ may be defined as:

$${r}_{x} = \frac{r}{scalar {S}_{1 x}} \qquad Eqn . 3$$

where

S₁=Set of OTUs passing Stage 1; and,

S₁ₓ=Set of OTUs passing Stage 1 with cross hybridization potential to the given probe pair

rₓ is proportional to the response of the probe pair and the specificity of the probe pair given the community observed during the first stage. rₓ value can range from 0 to 1. For each set of probe pairs associated with an OTU, rₓ are calculated for each probe pair and ranked within the set. Interquartile Q1, Q2, Q3 values for the distribution of rₓ value in each set of probe pairs are determined. The taxon represented by the OTU is considered to be present if Q1 is greater than Qₓ₁, Q2 is greater than Qₓ₂, or Q3 is greater than Qₓ₃. Qₓ₁ is at least about 0.5, at least about 0.55, at least about 0.6, at least about 0.65, at least about 0.7 at least about 0.75, at least at least about 0.8, at least about 0.85, at least about 0.90, at least about 0.95, or at least about 0.97. Qₓ₂ is at least about 0.5, at least about 0.55, at least about 0.6, at least about 0.65, at least about 0.7 at least about 0.75, at least at least about 0.8, at least about 0.85, at least about 0.90, at least about 0.95, or at least about 0.97. Qₓ₃ is at least about 0.5, at least about 0.55, at least about 0.6, at least about 0.65, at least about 0.7 at least about 0.75, at least at least about 0.8, at least about 0.85, at least about 0.90, at least about 0.95, or at least about 0.97. In one embodiment, Qₓ₁ is at least 0.66, that is, 75% of the probe pairs in the set of the probe pairs have a rₓ value that is at least 0.66.

A two stage hybridization signal analysis procedure can be performed on hybridization signals from any array or microparticle generated data set, including data generated from the use of any combination of probes selected using the disclosed methodologies. In some embodiments, the second stage of the procedure penalizes probes based on the number of cross-hybridizations, the intensity of the cross-hybridization signals or a combination of the two.

The method disclosed herein is useful for hierarchical probe set scoring. An OTU may be present at a node at any hierarchical level on a clustering tree. As used herein, an OTU is a group of one or more organisms, such as a domain, a sub-domain, a kingdom, a subkingdom, a phylum, a sub-phylum, a class, a sub-class, an order, a sub-order, a family, a subfamily, a genus, a subgenus, a species, or any cluster. In some embodiments, a R_{0.5} set is collected for each node on the phylogenetic tree and consists of all unique probes from subordinate R_{0.5} sets. For example, for calculating rₓ values for probe pairs in a R_{0.5} set for an OTU representing an “order,” the count of putatively cross-hybridizing equally-ranked taxa (i.e., “order” node) containing at least one sequence with cross-hybridization potential is used as the denominator in Eqn. 3.

In some embodiments, the OTUs at the leaf level (e.g., species, sub-genus or genus) are first analyzed. Then each successive level of nodes in the clustering tree is analyzed. In one embodiment, the analysis is performed up to the domain level. In another embodiment, the analysis is performed up to the phylum level. In yet another embodiment, the analysis is performed up to the kingdom level. Penalization for cross-hybridization in Eqn. 3 is only performed for probes on the same taxonomy level. All present taxa are quantified using the mean scaled PM probe intensity after discarding the highest and lowest value of the set R (HybScore). In some embodiments, only taxa present at a first level are analyzed further.

In some embodiments, a summary abundance score is determined. Corrected abundance scores are created based on G+C content and uracil incorporation. Generally, probes with higher G+C content produce a higher hybridization signal that is typically compensated for correcting the abundance scores.

The probability of detection for each taxonomic node is determined by summarizing terminal node detection and the breadth of cross-hybridization relationships. Hierarchical probes are scored for evidence of novel organisms based on cluster analysis.

In some embodiments, the system is capable of analyzing other data in conjunction with that obtained from the analysis of probe hybridization signal strength. In some embodiments, the system can analyze sequencing reaction data including that obtained with high-through put sequencing techniques. In some embodiments, the sequencing data is from same regions of the same highly conserved sequence analyzed by the method disclosed herein using probes.

### High Capacity Analysis System Applications

Numerous natural human created environments can be sampled and assayed to determine the environment's microbiome composition. By having an assay system capable of detecting in a single assay the presence or quantity of at least 10,000, 20,000, 30,000, 40,000, 50,000, 60,000, 70,000, 80,000, 90,000, 100,000, 200,000, 500,000 or 1,000,000 bacterial or archeal taxa, a complete picture of the prokaryotic ecosystem can be achieved quickly and at relatively low cost providing the ability to examine numerous environments of scientific, healthcare or regulatory interest.

The elucidation of a specific microbiome associated with an ecosystem, physical environment, crop, animal, human, organ system and the like allows for the generation of a “signature,” “biosignature,” or “fingerprint” of the particular environment sampled, terms used interchangeably herein. If the biosignature is from a normal or healthy system or individual, or is from a physical environment associated with the maintenance of healthy state of individuals that inhabit the physical environment or use items produced in the physical environment, then the biosignature of the normal or healthy place can be used as a reference for the comparison of later samples from the same environment to monitor for changes that are associated with an abnormal or unhealthy state or condition. For example, if a later biosignature of a water source shows that the microbiome has shifted away from that associated with potable water, then preemptive measures could be taken to prevent a continued shift, for example by identifying a contaminant and/or contamination source and taking steps to treat and/or remove it. As a further example, if a later fingerprint of an orchard shows that the microbiome has shifted away from that associated with healthy trees and high productivity, then preemptive measures could be taken to apply nutrients that favor the growth and maintenance of the healthy microorganism or alternatively, a compost tea can be applied to boost the number of healthy microorganisms.

Similarly, a biosignature of an environment can be compared to a biosignature generated from a pool of samples that represent an average or normal biosignature for a population or collection of environments. For example, a sample from an unhealthy individual could be assayed and the microbial biosignature compared to the biosignature seen in a healthy population at large. If one or more microorganisms are detected in the unhealthy individual that are either not seen in the general population or not seen at the same prevalence then therapeutic measures can be taken to selectively eliminate or reduce in number the microorganisms associated with the unhealthy state. For instance, the microflora of the gastrointestinal tract can be compared between children that suffer from allergies and healthy children. If the allergy sufferers are shown to have one or more dominant microorganisms in their gastrointestinal tracks compared to the other children, then an available drug and/or dietary therapy that specifically targets the prevalent, abnormal microorganisms can be administered. Alternatively or additionally, the gastrointestinal population in the allergy sufferer can be shifted through the introduction of large numbers of the microorganisms associated with healthy children such as through probiotic foods or supplements. Similarly, the allergy sufferer could be given nutritional supplements that promote the growth of the health microorganisms, or the child's parents can be directed to change the child's diet to foods that favor the growth of the healthy microorganisms over that of the unhealthy ones. Once a relationship is known between the prevalence of a particular microorganism or group of microorganisms and a disease state, then disease progression or treatment response can also be monitored using the present systems and methods.

Numerous microbiomes of animals or humans can be analyzed with the present systems and methods including the gut, respiratory system, urogenital tract, mammary glands, skin, oral cavity, auditory canal, and skin. Clinical samples such as blood, sputum, nares, feces, and urine can be used with the method. From the analysis of normal individuals and those suffering from a disease or condition, a large database of fingerprints or biosignature can be assembled. By comparing the biosignatures between healthy and disease related states, associations can be made as to the influence and importance of individual components of the microbiome.

Once these associations are made, treatments can be designed and tested to alter the composition of the microbiota seen in the disease state. Additionally, by regularly monitoring the microbial composition of an affected organ system in a diseased individual, disease progress or response to therapy can be observed and if need, additional therapeutic measures taken to alter the microbiome composition to one that is more representative of that seen in a healthy population.

An interesting property of bacteria that has great importance in healthcare, water quality and food safety is quorum sensing. Many bacteria are able to sense the presence of other members of their species or related species and upon reaching a specific density the bacteria start producing various virulence or pathogenicity factors. In other words, the bacteria's gene expression is coordinated as a group. For example, some bacteria produce exopolysaccharides that are known as “slime layers.” The secretion of exopolysaccharidse can decrease the ability of white blood cells to phagocytize the microorganisms and make the microorganisms more resistant to therapeutics or cleaning agents. Traditional methodologies require the detection of specific gene expression in order to detect or study quorum sensing and other population induced effects. The present systems and methods can be used to understand the changes that occur in a microbiome that are associated with a given effect such as biofilm formation or toxicity production. One can develop protocols with the present systems and methods to look for and determine conditions that lead to quorum sensing. For example, testing samples at various timepoints and under varying conditions can lead to determining how and when to intervene or reverse population induced expression of virulence or pathogenicity factors.

For example, the clean rooms used to assemble components of satellites and other space craft can be surveyed with the present systems and methods to understand what microbial communities are present and to develop better decontamination and cleaning techniques to prevent the introduction of terrestrial microbes to other planets or samples thereof or to develop methodologies to distinguish data generated by putative extraterrestrial microorganisms from that generated by contaminating terrestrial microorganisms.

For example, food preparation sites, intensive care facilities, clean room environments such as operating theaters, drug manufacturing facilities, medical device manufacturing facilities and the like can be surveyed with the present systems and methods to ascertain the composition the local microbial communities and the quantity of the individual taxa that comprise the microbial communities. Such testing can be instrumental in preventing contamination in manufacturing processes and subsequent recalls of contaminated consumer products or the spread of infection and disease.

In one embodiment, a method is provided to identify a new indicator species for an environmental or health condition with the present systems and methods. The condition can be that of a normal or healthy state. Alternatively, the indicator species can be for an unhealthy or abnormal condition. To indentify a new indicator species, a normal sample is simultaneously assayed to determine the presence or quantity of each OTU associated with all known bacteria, archae, or fungi; this test result is compared to the results achieved in the simultaneous assay of sample from the environment of the condition where the presence or quantity of each OTU associated with all known bacteria, archae, or fungi was determined. Microorganisms that change in abundance at least 2-fold, 3-fold, 4-fold, 5-fold, 10-fold, 20-fold, 50-fold or 100-fold, either increasing in abundance or decreasing in abundance represent putative indicator species for a condition.

In some embodiments, methods are provided for identifying indicator species associated with environmental change including root growth and changes in soil composition such as increased availability of carbon substrates in soil or the presence of heavy metal or uranium, changes in soil pH, and changes in precipitation amounts and patterns. In other embodiments, methods using the present systems and methods are provided for identifying indicator species associated with coral stress and coral bleaching or changes in other marine and other aquatic environments.

In other embodiments, methods are provided for identifying indicators species associated with a disease state, disease progression, treatment regimen, probiotic administration including progression of disease in CF patients and exacerbations of COPD. In other embodiments, methods are provided for monitoring a change in the environment or health status associated with introducing one or more new microorganisms into a community. For example, measures to increase a particular microorganism's percentage of the gut microbiome in an individual, such as feeding a person yogurt or a food supplement containing L. casei, can be monitored using the present methods and systems.

### Combined Analysis

The ability to identify and quantitate the microorganisms in a sample can be combined with a gene expression technology such as a functional gene array to correlate populations with observed gene expression. Similarly, microbiome composition analysis can be correlated with the presence of chemicals, proteins including enzymes, toxins, drugs, antibiotics or other sample constituents. For instance, nucleic acids isolated from a soil sample can be analyzed to elucidate the microbiome composition (e.g. biosignature) and also to identify expressed genes. In the bare, nutrient-poor soils on the Antarctic, this analysis associated chitinase and mannanase expression with Bacteroidetes and CH₄-related genes with Alphaproteobacteria. (Yergeau et al., Environmental microarray analyses of Antarctic soil microbial communities. ISME J. 3:340-351, 2009). Significant correlations were also found between taxon abundances and C- and N-cycle gene abundance. From this data, one can predict that certain organisms or groups of organisms are required or account for the majority of an expected or observed enzymatic or degradative process. For example, members of the Bacteroidetes phylum probably degrade the majority of environmental chitin, a major constituent of exoskeletons of insect and arthropods and also of fungi cell walls, at the sample locale.

This methodology can be used to identify new antibiotic producing organisms, even ones that are unculturable. For instance, soil extracts can be tested for antibiotic activity. If a positive extract is found, a sample of the soil from which a portion was extracted for antibiotic can be analyzed for microbial composition and perhaps gene expression. Major constituents of the microbiome could be correlated with antibiotic activity with the correlation strengthened through gene expression data allowing one to predict that a particular organism or group of organisms is responsible for the observed antibiotic activity.

In one aspect, the invention provides a method for determining a condition in a sample. In one embodiment, the method comprises a) contacting said sample with a plurality of different probes; b) determining hybridization signal strength for each of said probes, wherein said determination establishes a biosignature for said sample; and, c) comparing the biosignature of said sample to a biosignature for fecal contamination. In some embodiments, a method is provided for making a prediction about a sample comprising a) determining microorganism population data as the probability of the presence or absence of at least 100 OTUs of microorganisms in said sample; b) determining gene expression data of one or more genes by said microorganisms in said sample and c) using said expression data and population data to make a prediction about said sample. In some embodiments, the prediction entails the identity of a microorganism responsible for a characteristic or condition observed in the soil or local environment.

Other combined analysis methods include the use of a diffusion chamber to retain microorganisms in a water sample while one or more constituents or parameters of the water sample are changed. For instance, the salinity or pH of the water can be changed abruptly or gradually over time. Diffusion chambers are useful to mimic the conditions of a receiving water into which is placed, for example, raw sewage. Following specific time intervals, the microbiome of the water sample in the diffusion chamber can be determined. Microorganisms that cannot tolerate the new environment conditions will die, become reduced in number due to unfavorable conditions or predation, or remain static in their numbers. In contrast, microorganisms that can tolerate the new conditions will at least maintain their number or thrive, perhaps becoming a dominant population. Use of a diffusion chamber coupled with a system capable of detecting the presence or quantity of at least 10,000 OTUs can allow the identification of microorganisms that perish or fail to thrive when placed in a new environment. Such microorganisms are termed “transient”, meaning that their percent composition of the microbiome changes quickly. The identification of transient microorganisms can be used to ascertain the time and/or place they were introduced into an environment. For example, the identification in a sample of water of an appreciable quantity of transient microorganisms associated with contaminated water that have a half-life of around 4 hours, would indicate that the microorganisms were likely introduced into the body of water within the past day (6 half-lives). Different transient microorganisms can have different half-lives for a particular condition. Armed with the knowledge of the half-lives in a receiving water of various transient microorganisms associated with contaminated water, a time course of a spill, for example a sewage discharge, can be constructed. Use of the time course can be used to pinpoint the source of the discharge and in the case of illegal discharges, for example by a cruise or cargo ship, allow the identification and citation of the violator.

Diffusion chambers can also take the form of a semi-permeable capsule, tube, rod, or sphere or other solid or semi-solid object. A microbiome or a select group of bacteria can be placed inside the capsule, that is then sealed and introduced into an environment for a specified period of time. Upon removal, the capsule is opened and the microbiome or select group of bacteria sampled to ascertain changes in the presence or quantity of the individual constituents. For example, rather than placing a sample of raw sewage into a diffusion chamber, the raw sewage could be placed into a semi-permeable capsule that is then placed into a quantity of the receiving water or into the actual receiving body of water. The capsule can be removed once or periodically from the quantity of receiving water or body of water to sample the microbiome. Alternatively, multiple single use capsules with identical quantities of the microbiome can be used, each one removed and sampled at a different time point. Microbiomes placed in capsules or other semi-permeable containers can be introduced into a living organism, usually through an orifice, to measure changes to the microbiome composition associated with a particular organ or system environment. For example, a semi¬permeable capsule or tube containing a microbiome can be introduced into the gastrointestinal system through the mouth or anus. A microbiome from a healthy individual can be introduced in this manner into an unhealthy individual, say a patient suffering from Crohn's disease or irritable bowel syndrome to ascertain the effect of the unhealthy condition on the normal, healthy individual associated microbiome. In this manner, the efficacy of drug effectiveness and treatment protocols could also be evaluated based on the effects of the gut ecology on a known microbiome.

### Low Density-Special Purpose Detection Systems

In some embodiments, probes are selected for constructing special purpose systems including those with arrays or microparticles. Typically, special purpose “low density” systems, are designed for use in a specific environment or for a particular application and usually feature a reduced number of probes, “down-selected” probes, that are specific to organisms that are known or expected to be present in the particular environment, such as associated with a particular biosignature. In some cases the biosignature is fecal contamination. Typically, a low density system comprises no more than 10, 20, 50, 100, 200, 500, 1,000, 2,000, 5,000 or 10,000 down selected probes or 5, 10, 25, 50, 100, 250, 500, 1,000, 2,500 or 5,000 down selected probes probe pairs (PM and MM probes). In some embodiments, only 1, 2, 3, 4, 5, 6, 7, 8, 9, or 10 probes are used per OTU. In further embodiments, only PM probes are used. Generally, these down-selected probes have robust hybridization signals and few or no cross hybridizations. In some embodiments, the collection of down selected probes have a median cross hybridization potential number of less than 20, 15, 10, 8, 7, 6, 5, 4, 3, 2, or 1 per probe. Frequently the down selected probes belong to OTUs that have reduced numbers of probes. In some embodiments, the OTUs of a down select probe collection have a median number of less than 25, 20, 15, 14, 13, 12, 11, 10, 9, 8, 7, 6, 5, 4, 3 or 2 probes per OTU. Generally, low density systems feature probes that recognize no more than 10, 25, 50, 100, 250, 500, 1,000, 2,000, or 5,000 taxa. For a set number of probes, a number of design strategies can be employed for low density systems. One approach is to maximize the number of OTUs identified, e.g., use one probe per OTU with no mismatch probes. Another approach is to select probes based on the desired confidence level. Here, multiple probes for each OTU along with corresponding mismatch probes may be required to achieve at least 95% confidence level for the presence and quantity of each OTU. The probes for a particular low density application can be selected by applying a sample from an appropriate environment to a high density analysis system, e.g., a detection system that can in a single assay determine the probability of the presence or quantity of at least 10,000, 20,000, 30,000, 40,000, 50,000, 60,000, 70,000, 80,000, 90,000, 100,000, 250,000, 500,00 or 1,000,000 OTUs of a single domain, such as bacteria, archea, or fungi, or alternatively, for each known OTU of a single domain. Probes associated with prevalent OTUs can be selected for a low density system. Alternately, the OTUs seen in a sample of interest can be compared with a control sample and shared OTUs subtracted out with the probes associated with the remaining OTUs selected for the low density system. Additionally, probes can be selected based on a change in prevalence of OTUs between the environment of interest and a control environment. For example, OTUs that are at least 2-fold 5-fold, 10-fold, 100-fold or 1,000-fold more abundant in the sample of interest compared to the control sample are included in the down selected probe set. Using this information, a down selected array, bead multiplex system or other low density assay system is designed.

“Low density” assays systems can be used to identify select microorganisms and determine the percentage composition of various select microorganisms in relation to each other. Low density assay systems can be constructed using probes selected through the disclosed methodologies. These low density systems can identify at least 1, 2, 3, 4, 5, 6, 7, 8, 9, 10, 15, 20, 30, 40, 50, 60, 70, 80, 90, 100, 200, 500, 1000 or more microorganisms. Representative microorganisms to be identified or quantitated are listed in Table 2.

TABLE 2
Representative Microorganisms Recognized
by Low Density Assay Systems

| Species | Application |
|---|---|
| Listeria monocytogenes | Food safety, environmental surveillance |
| | of food processing plants |
| Salmonella enterica | Food safety, environmental surveillance |
| subsp. enterica | of food processing plants |
| serovar Enteritidis | |
| Pseudomonas aeruginosa | Pulmonary health |

Low density assays systems are useful for numerous environmental and clinical applications. Exemplary applications are listed in Table 2. These applications include water quality testing for fecal or other contamination, testing for animal or human pathogens, pinpointing sources of water contamination, testing reclaimed or recycled water, testing sewage discharge streams including ocean discharge plumes, monitoring of aquaculture facilities for pathogens, monitoring beaches, swimming areas or other water related recreational facilities and predicting toxic alga blooms. Other applications include making water management or treatment decisions based on the testing or monitoring results.

Food monitoring applications include the periodic testing of production lines at food processing plants, surveying slaughter houses, inspecting the kitchens and food storage areas of restaurants, hospitals, schools, correctional facilities and other institutions for food borne pathogens such as E. coli strains O157:H7 or O111:B4, Listeria monocytogenes, or Salmonella enterica subsp. enterica serovar Enteritidis. Shellfish and shellfish producing waters can be surveyed for alga responsible for paralytic shellfish poisoning, neurotoxic shellfish poisoning, diarrhetic shellfish poisoning and amnesic shellfish poisoning. Additionally, imported foodstuffs can be screened while in customs before release to ensure food security.

Plant pathogen monitoring applications include horticulture and nursery monitoring for instance the monitoring for Phytophthora ramorum, the microorganism responsible for Sudden Oak Death, crop pathogen surveillance and disease management and forestry pathogen surveillance and disease management.

Medical conditions that can be identified, diagnosed, prognoses, track, or treated based on data obtained with a low density system include but are not limited to, cystic fibrosis, chronic obstructive pulmonary disease, Crohn's Disease, irritable bowel syndrome, cancer, rhinitis, stomach ulcers, colitis, atopy, asthma, neonatal necrotizing enterocolitis, obesity, periodontal disease and any disease or disorder caused by, aggravated by or related to the presence, absence or population change of a microorganism. Through the judicious selection of OTUs to be included in a system, the system becomes a diagnostic device capable of diagnosing one or more conditions or diseases with a high level of confidence producing very low rates of false positive or false negative readings.

Manufacturing environments for pharmaceuticals, medical devices, and other consumables or critical components where microbial contamination is a major safety concern can be surveyed for the presence of specific pathogens like Pseudomonas aeruginosa, or Staphylococcus aureus, the presence of more common microorganisms associated with humans, microorganisms associated with the presence of water or others that represent the bioburden that was previously identified in that particular environment or in similar ones.

Similarly, the construction and assembly areas for sensitive equipment including space craft can be monitored for previously identified microorganism that are known to inhabit or are most commonly introduced into such environments.

National security applications include monitoring of air, water and buildings for known bioterrorist threats such as Francisella tularensis or Bacillus anthracis. Other uses include the testing of suspicious packages or mail.

Energy security can be increased through improved gas and oil exploration methodologies and by microbial enhanced oil recovery (MEOR). Oil and gas reservoirs often leak low molecular weight components of the accumulated hydrocarbons including methane, ethane, propane and butane. These hydrocarbons can serve as food sources for a variety of microorganisms. By sampling microbial communities overlying hydrocarbon accumulations and comparing the microbiome with the microbiome observed in similar environments that are devoid of hydrocarbons, indicator species can be discovered that can then be used to identify new areas for oil and gas exploration. Soil samples can be collected from a grid array in the prospective oilfield and based on the abundance of each hydrocarbon indicator microorganism, contoured surface maps can be constructed delineating the locations of hydrocarbon plumes.

Most conventional oil recovery processes are only able to retrieve from 15 to 50% of the available oil in the reservoir. Tertiary oil recovery generally entails more expensive methods extraction techniques such as thermal recovery, chemical flooding, or miscible displacement (gas injection) to extract a last fraction of a reserve. MEOR offers a lower cost tertiary recovery method because microbes can produce biosurfactants or gases in situ using simple and cheap nutrients. Additionally, certain microorganisms can metabolize long chain hydrocarbons to create smaller, less viscous hydrocarbons (biocracking) that are easier to pump out. The ability to measure or monitor the whole microbiome of an oil field can allow for the identification and isolation of microorganisms that are associated with more productive fields. Additionally, a whole microbiome approach allows for the monitoring of a MEOR field to optimize production by observing the microbiome and adjusting nutrient levels to induce or maintain an optimal community composition for oil extraction.

Forensic science requires reliable systems for determining when events occurred, such as time of death in a murder investigation. The collection and classification of insects is currently used, but changes in microbial populations can offer another avenue to determining the time and circumstances of death.

Successful bioremediation can require active monitoring and management of microbial populations to ensure that desired species are present at the start of the bioremediation project and that their numbers are adequately maintained, perhaps through timely supplementation of essential or preferred nutrients.

In some embodiments, the low density systems also feature confirmatory probes that are specific (complimentary) for genes or sequences expressed in specific organisms. For example, the cafl virulence gene of Yersinia pestis and the zonula occludens toxin (zot) gene of Vibrio cholerae and also confirmatory probes to Y. pestis or V. cholerae.

### Kits

As used herein a “kit” refers to any delivery system for delivering materials or reagents for carrying out a method of the invention. In the context of assays, such delivery systems include systems that allow for the storage, transport, or delivery of arrays or beads with probes, reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention.

In one aspect of the invention, kits for analysis of nucleic acid targets are provided. According to one embodiment, a kit includes a plurality of probes capable of determining the presence or quantity over 10, 20, 50, 100, 200, 500, 1,000, 2,000, 5,000, 10,000, 20,000, 30,000, 40,000 50,000 or 60,000 different OTUs in a single assay. Such probes can be coupled to, for example, an array or plurality of microbeads. In some aspects a kit comprises at least 5, 10, 15, 20, 50, 100, 200, 500, 1,000, 2,000, 5,000, 10,000, 20,000, 50,000, 100,000, 200,000, 500,000, 1,000,000 or 2,000,000 interrogation probes selected using the disclosed methodologies and/or for use in the identification and/or comparison of a biosignature of one or more samples.

The kit can also include reagents for sample processing. In some embodiments, the reagents comprise reagents for the PCR amplification of sample nucleic acids including primers to amplify regions of a highly conserved sequence such as regions of the 16S rRNA gene. In still other embodiments, the reagents comprise reagents for the direct labeling of rRNA. In further embodiments, the kit includes instructions for using the kit. In other embodiments, the kit includes a password or other permission for the electronic access to a remote data analysis and manipulation software program. Such kits will have a variety of uses, including environmental monitoring, diagnosing disease, monitoring disease progress or response to treatment, and identifying a contamination source and/or the presence, absence, or amount of one or more contaminants.

### Computer Implemented Methods

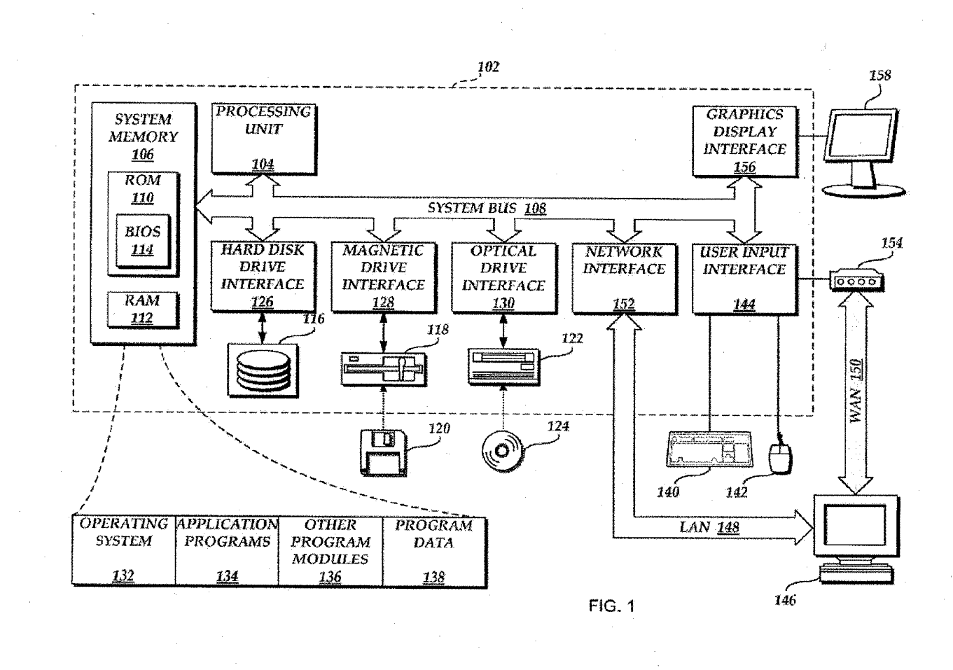

FIG. 1 illustrates an example of a suitable computing system environment or architecture in which computing subsystems may provide processing functionality to execute software embodiments of the present invention, including probe selection, analysis of samples, and remote networking. The method or system disclosed herein may also operational with numerous other general purpose or special purpose computing system including personal computers, server computers, hand-held or laptop devices, multiprocessor systems, and the like.

The method or system may be described in the general context of computer-executable instructions, such as program modules, being executed by a computer. The method or system may also be practiced in distributed computing environments where tasks are performed by remote processing devices that are linked through a communications network.

With reference to FIG. 1, an exemplary system for implementing the method or system includes a general purpose computing device in the form of a computer 102.

Components of computer 102 may include, but are not limited to, a processing unit 104, a system memory 106, and a system bus 108 that couples various system components including the system memory to the processing unit 104.

Computer 102 typically includes a variety of computer readable media. Computer readable media includes both volatile and nonvolatile media, removable and non-removable media and a may comprise computer storage media. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices.

The system memory 106 includes computer storage media in the form of volatile and/or nonvolatile memory such as read only memory (ROM) 110 and random access memory (RAM) 112. A basic input/output system 114 (BIOS), containing the basic routines that help to transfer information between elements within computer 102, such as during start-up, is typically stored in ROM 110. RAM 112 typically contains data and/or program modules that are immediately accessible to and/or presently being operated on by processing unit 104. FIG. 1 illustrates operating system 132, application programs 134 such as sequence analysis, probe selection, signal analysis and cross-hybridization analysis programs, other program modules 136, and program data 138.

The computer 102 may also include other removable/non-removable, volatile/nonvolatile computer storage media. By way of example only, FIG. 1 illustrates a hard disk drive 116 that reads from or writes to non-removable, nonvolatile magnetic media, a magnetic disk drive 118 that reads from or writes to a removable, nonvolatile magnetic disk 120, and an optical disk drive 122 that reads from or writes to a removable, nonvolatile optical disk 124 such as a CD ROM or other optical media. Other removable/non-removable, volatile/nonvolatile computer storage media that can be used in the exemplary operating environment include magnetic tape cassettes, flash memory cards, digital versatile disks, digital video tape, solid state RAM, solid state ROM, and the like. The hard disk drive 116 is typically connected to the system bus 108 through a non-removable memory interface such as interface 126, and magnetic disk drive 118 and optical disk drive 122 are typically connected to the system bus 108 by a removable memory interface, such as interface 128 or 130.

The drives and their associated computer storage media discussed above and illustrated in FIG. 1, provide storage of computer readable instructions, data structures, program modules and other data for the computer 102. In FIG. 1, for example, hard disk drive 116 is illustrated as storing operating system 132, application programs 134, other program modules 136, and program data 138. A user may enter commands and information into the computer 102 through input devices such as a keyboard 140 and a mouse, trackball or touch pad 142. These and other input devices are often connected to the processing unit 104 through a user input interface 144 that is coupled to the system bus, but may be connected by other interface and bus structures, such as a parallel port or a universal serial bus (USB). A monitor 158 or other type of display device is also connected to the system bus 108 via an interface, such as a video interface or graphics display interface 156. In addition to the monitor 158, computers may also include other peripheral output devices such as speakers (not shown) and printer (not shown), which may be connected through an output peripheral interface (not shown).

The computer 102 can be integrated into an analysis system, such as a microarray or other probe system described herein. Alternatively, the data generated by an analysis system can be imported into the computer system using various means known in the art.

The computer 102 may operate in a networked environment using logical connections to one or more remote computers or analysis systems. The remote computer may be a personal computer, a server, a router, a network PC, a peer device or other common network node, and typically includes many or all of the elements described above relative to the computer 102. The logical connections depicted in FIG. 1 include a local area network (LAN) 148 and a wide area network (WAN) 150, but may also include other networks. Such networking environments are commonplace in offices, enterprise-wide computer networks, intranets and the Internet. When used in a LAN networking environment, the computer 102 is connected to the LAN 148 through a network interface or adapter 152. When used in a WAN networking environment, the computer 102 typically includes a modem 154 or other means for establishing communications over the WAN 150, such as the Internet. The modem 154, which may be internal or external, may be connected to the system bus 108 via the user input interface 144, or other appropriate mechanism. In a networked environment, program modules depicted relative to the computer 102, or portions thereof, may be stored in the remote memory storage device.

In further aspects of the invention, computer-implemented methods are provided for analyzing the presence or quantity of over 20, 50, 100, 200, 500, 1,000, 2,000, 5,000, 10,000, 20,000, 30,000, 40,000 50,000 or 60,000 different OTUs in a single assay. In one embodiment, computer executable logic is provided for determining the presence or quantity of one or more microorganisms in a sample comprising: logic for analyzing intensities from a set of probes that selectively binds each of at least 20, 50, 100, 200, 500, 1,000, 2,000, 5,000, 10,000, 20,000, 30,000, 40,000 50,000 or 60,000 unique and highly conserved polynucleotides and determining the presence of at least 97% of all species present in said sample with at least 90%, 95%, 96%, 97%, 98%, 99% or 99.5% confidence level.

In one embodiment, computer executable logic is provided for determining probability that one or more organisms, from a set of different organisms, are present in a sample. The computer logic comprises processes or instructions for determining the likelihood that individual interrogation probe intensities are accurate based on comparison with intensities of negative control probes and positive control probes; a process or instructions for determining likelihood that an individual OTU is present based on intensities of interrogation probes from OTUs that pass a first quantile threshold; and a process or instructions for penalizing one or more OTUs that have passed the first quantile threshold based on their potential for cross-hybridizing with other probes that have also passed the first quantile threshold.

In a further embodiment, computer executable logic is provided for determining the presence of one or more microorganisms in a sample. The logic allows for the analysis of a set of at least 1000 different interrogation perfect probes. The logic further provides for the discarding of information from at least 10% of the interrogation perfect match probes in the process of making the determination. In some embodiments, the computer executable logic is stored on computer readable media and represents a computer software product.

In other embodiments, computer software products are provided wherein computer executable logic embodying aspects of the invention is stored on computer media like hard drives or optical drives. In one embodiment, the computer software products comprise instructions that when executed perform the methods described herein for determining candidate probes.

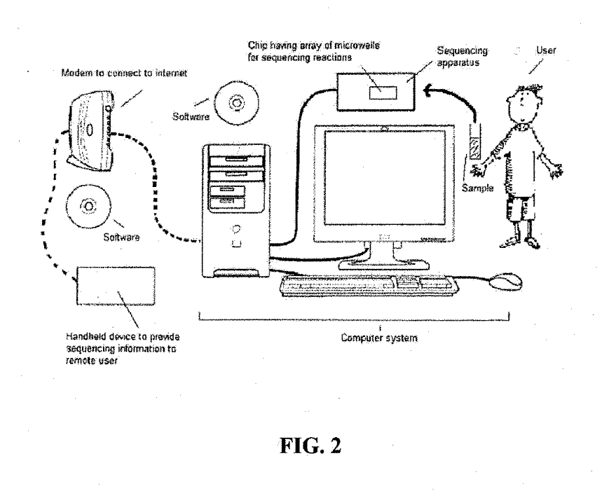

In further embodiments, computer systems are provided that can perform the methods of the inventions. In some embodiments, the computer system is integrated into and is part of an analysis system, like a flow cytometer or a microarray imaging device. In other embodiments, the computer system is connected to or ported to an analysis system. In some embodiments, the computer system is connected to an analysis system by a network connection. FIG. 2 illustrates one embodiment of a networked system for remote data acquisition or analysis that utilizes a computer system illustrated in FIG. 1. In this example, a sample is imaged using a commercially available imaging system and software. The data is outputted using a standard data format like a CEL file (AFFYMETRIX®), or a Feature Report file (NIMBLEGEN®). Then the data is sent to a remote or central location for analysis using a method of the invention. In some embodiments, a standardized analysis is performed providing signal normalization, OTU quantification, and visual analytics. In other embodiments, a customized analysis is performed using a fixed protocol designed for the user's particular needs. In still other embodiments, a user configurable analysis is used, include a protocol that allows for the user to adjust at least one variable before each analysis run.

After processing, the results are stored in an exchangeable binary format for later use or sharing. Additionally, hybridization scores and OTU probability values may be exported to a tab delimited file or in a format compatible with UniFrac (Lozupone, et al., UniFrac—an online tool for comparing microbial community diversity in a phylogenetic context, BMC Bioinformatics, 7, 371; 2006) for further statistical analysis of the detected sample communities.

In some embodiments, multiple, interactive views of the data are available, including taxonomic trees, heatmaps, hierarchical clustering, parallel coordinates (time series), bar plots, and multidimensional scaling scatterplots. In some embodiments, the taxonomy tree displays the mean intensities for each detected OTU and displays the leaves of the tree as a heatmap of samples. The tree may be dynamically pruned by filtering OTUs below a certain intensity or probability threshold. Additionally, the tree may be summarized at any level from phylum to subfamily. In other embodiments, the user can hierarchically cluster both OTUs and samples using any of the standard distance and linkage methods from the integrated C Clustering Library (de Hoon, et al., Open source clustering software, Bioinformatics, 20, 1453-1454; 2004), and the resulting dendrograms displayed in a secondary heatmap window. In some embodiments, a third window is provided that displays interactive bar plots of differential OTU intensities to facilitate pairwise comparison of samples. For any two samples, the height of the difference bars displays either the absolute or relative difference in mean intensity between OTUs. The bars may be grouped and sorted along the horizontal axis by any taxonomic rank for easy identification and comparison. Synchronized selection and filtering affords users the unique ability to seamlessly navigate between multiple views of the data. For example, users can select a cluster in the hierarchical clustering window and simultaneously view the selected organisms in the taxonomy tree, immediately revealing both their phylogenetic and environmental relationship. In further embodiments, the data from the analysis system, i.e., analysis system or flow cytometer, can be co-analyzed and displayed with high-throughput sequencing data. In some embodiments, for each organism identified as present in the sample, the user is able to view a list of other environments where the particular organism is found.

In some embodiments, the screen displays are dynamic and synchronized to allow the selection or filtration of OTUs with changes to any view simultaneously reflected in all other views. Additionally, OTUs confirmed by 16S rRNA gene, 18S rRNA gene, or 23S rRNA gene sequencing can be co-displayed in all views.

### Business Methods

In some aspects of the invention, a business method is provided wherein a client images an array or scans a lot of microparticles and sends a file containing the data to a service provider for analysis. The service provider analyzes the data and provides a report to the user in return for financial compensation. In some embodiments, the user has access to the service provider's analysis system and can manipulate and adjust the analysis parameters or the display of the results.

In another aspect of the invention, a business method is provided wherein a client sends a sample to be processed, imaged or scanned and the data analyzed for the presence or quantity of organisms. The service provider sends a report to the client in return for financial compensation. In some embodiments of the invention, the client has access to a suite of data analysis and display programs for the further analysis and viewing of the data. In further embodiments, the service provider first provides a system or kit to the client. The kit can include a system to assay a majority, or the entirety of the microbiome present or the system can contain “down-selected” probes designed for particular applications. After sample processing and imaging, the client sends the data for analysis by the service provider. In some embodiments of the invention, the client report is electronic. In other embodiments, the client is provided access to a suite of data analysis and display programs for the further viewing, manipulation, comparison and analysis of the data. In some embodiments, the client is provided access to a proprietary database in which to compare results. In other embodiments, the client is provided access to one or more public databases, or a combination of private and public database for the comparison of results. In some embodiments, the proprietary database includes the pooled results (fingerprints, biosignatures) for normal samples or the pooled results from particular abnormal situations such as a disease state. In some embodiments, the biosignatures are continuously and automatically updated upon receipt of a new sample analysis.

In some embodiments, the database further comprises highly conserved sequence listings. In some embodiments, the database is updated automatically as new sequence information becomes available, for instance, from the National Institutes of Health's Human Microbiome Project. In further embodiments, probe sets are automatically updated based on the new sequence information. Continuous upgrading of the sequence information and refinement of the probe sets allow for increasing accuracy and resolution in determining the composition of microbiomes and the quantity of their individual constituents. In some embodiments, the system compares earlier microbiome biosignatures with later microbiome biosignatures from the same or substantially similar environments and analyzes the changes in probe set composition and hybridization signal analysis parameters for information that is useful in improving or refining the discrimination between related OTUs, identification and quantification of microbiome constituents, or increasing accuracy of the determinations.

In some embodiments, the database compiles information about specific microbiomes, for example, the microbiota associated with healthy and unhealthy human intestinal microflora including, age, gender and general health status of host, geographical location of host, host's diet (i.e., Western, Asian or vegetarian), water source, host's occupation or social status, host's housing status.

In some embodiments, the reference healthy/normal signatures for adults, male and female, and children can be used as benchmarks to identify presymptomatic and symptomatic disease states, response to treatments/therapies, infection, and/or secondary infection associated with disease.

In some embodiments, the client is provided with a diagnosis or treatment recommendation based on the comparison between the client's sample microbiome and one or more reference microbiome.

In some embodiments, a database is maintained of aggregate results from routine food processing plant or slaughter house microbial inspections. A microbiome fingerprint from one or more samples from routine or emergency testing is compared against composite fingerprints of “clean plants”, “dirty plants” or plants known to have experienced a particular microbial contamination problem. The comparison results are then sent to the submitting entity.

In other embodiments, fisheries are managed based on the projected abundance of phytoplankton or absence of toxic alga blooms, such projections being derived from comparing current fingerprints of the fisheries against composite fingerprints of well managed fisheries, fisheries in decline, or known occurrences of toxic alga blooms. In other embodiments, aquaculture installations are monitored or managed by comparing a microbiome fingerprint against a database of fingerprints of healthy aquaculture installations and fingerprints of aquaculture installations during outbreaks of identified or suspected pathogens

In still other embodiments, the microbiome of a water sample from a watershed is compared to aggregated data from the entire watershed to inform management and remediation practices that optimize water quality, support fish populations, minimize toxic algae blooms or dead zones. In some embodiments, water testing is performed before and after the construction of treatment facilities to determine their effectiveness in reducing pollution and meeting regulatory standards. In still other embodiments, a sampling program is instituted wherein samples are regularly analyzed and an automated alert system notifies local, state or federal agencies when microbial levels exceed certain thresholds in recreational waters or waters sources used for domestic consumption.

Further examples of aggregate fingerprint collections include biosignatures of industrial run-off and effluent from manufacturing, processing or refinery facilities including paper and pulp mills, oil refineries, tanneries, sugar mills, chemical plants, and fecal contamination.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: PhyloChip Array Analysis

Following sample preparation, application, incubation and washing, using standard techniques, PhyloChip G3 arrays were scanned using a GeneArray Scanner from Affymetrix. The scan was captured as a pixel image using standard AFFYMETRIX® software (GCOS v1.6 using parameter: Percentile v6.0) that reduces the data to an individual row in a text-encoded table for each probe. See Table 3.

TABLE 3
Exemplary Display of Array Data
[INTENSITY]
NumberCells = 506944
CellHeader = XY

| NPIXELS | MEAN | STDV |
|---|---|---|
| 0 | 0 | 167.0 |
| 47.9 | 25 | |
| 1 | 0 | 4293.0 |
| 1060.2 | 25 | |
| 2 | 0 | 179.3 |
| 43.7 | 36 | |
| 3 | 0 | 4437.0 |
| 681.5 | 25 | |

Each analysis system had approximately 1,016,000 cells, with 1 probe sequence per cell. The analysis system scanner recorded the signal intensity across the array, which ranges from 0 to 65,000 arbitrary units (a.u) in a regular grid with −30-45 pixels per cell. A 2 pixel margin was used between adjacent cells, leaving approximately 25-40 pixels per probe of usable signal. From these pixels, the AFFYMETRIX® software computed the 75th percentile average pixel intensity (denoted as the “MEAN”), the standard deviation of signal intensity among the about 25-40 pixels (denoted as the “STDV”), and the number of pixels used per cell (denoted as “NPIXELS”). Any cells that had pixels that were three standard deviations apart in signal intensity were classified as outliers.

The analysis systems were divided into a user-defined number of horizontal and vertical divisions. By default, four horizontal and four vertical divisions were created resulting in 16 regularly spaced sectors for independent background subtraction. The background intensity was computed independently for each quadrant, as the average signal intensity of the least intense 2% (by default) of probes in that quadrant. The background intensity was then subtracted from all probes before further computation.

The noise value was estimated according to recommendations in the AFFYMETRIX® GeneChip User Guide v3.3. Noise (N) was due to variations in pixel intensity signals observed by the scanner as it read the array surface and was calculated as the standard deviation of the pixel intensities within each of the identified background cells divided by the square root of the number of pixels comprising that cell. The average of the resulting quotients was used for N in the calculations described below:

$$N = \frac{{\sum }_{j ∈ B} \frac{{S}_{i}}{\sqrt{{pix}_{i}}}}{scalar B}$$

where
B is a background cell
Sᵢ is the standard deviation among the pixels in B
pixᵢ is the count of pixels in B
scalarB is the count of all background cells, cumulative

The intensities of all probes were then scaled so that the average observed signal intensity of the spiked in probes had a pre-determined signal strength. This was accomplished by finding a scaling factor (Sf) in order to force the mean response of the corresponding PM probes to a target mean using the equation below:

$${S}_{f} = {\overline{e}}_{t} / \frac{{\sum }_{r ∈ Kpm} {e}_{i}}{scalar {K}_{pm}}$$

where
ēₜ=targeted mean intensity (default: 2500)
scalarKₚₘ=count of probes complementing any spike-in
S_{f}=scaling factor

Typically, the pre-determined signal strengths ranged from about 0 to about 65,000. Once the scaling factor was derived, all cell intensities were multiplied by the scaling factor.

The noise (N) was scaled by the same factor: Nₛ=N×S_{f}; where Nₛ=scaled noise, N=unscaled noise, and S_{f}=scaling factor.

As an alternative or optional step, MM probes with high hybridization signal responses were identified and the probe pair eliminated where:

$$[ ( \frac{MM}{PM} > {srt}_{r} ) ⋀ ( MM - PM > {N}_{s} \times {sdim}_{r} ) ] ⋁ [ PM ∈ O ] ⋁ [ MM ∈ O ]$$

where
PM=scaled intensity of the perfect match probe
MM=scaled intensity of the perfect match probe
srtᵣ=reverse standard ratio threshold (default:1.3)
sdtmᵣ=reverse standard difference threshold multiplier (default:130)
Nₛ=scaled noise
O=outlier set
   The remaining probe pairs were scored by:

$$( \frac{PM}{MM} > srt ) ⋀ ( PM - MM > {N}_{x}^{2} \times sdtm )$$

where
PM=scaled intensity of the perfect match probe
MM=scaled intensity of the perfect match probe
srt=standard ratio threshold (default:1.3)
sdtm=standard difference threshold multiplier (default:130)
Nₓ=scaled noise

After classifying an OTU as “present”, the present call was propagated upwards through the taxonomic hierarchy by considering any node (subfamily, family, order, etc.) as ‘present’ if at least one of its subordinate OTUs was present.

Hybridization intensity was the measure of OTU abundance and was calculated in arbitrary units for each probe set as the trimmed average (maximum and minimum values removed before averaging) of the PM minus MM intensity differences across the probe pairs in a given probe set.

### Example 2: Water Quality Testing—Fecal Contamination Assay

The dry weather water flow in the lower Mission Creek and Laguna watersheds of Santa Barbara, Calif., a place associated with elevated fecal indicator bacteria concentrations and human fecal contamination will be sampled with an array of the present invention. The goal is to characterize whole bacterial community composition and biogeographic pattern in an urbanized creek, 2) compare taxa detected by molecular methods to conventional fecal indicator bacteria, and 3) elucidate reliable groups of bacterial taxa to be used in culture-independent community-based fecal contamination monitoring (indicator species for fecal contamination).

The watersheds flow through an urbanized area of downtown Santa Barbara. Places to be sampled include storm drains, sections of the flowing creek, lagoon (M2, M4) and ocean. Additionally sites include where Old Mission Creek tributary discharges into Mission Creek. The dry creek flow can have many sources including underground springs in the upstream reaches, urban runoff associated with irrigation and washing, groundwater seepage, sump or basement pumps, and potentially illicit sewer connections. Sampling will be done during a period when there will not have been rain for at least 48 hours prior to or during the sampling. Besides the watershed samples, human feces and sewage will be sampled.

### Materials and Methods

Sample Description, Collection and Extraction.

Water samples are collected over 3-5 days from a watershed during a period of dry weather. Additionally, fecal samples including human feces sewage inflow are collected. Dissolved oxygen (DO), pH, temperature and salinity are measured along with each sampling. Water samples are filtered in the lab on 0.22 pm filters and extracted for DNA using the UltraClean Water DNA kit (MoBio Laboratories), and archived at −20° C. Concentrations (by IDEXX) of Total Coliforms, E. coli, and Enterococcus spp., as well as quantitative PCR (qPCR) measurements of Human-specific Bacteroides Marker (HBM) are also performed.

16S rRNA Gene Amplification for Analysis System Analysis.

The 16S rDNA is amplified from the gDNA using non-degenerate Bacterial primers 27F.jgi and 1492R. Polymerase chain reaction (PCR) is carried out using the TaKaRa Ex Taq system (Takara Bio Inc, Japan). The amplification protocol is previously described (Brodie et al., Application of a High Density Oligonucleotide Analysis system Approach to Study Bacterial Population Dynamics during Uranium Reduction and Reoxidation. Applied Environ Microbio. 72:6288¬6298, 2006).

Analysis System Processing, and Image Data Analysis.

Analysis system analysis is performed using a high-density phylogenetic analysis system (PhyloChip). The protocols are previously reported (Brodie et al., 2006). Briefly, amplicons are concentrated to a volume less than 40 μl by isopropanol precipitation. The DNA amplicons are fragmented with DNAse, biotin labeled, denatured, and hybridized to the DNA analysis system at 48° C. overnight (>16 hr). The arrays are subsequently washed and stained. Arrays are scanned using a GeneArray Scanner (Affymetrix, Santa Clara, Calif., USA). The CEL files obtained from the Affymetrix software that produces information about the fluorescence intensity of each probe (PM, MM, and control probes) are analyzed using the CELanalysis software designed by Todd DeSantis (LBNL, Berkeley, USA).

PhyloChip Data Normalization.

All statistical analyses are carried out in R (Team RCD (2008) R: A language and environment for statistical computing)). To correct for variation associated with quantification of amplicon target (quantification variation), and downstream variation associated with target fragmentation, labeling, hybridization, washing, staining and scanning (analysis system technical variation) a two-step normalization procedure is developed: First, for each PhyloChip experiment, a scaling factor best explaining the intensities of the spiked control probes under a multiplicative error model is estimated using a maximum-likelihood procedure. The intensities in each experiment are multiplied with its corresponding optimal scaling factor. In addition, the intensities for each experiment are corrected for the variation in total array intensity by dividing the intensities by its corresponding total array intensity separately for bacteria and archea.

Statistical Analysis.

All statistical analyses were carried out in R. Bray-Curtis distances were calculated using normalized fluorescence intensity with the bcdist function in the ecodist package (Goslee S C & Urban D L (2007) The ecodist package for dissimilarity-based analysis of ecological data. J Stat Softw 22(7):1-19). Mantel correlation between Bray-Curtis distance matrices of community data, geographical distance and environmental variables are calculated using the mantel function in the vegan package. Pearson's correlation is calculated with 1000 permutations of the Monte Carlo (randomization) test. Non-metric multidimensional scaling (NMDS) is performed using the metaMDS function of the vegan package. A relaxed neighbor-joining tree is generated using Clearcut (Evans J, Sheneman L, & Foster JA (2006) Relaxed neighbor-joining: a fast distance-based phylogenetic tree. Construction method. J Mol Evol 62:785-792.). Separate clearcut trees are generated for the ‘resident’ and ‘transient’ communities for each site. Unweighted UniFrac distances (Lozupone C & Knight R (2005) UniFrac: a new phylogenetic method for comparing microbial communities. Applied and Environmental Microbiology 71(12):8228-8235) are calculated for each of the sites.

### PhyloChip Derived Parameters

Fecal Taxa.

Taxa that are present in all three fecal samples, and in all 27 water samples are tabulated separately. The list of ‘Fecal Taxa’ is derived by removing those taxa found in all water samples from the taxa that are present in all three fecal samples.

Transient and Resident Subpopulations.

Taxa that are present in at least one sample from each site across the sampling period are tabulated and variances of the fluorescence intensities for those taxa are generated. The taxa in the top deciles are defined as the ‘transient’ subpopulation, and taxa in the bottom deciles were defined as the ‘resident’ subpopuation.

BBC:A.

The number of taxa in the classes of Bacilli, Bacteroidetes, Clostridia, and a -proteobacteria are tallied. The ratio is calculated using the following formula:

$$BBC : A = \frac{Bac + Bct + Cls}{A}$$

The count for unique taxa in each of the class is normalized by dividing by the total taxa in each class detected by the analysis system.

Aligned sequences from published studies are downloaded from Greengenes (DeSantis T Z, et al. (2006) Greengenes, a chimera-checked 16S rRNA gene database and workbench compatible with ARB. Applied and Environmental Microbiology 72(7):5069¬5072) and re-classified using PhyloChip taxonomy. The counts of unique taxa are tallied for each Bacterial class. BBC:A are calculated using the formula above. If no taxon is detect for a class, the count for the class is set as 0.5.

### Resolving Community Differences Among Habitats

Mission Creek samples are delineated into three habitat types: ocean, estuarine lagoon, and fresh water (creeks and storm drain effluent). Bray-Curtis distances of the watershed samples and three fecal samples (two sewage and one human feces) are calculated. Non-metric multidimensional scaling (NMDS) ordination and plotting of the first two axes are used to display the distances between samples. Bacterial communities are clearly separated by habitat types. The drain samples are most similar to the fecal samples. Lagoon samples are most similar to the ocean samples.

Signature taxa that account for the majority of differences in bacterial communities observed between habitats are identified by comparing the detected taxa at the class level among all habitat types. The number of taxas in each habitat type are divided by the total detected for each sample type to obtain a percent detection. Comparing the fecal samples to samples taken above the urban zone or those from the lagoon or ocean show that there are lower fractions of α¬proteobacteria and higher fractions of Bacilli and Clostridia. Moreover, five classes are only detected in the fecal samples: Solibacteres, Unclassified Acidobacteria, Chloroflexi-4, Coprothermobacteria and Fusobacteria. Chloroflexi-3 are only detected in creek samples, and Thermomicrobia, Unclassified Termite group 1, and Unclassified Chloroflexi only in the ocean samples. The top 10 classes with the highest standard deviations across the four habitats are (in descending order): Clostridia, α¬proteobacteria, Bacilli, γ-proteobacteria, β-proteobacteria, Actinobacteria, Flavobacteria, Bacteroidetes, Cyanobacteria, and c-proteobacteria. Of those classes, Clostridia, Bacilli, and Bacteroidetes fractions are higher, but a-proteobacteria fractions were lower. These four taxa can be used as indicators of fecal contamination.

### “Transient” and “Resident” Subpopulations

Subpopulations of taxa are identified that fluctuate the most between samplings. These are term “transient” populations. Populations that remain stable the sampling period are term “resident” populations. A comparison of taxa found in the “transient” and “resident” subpopulations illustrate differences in community composition from site to site. The six major orders (Enterobacteriales, Lactobacillales, Actinomycetales, Bacteroidales, Clostridiales and Bacillales) of the Fecal Taxa are compared to further dissect the distribution of fecal bacteria over time. The number of transient Enterobacteriales in samples from some sites are extremely high compare to the rest of the sites. While others have high resident subpopulations of Bacillales. Bacteria are identified that are ubiquitous and not affected by changes in the environmental variables measured, as measured by PhyloChip. Bacteria classes that have similar numbers of taxa throughout the watershed and fecal samples included Verrucomicrobiae, Planctomycetacia, α-proteobacteria, Anaerolinaea, Acidobacteria, Sphingobacteria, and Spirochaetes

Bacilli, Bacteroidetes and Clostridia to α-proteobacteria ratio

Four bacterial classes: Bacilli, Bacteroidetes, Clostridia and α-Proteobacteria are identified as having the highest variance among the habitat types and are further developed as fecal indicators.

The combined percentage of Bacilli, Bacteroidetes and Clostridia represent about 20-35% of total classes detected in the fecal samples, whereas their percentages at sites with expected cleaner water such as creek, lagoon and ocean are less than 10-15%. At least 45% of the taxa detected in creek water, lagoon and ocean samples are α-Proteobacteria. These microorganisms were classified as Clean Water Taxa (Table 11) as the percentage of Proteobacteria found in fecal samples is significantly lower at about 35-45%. The ratio of Bacilli, Bacteroidetes and Clostridia to α-proteobacteria (BBC:A) for fecal samples is about 3-5-fold higher than the ratios found in other habitat types. The BBC:A ratios are calculated for each site, and exhibit the same pattern as Fecal Taxa counts across all sites with ocean water having the lowest BBC:A of about 0.75-0.90 with samples close to observed sites of fecal contamination at around 1.50 to about 1.90.

This ratio contains non-coliform associated bacteria, and avoids the potential of false positive fecal detection due to growth of coliforms in the environment. Bacteroidetes and Clostridia are well known fecal-associated anaerobic bacteria. Bacilli are not especially fecal-associated but have been found in aerobic thermophilic swine wastewater bioreactors (Juteau P, Tremblay D, Villemur R, Bisaillon JG, & Beaudet R (2005) Analysis of the bacterial community inhabiting an aerobic thermophilic sequencing batch reactor (AT-SBR) treating swine waste Applied Microbiology and Biotechnology 66:115-122.). Therefore, the presence of Bacilli, Bacteroides and Clostridiales is a good indication of wastewater-, waste treatment-, and human-derived fecal pollution. α-proteobacteria are mostly phototrophic bacteria that are abundant in the environment, and play key roles in global carbon, sulfur and nitrogen cycles. Many α-proteobacteria thrive under low-nutrient conditions, and will be a good proxy for non-fecal bacteria found in non contaminated aquatic environments.

The results compare well to BBC:A found in other fecal-associated sources that are analyzed by the PhyloChip with mouse cecum, cow colon, sewage contaminated groundwater, human colon, and secondary sewage. These sources have BBC:A of above 1.2. In contrast, anaerobic groundwater has a BBC:A of 0.80-0.99.

To confirm the value of the BBC:A ratio for detecting fecal contamination, published studies of bacterial communities obtained by sequencing are analyzed. Ratios from mammalian guts, anaerobic digester sludge, ocean, Antarctic lake ice, and drinking water also demonstrate that there are differences between fecal and non-fecal samples. Mammalian gut samples have BBC:A ranging from about 10 to about 260. Anaerobic digester sludge samples have BBC:A of at least 1 to about 10. These results may reflect the highly-selected community in anaerobically-digested waste activated sludge in wastewater treatment. Non-fecal samples have BBC:A from 0 to 0.94. The sequencing results confirm that a BBC:A threshold of 1.0 can be used as a cutoff for identifying fecal pollution in water with values of 1 and above indicating polluted water. This method of calculating a BBC:A value offers numerous advantages including speed, as culturing is not required, greater detection ability as it can detect microorganisms that are currently unculturable and also avoids expense and technical problems associated with PCR cloning and high through-put sequencing.

The BBC:A ratio can be used to track the source of fecal pollution as the number usually increases in samples obtained from sites closer to a source of fecal pollution.

### Example 3: Fecal Sample Associated Taxa

Three fecal samples (human feces, from Santa Barbara, and two raw sewage, from the influent at the El Estero Wastewater Treatment plant, Santa Barbara, Calif.) were collected. Water column samples from nine locations were also collected within the Mission Creek and Laguna watersheds in Santa Barbara County, California. Taxa were present, as indicated by analysis using the PhyloChip assay, in all three fecal samples, and in all 27 water samples. The results were tabulated separately.

The list of 503 taxa are shown in Table 4 and was derived by removing those taxa found in all 27 water samples from the taxa that were present in all three fecal samples. These 503 taxa could potentially represent bacteria that are common in the human feces and sewage samples analyzed, but not found in the background environment. The similarity of the whole bacterial community composition to the fecal-associated subpopulation is useful as an indication of fecal pollution.

TABLE 4
Fecal Taxa

Bacteria; OD1; OP11-5; Unclassified; Unclassified; sf_1; 515
Bacteria; NC10; NC10-1; Unclassified; Unclassified; sf_1; 452
Bacteria; Acidobacteria; Acidobacteria-6; Unclassified; Unclassified; sf_1; 897
Bacteria; Bacteroidetes; Bacteroidetes; Bacteroidales; Unclassified; sf_15; 6233
Bacteria; Bacteroidetes; Bacteroidetes; Bacteroidales; Prevotellaceae; sf_1; 6011
Bacteria; Bacteroidetes; Bacteroidetes; Bacteroidales; Porphyromonadaceae; sf_1; 5460
Bacteria; Bacteroidetes; Bacteroidetes; Bacteroidales; Prevotellaceae; sf_1; 6047
Bacteria; Bacteroidetes; Flavobacteria; Flavobacteriales; Flavobacteriaceae; sf_1; 5942
Bacteria; Bacteroidetes; Flavobacteria; Flavobacteriales; Flavobacteriaceae; sf_1; 5589
Bacteria; Bacteroidetes; Unclassified; Unclassified; Unclassified; sf_4; 5703
Bacteria; Bacteroidetes; Sphingobacteria; Sphingobacteriales; Sphingobacteriaceae; sf_1; 5459
Bacteria; Bacteroidetes; Sphingobacteria; Sphingobacteriales; Sphingobacteriaceae; sf_1; 5492
Bacteria; Bacteroidetes; Bacteroidetes; Bacteroidales; Bacteroidaceae; sf_6; 5792
Bacteria; Bacteroidetes; Sphingobacteria; Sphingobacteriales; Crenotrichaceae; sf_11; 5619
Bacteria; Bacteroidetes; Sphingobacteria; Sphingobacteriales; Crenotrichaceae; sf_11; 6123
Bacteria; Bacteroidetes; Sphingobacteria; Sphingobacteriales; Flexibacteraceae; sf_19; 5667
Bacteria; Chlamydiae; Chlamydiae; Chlamydiales; Chlamydiaceae; sf_1; 4820
Bacteria; Cyanobacteria; Cyanobacteria; Chloroplasts; Chloroplasts; sf_11; 5123
Bacteria; marine group A; mgA-1; Unclassified; Unclassified; sf_1; 6408
Bacteria; Spirochaetes; Spirochaetes; Spirochaetales; Spirochaetaceae; sf_1; 6502
Bacteria; Spirochaetes; Spirochaetes; Spirochaetales; Spirochaetaceae; sf_1; 6494
Bacteria; Spirochaetes; Spirochaetes; Spirochaetales; Spirochaetaceae; sf_1; 6583
Bacteria; Spirochaetes; Spirochaetes; Spirochaetales; Spirochaetaceae; sf_1; 6476
Bacteria; Spirochaetes; Spirochaetes; Spirochaetales; Spirochaetaceae; sf_1; 6490
Bacteria; Spirochaetes; Spirochaetes; Spirochaetales; Spirochaetaceae; sf_1; 6506
Bacteria; Spirochaetes; Spirochaetes; Spirochaetales; Spirochaetaceae; sf_1; 6571
Bacteria; Proteobacteria; Alphaproteobacteria; Acetobacterales; Roseococcaceae; sf_1; 7500
Bacteria; Proteobacteria; Alphaproteobacteria; Acetobacterales; Acetobacteraceae; sf_1; 7600
Bacteria; Proteobacteria; Alphaproteobacteria; Azospirillales; Azospirillaceae; sf_1; 6959
Bacteria; Proteobacteria; Alphaproteobacteria; Unclassified; Unclassified; sf_6; 7312
Bacteria; Proteobacteria; Alphaproteobacteria; Unclassified; Unclassified; sf_2; 6697
Bacteria; Proteobacteria; Betaproteobacteria; Neisseriales; Neisseriaceae; sf_1; 7675
Bacteria; Proteobacteria; Betaproteobacteria; MND1 clone group; Unclassified; sf_1; 7808
Bacteria; Proteobacteria; Betaproteobacteria; Methylophilales; Methylophilaceae; sf_1; 8137
Bacteria; Proteobacteria; Betaproteobacteria; Rhodocyclales; Rhodocyclaceae; sf_1; 7817
Bacteria; Proteobacteria; Betaproteobacteria; Unclassified; Unclassified; sf_3; 8036
Bacteria; Proteobacteria; Betaproteobacteria; Burkholderiales; Alcaligenaceae; sf_1; 7768
Bacteria; Proteobacteria; Betaproteobacteria; Burkholderiales; Comamonadaceae; sf_1; 7942
Bacteria; Proteobacteria; Betaproteobacteria; Burkholderiales; Comamonadaceae; sf_1; 7847
Bacteria; Proteobacteria; Betaproteobacteria; Burkholderiales; Comamonadaceae; sf_1; 7941
Bacteria; Proteobacteria; Betaproteobacteria; Unclassified; Unclassified; sf_3; 8045
Bacteria; Proteobacteria; Betaproteobacteria; Burkholderiales; Comamonadaceae; sf_1; 7745
Bacteria; Proleobacteria; Betaproteobacteria; Burkholderiales; Ralstoniaceae; sf_1; 7778
Bacteria; Proteobacteria; Gammaproteobacteria; GAO cluster; Unclassified; sf_1; 9059
Bacteria; Proteobacteria; Gammaproteobacteria; Thiotrichales; Thiotrichaceae; sf_3; 8741
Bacteria; Proteobacteria; Gammaproteobacteria; uranium waste clones; Unclassified; sf_1; 8231
Bacteria; Proteobacteria; Gammaproteobacteria; Oceanospirillales; Oceanospirillaceae; sf_1; 8596
Bacteria; Proteobacteria; Gammaproteobacteria; Legionellales; Coxiellaceae; sf_3; 9444
Bacteria; Proteobacteria; Gammaproteobacteria; Oceanospirillales; Alcanivoraceae; sf_1; 9658
Bacteria; Proteobacteria; Gammaproteobacteria; Pseudomonadales; Pseudomonadaceae; sf_1; 8601
Bacteria; Proteobacteria; Gammaproteobacteria; Unclassified; Unclassified; sf_3; 8959
Bacteria; Proteobacteria; Gammaproteobacteria; Alteromonadales; Alteromonadaceae; sf_1; 9486
Bacteria; Proteobacteria; Gammaproteobacteria; Alteromonadales; Alteromonadaceae; sf_1; 8863
Bacteria; Proteobacteria; Gammaproteobacteria; Alteromonadales; Alteromonadaceae; sf_1; 9501
Bacteria; Proteobacteria; Gammaproteobacteria; Alteromonadales; Shewanellaceae; sf_1; 8581
Bacteria; Proteobacteria; Gammaproteobacteria; Pasteurellales; Pasteurellaceae; sf_1; 9237
Bacteria; Proteobacteria; Gammaproteobacteria; Enterobacteriales; Enterobacteriaceae; sf_1; 8554
Bacteria; Proteobacteria; Gammaproteobacteria; Enterobacteriales; Enterobacteriaceae; sf_1; 8885
Bacteria; Proteobacteria; Gammaproteobacteria; Enterobacteriales; Enterobacteriaceae; sf_1; 8700
Bacteria; Proteobacteria; Gammaproteobacteria; Enterobacteriales; Enterobacteriaceae; sf_1; 8529
Bacteria; Proteobacteria; Gammaproteobacteria; Enterobacteriales; Enterobacteriaceae; sf_1; 8770
Bacteria; Proteobacteria; Gammaproteobacteria; Enterobacteriales; Enterobacteriaceae; sf_1; 8225
Bacteria; Unclassified; Unclassified; Unclassified; Unclassified; sf_160; 10012
Bacteria; Proteobacteria; Deltaproteobacteria; Desulfovibrionales; Desulfovibrionaceae; sf_1; 10189
Bacteria; Proteobacteria; Deltaproteobacteria; Syntrophobacterales; Syntrophaceae; sf_3; 9665
Bacteria; Proteobacteria; Epsilonproteobacteria; Campylobacterales; Helicobacteraceae; sf_23; 10443
Bacteria; Proteobacteria; Epsilonproteobacteria; Campylobacterales; Helicobacteraceae; sf_3; 10576
Bacteria; Proteobacteria; Epsilonproteobacteria; Campylobacterales; Unclassified; sf_1; 10407
Bacteria; Gemmatimonadetes; Unclassified; Unclassified; Unclassified; sf_5; 317
Bacteria; Actinobacteria; Actinobacteria; Rubrobacterales; Rubrobacteraceae; sf_1; 1551
Bacteria; Actinobacteria; Actinobacteria; Acidimicrobiales; Unclassified; sf_1; 1666
Bacteria; Actinobacteria; BD2-10 group; Unclassified; Unclassified; sf_2; 1652
Bacteria; Actinobacteria; Actinobacteria; Actinomycetales; Unclassified; sf_3; 2045
Bacteria; Actinobacteria; Actinobacteria; Actinomycetales; Cellulomonadaceae; sf_1; 1748
Bacteria; Actinobacteria; Actinobacteria; Actinomycetales; Actinomycetaceae; sf_1; 1684
Bacteria; Actinobacteria; Actinobacteria; Bifidobacteriales; Bifidobacteriaceae; sf_1; 1444
Bacteria; Actinobacteria; Actinobacteria; Actinomycetales; Kineosporiaceae; sf_1; 1598
Bacteria; Actinobacteria; Actinobacteria; Actinomycetales; Kineosporiaceae; sf_1; 1961
Bacteria; Actinobacteria; Actinobacteria; Actinomycetales; Corynebacteriaceae; sf_1; 1517
Bacteria; Actinobacteria; Actinobacteria; Actinomycetales; Corynebacteriaceae; sf_1; 1803
Bacteria; Actinobacteria; Actinobacteria; Actinomycetales; Dietziaceae; sf_1; 1970
Bacteria; Firmicutes; Unclassified; Unclassified; Unclassified; sf_8; 2433
Bacteria; Firmicutes; Clostridia; Unclassified; Unclassified; sf_4; 2398
Bacteria; Chloroflexi; Dehalococcoidetes; Unclassified; Unclassified; sf_1; 2339
Bacteria; Chloroflexi; Dehalococcoidetes; Unclassified; Unclassified; sf_1; 2497
Bacteria; Firmicutes; Clostridia; Clostridiales; Peptococc/Acidaminococc; sf_11; 709
Bacteria; Firmicutes; Clostridia; Clostridiales; Peptococc/Acidaminococc; sf_11; 242
Bacteria; Firmicutes; Clostridia; Clostridiales; Lachnospiraceae; sf_5; 3042
Bacteria; Firmicutes; Clostridia; Clostridiales; Lachnospiraceae; sf_5; 3076
Bacteria; Firmicutes; Clostridia; Clostridiales; Lachnospiraceae; sf_5; 3171
Bacteria; Firmicutes; Clostridia; Clostridiales; Lachnospiraceae; sf_5; 2681
Bacteria; Firmicutes; Clostridia; Clostridiales; Peptostreptococcaceae; sf_5; 2721
Bacteria; Firmicutes; Clostridia; Clostridiales; Peptostreptococcaceae; sf_5; 2796
Bacteria; Firmicutes; Clostridia; Clostridiales; Clostridiaceae; sf_12; 2915
Bacteria; TM7; Unclassified; Unclassified; Unclassified; sf_1; 3025
Bacteria; Firmicutes; Bacilli; Bacillales; Paenibacillaceae; sf_1; 3299
Bacteria; Firmicutes; Bacilli; Bacillales; Halobacillaceae; sf_1; 3344
Bacteria; Firmicutes; Bacilli; Lactobacillales; Streptococcaceae; sf_1; 3722
Bacteria; Firmicutes; Mollicutes; Acholeplasmatales; Acholeplasmataceae; sf_1; 3976
Bacteria; Acidobacteria; Unclassified; Unclassified; Unclassified; sf_1; 4222
Bacteria; Firmicutes; Clostridia; Clostridiales; Clostridiaceae; sf_12; 4406
Bacteria; Firmicutes; Clostridia; Clostridiales; Lachnospiraceae; sf_5; 4212
Bacteria; Firmicutes; Clostridia; Clostridiales; Clostridiaceae; sf_12; 4359
Bacteria; Firmicutes; Clostridia; Clostridiales; Clostridiaceae; sf_12; 4475
Bacteria; Unclassified; Unclassified; Unclassified; Unclassified; sf_160; 4410
Bacteria; Firmicutes; Clostridia; Clostridiales; Clostridiaceae; sf_12; 4306
Bacteria; Firmicutes; Clostridia; Clostridiales; Clostridiaceae; sf_12; 4427
Bacteria; Firmicutes; Clostridia; Clostridiales; Clostridiaceae; sf_12; 4296
Bacteria; Verrucomicrobia; Verrucomicrobiae; Verrucomicrobiales; Verrucomicrobia subdivision 7; sf_1; 559
Bacteria; Bacteroidetes; Flavobacteria; Flavobacteriales; Flavobacteriaceae; sf_1; 6200
Bacteria; Proteobacteria; Betaproteobacteria; Burkholderiales; Comamonadaceae; sf_1; 7971
Bacteria; Verrucomicrobia; Verrucomicrobiae; Verrucomicrobiales; Verrucomicrobia subdivision 5; sf_1; 533
Bacteria; Verrucomicrobia; Unclassified; Unclassified; Unclassified; sf_4; 288
Bacteria; Bacteroidetes; Bacteroidetes; Bacteroidales; Bacteroidaceae; sf_12; 5320
Bacteria; Bacteroidetes; Bacteroidetes; Bacteroidales; Bacteroidaceae; sf_12; 5950
Bacteria; Bacteroidetes; Bacteroidetes; Bacteroidales; Prevotellaceae; sf_1; 5905
Bacteria; Cyanobacteria; Cyanobacteria; Nostocales; Unclassified; sf_1; 5047
Bacteria; Cyanobacteria; Cyanobacteria; Nostocales; Unclassified; sf_1; 5072
Bacteria; Cyanobacteria; Cyanobacteria; Nostocales; Unclassified; sf_1; 5191
Bacteria; Cyanobacteria; Cyanobacteria; Nostocales; Unclassified; sf_1; 5199
Bacteria; BRC1; Unclassified; Unclassified; Unclassified; sf_1; 5051
Bacteria; Cyanobacteria; Cyanobacteria; Chloroplasts; Chloroplasts; sf_5; 5130
Bacteria; Unclassified; Unclassified; Unclassified; Unclassified; sf_160; 6337
Bacteria; Unclassified; Unclassified; Unclassified; Unclassified; sf_160; 6360
Bacteria; Acidobacteria; Acidobacteria; Acidobacteriales; Acidobacteriaceae; sf_14; 6425
Bacteria; Spirochaetes; Spirochaetes; Spirochaetales; Spirochaetaceae; sf_1; 6529
Bacteria; Proteobacteria; Alphaproteobacteria; Acetobacterales; Acetobacteraceae; sf_1; 7529
Bacteria; Proteobacteria; Betaproteobacteria; Unclassified; Unclassified; sf_3; 8007
Bacteria; Proteobacteria; Betaproteobacteria; MND1 clone group; Unclassified; sf_1; 7993
Bacteria; Proteobacteria; Gammaproteobacteria; Alteromonadales; Alteromonadaceae; sf_1; 9491
Bacteria; Proteobacteria; Gammaproteobacteria; Alteromonadales; Shewanellaceae; sf_1; 8201
Bacteria; Proteobacteria; Gammaproteobacteria; Pasteurellales; Pasteurellaceae; sf_1; 8409
Bacteria; Proteobacteria; Gammaproteobacteria; Enterobacteriales; Enterobacteriaceae; sf_1; 9363
Bacteria; Proteobacteria; Gammaproteobacteria; Enterobacteriales; Enterobacteriaceae; sf_1; 8934
Bacteria; Proteobacteria; Gammaproteobacteria; Enterobacteriales; Enterobacteriaceae; sf_1; 8467
Bacteria; Proteobacteria; Gammaproteobacteria; Enterobacteriales; Enterobacteriaceae; sf_1; 8530
Bacteria; Proteobacteria; Gammaproteobacteria; Enterobacteriales; Enterobacteriaceae; sf_1; 9390
Bacteria; Proteobacteria; Gammaproteobacteria; Enterobacteriales; Enterobacteriaceae; sf_1; 8251
Bacteria; Proteobacteria; Gammaproteobacteria; Enterobacteriales; Enterobacteriaceae; sf_1; 8890
Bacteria; Proteobacteria; Gammaproteobacteria; Enterobacteriales; Enterobacteriaceae; sf_1; 8362
Bacteria; Proteobacteria; Gammaproteobacteria; Enterobacteriales; Enterobacteriaceae; sf_1; 8510
Bacteria; Proteobacteria; Gammaproteobacteria; Enterobacteriales; Enterobacteriaceae; sf_1; 8711
Bacteria; Proteobacteria; Gammaproteobacteria; Enterobacteriales; Enterobacteriaceae; sf_1; 8712
Bacteria; Proteobacteria; Gammaproteobacteria; Enterobacteriales; Enterobacteriaceae; sf_1; 8739
Bacteria; Proteobacteria; Gammaproteobacteria; Enterobacteriales; Enterobacteriaceae; sf_1; 9417
Bacteria; Proteobacteria; Gammaproteobacteria; Enterobacteriales; Enterobacteriaceae; sf_1; 8473
Bacteria; Proteobacteria; Deltaproteobacteria; Myxococcales; Polyangiaceae; sf_3; 10082
Bacteria; Proteobacteria; Deltaproteobacteria; Syntrophobacterales; Syntrophobacteraceae; sf_1; 9864
Bacteria; Proteobacteria; Deltaproteobacteria; Syntrophobacterales; Syntrophobacteraceae; sf_1; 9731
Bacteria; Actinobacteria; Actinobacteria; Actinomycetales; Frankiaceae; sf_1; 1286
Bacteria; Actinobacteria; Actinobacteria; Actinomycetales; Dietziaceae; sf_1; 1872
Bacteria; Chloroflexi; Dehalococcoidetes; Unclassified; Unclassified; sf_1; 2397
Bacteria; Chloroflexi; Unclassified; Unclassified; Unclassified; sf_1; 2534
Bacteria; Firmicutes; Clostridia; Clostridiales; Peptococc/Acidaminococc; sf_11; 710
Bacteria; Firmicutes; Clostridia; Clostridiales; Peptococc/Acidaminococc; sf_11; 300
Bacteria; Firmicutes; Clostridia; Clostridiales; Lachnospiraceae; sf_5; 3218
Bacteria; Firmicutes; Catabacter; Unclassified; Unclassified; sf_4; 2716
Bacteria; Firmicutes; Clostridia; Clostridiales; Peptostreptococcaceae; sf_5; 2679
Bacteria; Firmicutes; Clostridia; Clostridiales; Peptostreptococcaceae; sf_5; 2714
Bacteria; Firmicutes; Clostridia; Clostridiales; Peptostreptococcaceae; sf_5; 2722
Bacteria; Firmicutes; Clostridia; Clostridiales; Peptostreptococcaceae; sf_5; 2993
Bacteria; Firmicutes; Clostridia; Clostridiales; Clostridiaceae; sf_12; 3021
Bacteria; Firmicutes; Bacilli; Lactobacillales; Carnobacteriaceae; sf_1; 3536
Bacteria; Firmicutes; Bacilli; Lactobacillales; Streptococcaceae; sf_1; 3869
Bacteria; Firmicutes; Bacilli; Lactobacillales; Streptococcaceae; sf_1; 3588
Bacteria; Firmicutes; Mollicutes; Anaeroplasmatales; Erysipelotrichaceae; sf_3; 3981
Bacteria; Firmicutes; Catabacter; Unclassified; Unclassified; sf_1; 4261
Bacteria; Firmicutes; Clostridia; Clostridiales; Lachnospiraceae; sf_5; 4571
Bacteria; Firmicutes; Clostridia; Clostridiales; Lachnospiraceae; sf_5; 4623
Bacteria; Firmicutes; Clostridia; Clostridiales; Clostridiaceae; sf_12; 4589
Bacteria; Bacteroidetes; Bacteroidetes; Bacteroidales; Unclassified; sf_15; 5511
Bacteria; Proteobacteria; Gammaproteobacteria; Enterobacteriales; Enterobacteriaceae; sf_1; 8286
Bacteria; Proteobacteria; Deltaproteobacteria; Desulfobacterales; Desulfobacteraceae; sf_5; 10136
Bacteria; Aquificae; Aquificae; Aquificales; Unclassified; sf_1; 2364
Bacteria; Verrucomicrobia; Verrucomicrobiae; Verrucomicrobiales; Verrucomicrobiaceae; sf_6; 871
Bacteria; Verrucomicrobia; Verrucomicrobiae; Verrucomicrobiales; Verrucomicrobiaceae; sf_1; 1024
Bacteria; Bacteroidetes; Sphingobacteria; Sphingobacteriales; Crenotrichaceae; sf_11; 5334
Bacteria; Chloroflexi; Anaerolineae; Unclassified; Unclassified; sf_9; 72
Bacteria; Cyanobacteria; Cyanobacteria; Nostocales; Unclassified; sf_1; 5004
Bacteria; Acidobacteria; Solibacteres; Unclassified; Unclassified; sf_1; 6426
Bacteria; Spirochaetes; Spirochaetes; Spirochaetales; Spirochaetaceae; sf_1; 6507
Bacteria; Spirochaetes; Spirochaetes; Spirochaetales; Spirochaetaceae; sf_1; 6460
Bacteria; Spirochaetes; Spirochaetes; Spirochaetales; Spirochaetaceae; sf_1; 6579
Bacteria; Spirochaetes; Spirochaetes; Spirochaetales; Leptospiraceae; sf_3; 6470
Bacteria; Proteobacteria; Alphaproteobacteria; Unclassified; Unclassified; sf_6; 7647
Bacteria; Proteobacteria; Betaproteobacteria; Nitrosomonadales; Nitrosomonadaceae; sf_1; 8145
Bacteria; Proteobacteria; Betaproteobacteria; Burkholderiales; Comamonadaceae; sf_1; 7822
Bacteria; Proteobacteria; Betaproteobacteria; Unclassified; Unclassified; sf_3; 7954
Bacteria; Proteobacteria; Gammaproteobacteria; Thiotrichales; Thiotrichaceae; sf_3; 8321
Bacteria; Proteobacteria; Gaminaproteobacteria; Thiotrichales; Francisellaceae; sf_1; 9554
Bacteria; Proteobacteria; Gammaproteobacteria; Xanthomonadales; Xanthomonadaceae; sf_3; 8983
Bacteria; Proteobacteria; Gammaproteobacteria; Legionellales; Coxiellaceae; sf_3; 8969
Bacteria; Proteobacteria; Gammaproteobacteria; Oceanospirillales; Halomonadaceae; sf_1; 8598
Bacteria; Proteobacteria; Gammaproteobacteria; Alteromonadales; Alteromonadaceae; sf_1; 9236
Bacteria; Proteobacteria; Gammaproteobacteria; Enterobacteriales; Enterobacteriaceae; sf_1; 8742
Bacteria; Proteobacteria; Gammaproteobacteria; Enterobacteriales; Enterobacteriaceae; sf_1; 9135
Bacteria; Proteobacteria; Gammaproteobacteria; Enterobacteriales; Enterobacteriaceae; sf_1; 9496
Bacteria; Proteobacteria; Gammaproteobacteria; Enterobacteriales; Enterobacteriaceae; sf_1; 8886
Bacteria; Proteobacteria; Gammaproteobacteria; Enterobacteriales; Enterobacteriaceae; sf_1; 9651
Bacteria; Proteobacteria; Gammaproteobacteria; Enterobacteriales; Enterobacteriaceae; sf_1; 8379
Bacteria; Proteobacteria; Gammaproteobacteria; Enterobacteriales; Enterobacteriaceae; sf_1; 9142
Bacteria; Proteobacteria; Gammaproteobacteria; Enterobacteriales; Enterobacteriaceae; sf_1; 9345
Bacteria; Proteobacteria; Gammaproteobacteria; Enterobacteriales; Enterobacteriaceae; sf_1; 8282
Bacteria; Proteobacteria; Gammaproteobacteria; Enterobacteriales; Unclassified; sf_1; 8430
Bacteria; Proteobacteria; Gammaproteobacteria; Enterobacteriales; Enterobacteriaceae; sf_1; 8505
Bacteria; Proteobacteria; Gammaproteobacteria; Enterobacteriales; Enterobacteriaceae; sf_1; 8528
Bacteria; Proteobacteria; Gammaproteobacteria; Enterobacteriales; Enterobacteriaceae; sf_1; 8936
Bacteria; Proteobacteria; Gammaproteobacteria; Enterobacteriales; Enterobacteriaceae; sf_1; 9060
Bacteria; Proteobacteria; Gammaproteobacteria; Enterobacteriales; Enterobacteriaceae; sf_1; 9274
Bacteria; Proteobacteria; Deltaproteobacteria; Desulfovibrionales; Desulfovibrionaceae; sf_1; 10212
Bacteria; Proteobacteria; Deltaproteobacteria; EB1021 group; Unclassified; sf_4; 10024
Bacteria; Proteobacteria; Epsilonproteobacteria; Campylobacterales; Campylobacteraceae; sf_3; 10397
Bacteria; Actinobacteria; Actinobacteria; Acidimicrobiales; Microthrixineae; sf_1; 1576
Bacteria; Actinobacteria; Actinobacteria; Actinomycetales; Pseudonocardiaceae; sf_1; 1863
Bacteria; Firmicutes; Clostridia; Clostridiales; Clostridiaceae; sf_12; 252
Bacteria; Firmicutes; Clostridia; Clostridiales; Lachnospiraceae; sf_5; 2709
Bacteria; Firmicutes; Clostridia; Clostridiales; Lachnospiraceae; sf_5; 3060
Bacteria; Firmicutes; Clostridia; Clostridiales; Peptostreptococcaceae; sf_5; 2729
Bacteria; Firmicutes; Bacilli; Bacillales; Bacillaceae; sf_1; 234
Bacteria; Firmicutes; Bacilli; Bacillales; Bacillaceae; sf_1; 3460
Bacteria; Firmicutes; Bacilli; Bacillales; Halobacillaceae; sf_1; 3769
Bacteria; Firmicutes; Bacilli; Bacillales; Bacillaceae; sf_1; 3900
Bacteria; Firmicutes; Bacilli; Bacillales; Caryophanaceae; sf_1; 3285
Bacteria; Firmicutes; Bacilli; Lactobacillales; Lactobacillaceae; sf_1; 3768
Bacteria; Firmicutes; Mollicutes; Acholeplasmatales; Acholeplasmataceae; sf_1; 4044
Bacteria; Firmicutes; Mollicutes; Acholeplasmatales; Acholeplasmataceae; sf_1; 4045
Bacteria; Firmicutes; Mollicutes; Anaeroplasmatales; Erysipelotrichaceae; sf_3; 3965
Bacteria; Firmicutes; Clostridia; Clostridiales; Clostridiaceae; sf_12; 4614
Bacteria; Firmicutes; Clostridia; Clostridiales; Clostridiaceae; sf_12; 4415
Bacteria; Firmicutes; Clostridia; Clostridiales; Clostridiaceae; sf_12; 4548
Bacteria; Firmicutes; Clostridia; Clostridiales; Clostridiaceae; sf_12; 4555
Bacteria; Nitrospira; Nitrospira; Nitrospirales; Nitrospiraceae; sf_2; 542
Bacteria; Nitrospira; Nitrospira; Nitrospirales; Nitrospiraceae; sf_2; 697
Bacteria; Natronoanaerobium; Unclassified; Unclassified; Unclassified; sf_1; 769
Bacteria; Acidobacteria; Acidobacteria-4; Ellin6075/11-25; Unclassified; sf_1; 435
Bacteria; Bacteroidetes; Bacteroidetes; Bacteroidales; Prevotellaceae; sf_1; 5484
Bacteria; Cyanobacteria; Cyanobacteria; Pseudanabaena; Unclassified; sf_1; 5008
Bacteria; marine group A; mgA-1; Unclassified; Unclassified; sf_1; 6454
Bacteria; Proteobacteria; Alphaproteobacteria; Verorhodospirilla; Unclassified; sf_1; 7109
Bacteria; Proteobacteria; Alphaproteobacteria; Bradyrhizobiales; Beijerinck/Rhodoplan/Methylocyst; sf_3; 7401
Bacteria; Proteobacteria; Betaproteobacteria; Rhodocyclales; Rhodocyclaceae; sf_1; 7951
Bacteria; Proteobacteria; Gammaproteobacteria; Thiotrichales; Thiotrichaceae; sf_3; 9321
Bacteria; Proteobacteria; Gammaproteobacteria; Oceanospirillales; Halomonadaceae; sf_1; 8317
Bacteria; Proteobacteria; Gammaproteobacteria; Pseudomonadales; Moraxellaceae; sf_3; 9359
Bacteria; Proteobacteria; Gammaproteobacteria; Alteromonadales; Alteromonadaceae; sf_1; 8533
Bacteria; Proteobacteria; Gammaproteobacteria; Enterobacteriales; Enterobacteriaceae; sf_1; 9358
Bacteria; Proteobacteria; Gammaproteobacteria; Enterobacteriales; Enterobacteriaceae; sf_1; 9302
Bacteria; Proteobacteria; Gammaproteobacteria; Enterobacteriales; Enterobacteriaceae; sf_1; 8603
Bacteria; Proteobacteria; Gammaproteobacteria; Enterobacteriales; Enterobacteriaceae; sf_1; 9265
Bacteria; Proteobacteria; Deltaproteobacteria; Myxococcales; Unclassified; sf_1; 10259
Bacteria; Proteobacteria; Deltaproteobacteria; Unclassified; Unclassified; sf_7; 10048
Bacteria; Proteobacteria; Deltaproteobacteria; EB1021group; Unclassified; sf_4; 9741
Bacteria; Chloroflexi; Chloroflexi-4; Unclassified; Unclassified; sf_2; 2344
Bacteria; Firmicutes; Clostridia; Clostridiales; Peptococc/Acidaminococc; sf_11; 39
Bacteria; Firmicutes; Clostridia; Clostridiales; Lachnospiraceae; sf_5; 3036
Bacteria; Firmicutes; Clostridia; Clostridiales; Lachnospiraceae; sf_5; 2825
Bacteria; Firmicutes; Clostridia; Clostridiales; Peptostreptococcaceae; sf_5; 58
Bacteria; Firmicutes; Bacilli; Lactobacillales; Lactobacillaceae; sf_1; 3566
Bacteria; Firmicutes; Bacilli; Lactobacillales; Streptococcaceae; sf_1; 3251
Bacteria; Firmicutes; Mollicutes; Anaeroplasmatales; Erysipelotrichaceae; sf_3; 768
Bacteria; Firmicutes; Clostridia; Clostridiales; Clostridiaceae; sf_12; 4297
Bacteria; Firmicutes; Clostridia; Clostridiales; Clostridiaceae; sf_12; 4299
Bacteria; Firmicutes; Clostridia; Clostridiales; Clostridiaceae; sf_12; 4502
Bacteria; Firmicutes; Clostridia; Clostridiales; Clostridiaceae; sf_12; 4554
Bacteria; Firmicutes; Clostridia; Clostridiales; Clostridiaceae; sf_12; 4157
Bacteria; Firmicutes; Clostridia; Clostridiales; Clostridiaceae; sf_12; 4267
Bacteria; Bacteroidetes; Bacteroidetes; Bacteroidales; Porphyromonadaceae; sf_1; 5961
Bacteria; Bacteroidetes; Bacteroidetes; Bacteroidales; Prevotellaceae; sf_1; 5916
Bacteria; Bacteroidetes; Flavobacteria; Flavobacteriales; Flavobacteriaceae; sf_1; 5473
Bacteria; Cyanobacteria; Cyanobacteria; Nostocales; Unclassified; sf_1; 5028
Bacteria; Cyanobacteria; Cyanobacteria; Nostocales; Unclassified; sf_1; 5174
Bacteria; Cyanobacteria; Cyanobacteria; Nostocales; Unclassified; sf_1; 5175
Bacteria; TM7; Unclassified; Unclassified; Unclassified; sf_1; 5061
Bacteria; Proteobacteria; Betaproteobacteria; Burkholderiales; Burkholderiaceae; sf_1; 7782
Bacteria; Proteobacteria; Gammaproteobacteria; Chromatiales; Unclassified; sf_1; 9282
Bacteria; Proteobacteria; Gammaproteobacteria; Oceanospirillales; Halomonadaceae; sf_1; 8854
Bacteria; Proteobacteria; Gammaproteobacteria; Pseudomonadales; Pseudomonadaceae; sf_1; 8209
Bacteria; Proteobacteria; Gammaproteobacteria; Enterobacteriales; Enterobacteriaceae; sf_6; 8783
Bacteria; Firmicutes; Clostridia; Clostridiales; Peptococc/Acidaminococc; sf_11; 304
Bacteria; Firmicutes; Clostridia; Clostridiales; Peptococc/Acidaminococc; sf_11; 131
Bacteria; Firmicutes; Clostridia; Clostridiales; Clostridiaceae; sf_12; 206
Bacteria; Firmicutes; Clostridia; Clostridiales; Lachnospiraceae; sf_5; 2834
Bacteria; Firmicutes; Clostridia; Clostridiales; Lachnospiraceae; sf_5; 2844
Bacteria; Firmicutes; Clostridia; Clostridiales; Peptostreptococcaceae; sf_5; 2694
Bacteria; Firmicutes; Clostridia; Clostridiales; Peptostreptococcaceae; sf_5; 3080
Bacteria; Firmicutes; Clostridia; Clostridiales; Peptostreptococcaceae; sf_5; 3182
Bacteria; Firmicutes; Clostridia; Clostridiales; Peptostreptococcaceae; sf_5; 619
Bacteria; Firmicutes; Bacilli; Bacillales; Bacillaceae; sf_1; 305
Bacteria; Firmicutes; Bacilli; Bacillales; Bacillaceae; sf_1; 3836
Bacteria; Firmicutes; Bacilli; Bacillales; Bacillaceae; sf_1; 462
Bacteria; Firmicutes; Bacilli; Bacillales; Bacillaceae; sf_1; 3831
Bacteria; Firmicutes; Bacilli; Lactobacillales; Enterococcaceae; sf_1; 3288
Bacteria; Firmicutes; Bacilli; Lactobacillales; Enterococcaceae; sf_1; 3598
Bacteria; Firmicutes; Mollicutes; Acholeplasmatales; Acholeplasmataceae; sf_1; 3961
Bacteria; Firmicutes; Mollicutes; Acholeplasmatales; Acholeplasmataceae; sf_1; 3975
Bacteria; Firmicutes; Mollicutes; Anaeroplasmatales; Erysipelotrichaceae; sf_3; 3952
Bacteria; Firmicutes; Clostridia; Clostridiales; Clostridiaceae; sf_12; 4584
Bacteria; Firmicutes; Clostridia; Clostridiales; Clostridiaceae; sf_12; 4459
Bacteria; Firmicutes; Clostridia; Clostridiales; Lachnospiraceae; sf_5; 4533
Bacteria; Firmicutes; Clostridia; Clostridiales; Lachnospiraceae; sf_5; 4539
Bacteria; Firmicutes; Clostridia; Clostridiales; Clostridiaceae; sf_12; 4637
Bacteria; Firmicutes; Catabacter; Unclassified; Unclassified; sf_4; 4526
Bacteria; Firmicutes; Clostridia; Clostridiales; Clostridiaceae; sf_12; 4560
Bacteria; Firmicutes; Clostridia; Clostridiales; Clostridiaceae; sf_12; 4310
Bacteria; Proteobacteria; Betaproteobacteria; Burkholderiales; Unclassified; sf_1; 7879
Bacteria; Proteobacteria; Gammaproteobacteria; Xanthomonadales; Xanthomonadaceae; sf_3; 9211
Bacteria; Proteobacteria; Gammaproteobacteria; Alteromonadales; Pseudoalteromonadaceae; sf_1; 9339
Bacteria; Proteobacteria; Deltaproteobacteria; Myxococcales; Polyangiaceae; sf_3; 10065
Bacteria; Proteobacteria; Deltaproteobacteria; Unclassified; Unclassified; sf_9; 9738
Bacteria; Proteobacteria; Epsilonproteobacteria; Campylobacterales; Helicobacteraceae; sf_3; 10572
Bacteria; Actinobacteria; Actinobacteria; Actinomycetales; Nocardiopsaceae; sf_1; 1385
Bacteria; Firmicutes; Clostridia; Clostridiales; Peptococc/Acidaminococc; sf_11; 71
Bacteria; Firmicutes; Clostridia; Clostridiales; Lachnospiraceae; sf_5; 3059
Bacteria; TM7; TM7-3; Unclassified; Unclassified; sf_1; 2697
Bacteria; Firmicutes; Bacilli; Bacillales; Paenibacillaceae; sf_1; 3630
Bacteria; Firmicutes; Bacilli; Bacillales; Bacillaceae; sf_1; 3424
Bacteria; Firmicutes; Bacilli; Bacillales; Bacillaceae; sf_1; 3661
Bacteria; Firmicutes; Bacilli; Bacillales; Bacillaceae; sf_1; 283
Bacteria; Firmicutes; Bacilli; Bacillales; Bacillaceae; sf_1; 829
Bacteria; Firmicutes; Bacilli; Bacillales; Bacillaceae; sf_1; 3675
Bacteria; Firmicutes; Mollicutes; Entomoplasmatales; Entomoplasmataceae; sf_1; 4074
Bacteria; Firmicutes; Clostridia; Clostridiales; Clostridiaceae; sf_12; 4156
Bacteria; Firmicutes; Clostridia; Clostridiales; Clostridiaceae; sf_12; 4575
Bacteria; Proteobacteria; Gammaproteobacteria; Enterobacteriales; Enterobacteriaceae; sf_1; 8631
Bacteria; Proteobacteria; Epsilonproteobacteria; Campylobacterales; Helicobacteraceae; sf_3; 10534
Bacteria; Nitrospira; Nitrospira; Nitrospirales; Nitrospiraceae; sf_1; 179
Bacteria; Verrucomicrobia; Verrucomicrobiae; Verrucomicrobiales; Verrucomicrobia subdivision 7; sf_1; 446
Bacteria; Bacteroidetes; Sphingobacteria; Sphingobacteriales; Sphingobacteriaceae; sf_1; 6272
Bacteria; Spirochaetes; Spirochaetes; Spirochaetales; Spirochaetaceae; sf_1; 6487
Bacteria; Spirochaetes; Spirochaetes; Spirochaetales; Spirochaetaceae; sf_1; 6554
Bacteria; Proteobacteria; Betaproteobacteria; Nitrosomonadales; Nitrosomonadaceae; sf_1; 7931
Bacteria; Firmicutes; Clostridia; Clostridiales; Lachnospiraceae; sf_5; 3111
Bacteria; Firmicutes; Clostridia; Clostridiales; Lachnospiraceae; sf_5; 2693
Bacteria; Firmicutes; Clostridia; Clostridiales; Peptostreptococcaceae; sf_5; 2913
Bacteria; Firmicutes; Clostridia; Clostridiales; Peptostreptococcaceae; sf_5; 1037
Bacteria; Firmicutes; Bacilli; Bacillales; Sporolactobacillaceae; sf_1; 3365
Bacteria; Firmicutes; Bacilli; Bacillales; Bacillaceae; sf_1; 3419
Bacteria; Firmicutes; Bacilli; Bacillales; Halobacillaceae; sf_1; 3756
Bacteria; Firmicutes; Bacilli; Bacillales; Halobacillaceae; sf_1; 3849
Bacteria; Firmicutes; Bacilli; Lactobacillales; Enterococcaceae; sf_1; 3881
Bacteria; Firmicutes; Bacilli; Lactobacillales; Streptococcaceae; sf_1; 3629
Bacteria; Firmicutes; Mollicutes; Anaeroplasmatales; Erysipelotrichaceae; sf_3; 144
Bacteria; Firmicutes; Clostridia; Clostridiales; Clostridiaceae; sf_12; 4632
Bacteria; Bacteroidetes; Flavobacteria; Flavobacteriales; Flavobacteriaceae; sf_1; 5509
Bacteria; Proteobacteria; Deltaproteobacteria; Myxococcales; Polyangiaceae; sf_3; 9912
Bacteria; NC10; NC10-1; Unclassified; Unclassified; sf_1; 536
Bacteria; Proteobacteria; Gammaproteobacteria; Enterobacteriales; Enterobacteriaceae; sf_1; 8640
Bacteria; Actinobacteria; Actinobacteria; Actinomycetales; Unclassified; sf_4; 1337
Bacteria; Actinobacteria; Actinobacteria; Actinomycetales; Kineosporiaceae; sf_1; 1087
Bacteria; Firmicutes; Clostridia; Clostridiales; Clostridiaceae; sf_12; 2786
Bacteria; Firmicutes; Clostridia; Clostridiales; Eubacteriaceae; sf_1; 28
Bacteria; Firmicutes; Bacilli; Bacillales; Bacillaceae; sf_1; 3540
Bacteria; Firmicutes; Bacilli; Bacillales; Bacillaceae; sf_1; 3827
Bacteria; Firmicutes; Bacilli; Lactobacillales; Lactobacillaceae; sf_1; 3703
Bacteria; Firmicutes; gut clone group; Unclassified; Unclassified; sf_1; 4298
Bacteria; Firmicutes; Catabacter; Unclassified; Unclassified; sf_4; 4325
Bacteria; Unclassified; Unclassified; Unclassified; Unclassified; sf_92; 9999
Bacteria; Bacteroidetes; Bacteroidetes; Bacteroidales; Bacteroidaceae; sf_12; 5621
Bacteria; BRC1; Unclassified; Unclassified; Unclassified; sf_1; 5143
Bacteria; Proteobacteria; Betaproteobacteria; Rhodocyclales; Rhodocyclaceae; sf_1; 8052
Bacteria; Proteobacteria; Gammaproteobacteria; Alteromonadales; Alteromonadaceae; sf_1; 8904
Bacteria; Proteobacteria; Deltaproteobacteria; Myxococcales; Polyangiaceae; sf_3; 10353
Bacteria; Firmicutes; Bacilli; Bacillales; Bacillaceae; sf_1; 3283
Bacteria; Firmicutes; Bacilli; Bacillales; Staphylococcaceae; sf_1; 3258
Bacteria; Firmicutes; Bacilli; Bacillales; Staphylococcaceae; sf_1; 3605
Bacteria; Firmicutes; Bacilli; Lactobacillales; Leuconostocaceae; sf_1; 3497
Bacteria; Firmicutes; Bacilli; Lactobacillales; Streptococcaceae; sf_1; 3290
Bacteria; Firmicutes; Unclassified; Unclassified; Unclassified; sf_8; 4536
Bacteria; Firmicutes; Clostridia; Clostridiales; Lachnospiraceae; sf_5; 4155
Bacteria; Firmicutes; Clostridia; Clostridiales; Clostridiaceae; sf_12; 4378
Bacteria; Verrucomicrobia; Verrucomicrobiae; Verrucomicrobiales; Unclassified; sf_3; 11
Bacteria; Acidobacteria; Acidobacteria; Acidobacteriales; Acidobacteriaceae; sf_14; 208
Bacteria; Bacteroidetes; Bacteroidetes; Bacteroidales; Bacteroidaceae; sf_12; 5275
Bacteria; Bacteroidetes; Flavobacteria; Flavobacteriales; Flavobacteriaceae; sf_1; 5423
Bacteria; Proteobacteria; Betaproteobacteria; Nitrosomonadales; Nitrosomonadaceae; sf_1; 7805
Bacteria; Proteobacteria; Betaproteobacteria; Nitrosomonadales; Nitrosomonadaceae; sf_1; 7858
Bacteria; Actinobacteria; Actinobacteria; Actinomycetales; Frankiaceae; sf_1; 1105
Bacteria; Gemmatimonadetes; Unclassified; Unclassified; Unclassified; sf_5; 1565
Bacteria; Actinobacteria; Actinobacteria; Actinomycetales; Micrococcaceae; sf_1; 1213
Bacteria; Firmicutes; Clostridia; Clostridiales; Lachnospiraceae; sf_5; 2804
Bacteria; Firmicutes; Bacilli; Bacillales; Staphylococcaceae; sf_1; 3284
Bacteria; Firmicutes; Bacilli; Bacillales; Staphylococcaceae; sf_1; 3628
Bacteria; Firmicutes; Bacilli; Lactobacillales; Lactobacillaceae; sf_1; 3547
Bacteria; Firmicutes; Bacilli; Lactobacillales; Lactobacillaceae; sf_1; 3634
Bacteria; Firmicutes; Bacilli; Lactobacillales; Enterococcaceae; sf_1; 3261
Bacteria; Firmicutes; Clostridia; Clostridiales; Clostridiaceae; sf_12; 4638
Bacteria; Firmicutes; Clostridia; Clostridiales; Clostridiaceae; sf_12; 4275
Bacteria; Firmicutes; Clostridia; Clostridiales; Peptococc/Acidaminococc; sf_11; 489
Bacteria; Proteobacteria; Betaproteobacteria; Unclassified; Unclassified; sf_3; 7765
Bacteria; NC10; NC10-2; Unclassified; Unclassified; sf_1; 10254
Bacteria; Actinobacteria; Actinobacteria; Actinomycetales; Mycobacteriaceae; sf_1; 1365
Bacteria; Firmicutes; Clostridia; Clostridiales; Peptostreptococcaceae; sf_5; 3112
Bacteria; Firmicutes; Clostridia; Clostridiales; Clostridiaceae; sf_12; 3219
Bacteria; Firmicutes; Bacilli; Bacillales; Bacillaceae; sf_1; 385
Bacteria; Firmicutes; Bacilli; Bacillales; Bacillaceae; sf_1; 571
Bacteria; Firmicutes; Bacilli; Bacillales; Staphylococcaceae; sf_1; 3684
Bacteria; Firmicutes; Bacilli; Lactobacillales; Enterococcaceae; sf_1; 3433
Bacteria; Proteobacteria; Betaproteobacteria; Nitrosomonadales; Nitrosomonadaceae; sf_1; 7831
Bacteria; Firmicutes; Bacilli; Lactobacillales; Lactobacillaceae; sf_1; 3330
Bacteria; Proteobacteria; Gammaproteobacteria; GAO cluster; Unclassified; sf_1; 8980
Bacteria; Firmicutes; Clostridia; Clostridiales; Lachnospiraceae; sf_5; 2756
Bacteria; Firmicutes; Bacilli; Bacillales; Staphylococcaceae; sf_1; 3545
Bacteria; Firmicutes; Bacilli; Lactobacillales; Enterococcaceae; sf_1; 3298
Bacteria; Nitrospira; Nitrospira; Nitrospirales; Nitrospiraceae; sf_3; 833
Bacteria; Bacteroidetes; Bacteroidetes; Bacteroidales; Bacteroidaceae; sf_12; 5474
Bacteria; Bacteroidetes; Unclassified; Unclassified; Unclassified; sf_1; 5745
Bacteria; Bacteroidetes; Bacteroidetes; Bacteroidales; Prevotellaceae; sf_1; 5769
Bacteria; Cyanobacteria; Cyanobacteria; Oscillatoriales; Unclassified; sf_1; 5184
Bacteria; Proteobacteria; Gammaproteobacteria; GAO cluster; Unclassified; sf_1; 9468
Bacteria; Proteobacteria; Deltaproteobacteria; Desulfuromonadales; Geobacteraceae; sf_1; 9956
Bacteria; Firmicutes; Clostridia; Clostridiales; Lachnospiraceae; sf_5; 3066
Bacteria; Firmicutes; Clostridia; Clostridiales; Lachnospiraceae; sf_5; 3088
Bacteria; Firmicutes; Clostridia; Clostridiales; Lachnospiraceae; sf_5; 3075
Bacteria; Firmicutes; Bacilli; Bacillales; Bacillaceae; sf_1; 3688
Bacteria; Firmicutes; Bacilli; Bacillales; Staphylococcaceae; sf_1; 3822
Bacteria; Firmicutes; Clostridia; Clostridiales; Lachnospiraceae; sf_5; 4167
Bacteria; Firmicutes; Bacilli; Bacillales; Thermoactinomycetaceae; sf_1; 3539
Bacteria; Bacteroidetes; Bacteroidetes; Bacteroidales; Rikenellaceae; sf_5; 5889
Bacteria; Bacteroidetes; Bacteroidetes; Bacteroidales; Porphyromonadaceae; sf_1; 5932
Bacteria; Bacteroidetes; Bacteroidetes; Bacteroidales; Prevotellaceae; sf_1; 5437
Bacteria; Firmicutes; Clostridia; Clostridiales; Lachnospiraceae; sf_5; 3089
Bacteria; Firmicutes; Bacilli; Bacillales; Staphylococcaceae; sf_1; 3569
Bacteria; Firmicutes; Bacilli; Lactobacillales; Lactobacillaceae; sf_1; 3767
Bacteria; Firmicutes; Bacilli; Lactobacillales; Enterococcaceae; sf_1; 3713
Bacteria; Firmicutes; Mollicutes; Unclassified; Unclassified; sf_6; 149
Bacteria; Chloroflexi; Dehalococcoidetes; Unclassified; Unclassified; sf_1; 2487
Bacteria; Firmicutes; Clostridia; Clostridiales; Lachnospiraceae; sf_5; 2784
Bacteria; Firmicutes; Clostridia; Clostridiales; Lachnospiraceae; sf_5; 2937
Bacteria; Firmicutes; Bacilli; Bacillales; Staphylococcaceae; sf_1; 3794
Bacteria; Firmicutes; Bacilli; Lactobacillales; Enterococcaceae; sf_1; 3382
Bacteria; Firmicutes; Bacilli; Lactobacillales; Enterococcaceae; sf_1; 3318
Bacteria; Firmicutes; Bacilli; Lactobacillales; Streptococcaceae; sf_1; 3397
Bacteria; Firmicutes; Bacilli; Lactobacillales; Streptococcaceae; sf_1; 3446
Bacteria; Bacteroidetes; Bacteroidetes; Bacteroidales; Prevotellaceae; sf_1; 5946
Bacteria; Actinobacteria; Actinobacteria; Actinomycetales; Thermomonosporaceae; sf_1; 1406
Bacteria; Actinobacteria; Actinobacteria; Actinomycetales; Corynebacteriaceae; sf_1; 1428
Bacteria; Firmicutes; Bacilli; Lactobacillales; Enterococcaceae; sf_1; 3392
Bacteria; Firmicutes; Bacilli; Lactobacillales; Enterococcaceae; sf_1; 3680
Bacteria; Firmicutes; Mollicutes; Anaeroplasmatales; Erysipelotrichaceae; sf_3; 3943
Bacteria; Bacteroidetes; Flavobacteria; Flavobacteriales; Flavobacteriaceae; sf_1; 5339
Bacteria; Cyanobacteria; Cyanobacteria; Oscillatoriales; Unclassified; sf_1; 5215
Bacteria; Actinobacteria; Actinobacteria; Actinomycetales; Pseudonocardiaceae; sf_1; 1402
Bacteria; Firmicutes; Bacilli; Lactobacillales; Lactobacillaceae; sf_1; 3521
Bacteria; Firmicutes; Bacilli; Lactobacillales; Lactobacillaceae; sf_1; 3885
Bacteria; Firmicutes; Bacilli; Lactobacillales; Streptococcaceae; sf_1; 3250
Bacteria; Firmicutes; Bacilli; Lactobacillales; Streptococcaceae; sf_1; 3906
Bacteria; Firmicutes; Bacilli; Lactobacillales; Streptococcaceae; sf_1; 3287
Bacteria; Firmicutes; Bacilli; Lactobacillales; Unclassified; sf_1; 3481
Bacteria; Firmicutes; Clostridia; Clostridiales; Clostridiaceae; sf_12; 4173
Bacteria; Proteobacteria; Deltaproteobacteria; Desulfobacterales; Desulfobacteraceae; sf_5; 10275
Bacteria; Bacteroidetes; Bacteroidetes; Bacteroidales; Prevotellaceae; sf_1; 5940
Bacteria; Firmicutes; Clostridia; Clostridiales; Lachnospiraceae; sf_5; 3087
Bacteria; Firmicutes; Clostridia; Clostridiales; Lachnospiraceae; sf_5; 2991
Bacteria; Firmicutes; Clostridia; Clostridiales; Lachnospiraceae; sf_5; 4381
Bacteria; Firmicutes; Bacilli; Lactobacillales; Aerococcaceae; sf_1; 3504
Bacteria; Firmicutes; Clostridia; Clostridiales; Lachnospiraceae; sf_5; 4443
Bacteria; Bacteroidetes; Bacteroidetes; Bacteroidales; Prevotellaceae; sf_1; 5398
Bacteria; Firmicutes; Clostridia; Clostridiales; Lachnospiraceae; sf_5; 2849
Bacteria; Proteobacteria; Betaproteobacteria; Burkholderiales; Comamonadaceae; sf_1; 7834
Bacteria; Proteobacteria; Gammaproteobacteria; Pasteurellales; Pasteurellaceae; sf_1; 9263
Bacteria; Firmicutes; Clostridia; Clostridiales; Lachnospiraceae; sf_5; 2726
Bacteria; Firmicutes; Clostridia; Clostridiales; Unclassified; sf_17; 2683
Bacteria; Firmicutes; Clostridia; Clostridiales; Lachnospiraceae; sf_5; 3107
Bacteria; Firmicutes; Clostridia; Clostridiales; Lachnospiraceae; sf_5; 3033
Bacteria; Firmicutes; Clostridia; Clostridiales; Clostridiaceae; sf_12; 2736
Bacteria; Firmicutes; Clostridia; Clostridiales; Lachnospiraceae; sf_5; 4538
Bacteria; Firmicutes; Clostridia; Clostridiales; Lachnospiraceae; sf_5; 2808
Bacteria; Firmicutes; Clostridia; Clostridiales; Lachnospiraceae; sf_5; 2733
Bacteria; Firmicutes; Clostridia; Clostridiales; Clostridiaceae; sf_12; 3019
Bacteria; Firmicutes; Clostridia; Clostridiales; Lachnospiraceae; sf_5; 2747
Bacteria; Firmicutes; Clostridia; Clostridiales; Lachnospiraceae; sf_5; 2793
Bacteria; Firmicutes; Clostridia; Clostridiales; Lachnospiraceae; sf_5; 4563
Bacteria; Fusobacteria; Fusobacteria; Fusobacterales; Fusobacteriaceae; sf_1; 488
Bacteria; Firmicutes; Clostridia; Clostridiales; Lachnospiraceae; sf_5; 3149
Bacteria; Firmicutes; Mollicutes; Anaeroplasmatales; Erysipelotrichaceae; sf_3; 3956
Bacteria; Bacteroidetes; Bacteroidetes; Bacteroidales; Rikenellaceae; sf_5; 6032
Bacteria; Bacteroidetes; Bacteroidetes; Bacteroidales; Bacteroidaceae; sf_12; 5285
Bacteria; Bacteroidetes; Bacteroidetes; Bacteroidales; Unclassified; sf_15; 5299
Bacteria; Bacteroidetes; Bacteroidetes; Bacteroidales; Bacteroidaceae; sf_12; 5424
Bacteria; Bacteroidetes; Bacteroidetes; Bacteroidales; Bacteroidaceae; sf_12; 5551
Bacteria; Bacteroidetes; Bacteroidetes; Bacteroidales; Bacteroidaceae; sf_12; 5979
Bacteria; Bacteroidetes; Bacteroidetes; Bacteroidales; Bacteroidaceae; sf_12; 6064
Bacteria; Proteobacteria; Gammaproteobacteria; Pasteurellales; Pasteurellaceae; sf_1; 9360
Bacteria; Proteobacteria; Gammaproteobacteria; Pasteurellales; Pasteurellaceae; sf_1; 8228
Bacteria; Proteobacteria; Gammaproteobacteria; Pasteurellales; Pasteurellaceae; sf_1; 8861
Bacteria; Fusobacteria; Fusobacteria; Fusobacterales; Fusobacteriaceae; sf_3; 558
Bacteria; Firmicutes; Clostridia; Clostridiales; Peptococc/Acidaminococc; sf_11; 181
Bacteria; Firmicutes; Clostridia; Clostridiales; Lachnospiraceae; sf_5; 2731
Bacteria; Firmicutes; Clostridia; Clostridiales; Lachnospiraceae; sf_5; 3032
Bacteria; Firmicutes; Clostridia; Clostridiales; Unclassified; sf_17; 2730
Bacteria; Firmicutes; Clostridia; Clostridiales; Lachnospiraceae; sf_5; 2769
Bacteria; Firmicutes; Clostridia; Clostridiales; Lachnospiraceae; sf_5; 2928
Bacteria; Firmicutes; Clostridia; Clostridiales; Lachnospiraceae; sf_5; 2753
Bacteria; Firmicutes; Clostridia; Clostridiales; Clostridiaceae; sf_12; 2898
Bacteria; Firmicutes; Clostridia; Clostridiales; Lachnospiraceae; sf_5; 2965
Bacteria; Firmicutes; Clostridia; Clostridiales; Lachnospiraceae; sf_5; 2737
Bacteria; Firmicutes; Clostridia; Clostridiales; Lachnospiraceae; sf_5; 3016
Bacteria; Firmicutes; Clostridia; Clostridiales; Lachnospiraceae; sf_5; 3185
Bacteria; Firmicutes; Clostridia; Clostridiales; Unclassified; sf_17; 2912
Bacteria; Firmicutes; Bacilli; Lactobacillales; Streptococcaceae; sf_1; 3253
Bacteria; Firmicutes; Mollicutes; Unclassified; Unclassified; sf_6; 196
Bacteria; Firmicutes; Clostridia; Clostridiales; Lachnospiraceae; sf_5; 4500
Bacteria; Firmicutes; Clostridia; Clostridiales; Clostridiaceae; sf_12; 4570

Fecal Taxa were found consisting of Firmicutes, Proteobacteria, Bacteroidetes and Actinobacteria. Of the Firmicutes most are from the order Clostridiales including the families Lachnospiraceae, Peptostreptococcaceae, Acidaminococci, and Clostridiaceae; a smaller percentage of bacteria from the order Bacillales are present including Bacillaceae, Halobacillaceae, Staphylococcaceae; as well as a similar precentage of Lactobacillales including the families of Lactobacillaceae, Enterococcaceae and Streptococcaceae. In the Proteobacteria phylum about a third are from Enterobacteriales including Enterobacteriaceae; with small percentatges of Alteromonadales including Alteromonadaceae, and Shewanellaceae. Other smaller constituent populations include taxa from the Order Burkholderiales including Burkholderiaceae, Comamonadaceae, Alcaligenaceae, Oxalobacteraceae, and Ralstoniaceae.

In some embodiments, a system is provided for detecting the presence or quantity of at least 10, 25, 50, 100, 200, 300, 400, or 500 different fecal taxa selected from Table 4 in a single assay. In further embodiments, the system comprises probes that selectively hybridize to each of the at least 10, 25, 50, 100, 200, 300, 400, or 500 different fecal taxa. In other embodiments, a method is provided for detecting fecal contamination in water comprising detecting the presence or quantity of one or more nucleic acid sequence selected from the group consisting of the 16S RNA sequences for fecal taxa listed in Table 4 in a water sample. In further embodiments, the detection method relies on detecting one or more 16S RNA sequences for clean water taxa listed in Table 11. In still further embodiments, the water sample is contacted with a plurality of probes that selectively hybridize to the one or more clean water taxa. Useful probes include those that can be used to identify organisms or taxa listed in Table 11.

### Example 4: Water Quality Testing, Fecal Contamination, and Flow Cytometry

Water quality is tested using a microparticle based multiplex system. A plurality of probes that recognize a collection of core microrganisms (Bacilli, Bacteroides and Clostridiales) that are associated with fecal contaminated water are selected from Table 4. An additional plurality of probes that recognize a collection of core microorganisms (α-proteobacteria) associated with clean water are also selected from a plurality of probes that identify the organisms or taxa listed in Table 11. A sublot of labeled microparticles is made for each probe within the two collections of plurality of probes. The probes are coupled to 3.0 micrometers latex microspheres (manufactured by Interfacial Dynamics) by carbodiimide coupling. After coupling the sublots are combined. Next negative control probe-coupled microparticles and positive control probe-coupled microparticles are added to make a finished lot of labeled microparticles.

Water samples are filtered on 0.22 μm filters and extracted for DNA using the UltraClean Water DNA kit (MoBio Laboratories). 16S rRNA genes are PCR amplified using universal bacterial primers 27F and 1492R. Eight replicate reactions across a temperature gradient (48-58° C.) are performed for each sample to minimize potential PCR amplification bias. The pooled amplicon of each sample (250 ng) is spiked with internal QS standards to permit normalization of assay hybridization signals. This mix is fragmented, biotin labeled and hybridized to the microparticles by combining approximately 40 picomoles of the bead-attached oligos with approximately 2-fold higher amount of biotin labeled amplicon in 2.3×SSC buffer at approximately 25° C. This mixture is incubated for two hours at room temperature followed by washing, dilution with 300 microlitres of saline pH 7.3, and analysis on the “FAGSCAN” (manufactured by Becton-Dickinson Immunocytometry Systems). The results demonstrate a ratio of BBC:A of 1.05 indicating that the water sample is contaminated with fecal matter.

### Example 5: Fecal Contamination Associated Taxa

A biosignature can be determined for fecal contamination by analyzing a sample suspected of fecal contamination using the systems and methods of the invention. DNA is extracted from the sample using standard techniques. 16S rDNA can then be amplified, processed, and analyzed as described in Example 2. Analysis by probe hybridization can be conducted using an array, as described in Example 2, or by using a flow cytometry method similar to that in Example 4, with probes bound to beads. The presence, absence, and/or level can be scored for each probe evaluated, and/or for each OTU represented by the probes evaluated. This collection of data, or a subset thereof, can then serve as a biosignature for contamination by fecal contamination, to which the biosignatures of test samples can be compared.

A water sample taken near a recreational beach is identified as having an unacceptably high level of fecal contamination. A series of water samples are collected near the beach and up the watershed of a nearby creek. The water samples are processed and then assayed on low density water quality arrays. After imaging and signal processing, the BBC:A ratios are calculated for each sample. The BBC:A signal is about 1.05-1.10 near the beach and increases up the watershed and then abruptly drops below 0.95 signifying clean water. The site surrounding location that has the highest BBC:A reading is searched and a ruptured sewer line is found. Repair of the sewer line increases the water quality in the creek watershed.

Fecal Taxa are found consisting of Firmicutes, Proteobacteria, Bacteroidetes and Actinobacteria. Of the Firmicutes most are from the order Clostridiales including the families Lachnospiraceae, Peptostreptococcaceae, Acidaminococci, and Clostridiaceae; a smaller percentage of bacteria from the order Bacillales are present including Bacillaceae, Halobacillaceae, Staphylococcaceae; as well as a similar precentage of Lactobacillales including the families of Lactobacillaceae, Enterococcaceae and Streptococcaceae. In the Proteobacteria phylum about a third are from Enterobacteriales including Enterobacteriaceae; with small percentatges of Alteromonadales including Alteromonadaceae, and Shewanellaceae. Other smaller constituent populations include taxa from the Order Burkholderiales including Burkholderiaceae, Comamonadaceae, Alcaligenaceae, Oxalobacteraceae, and Ralstoniaceae.

In some embodiments, a system is provided for detecting the presence or quantity of at least 10, 25, 50, 100, 200, 300, 400, or 500 different fecal taxa selected from Table 4 in a single water quality test assay. In further embodiments, the system comprises probes that selectively hybridize to each of the at least 10, 25, 50, 100, 200, 300, 400, or 500 different fetal taxa. In other embodiments, a method is provided for detecting fecal contamination in water comprising detecting the presence or quantity of one or more nucleic acid sequence selected from the group consisting of the 16S RNA sequences for fecal taxa listed in Table 4 in a water sample. In further embodiments, the detection method relies on detecting one or more 16S RNA sequences for clean water taxa listed in Table 11. In still further embodiments, the water sample is contacted with a plurality of probes that selectively hybridize to the one or more clean water taxa. Useful probes include those can be used to identify the organisms or taxa listed in Table 11.

### Example 6: Toxic Alga Bloom

Cyanobacteria, also known as blue-green algae, represent a major constituent of aquatic microbiomes. Under appropriate conditions, usually plentiful availability of nutrients, their numbers can increase rapidly resulting in an alga bloom. Once the nutrients are used up, the blooms die and then undergo bacterial decomposition that can consume all of the available dissolved oxygen leading to dead zones that are devoid of macroscopic life. Also worrisome is the ability of these cyanobacteria to sense the presence of others cyanobacteria or bacteria (quorum sensing) and at the specific density produce neurotoxins. Ingestion of water containing the cyanobacteria or their neurotoxins or seafood, particularly shellfish from areas with toxic alga blooms can cause serious injury or death. Methods to predict the probability of alga blooms, including toxic alga blooms are needed to protect the public health and ensure the safety of drinking water and seafood.

In some embodiments, a method is provided for predicting the likelihood of a toxic alga bloom comprising a) contacting a water sample with a plurality of probes that selectively bind to nucleic acids derived from cyanobacteria selected from Table 6; b) using hybridization data to determine the quantity and composition of cyanobacteria in the water sample; c) measuring environmental conditions; and d) predicting the likelihood of a toxic alga bloom based on cyanobacteria quantity and composition and environmental conditions. In further embodiments, the probes are selected by the methods discussed above to detect the genera listed in Table 6. Useful environmental conditions to monitor include water temperature, turbidity, nitrogen, phosphate, or iron concentration or sunlight intensity. In further embodiments, the presence or quantity of other microorganisms, particularly bacterial organisms is determined. Frequently, toxic bloom producing cyanobacteria live symbiotically with certain bacteria that use quorum sensing. Cyanobacteria may be able to read or hijack the bacterial quorum sensing, therefore knowledge of quantities of the symbiotic bacteria may be important for toxin expression (e.g. may influence, catalyze, or control toxin levels). Knowledge of the relationships of the populations present in an aquatic microbiome, include knowledge of the bacteria and cyanobacteria that are capable of quorum sensing and the densities at which this phenomena occurs can allow one to predict when a toxic alga bloom may occur. Armed with this predictive power, water management decisions can be made based on the likelihood of a toxic alga bloom, including banning swimming or shellfish collecting in areas likely to experience a bloom, or switching a municipal water supply over to an alternate water source like well water.

TABLE 6
Toxic Alga Bloom Cyanobacteria Genera
Genera

Microcystis
Anabaena
Planktothrix (Oscillatoria)
Nostoc
Hapalosiphon
Anabaenopsis
Nodularia
Aphanizomenon
Lyngbya
Schizothrix
Cylindrospermopsis
Aphanizomemon
Umezakia

A water sample from a recreational area at a local lake is applied to a down-selected phylogenetic array with probes selected as discussed above to detect nucleic acids from 100 OTUs of cyanobacteria associated with toxic alga blooms. Three cyanobacteria OTUs are detected and quantified that correlate to cyanobacteria densities above 50,000 cyanobacteriums per ml of water. The water temperature is 70° F., clarity is poor with a Secchi disk visible until 14 inches of depth, with bright sunshine predicted for the next 5 days with ambient outdoor daytime temperatures expected to climb into the nineties. The probability of a toxic alga bloom is over 90%. Preparations are made to close the swimming area at the recreational area and the managers of the municipal water supply are notified to switch over from surface water to well water in two days based on detection of cyanobacteria in a water sample.

### Example 7: PhyloChip Array

An array system, “PhyloChip”, was fabricated with some of the organism-specific and OTU-specific 16s rRNA probes selected by the methods described herein. The PhyloChip array consisted of 1,016,064 probe features, arranged as a grid of 1,008 rows and columns. Of these features, 90% were oligonucleotide PM or MM probes with exact or inexact complementarity, respectively, to 16s rRNA genes. Each probe is paired with a mismatch control probe to distinguish target-specific hybridization from background and non-target cross-hybridization. The remaining probes were used for image orientation, normalization controls, or for pathogen-specific signature amplicon detection using additional targeted regions of the chromosome. Each high-density 16s rRNA gene microarray was designed with additional probes that (1) targeted amplicons of prokaryotic metabolic genes spiked into the 16s rRNA gene amplicon mix in defined quantities just prior to fragmentation and (2) were complementary to pre-labelled oligonucleotides added into the hybridization mix. The first control collectively tested the target fragmentation, labeling by biotinylation, array hybridization, and staining/scanning efficiency. It also allowed the overall fluorescent intensity to be normalized across all the arrays in an experiment. The second control directly assayed the hybridization, staining and scanning

Complementary targets to the probe sequences hybridize to the array and fluorescent signals were captured as pixel images using standard AFFYMETRIX® software (GeneChip Microarray Analysis Suite, version 5.1) that reduced the data to an individual signal value for each probe and was typically exported as a human readable CEL′ file. Background probes were identified from the CEL file as those producing intensities in the lowest 2% of all intensities. The average intensity of the background probes was subtracted from the fluorescence intensity of all probes. The noise value (N) was the variation in pixel intensity signals observed by the scanner as it reads the array surface. The standard deviation of the pixel intensities within each of the identified background probe intensities was divided by the square root of the number of pixels comprising that feature. The average of the resulting quotients was used for N in the calculations described below.

Using previous methods, probe pairs scored as positive are those that meet two criteria: (i) the fluorescence intensity from the perfectly matched probe (PM) was at least 1.3 times greater than the intensity from the mismatched control (MM), and (ii) the difference in intensity, PM minus MM, was at least 130 times greater than the squared noise value (>130 N2). The positive fraction (PosFrac) was calculated for each probe set as the number of positive probe pairs divided by the total number of probe pairs in a probe set. An OTU was considered ‘present’ when its PosFrac for the corresponding probe set was >0.92 (based on empirical data from clone library analyses). Replicate arrays cuold be used collectively in determining the presence of each OTU by requiring each to exceed a PosFrac threshold. Present calls were propagated upwards through the taxonomic hierarchy by considering any node (subfamily, family, order, etc.) as ‘present’ if at least one of its subordinate OTUs was present.

Hybridization intensity was the measure of OTU abundance and was calculated in arbitrary units for each probe set as the trimmed average (maximum and minimum values removed before averaging) of the PM minus MM intensity differences across the probe pairs in a given probe set. All intensities <1 were shifted to 1 to avoid errors in subsequent logarithmic transformations.

The analysis methods described in Example 1 can also be applied to a sample that has been applied to the presently described PhyloChip G3 array.

A Latin Square Validation was carried out on the PhyloChip G3 array. The novel PhyloChip microarray (G3) was manufactured containing multiple probes for each known Bacterial and Archaeal taxon. The array was challenged with triplicate mixtures of 26 organisms combined in known but randomly assigned concentrations spanning over several orders of magnitude using a Latin Square experimental design. Probe-target complexes were quantified by flourescence intensity. To monitor community dynamics within the environment, water samples were taken from the San Francisco Bay (CA) at two time points following a point-source sewage spill. Entire 16S rRNA gene amplicon pools (˜100 billion molecules/time point) were evaluated with the array. Three replicates were tested on different days with 78 Latin Square chips and 1 Quantitative Standards only control. The amplicon concentration range was >4.5 log₁₀. The target concentration was from 0.25 pM to 477.79 pM, increasing 37% per step plus a 0 pM (26 different concentrations). Each chip contained all 26 targets, each with a different concentration 0-66 ng each for 243 ng total spike. The Latin Square matrix is not shown.

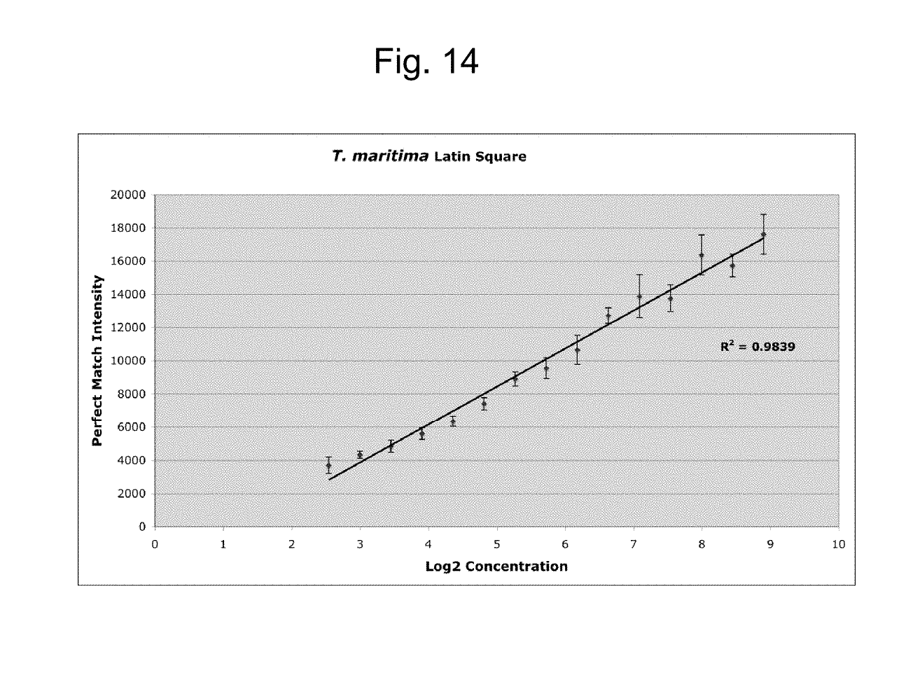

FIG. 14 is a chart showing the concentration of 16S amplicon versus PhyloChip response. Concentration is displayed as the log base 2 picomolar concentration within the PhyloChip hybridization chamber. The y-axis is the average of the multiple perfect match probes in the probe set. The vertical error bars denote the standard deviation of 3 replicate trials. The r-squared value over 0.98 indicates that the PhyloChip G3 array is quantitative in its ability to track changes in concentration.

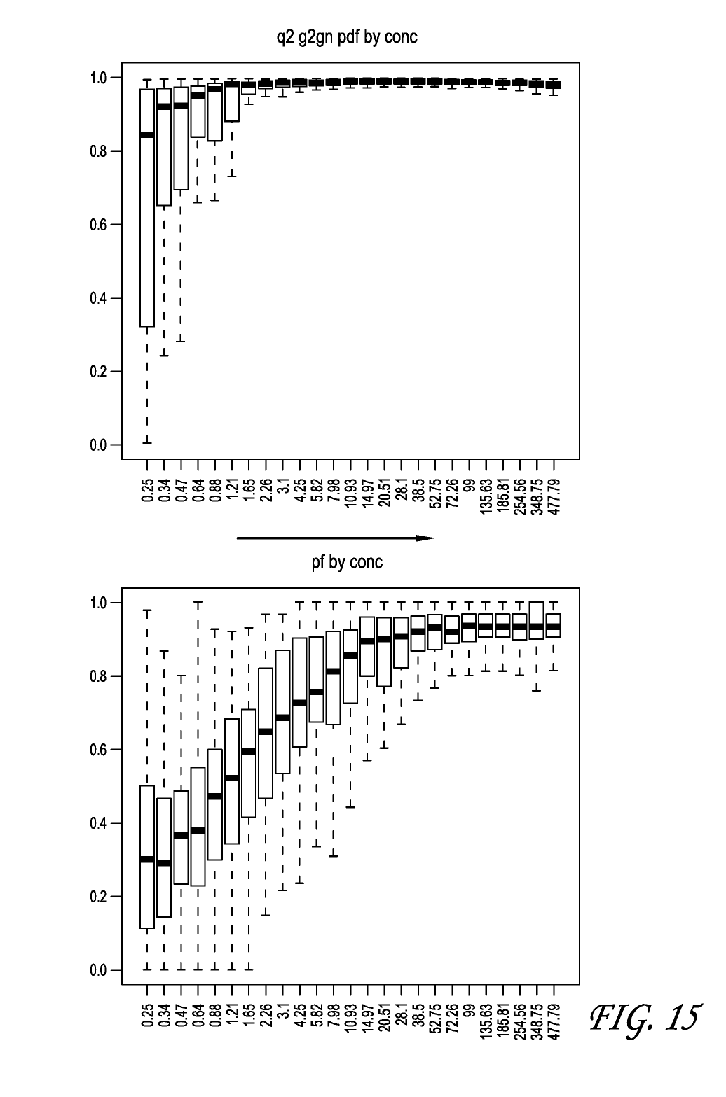

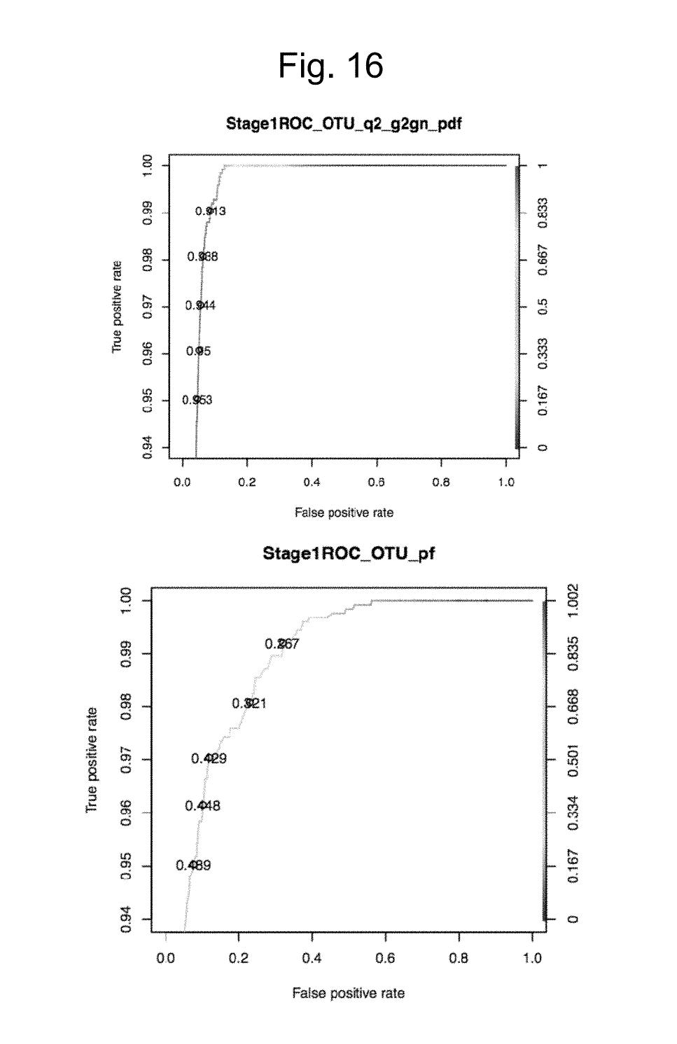

FIGS. 15 and 16 shows that model-based detection is an improvement over positive fraction detection of probe sets. Low concentrations (down to 2 pM) are differentiated from background in Latin Square.

FIG. 15 is boxplot comparison of the detection algorithm based on pair “response score”,r, distribution (novel) versus the positive fraction calculation (previously used with the G2 PhyloChip). In both plots the x-axis is the concentration of the spiked-in 16S amplicon (The arrow begins at 2 picomolar and extends through 500 picomolar). The y-axis ranges between 0 and 1 in both plots. The top plot's y-axis displays the median r score of all the probes within a probe set whereas the bottom plot's y-axis displays the positive fraction from the same data set. At low concentrations, 0.25 pM, both plots show a wide distribution of scores (see long whiskers), at 2 pM the top boxplots have short whiskers indicating that multiple measurements using a variety of bacterial and archaeal species all have very similar median r scores. The corresponding concentration on the positive fraction graph has a wide range of positive fraction scores. At nearly all concentrations, the r score outperforms the positive fraction.

FIG. 16 is two graphs that show the comparison of the r score metric versus the pf by receiver operator characteristic (R.O.C) plots. The steeper slope of the top curve compared to the bottom curve demonstrates that the r score metric can differentiate true positives from false positives more efficiently than the pf metric. The grayscale bar indicates the cutoff values (for either r scores or pf) at each point along the curve.

The validation shows that the novel PhyloChip G3 array is capable of excellent organism detection and quantification in a sample over the prior G2 array.

### Example 8: Water Quality Testing—Contamination Source Identified

A water sample can be assayed for contamination by fecal contamination by obtaining a biosignature for the water sample and comparing it to a biosignature for fecal contamination, such as the biosignature described in Example 5, using the systems and methods of the invention. DNA can be extracted from the sample, amplified, processed, and analyzed as in Example 2. Analysis by probe hybridization can be conducted using an array, as described in Example 2, or by using a flow cytometry method similar to that in Example 4, with probes bound to beads. The presence, absence, and/or level can be scored for each probe evaluated, and/or for each OTU represented by the probes evaluated. This data can then be compared to one or more biosignatures for one or more contaminants, including fecal contamination. If the degree of similarity between the biosignature of the test sample and the biosignature of fecal contamination is high, the sample is determined to contain fecal contamination. If the degree of similarity between the biosignatures is low, the sample is determined not to contain the fecal contamination.

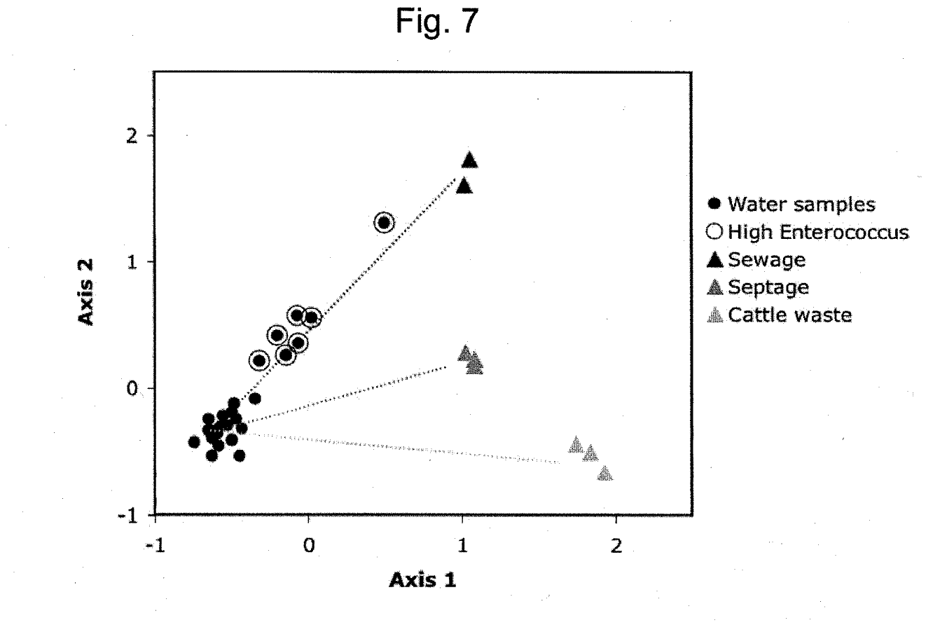

In a real-world scenario, the PhyloChip was used to compare the microbial community composition in polluted water samples compared to three potential pollution sources: sewage, septage and cattle waste, to determine which of the three sources most likely contributed to the pollution. FIG. 7 plots each PhyloChip result in 2D space. The plot revealed that the contaminated water samples (High Enterococus) fall along a vector toward the source community, sewage in this case.

This example illustrates the power of community analysis using the PhyloChip to identify the cause of Enterococcus exceedences in public waterways when the source is otherwise unknown.

In another example, two water samples were collected in Richardson Bay near the site of a 764,000 gallon sewage spill of primary-treated sewage from the Sausalito-Marin City Sanitary District in February 2009. One sample (#3) was collected directly adjacent to the plant 24 hours after the spill began and greatly exceeded water quality criteria for culture-based fecal indicator tests (IDEXX) for enterococcus, total coliforms and E. coli. The second sample (#26) was collected 150 m offshore 72 hours after the spill began and contained negligible (below detection limit) numbers of all fecal indicator bacteria. Samples of surface water were collected with 1 liter sterile bottles and stored at 4° C. until filtration (within 5 hours of collection) at Lawrence Berkeley National Laboratory. 750 ml of sample were vacuum filtered through Whatman Anodisc membrane filters (47 mm dia., 0.2 μm pore size) and immediately stored at −80° C. until DNA extraction.

Genomic DNA was extracted from filters using a bead beating and phenol/chloroform extraction method. 16S ribosomal RNA genes were amplified by PCR using universal primers 27F (5′-AGAGTTTGATCCTGGCTCAG-3′) and 1492R (5′-GGTTACCTTGTTACGACTT-3′) for bacteria, and 4Fa (5′-TCCGGTTGATCCTGCCRG-3′) and 1492R for archaea. Each PCR reaction contained 1×Ex Taq buffer (Takara Bio Inc., Japan), 0.125 units/μl Ex Taq polymerase, 0.8 mM dNTP mixture, 1.0 μg/μl BSA, and 300 nM each primer and 0.5 μl template. PCR conditions were 95° C. (3 min), followed by 30 cycles 95° C. (30 s), 48-58° C. (25 s), 72° C. (2 min), followed by a final extension 72° C. (10 min). Each DNA extract was amplified in 8 replicate 25 μl reactions spanning a range of annealing temperatures between 48-58° C. PCR products from different annealing temperature were combined for each sample and concentrated using Microcon YM-100 filters (Millipore).

Following gel quantification, 500 ng of bacterial 16S rRNA gene amplicons and 50 ng of archaeal amplicons were processed for PhyloChip analysis. PCR products were spiked with control amplicons derived from prokaryotic and eukaryotic metabolic genes and also synthetic 16S-like genes. This mix was fragmented to 50-200 bp using DNase I (0.02 U/μg DNA; Invitrogen) and One-Phor-All buffer by incubating at 20° C. for 10 min and 98° C. for 10 min. Terminal labeling of fragments was accomplished using GeneChip WT Double Stranded DNA Terminal Labeling kit (Affymetrix #900812) per manufacturer's instructions. Fragmented sample was labeled using terminal deoxynucleotidyl transferase and Affymetrix DNA Labeling Reagent by incubating at 37° C. for 60 min, followed by a 10 min 70° C. step. Hybridization to the array was carried out using the GeneChip Hybridization, Wash, and Stain Kit (Affymetrix #900720). Labeled DNA (42) was combined with Control Oligonucleotide B2 (Affymetrix #900301), DMSO (final concentration 15.7%) and MES buffer to a final volume of 130 and denatured at 99° C. for 5 minutes followed by 48° C. for 5 minutes. The entire reaction mixture was then added to the PhyloChip and incubated at 48° C. overnight (>16 h) at 60 rpm. The PhyloChips were subsequently washed and stained per the Affymetrix protocol using a GeneChip Fluidics Station 450 and then scanned using a GeneArray Scanner. The scan was captured as a pixel image using standard Affymetrix software (GeneChip Operating Software, version 1.0) that reduced the data to an individual signal value for each probe. Using analysis algorithms described here, a large number of taxa were identified as being present in either samples. In addition, many distinctive taxa were found to be unique to the water sample directly adjacent to the sewage spill within the first 24 hours of the spill and many different distinctive taxa were identified in the putatively non-polluted sample taken 150 meters offshore 72 hours after the spill. The taxa identified from the sewage spill site (sample 3), as well as their associated probes, can be used as a basis for the identification of fecal contamination in associated receiving waters.

A Fecalbacterium probe set and the individual probes of this probe set were analyzed at every step of the process using the methods of Example 1. A summary statistic of all probe sets identified as positive in each of the 2 samples and what was different was determined (not shown)

The use of the PhyloChip with diffusion chamber tests can give important information on the fate of a given microbiome such as the gut bacteria of animals etc. in a given receiving water. By using diffusion chambers to look at the survival rates of the members of the microbiome in a second environment such as different receiving waters to drive the selection of appropriate indicator organisms. There is a big difference in microbiome survival profiles between salt and fresh water. Also, it may be possible to ascertain the age of a spill, e.g., ongoing vs. several days old, by comparing the different survival rates of selected organisms. While use of a few organisms in a diffusion chamber test has been well known, the ability of the PhyloChip to perform a whole microbiome analysis will lead to previously unattainable results.

The sewage samples above were also submitted to diffusion tests using a diffusion chamber. The sewage microbiome along with the sewage microbiome mixed with the receiving waters were each tested so that effects of predation from organisms in the receiving waters could be accounted for.

### Example 9: Evaluating Sets of Probe Pair Responses to Determine the Presence or Absence of an OTU

Two bay water samples were taken at two time points after a water sewage leak. DNA from each sample was extracted, PCR amplified, digested, labeled and hybridized to PhyloChips. The response patterns from the probe sets for two selected human fecal OTUs were carefully examined as an illustrative example.

Spill 3-24 hours after start of spill, ankle deep directly in front of plant

Spi1126-72 hours after start of the spill, 500 ft offshore

OTU:36742

ss_id: 2036742 Bacteria; Firmicutes; Clostridia_SP; Clostridiales_CL; Clostridiales;

Faecalibacterium_FM; sfA; OTU:36742

One sequence in this OTU:

DQ805677.1 gg_id:185502 human fecal clone RL306aa189f12

OTU:38712

ss_id: 2038712 Bacteria; Firmicutes; Clostridia_SP; Clostridiales_CL; Clostridiales;

Ruminococcus_FM; sfA; OTU:38712;

One sequence in this OTU:

DQ797288.1 gg_id:188731 human fecal clone RL248_aai97d06

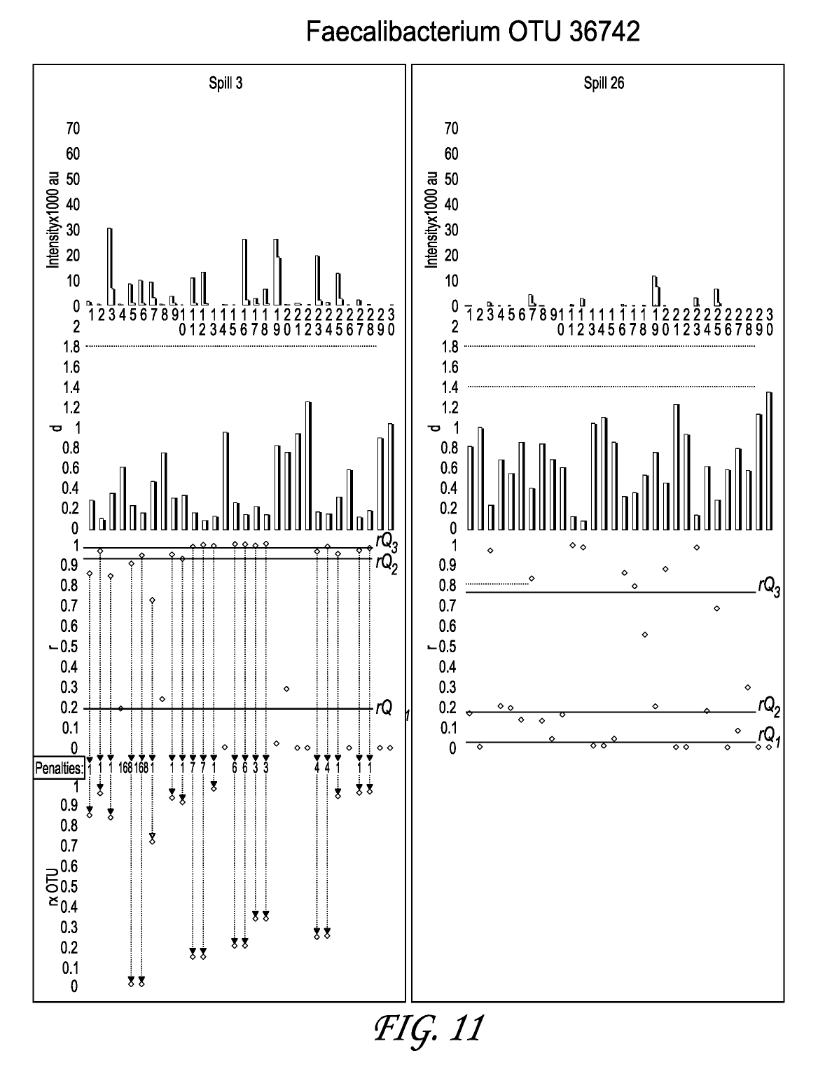

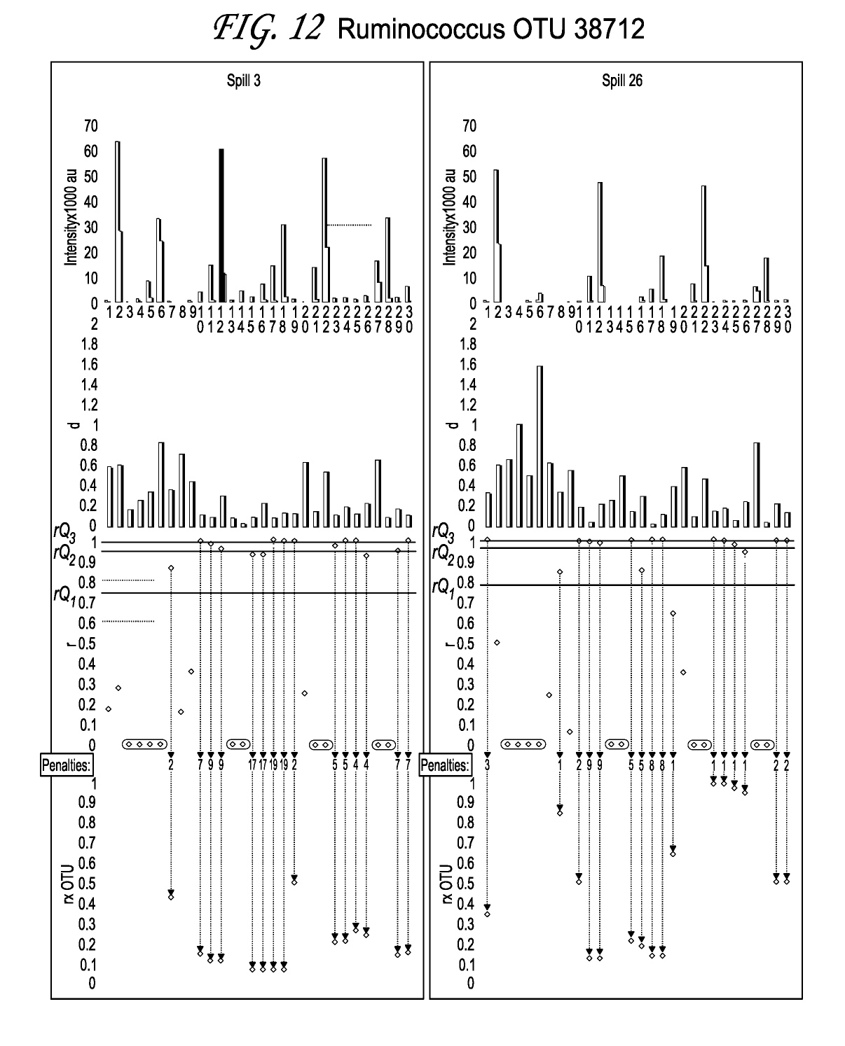

In FIGS. 11 and 12, the probe responses are presented and the uses of thresholds are demonstrated for both these OTUs. The PhyloChip is designed to contain multiple DNA probes complementary to specific DNA targets within the OTU. Each of these targets may have different A+T content, different T content, and may have putative cross-hybridization potential to other OTUs. These three factors are utilized for de-convolution of probe intensity measurements into presence or absence calls for an OUT.

After the scans were collected, probe intensities were background-subtracted and scaled to the spike-ins.

FIG. 11 compares the probe responses to Faecalibacterium OTU 36742 observed on two different PhyloChip experiments. The “Intensity” bar charts display the intensity from each PM and MM probe in blue and red, respectively, grouped as pairs. OTU 36742 has 30 probe pairs. The intensity measurements range from 5.7 to 30334.3 a.u. (arbitrary units). Next we calculate the pair difference score, d, for each probe pair by comparing the PM and MM intensities. For example, pair #6 reported a PM intensity of 9941.4 and a MM intensity of 903.4 for Spill 3.

$$d = 1 - ( \frac{PM - MM}{ PM + MM} ) = 1 - ( \frac{9941.4 - 903.4}{ 9941.4 + 903.4} ) = 0.166$$

Performing this transformation allows the difference between PM and MM probes to be expressed with a single number. The possible range of d is 0 to 2 and d approaches 0 when PM>>MM, d=1 when PM=MM and d approaches 2 when PM<<MM. Thus pair #6 displayed a sequence-specific interaction in Spill 3 since 0.166 is close to 0. The d values are plotted on the bar graphs labeled ‘d’, directly below their respective probe pairs. Notice that the same probe pair (#6) in Spill 26 produced a d value of 0.870. This is indicative of less separation between PM and MM values since 0.870 is further from zero than 0.166. Comparing d scores from the same probe pair across different chips is equitable since the probe composition is exactly the same (it is the same probe pair viewed under different experimental conditions).

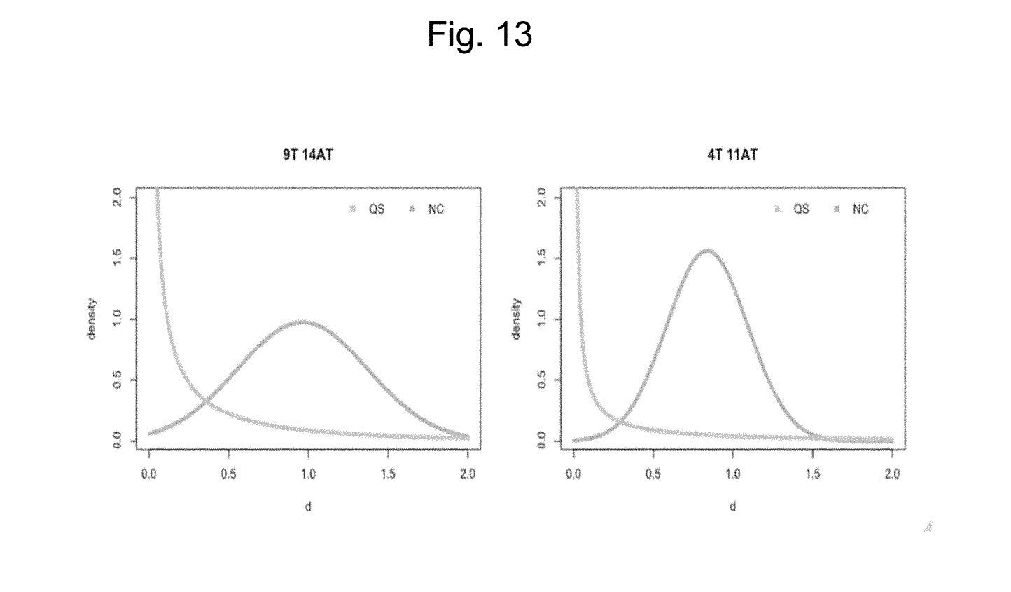

In the next step, the d scores are normalized to enable comparison of probe pairs with various nucleotide compositions. The goal in this transformation is to determine if the d value for a pair is more similar to d values derived from negative controls (NC, probe pairs with no potential cross-hybridization to any 16S rRNA sequence) or from positive controls which are the Quantitative Standards (QS, probe pairs with PM's matching the non-16S rRNA genes which are spiked into the experiment). Because the d_{QS} values are dependent on their target's A+T count and T count, the QS pairs are grouped by these attributes into classes and a separate distribution of d_{QS} values are found for each. The d_{NC} values are grouped in the same way. Because there is variation in the responses within each class, a distribution is estimated from the observations. Examples are shown below for Spill 3. Notice the different shape of the orange density plots which demonstrate the d observation of the Negative Control probes which are normally distributed. As shown in FIG. 13, in class “9T 14AT,” the mean d_{NC} is greater than class “4T 11AT”, also the variance is greater for class “9T 14AT.” Comparing the green density plots (estimated to follow a gamma distribution), quantitative standards for class “4T 11AT” nearly always produce d scores close to zero whereas class “9T 14AT” contains more observations of higher d scores (less distinction between PM and MM). In this example it can be seen that class “9T 14AT” has a larger range of d scores shared by both NC and QS (FIG. 13).

Next, each d value from an OTU probe set is compared to the distributions of d_{QS} and d_{NC} from the same class. For example, in OTU 36742 probe #6 has 9 thymine bases and 14 bases that are either thymine or adenine (Table 7). In Spill 3 this pair achieved a d value of 0.166.

TABLE 7
PM targets and the their counts of T and A + T
for OTU 36742

| pair | | T | A + T |
|---|---|---|---|
| # | PM target seq | count | count |
| 1 | TGATTACCTAGGTGTTGGAGGATTG | 9 | 14 |
| 2 | CAATCCTCCAACACCTAGGTAATCA | 5 | 14 |
| 3 | ACGCCGCGTAGAGGAAGAAGGTCTT | 4 | 11 |
| 4 | AAGACCTTCTTCCTCTACGCGGCGT | 7 | 11 |
| 5 | ATCCTGCGACGCACATAGAAATATG | 5 | 14 |
| 6 | CATATTTCTATGTGCGTCGCAGGAT | 9 | 14 |
| 7 | GACACGGCCCAGATTCTTACGGGAG | 4 | 10 |
| 8 | CTCCCGTAAGAATCTGGGCCGTGTC | 6 | 10 |
| 9 | TTTTCCTGGTAGTGCAGAGGTAGGC | 8 | 12 |
| 10 | GCCTACCTCTGCACTACCAGGAAAA | 4 | 12 |
| 11 | ACCAACTGACGCTGAGGCTTGAAAG | 4 | 12 |
| 12 | CTTTCAAGCCTCAGCGTCAGTTGGT | 8 | 12 |
| 13 | TTGCTTCCTCCATCTAGTGGACAAC | 8 | 13 |
| 14 | GTTGTCCACTAGATGGAGGAAGCAA | 5 | 13 |
| 15 | GAAACAACGTCCCAGTTTGGACTGC | 5 | 12 |
| 16 | GCAGTCCAAACTGGGACGTTGTTTC | 7 | 12 |
| 17 | TGTTTCTTTCGGGACGCAGAGACAG | 7 | 12 |
| 18 | CTGTCTCTGCGTCCCGAAAGAAACA | 5 | 12 |
| 19 | GGCCCAGATTCTTACGGGAGGCAGC | 4 | 9 |
| 20 | GCTGCCTCCCGTAAGAATCTGGGCC | 5 | 9 |
| 21 | CTAATACCGCATTAGAGCCCACAGG | 4 | 12 |
| 22 | CCTGTGGGCTCTAATGCGGTATTAG | 8 | 12 |
| 23 | AGGCTTGAAAGTGTGGGTAGCAAAC | 5 | 13 |
| 24 | GTTTGCTACCCACACTTTCAAGCCT | 8 | 13 |
| 25 | AGTGGACAACGGGTGAGTAACACAT | 4 | 13 |
| 26 | ATGTGTTACTCACCCGTTGTCCACT | 9 | 13 |
| 27 | GATTACCTAGGTGTTGGAGGATTGA | 8 | 14 |
| 28 | TCAATCCTCCAACACCTAGGTAATC | 6 | 14 |
| 29 | ACATGAGGAACCTGCCACATACAGG | 3 | 12 |
| 30 | CCTGTATGTGGCAGGTTCCTCATGT | 9 | 12 |

To determine the response score, r for probe #6, we find the probability that a probe with d=0.166 would be found among the normal distribution of NC (orange in density plots below) then find the probability that a probe pair of d=0.166 would be found among the gamma distribution of the QS, then ultimately record a ratio as the response score r according to the following equation:

$$r = ( \frac{{pdf}_{γ} ( X = d )}{{pdf}_{γ} ( X = d ) + {pdf}_{norm} ( X = d )} )$$

where:
r=response score to measure the potential that the probe pair is responding to a target and not the background
pdf_{γ}(X=d)_{γ=}probability that d could be drawn from the gamma distribution estimated for the target class ATx Ty
pdfₙₒᵣₘ (X=d)_{γ=}probability that d could be drawn from the normal distribution estimated for the target class ATx Ty
   The response score, r, ranges from 0.1 where 1 indicates that a probe pair was observed to have an unambiguous positive response. When r=0.5, the probe pair response resembles the NC and the QS equally and thus we can consider the response ambiguous. Continuing with our example probe #6 from OTU 36742:

$${pdf}_{γ} ( X = 0.166 ) = 0.951$$

$${pdf}_{norm} ( X = 0.166 ) = 0.058$$

$$r = ( \frac{0.951}{0.951 + 0.058} ) = 0.943$$

The r value for OTU 36742 #6 is plotted in FIG. 11 according to its response in both experiments. In Spill 26, this probe pair was less “positive” then in Spill 3.

There is an option in r scoring for certain probe pairs. The first, more-stringent option, calculates r only if sufficient observations (user defined threshold) from the QS or the NC are recorded to estimate the distributions described above. This first option is demonstrated in FIG. 12 OTU 38717 on the plots for r. The probe pairs circled in red were not used in finding rQ₁, rQ₂ and rQ₃ as described below. The second option calculates r scores for all probe pairs, using the general d_{QS} and d_{NC} model (using all QS and NC pairs irrespective of their class), whenever the class-specific model is not determined. This option is not shown in FIG. 12. The advantage of the second option is to increase the number of probe-pairs used in the analysis. A third option allows the nearest-class model to be used when a pair's specific class model is not determined for a given array. For example, if an experimental scan of a PhyloChip resulted in masking “outlier” probe pairs and this resulted in an insufficient pair count for the QS or NC for class “4T 12AT”, pairs of this class could be compared to the “5T 12AT” model. This hybrid of the two options allows both a high number of pairs to be observed and allows near class-specific response scoring. This option is also not shown in FIG. 12.

Next, all the r scores for a probe set are considered collectively in “Stage 1” probe set Presence/Absence scoring. Of the 30 probe pairs for OTU 36742, notice many of the r scores are near 1 in Spill 3 but few are near 1 in Spill 26 (FIG. 11). To quantitatively differentiate these distributions, the r scores are ranked and the breakpoints (quartiles), rQ₁, rQ₂ and rQ₃ are found by dividing the ranked observations into 4 equally-sized bins. The calculated quartiles for two OTUs across 2 experiments are shown in Table 8. This table describes the probe set performance Spi113 OTU 36742 rQ2=0.934 can be read as “Of the set of probe pairs targeting OTU 36742, half produced r scores over 0.934”.

TABLE 8
“Stage 1” results for 2 OTUs compared across 2 experiments.

| Experiment | OTU | rQ₁ | rQ₂ | rQ₃ |
|---|---|---|---|---|
| Spill 3 | 36742 | 0.207 | 0.934 | 0.983 |
| Spill 26 | 36742 | 0.015 | 0.172 | 0.763 |
| Spill 3 | 38712 | 0.738 | 0.953 | 0.991 |
| Spill 26 | 38712 | 0.789 | 0.985 | 0.996 |

The quartiles are illustrated as green lines in FIGS. 11 and 12 on each plot of the response scores (r). For an OTU to pass “Stage 1”, all three of the following criteria must be met: rQ₁≥0.200, rQ₂≥0.920, and rQ₃≥0.977. These criteria were learned from the Latin Square Data (not shown in this document). From Table 8, all four OTUs pass Stage 1 except OTU 36742 in Spill 26.

Only the OTUs which pass Stage 1 are considered in Stage 2 scoring. The objective in Stage 2 is to estimate the specificity of each responsive probe pair (where r>0.5) in consideration of the community of OTUs that pass Stage 1 on the same array. This is accomplished by penalizing each r score according to its putative cross-hybridization potential. Probe pairs that have putative cross-hybridization potential to many OTUs passing Stage 1 will be penalized by a greater factor than those with putative cross-hybridization to few OTUs passing Stage 1. The penalized score, rₓ, is calculated as

$${r}_{Xi} = \frac{{r}_{i}}{scalar ( {O}_{S 1} ⋂ {O}_{hi} )}$$

where:
O_{S1}=the set of OTUs passing Stage 1
Oₕᵢ=the set of OTUs with putative ability to hybridize to PM probe
scalar(O_{S1}∩Oₕᵢ)=the count of OTUs with hybridization potential and passing Stage 1

Probe pair 10 (pp10) in FIG. 12 exemplifies this effect. In Spill 3 pp10 achieved a high r score (0.997). The PM of pp10 can potentially hybridize to sequences in 11 different OTUs, 7 of these 11 passed Stage 1 (see row of numbers labeled “Penalties”). Thus r score is divided by 7 to yield

rₓ=0.142. The downward pointing arrows on FIGS. 11 and 12 demonstrate the magnitude of the penalty for each probe pair. After all penalties are considered, the r, values are ranked and quartiles found as above (rₓQ₁, rₓQ₂, rₓQ₃). Examples are shown in Table 9.

TABLE 9
“Stage 1” and “Stage 2” results for
2 OTUs compared across 2 experiments.

| Experiment | OTU | rQ₁ | rQ₂ | rQ₃ | rₓQ₁ | rₓQ₂ | rₓQ₃ |
|---|---|---|---|---|---|---|---|
| Spill 3 | 36742 | 0.207 | 0.934 | 0.983 | 0.200 | 0.529 | 0.947 |
| Spill 26 | 36742 | 0.015 | 0.172 | 0.763 | NA | NA | NA |
| Spill 3 | 38712 | 0.738 | 0.953 | 0.991 | 0.080 | 0.142 | 0.214 |
| Spill 26 | 38712 | 0.789 | 0.985 | 0.996 | 0.158 | 0.496 | 0.864 |

In the specific example described here we can conclude that Faecalibacterium OTU 36742 was present in Spill 3 but not Spill 26 based on responsiveness alone. Only in Spill 3 did Faecalibacterium OTU 36742 pass Stage 1. Conversely, the probe set for Ruminococcus OTU 38712 was responsive in Stage 1 analysis for both Spills but after further automated analysis refinement in Stage 2, it was determined as present in only Spill 26. Cutoff values for Stage 2: rₓQ₁≥0.100, rₓQ₂≥0.200, and rₓQ₃≥0.300, as empirically determined from the Latin Square Data (not shown in this document). As shown in Table 9, OTU 38712 did not meet these cutoff values in Spill 3.

### Example 10: Microbiome Signatures of Clean Ocean Water and Treated Wastewater Provide Effluent and Ocean Associated Taxa

Samples of dechlorinated effluent collected from the Montecito Sanitary District Wastewater Treatment Plant (Santa Barbara, Calif.) and samples of clean ocean water (1000m offshore, Santa Barbara, Calif.) were collected over a period of a year. The dechlorinated effluent samples were combined before processing and analysis as were the clean ocean water samples. Sample processing and analysis was performed as described in Example 2. The microbiome signatures for the dechlorinated effluent and the clean ocean water were compared. The effluent microbiome comprised of 266 taxa (Table 10) that were not found in the clean ocean water microbiome. The clean ocean water microbiome comprised of 231 taxa (Table 11) that were not found in the effluent samples.

The identified taxa represent “signature taxa” for treated effluent and clean ocean water respectively. Signature taxa can be identified from numerous environments, such as raw sewage, healthy, sick or diseased patients, food processing plants that repeatedly pass food safety inspections and those that routinely receive citations. Signature taxa have many uses. For instance, the presence or a specific abundance of different raw sewage signature taxa in the microbiome generated from a fresh water sample can signify insufficient processing at an upstream water treatment plant, something that can occur when large volumes of water are sent to a water treatment facility via storm drains. The presence or abundance of raw sewage taxa in a fresh water microbiome can also signify a leaking sewer pipe, seepage from an improperly maintained septic field or an illegal discharge.

TABLE 10
Effluent Microbiome
Taxa

Bacteria; Proteobacteria; Alphaproteobacteria; Rhodobacterales; Rhodobacteraceae; sf_1; 6848
Bacteria; Proteobacteria; Alphaproteobacteria; Rhodobacterales; Rhodobacteraceae; sf_1; 7602
Bacteria; Proteobacteria; Alphaproteobacteria; Rhodobacterales; Rhodobacteraceae; sf_1; 6883
Bacteria; Bacteroidetes; Flavobacteria; Flavobacteriales; Flavobacteriaceae; sf_1; 5671
Bacteria; Bacteroidetes; Flavobacteria; Flavobacteriales; Flavobacteriaceae; sf_1; 5695
Bacteria; Bacteroidetes; Flavobacteria; Flavobacteriales; Flavobacteriaceae; sf_1; 5896
Bacteria; Proteobacteria; Alphaproteobacteria; Rhodobacterales; Rhodobacteraceae; sf_1; 7596
Bacteria; Proteobacteria; Alphaproteobacteria; Rhodobacterales; Rhodobacteraceae; sf_1; 6982
Bacteria; Proteobacteria; Alphaproteobacteria; Unclassified; Unclassified; sf_6; 7252
Bacteria; Proteobacteria; Alphaproteobacteria; Rhodobacterales; Rhodobacteraceae; sf_1; 7050
Bacteria; Bacteroidetes; Flavobacteria; Flavobacteriales; Flavobacteriaceae; sf_1; 5919
Bacteria; Proteobacteria; Alphaproteobacteria; Rhodobacterales; Rhodobacteraceae; sf_1; 7288
Bacteria; Proteobacteria; Alphaproteobacteria; Rhodobacterales; Rhodobacteraceae; sf_1; 7432
Bacteria; Proteobacteria; Alphaproteobacteria; Rhodobacterales; Rhodobacteraceae; sf_1; 6664
Bacteria; Cyanobacteria; Cyanobacteria; Prochlorales; Unclassified; sf_1; 5076
Bacteria; Proteobacteria; Alphaproteobacteria; Unclassified; Unclassified; sf_6; 7196
Bacteria; Proteobacteria; Gammaproteobacteria; Alteromonadales; Alteromonadaceae; sf_1; 8517
Bacteria; Proteobacteria; Gammaproteobacteria; SAR86; Unclassified; sf_1; 9648
Bacteria; Proteobacteria; Unclassified; Unclassified; Unclassified; sf_20; 7365
Bacteria; Actinobacteria; BD2-10 group; Unclassified; Unclassified; sf_1; 1675
Bacteria; Cyanobacteria; Cyanobacteria; Chloroplasts; Chloroplasts; sf_5; 5007
Bacteria; Proteobacteria; Alphaproteobacteria; Unclassified; Unclassified; sf_6; 7510
Bacteria; Proteobacteria; Gammaproteobacteria; SAR86; Unclassified; sf_1; 9620
Bacteria; Unclassified; Unclassified; Unclassified; Unclassified; sf_148; 5235
Bacteria; Cyanobacteria; Cyanobacteria; Thermosynechococcus; Unclassified; sf_1; 5012
Bacteria; Proteobacteria; Gammaproteobacteria; Chromatiales; Ectothiorhodospiraceae; sf_1; 9387
Bacteria; Proteobacteria; Gammaproteobacteria; Unclassified; Unclassified; sf_3; 8647
Bacteria; Proteobacteria; Alphaproteobacteria; Rhodobacterales; Rhodobacteraceae; sf_1; 7054
Bacteria; Proteobacteria; Alphaproteobacteria; Rhodobacterales; Rhodobacteraceae; sf_1; 7233
Bacteria; Proteobacteria; Alphaproteobacteria; Rhodobacterales; Rhodobacteraceae; sf_1; 7045
Bacteria; Proteobacteria; Alphaproteobacteria; Rhodobacterales; Rhodobacteraceae; sf_1; 6960
Bacteria; Proteobacteria; Alphaproteobacteria; Rhodobacterales; Rhodobacteraceae; sf_1; 7405
Bacteria; Proteobacteria; Alphaproteobacteria; Rhodobacterales; Rhodobacteraceae; sf_1; 7329
Bacteria; Proteobacteria; Gammaproteobacteria; Oceanospirillales; Alcanivoraceae; sf_1; 9043
Bacteria; Proteobacteria; Alphaproteobacteria; Unclassified; Unclassified; sf_6; 7520
Bacteria; Proteobacteria; Alphaproteobacteria; Rhodobacterales; Rhodobacteraceae; sf_1; 7499
Bacteria; Proteobacteria; Gammaproteobacteria; SUP05; Unclassified; sf_1; 8953
Bacteria; Proteobacteria; Alphaproteobacteria; Rhodobacterales; Rhodobacteraceae; sf_1; 7649
Bacteria; Proteobacteria; Alphaproteobacteria; Bradyrhizobiales; Unclassified; sf_1; 7143
Bacteria; Actinobacteria; BD2-10 group; Unclassified; Unclassified; sf_1; 1732
Bacteria; Proteobacteria; Gammaproteobacteria; Unclassified; Unclassified; sf_3; 9016
Bacteria; Planctomycetes; Planctomycetacia; Planctomycetales; Planctomycetaceae; sf_3; 4654
Bacteria; Unclassified; Unclassified; Unclassified; Unclassified; sf_148; 4970
Bacteria; Proteobacteria; Alphaproteobacteria; Rhodobacterales; Rhodobacteraceae; sf_1; 7429
Bacteria; Actinobacteria; Actinobacteria; Actinomycetales; Acidothermaceae; sf_1; 1399
Bacteria; Proteobacteria; Alphaproteobacteria; Rhodobacterales; Rhodobacteraceae; sf_1; 6894
Bacteria; Actinobacteria; Actinobacteria; Acidimicrobiales; Acidimicrobiaceae; sf_1; 1282
Bacteria; Proteobacteria; Alphaproteobacteria; Rhodobacterales; Rhodobacteraceae; sf_1; 7033
Bacteria; Proteobacteria; Alphaproteobacteria; Rhodobacterales; Rhodobacteraceae; sf_1; 7140
Bacteria; Proteobacteria; Alphaproteobacteria; Rhodobacterales; Rhodobacteraceae; sf_1; 7085
Bacteria; Proteobacteria; Alphaproteobacteria; Unclassified; Unclassified; sf_6; 7421
Bacteria; Proteobacteria; Alphaproteobacteria; Unclassified; Unclassified; sf_6; 6858
Bacteria; Proteobacteria; Gammaproteobacteria; Unclassified; Unclassified; sf_3; 8333
Bacteria; Proteobacteria; Unclassified; Unclassified; Unclassified; sf_20; 7541
Bacteria; Proteobacteria; Gammaproteobacteria; Unclassified; Unclassified; sf_3; 9061
Bacteria; Proteobacteria; Alphaproteobacteria; Rhodobacterales; Rhodobacteraceae; sf_1; 6796
Bacteria; Firmicutes; Clostridia; Halanaerobiales; Halobacteroidaceae; sf_1; 887
Bacteria; Proteobacteria; Alphaproteobacteria; Rhodobacterales; Rhodobacteraceae; sf_1; 6714
Bacteria; Bacteroidetes; Sphingobacteria; Sphingobacteriales; Unclassified; sf_3; 5799
Bacteria; Planctomycetes; Planctomycetacia; Planctomycetales; Pirellulae; sf_3; 4801
Bacteria; Bacteroidetes; Bacteroidetes; Bacteroidales; Rikenellaceae; sf_5; 5889
Bacteria; Bacteroidetes; Flavobacteria; Flavobacteriales; Unclassified; sf_3; 5900
Bacteria; Cyanobacteria; Cyanobacteria; Chloroplasts; Chloroplasts; sf_5; 4983
Bacteria; Cyanobacteria; Cyanobacteria; Chloroplasts; Chloroplasts; sf_5; 5111
Bacteria; Cyanobacteria; Cyanobacteria; Chloroplasts; Chloroplasts; sf_5; 5156
Bacteria; Proteobacteria; Gammaproteobacteria; Unclassified; Unclassified; sf_3; 8805
Bacteria; Bacteroidetes; Sphingobacteria; Sphingobacteriales; Flexibacteraceae; sf_19; 5404
Bacteria; Aquificae; Aquificae; Aquificales; Hydrogenothermaceae; sf_1; 737
Bacteria; Proteobacteria; Alphaproteobacteria; Consistiales; Unclassified; sf_5; 7504
Bacteria; Bacteroidetes; Bacteroidetes; Bacteroidales; Rikenellaceae; sf_5; 5945
Bacteria; Proteobacteria; Alphaproteobacteria; Rhodobacterales; Rhodobacteraceae; sf_1; 7224
Bacteria; Proteobacteria; Betaproteobacteria; Unclassified; Unclassified; sf_3; 7923
Bacteria; Bacteroidetes; Unclassified; Unclassified; Unclassified; sf_4; 6190
Bacteria; Proteobacteria; Alphaproteobacteria; Consistiales; Unclassified; sf_5; 7203
Bacteria; Proteobacteria; Alphaproteobacteria; Consistiales; SAR11; sf_1; 7376
Bacteria; Proteobacteria; Alphaproteobacteria; Rhodobacterales; Rhodobacteraceae; sf_1; 7590
Bacteria; Proteobacteria; Alphaproteobacteria; Rhizobiales; Unclassified; sf_1; 7012
Bacteria; Proteobacteria; Gammaproteobacteria; Unclassified; Unclassified; sf_3; 8933
Bacteria; Proteobacteria; Alphaproteobacteria; Rhodobacterales; Rhodobacteraceae; sf_1; 6866
Bacteria; Cyanobacteria; Cyanobacteria; Chloroplasts; Chloroplasts; sf_5; 5166
Bacteria; Bacteroidetes; Flavobacteria; Flavobacteriales; Flavobacteriaceae; sf_1; 6104
Bacteria; Cyanobacteria; Cyanobacteria; Chloroplasts; Chloroplasts; sf_5; 5221
Bacteria; Cyanobacteria; Cyanobacteria; Chloroplasts; Chloroplasts; sf_5; 5120
Bacteria; Bacteroidetes; Bacteroidetes; Bacteroidales; Rikenellaceae; sf_5; 5947
Bacteria; Bacteroidetes; Bacteroidetes; Bacteroidales; Unclassified; sf_15; 6078
Bacteria; Proteobacteria; Gammaproteobacteria; Oceanospirillales; Unclassified; sf_3; 8961
Bacteria; Bacteroidetes; Flavobacteria; Flavobacteriales; Flavobacteriaceae; sf_1; 5641
Bacteria; Proteobacteria; Gammaproteobacteria; Methylococcales; Methylococcaceae; sf_1; 8821
Bacteria; Proteobacteria; Gammaproteobacteria; Acidithiobacillales; Acidithiobacillaceae; sf_1; 8913
Bacteria; Proteobacteria; Gammaproteobacteria; Unclassified; Unclassified; sf_3; 9456
Bacteria; Proteobacteria; Alphaproteobacteria; Rhodobacterales; Hyphomonadaceae; sf_1; 7584
Bacteria; Cyanobacteria; Unclassified; Unclassified; Unclassified; sf_5; 4993
Bacteria; Proteobacteria; Gammaproteobacteria; Oceanospirillales; Halomonadaceae; sf_1; 9141
Bacteria; Cyanobacteria; Cyanobacteria; Geitlerinema; Unclassified; sf_1; 4999
Bacteria; Proteobacteria; Alphaproteobacteria; Rhodobacterales; Rhodobacteraceae; sf_1; 6771
Bacteria; Proteobacteria; Gammaproteobacteria; Oceanospirillales; Unclassified; sf_3; 9010
Bacteria; Acidobacteria; Acidobacteria-9; Unclassified; Unclassified; sf_1; 704
Bacteria; OP10; Unclassified; Unclassified; Unclassified; sf_4; 728
Bacteria; Proteobacteria; Alphaproteobacteria; Rhodobacterales; Rhodobacteraceae; sf_1; 7508
Bacteria; Bacteroidetes; Flavobacteria; Flavobacteriales; Flavobacteriaceae; sf_1; 5559
Bacteria; Bacteroidetes; Bacteroidetes; Bacteroidales; Unclassified; sf_15; 5998
Bacteria; Proteobacteria; Gammaproteobacteria; Chromatiales; Chromatiaceae; sf_1; 8407
Bacteria; Proteobacteria; Gammaproteobacteria; Alteromonadales; Alteromonadaceae; sf_1; 9442
Bacteria; Gemmatimonadetes; Unclassified; Unclassified; Unclassified; sf_6; 2554
Bacteria; Proteobacteria; Alphaproteobacteria; Bradyrhizobiales; Unclassified; sf_1; 7255
Bacteria; Bacteroidetes; Bacteroidetes; Bacteroidales; Rikenellaceae; sf_5; 6317
Bacteria; Actinobacteria; Actinobacteria; Actinomycetales; Micrococcaceae; sf_1; 1266
Bacteria; Proteobacteria; Alphaproteobacteria; Rhodobacterales; Rhodobacteraceae; sf_1; 7049
Bacteria; Proteobacteria; Epsilonproteobacteria; Campylobacterales; Helicobacteraceae; sf_3; 10534
Bacteria; Proteobacteria; Alphaproteobacteria; Rhodobacterales; Rhodobacteraceae; sf_1; 7362
Bacteria; Bacteroidetes; Flavobacteria; Flavobacteriales; Flavobacteriaceae; sf_1; 5955
Bacteria; Proteobacteria; Unclassified; Unclassified; Unclassified; sf_21; 8509
Bacteria; Proteobacteria; Alphaproteobacteria; Rhodobacterales; Rhodobacteraceae; sf_1; 7373
Bacteria; Proteobacteria; Gammaproteobacteria; GAO cluster; Unclassified; sf_1; 9008
Bacteria; Proteobacteria; Alphaproteobacteria; Rhodobacterales; Rhodobacteraceae; sf_1; 7032
Bacteria; Proteobacteria; Alphaproteobacteria; Rhodobacterales; Rhodobacteraceae; sf_1; 6661
Bacteria; Bacteroidetes; Unclassified; Unclassified; Unclassified; sf_4; 5637
Bacteria; Proteobacteria; Gammaproteobacteria; Enterobacteriales; Enterobacteriaceae; sf_1; 9309
Bacteria; Proteobacteria; Alphaproteobacteria; Rhodobacterales; Rhodobacteraceae; sf_1; 6979
Bacteria; Proteobacteria; Gammaproteobacteria; Alteromonadales; Alteromonadaceae; sf_1; 9236
Bacteria; Proteobacteria; Alphaproteobacteria; Rhizobiales; Phyllobacteriaceae; sf_1; 7009
Bacteria; Proteobacteria; Gammaproteobacteria; Alteromonadales; Alteromonadaceae; sf_1; 9486
Bacteria; Cyanobacteria; Cyanobacteria; Nostocales; Unclassified; sf_1; 5174
Bacteria; Cyanobacteria; Cyanobacteria; Nostocales; Unclassified; sf_1; 5028
Bacteria; Proteobacteria; Gammaproteobacteria; Unclassified; Unclassified; sf_3; 8883
Bacteria; Chloroflexi; Anaerolineae; Unclassified; Unclassified; sf_9; 94
Bacteria; Proteobacteria; Alphaproteobacteria; Rhodobacterales; Rhodobacteraceae; sf_1; 7523
Bacteria; Bacteroidetes; Flavobacteria; Flavobacteriales; Flavobacteriaceae; sf_1; 5490
Bacteria; Cyanobacteria; Cyanobacteria; Nostocales; Unclassified; sf_1; 5175
Bacteria; Verrucomicrobia; Verrucomicrobiae; Verrucomicrobiales; Verrucomicrobia subdivision 7; sf_1; 760
Bacteria; Proteobacteria; Alphaproteobacteria; Consistiales; SAR11; sf_2; 7043
Bacteria; Chloroflexi; Anaerolineae; Chloroflexi-1f; Unclassified; sf_1; 765
Bacteria; Proteobacteria; Unclassified; Unclassified; Unclassified; sf_28; 10091
Bacteria; Proteobacteria; Gammaproteobacteria; GAO cluster; Unclassified; sf_1; 8980
Bacteria; Aquificae; Aquificae; Aquificales; Hydrogenothermaceae; sf_1; 220
Bacteria; Bacteroidetes; Sphingobacteria; Sphingobacteriales; Sphingobacteriaceae; sf_1; 5492
Bacteria; Proteobacteria; Gammaproteobacteria; Alteromonadales; Alteromonadaceae; sf_1; 8863
Bacteria; Cyanobacteria; Cyanobacteria; Spirulina; Unclassified; sf_1; 5034
Bacteria; Bacteroidetes; Flavobacteria; Flavobacteriales; Flavobacteriaceae; sf_1; 5499
Bacteria; Gemmatimonadetes; Unclassified; Unclassified; Unclassified; sf_5; 227
Bacteria; Proteobacteria; Alphaproteobacteria; Sphingomonadales; Sphingomonadaceae; sf_1; 7110
Bacteria; Proteobacteria; Alphaproteobacteria; Rhodobacterales; Rhodobacteraceae; sf_1; 7125
Bacteria; Cyanobacteria; Cyanobacteria; Chloroplasts; Chloroplasts; sf_5; 5130
Bacteria; Proteobacteria; Alphaproteobacteria; Rhodobacterales; Rhodobacteraceae; sf_1; 7536
Bacteria; Unclassified; Unclassified; Unclassified; Unclassified; sf_92; 9999
Bacteria; Proteobacteria; Deltaproteobacteria; Unclassified; Unclassified; sf_9; 9993
Bacteria; Proteobacteria; Alphaproteobacteria; Rhodobacterales; Rhodobacteraceae; sf_1; 6805
Bacteria; Unclassified; Unclassified; Unclassified; Unclassified; sf_148; 5022
Bacteria; Bacteroidetes; Bacteroidetes; Bacteroidales; Bacteroidaceae; sf_12; 5950
Bacteria; Proteobacteria; Alphaproteobacteria; Rhodobacterales; Rhodobacteraceae; sf_1; 7493
Bacteria; Verrucomicrobia; Verrucomicrobiae; Verrucomicrobiales; Verrucomicrobia subdivision 5; sf_1; 533
Bacteria; Proteobacteria; Deltaproteobacteria; Desulfobacterales; Desulfobacteraceae; sf_5; 9777
Bacteria; Proteobacteria; Alphaproteobacteria; Unclassified; Unclassified; sf_6; 6986
Bacteria; Proteobacteria; Alphaproteobacteria; Rhodobacterales; Rhodobacteraceae; sf_1; 6679
Bacteria; Cyanobacteria; Cyanobacteria; Nostocales; Unclassified; sf_1; 5072
Bacteria; Cyanobacteria; Cyanobacteria; Nostocales; Unclassified; sf_1; 5199
Bacteria; Cyanobacteria; Cyanobacteria; Nostocales; Unclassified; sf_1; 5191
Bacteria; Cyanobacteria; Cyanobacteria; Nostocales; Unclassified; sf_1; 5047
Bacteria; Bacteroidetes; Flavobacteria; Flavobacteriales; Flavobacteriaceae; sf_1; 5509
Bacteria; Bacteroidetes; Flavobacteria; Flavobacteriales; Cryomorphaceae; sf_1; 5400
Bacteria; Bacteroidetes; Flavobacteria; Flavobacteriales; Flavobacteriaceae; sf_1; 5301
Bacteria; Proteobacteria; Alphaproteobacteria; Fulvimarina; Unclassified; sf_1; 7281
Bacteria; Proteobacteria; Epsilonproteobacteria; Campylobacterales; Helicobacteraceae; sf_3; 10614
Bacteria; Firmicutes; Mollicutes; Mycoplasmatales; Mycoplasmataceae; sf_1; 4102
Bacteria; Dictyoglomi; Dictyoglomi; Dictyoglomales; Dictyoglomaceae; sf_9; 7579
Bacteria; Proteobacteria; Gammaproteobacteria; Alteromonadales; Alteromonadaceae; sf_1; 9586
Bacteria; Cyanobacteria; Cyanobacteria; Nostocales; Unclassified; sf_1; 5004
Bacteria; Proteobacteria; Alphaproteobacteria; Rhodobacterales; Rhodobacteraceae; sf_1; 7383
Bacteria; Proteobacteria; Gammaproteobacteria; Alteromonadales; Alteromonadaceae; sf_1; 8533
Bacteria; Proteobacteria; Gammaproteobacteria; Alteromonadales; Alteromonadaceae; sf_1; 9247
Bacteria; Proteobacteria; Gammaproteobacteria; Alteromonadales; Alteromonadaceae; sf_1; 8600
Bacteria; Proteobacteria; Alphaproteobacteria; Rhodobacterales; Rhodobacteraceae; sf_1; 312
Bacteria; Verrucomicrobia; Verrucomicrobiae; Verrucomicrobiales; Verrucomicrobiaceae; sf_6; 203
Bacteria; Actinobacteria; Actinobacteria; Actinomycetales; Microbacteriaceae; sf_1; 1135
Bacteria; Firmicutes; Clostridia; Clostridiales; Eubacteriaceae; sf_1; 28
Bacteria; Cyanobacteria; Cyanobacteria; Pseudanabaena; Unclassified; sf_1; 5008
Bacteria; Proteobacteria; Alphaproteobacteria; Rhodobacterales; Rhodobacteraceae; sf_1; 6955
Bacteria; Proteobacteria; Alphaproteobacteria; Rhodobacterales; Rhodobacteraceae; sf_1; 7084
Bacteria; Bacteroidetes; Sphingobacteria; Sphingobacteriales; Sphingobacteriaceae; sf_1; 6250
Bacteria; Proteobacteria; Alphaproteobacteria; Rhodobacterales; Rhodobacteraceae; sf_1; 7560
Bacteria; Proteobacteria; Alphaproteobacteria; Rhodobacterales; Rhodobacteraceae; sf_1; 7211
Bacteria; Proteobacteria; Alphaproteobacteria; Rhodobacterales; Rhodobacteraceae; sf_1; 6784
Bacteria; Bacteroidetes; Sphingobacteria; Sphingobacteriales; Flexibacteraceae; sf_19; 6261
Bacteria; Proteobacteria; Alphaproteobacteria; Rhodobacterales; Rhodobacteraceae; sf_1; 6827
Bacteria; Cyanobacteria; Cyanobacteria; Chloroplasts; Chloroplasts; sf_5; 5060
Bacteria; OD1; OP11-5; Unclassified; Unclassified; sf_1; 515
Bacteria; Proteobacteria; Alphaproteobacteria; Unclassified; Unclassified; sf_6; 7107
Bacteria; Proteobacteria; Deltaproteobacteria; Myxococcales; Polyangiaceae; sf_3; 10298
Bacteria; Actinobacteria; Actinobacteria; Unclassified; Unclassified; sf_1; 1370
Bacteria; Chloroflexi; Thermomicrobia; Unclassified; Unclassified; sf_2; 652
Bacteria; Bacteroidetes; Bacteroidetes; Bacteroidales; Prevotellaceae; sf_1; 6152
Bacteria; Spirochaetes; Spirochaetes; Spirochaetales; Spirochaetaceae; sf_1; 6458
Bacteria; Proteobacteria; Alphaproteobacteria; Rhodobacterales; Rhodobacteraceae; sf_1; 7262
Bacteria; Verrucomicrobia; Verrucomicrobiae; Verrucomicrobiales; Verrucomicrobiaceae; sf_6; 871
Bacteria; Proteobacteria; Gammaproteobacteria; Alteromonadales; Alteromonadaceae; sf_1; 9491
Bacteria; Bacteroidetes; Sphingobacteria; Sphingobacteriales; Flexibacteraceae; sf_19; 5728
Bacteria; Proteobacteria; Alphaproteobacteria; Sphingomonadales; Sphingomonadaceae; sf_1; 7576
Bacteria; Verrucomicrobia; Verrucomicrobiae; Verrucomicrobiales; Verrucomicrobiaceae; sf_7; 29
Bacteria; Chlorobi; Unclassified; Unclassified; Unclassified; sf_6; 5294
Bacteria; Cyanobacteria; Cyanobacteria; Chloroplasts; Chloroplasts; sf_5; 5039
Bacteria; Bacteroidetes; Flavobacteria; Flavobacteriales; Flavobacteriaceae; sf_1; 5758
Bacteria; Proteobacteria; Gammaproteobacteria; Oceanospirillales; Halomonadaceae; sf_1; 9446
Bacteria; Gemmatimonadetes; Unclassified; Unclassified; Unclassified; sf_5; 1127
Bacteria; Firmicutes; Clostridia; Clostridiales; Clostridiaceae; sf_12; 4156
Bacteria; Bacteroidetes; Sphingobacteria; Sphingobacteriales; Crenotrichaceae; sf_11; 5463
Bacteria; Cyanobacteria; Cyanobacteria; Plectonema; Unclassified; sf_1; 5010
Bacteria; Bacteroidetes; Sphingobacteria; Sphingobacteriales; Flexibacteraceae; sf_19; 5994
Bacteria; Proteobacteria; Gammaproteobacteria; Enterobacteriales; Enterobacteriaceae; sf_1; 8173
Bacteria; TM7; Unclassified; Unclassified; Unclassified; sf_1; 3025
Bacteria; Proteobacteria; Alphaproteobacteria; Rhizobiales; Unclassified; sf_1; 7339
Bacteria; Proteobacteria; Gammaproteobacteria; Oceanospirillales; Halomonadaceae; sf_1; 8598
Bacteria; Proteobacteria; Alphaproteobacteria; Rhizobiales; Bradyrhizobiaceae; sf_1; 7096
Bacteria; Cyanobacteria; Cyanobacteria; Chloroplasts; Chloroplasts; sf_5; 5006
Bacteria; Unclassified; Unclassified; Unclassified; Unclassified; sf_132; 9820
Bacteria; Proteobacteria; Alphaproteobacteria; Rhodobacterales; Rhodobacteraceae; sf_1; 6862
Bacteria; Cyanobacteria; Cyanobacteria; Plectonema; Unclassified; sf_1; 5210
Bacteria; Gemmatimonadetes; Unclassified; Unclassified; Unclassified; sf_5; 2047
Bacteria; Actinobacteria; Actinobacteria; Actinomycetales; Microbacteriaceae; sf_1; 1186
Bacteria; Proteobacteria; Alphaproteobacteria; Rhodobacterales; Rhodobacteraceae; sf_1; 7364
Bacteria; Proteobacteria; Alphaproteobacteria; Rhodobacterales; Rhodobacteraceae; sf_1; 7453
Bacteria; Proteobacteria; Alphaproteobacteria; Rhizobiales; Brucellaceae; sf_1; 6757
Bacteria; Proteobacteria; Deltaproteobacteria; Desulfuromonadales; Geobacteraceae; sf_1; 482
Bacteria; Proteobacteria; Deltaproteobacteria; Desulfobacterales; Desulfobacteraceae; sf_5; 10136
Bacteria; Cyanobacteria; Cyanobacteria; Chroococcales; Unclassified; sf_1; 5219
Bacteria; Chlorobi; Chlorobia; Chlorobiales; Chlorobiaceae; sf_1; 995
Bacteria; Acidobacteria; Acidobacteria; Acidobacteriales; Acidobacteriaceae; sf_16; 6414
Bacteria; Verrucomicrobia; Verrucomicrobiae; Verrucomicrobiales; Verrucomicrobiaceae; sf_6; 613
Bacteria; Proteobacteria; Alphaproteobacteria; Consistiales; Unclassified; sf_5; 7592
Bacteria; Proteobacteria; Alphaproteobacteria; Rhizobiales; Unclassified; sf_1; 6726
Bacteria; Cyanobacteria; Cyanobacteria; Chloroplasts; Chloroplasts; sf_5; 5182
Bacteria; Actinobacteria; Actinobacteria; Actinomycetales; Kineosporiaceae; sf_1; 1598

TABLE 11
Clean Ocean Water Microbiome
Taxa

Bacteria; Proteobacteria; Alphaproteobacteria; Rhodobacterales; Rhodobacteraceae; sf_1; 6848
Bacteria; Proteobacteria; Alphaproteobacteria; Rhodobacterales; Rhodobacteraceae; sf_1; 7602
Bacteria; Proteobacteria; Alphaproteobacteria; Rhodobacterales; Rhodobacteraceae; sf_1; 6883
Bacteria; Bacteroidetes; Flavobacteria; Flavobacteriales; Flavobacteriaceae; sf_1; 5671
Bacteria; Bacteroidetes; Flavobacteria; Flavobacteriales; Flavobacteriaceae; sf_1; 5695
Bacteria; Bacteroidetes; Flavobacteria; Flavobacteriales; Flavobacteriaceae; sf_1; 5896
Bacteria; Proteobacteria; Alphaproteobacteria; Rhodobacterales; Rhodobacteraceae; sf_1; 7596
Bacteria; Proteobacteria; Alphaproteobacteria; Rhodobacterales; Rhodobacteraceae; sf_1; 6982
Bacteria; Proteobacteria; Alphaproteobacteria; Unclassified; Unclassified; sf_6; 7252
Bacteria; Proteobacteria; Alphaproteobacteria; Rhodobacterales; Rhodobacteraceae; sf_1; 7050
Bacteria; Bacteroidetes; Flavobacteria; Flavobacteriales; Flavobacteriaceae; sf_1; 5919
Bacteria; Proteobacteria; Alphaproteobacteria; Rhodobacterales; Rhodobacteraceae; sf_1; 7288
Bacteria; Proteobacteria; Alphaproteobacteria; Rhodobacterales; Rhodobacteraceae; sf_1; 7432
Bacteria; Proteobacteria; Alphaproteobacteria; Rhodobacterales; Rhodobacteraceae; sf_1; 6664
Bacteria; Cyanobacteria; Cyanobacteria; Prochlorales; Unclassified; sf_1; 5076
Bacteria; Proteobacteria; Alphaproteobacteria; Unclassified; Unclassified; sf_6; 7196
Bacteria; Proteobacteria; Gammaproteobacteria; Alteromonadales; Alteromonadaceae; sf_1; 8517
Bacteria; Proteobacteria; Gammaproteobacteria; SAR86; Unclassified; sf_1; 9648
Bacteria; Proteobacteria; Unclassified; Unclassified; Unclassified; sf_20; 7365
Bacteria; Actinobacteria; BD2-10 group; Unclassified; Unclassified; sf_1; 1675
Bacteria; Cyanobacteria; Cyanobacteria; Chloroplasts; Chloroplasts; sf_5; 5007
Bacteria; Proteobacteria; Alphaproteobacteria; Unclassified; Unclassified; sf_6; 7510
Bacteria; Proteobacteria; Gammaproteobacteria; SAR86; Unclassified; sf_1; 9620
Bacteria; Unclassified; Unclassified; Unclassified; Unclassified; sf_148; 5235
Bacteria; Cyanobacteria; Cyanobacteria; Thermosynechococcus; Unclassified; sf_1; 5012
Bacteria; Proteobacteria; Gammaproteobacteria; Chromatiales; Ectothiorhodospiraceae; sf_1; 9387
Bacteria; Proteobacteria; Gammaproteobacteria; Unclassified; Unclassified; sf_3; 8647
Bacteria; Proteobacteria; Alphaproteobacteria; Rhodobacterales; Rhodobacteraceae; sf_1; 7054
Bacteria; Proteobacteria; Alphaproteobacteria; Rhodobacterales; Rhodobacteraceae; sf_1; 7233
Bacteria; Proteobacteria; Alphaproteobacteria; Rhodobacterales; Rhodobacteraceae; sf_1; 7045
Bacteria; Proteobacteria; Alphaproteobacteria; Rhodobacterales; Rhodobacteraceae; sf_1; 6960
Bacteria; Proteobacteria; Alphaproteobacteria; Rhodobacterales; Rhodobacteraceae; sf_1; 7405
Bacteria; Proteobacteria; Alphaproteobacteria; Rhodobacterales; Rhodobacteraceae; sf_1; 7329
Bacteria; Proteobacteria; Gammaproteobacteria; Oceanospirillales; Alcanivoraceae; sf_1; 9043
Bacteria; Proteobacteria; Alphaproteobacteria; Unclassified; Unclassified; sf_6; 7520
Bacteria; Proteobacteria; Alphaproteobacteria; Rhodobacterales; Rhodobacteraceae; sf_1; 7499
Bacteria; Proteobacteria; Gammaproteobacteria; SUP05; Unclassified; sf_1; 8953
Bacteria; Proteobacteria; Alphaproteobacteria; Rhodobacterales; Rhodobacteraceae; sf_1; 7649
Bacteria; Proteobacteria; Alphaproteobacteria; Bradyrhizobiales; Unclassified; sf_1; 7143
Bacteria; Actinobacteria; BD2-10 group; Unclassified; Unclassified; sf_1; 1732
Bacteria; Proteobacteria; Gammaproteobacteria; Unclassified; Unclassified; sf_3; 9016
Bacteria; Planctomycetes; Planctomycetacia; Planctomycetales; Planctomycetaceae; sf_3; 4654
Bacteria; Unclassified; Unclassified; Unclassified; Unclassified; sf_148; 4970
Bacteria; Proteobacteria; Alphaproteobacteria; Rhodobacterales; Rhodobacteraceae; sf_1; 7429
Bacteria; Actinobacteria; Actinobacteria; Actinomycetales; Acidothermaceae; sf_1; 1399
Bacteria; Proteobacteria; Alphaproteobacteria; Rhodobacterales; Rhodobacteraceae; sf_1; 6894
Bacteria; Actinobacteria; Actinobacteria; Acidimicrobiales; Acidimicrobiaceae; sf_1; 1282
Bacteria; Proteobacteria; Alphaproteobacteria; Rhodobacterales; Rhodobacteraceae; sf_1; 7033
Bacteria; Proteobacteria; Alphaproteobacteria; Rhodobacterales; Rhodobacteraceae; sf_1; 7140
Bacteria; Proteobacteria; Alphaproteobacteria; Rhodobacterales; Rhodobacteraceae; sf_1; 7085
Bacteria; Proteobacteria; Alphaproteobacteria; Unclassified; Unclassified; sf_6; 7421
Bacteria; Proteobacteria; Alphaproteobacteria; Unclassified; Unclassified; sf_6; 6858
Bacteria; Proteobacteria; Gammaproteobacteria; Unclassified; Unclassified; sf_3; 8333
Bacteria; Proteobacteria; Unclassified; Unclassified; Unclassified; sf_20; 7541
Bacteria; Proteobacteria; Gammaproteobacteria; Unclassified; Unclassified; sf_3; 9061
Bacteria; Proteobacteria; Alphaproteobacteria; Rhodobacterales; Rhodobacteraceae; sf_1; 6796
Bacteria; Firmicutes; Clostridia; Halanaerobiales; Halobacteroidaceae; sf_1; 887
Bacteria; Proteobacteria; Alphaproteobacteria; Rhodobacterales; Rhodobacteraceae; sf_1; 6714
Bacteria; Bacteroidetes; Sphingobacteria; Sphingobacteriales; Unclassified; sf_3; 5799
Bacteria; Planctomycetes; Planctomycetacia; Planctomycetales; Pirellulae; sf_3; 4801
Bacteria; Bacteroidetes; Bacteroidetes; Bacteroidales; Rikenellaceae; sf_5; 5889
Bacteria; Bacteroidetes; Flavobacteria; Flavobacteriales; Unclassified; sf_3; 5900
Bacteria; Cyanobacteria; Cyanobacteria; Chloroplasts; Chloroplasts; sf_5; 4983
Bacteria; Cyanobacteria; Cyanobacteria; Chloroplasts; Chloroplasts; sf_5; 5111
Bacteria; Cyanobacteria; Cyanobacteria; Chloroplasts; Chloroplasts; sf_5; 5156
Bacteria; Proteobacteria; Gammaproteobacteria; Unclassified; Unclassified; sf_3; 8805
Bacteria; Bacteroidetes; Sphingobacteria; Sphingobacteriales; Flexibacteraceae; sf_19; 5404
Bacteria; Aquificae; Aquificae; Aquificales; Hydrogenothermaceae; sf_1; 737
Bacteria; Proteobacteria; Alphaproteobacteria; Consistiales; Unclassified; sf_5; 7504
Bacteria; Bacteroidetes; Bacteroidetes; Bacteroidales; Rikenellaceae; sf_5; 5945
Bacteria; Proteobacteria; Alphaproteobacteria; Rhodobacterales; Rhodobacteraceae; sf_1; 7224
Bacteria; Proteobacteria; Betaproteobacteria; Unclassified; Unclassified; sf_3; 7923
Bacteria; Bacteroidetes; Unclassified; Unclassified; Unclassified; sf_4; 6190
Bacteria; Proteobacteria; Alphaproteobacteria; Consistiales; Unclassified; sf_5; 7203
Bacteria; Proteobacteria; Alphaproteobacteria; Consistiales; SAR11; sf_1; 7376
Bacteria; Proteobacteria; Alphaproteobacteria; Rhodobacterales; Rhodobacteraceae; sf_1; 7590
Bacteria; Proteobacteria; Alphaproteobacteria; Rhizobiales; Unclassified; sf_1; 7012
Bacteria; Proteobacteria; Gammaproteobacteria; Unclassified; Unclassified; sf_3; 8933
Bacteria; Proteobacteria; Alphaproteobacteria; Rhodobacterales; Rhodobacteraceae; sf_1; 6866
Bacteria; Cyanobacteria; Cyanobacteria; Chloroplasts; Chloroplasts; sf_5; 5166
Bacteria; Bacteroidetes; Flavobacteria; Flavobacteriales; Flavobacteriaceae; sf_1; 6104
Bacteria; Cyanobacteria; Cyanobacteria; Chloroplasts; Chloroplasts; sf_5; 5221
Bacteria; Cyanobacteria; Cyanobacteria; Chloroplasts; Chloroplasts; sf_5; 5120
Bacteria; Bacteroidetes; Bacteroidetes; Bacteroidales; Rikenellaceae; sf_5; 5947
Bacteria; Bacteroidetes; Bacteroidetes; Bacteroidales; Unclassified; sf_15; 6078
Bacteria; Proteobacteria; Gammaproteobacteria; Oceanospirillales; Unclassified; sf_3; 8961
Bacteria; Bacteroidetes; Flavobacteria; Flavobacteriales; Flavobacteriaceae; sf_1; 5641
Bacteria; Proteobacteria; Gammaproteobacteria; Methylococcales; Methylococcaceae; sf_1; 8821
Bacteria; Proteobacteria; Gammaproteobacteria; Acidithiobacillales; Acidithiobacillaceae; sf_1; 8913
Bacteria; Proteobacteria; Gammaproteobacteria; Unclassified; Unclassified; sf_3; 9456
Bacteria; Proteobacteria; Alphaproteobacteria; Rhodobacterales; Hyphomonadaceae; sf_1; 7584
Bacteria; Cyanobacteria; Unclassified; Unclassified; Unclassified; sf_5; 4993
Bacteria; Proteobacteria; Gammaproteobacteria; Oceanospirillales; Halomonadaceae; sf_1; 9141
Bacteria; Cyanobacteria; Cyanobacteria; Geitlerinema; Unclassified; sf_1; 4999
Bacteria; Proteobacteria; Alphaproteobacteria; Rhodobacterales; Rhodobacteraceae; sf_1; 6771
Bacteria; Proteobacteria; Gammaproteobacteria; Oceanospirillales; Unclassified; sf_3; 9010
Bacteria; Acidobacteria; Acidobacteria-9; Unclassified; Unclassified; sf_1; 704
Bacteria; OP10; Unclassified; Unclassified; Unclassified; sf_4; 728
Bacteria; Proteobacteria; Alphaproteobacteria; Rhodobacterales; Rhodobacteraceae; sf_1; 7508
Bacteria; Bacteroidetes; Flavobacteria; Flavobacteriales; Flavobacteriaceae; sf_1; 5559
Bacteria; Bacteroidetes; Bacteroidetes; Bacteroidales; Unclassified; sf_15; 5998
Bacteria; Proteobacteria; Gammaproteobacteria; Chromatiales; Chromatiaceae; sf_1; 8407
Bacteria; Proteobacteria; Gammaproteobacteria; Alteromonadales; Alteromonadaceae; sf_1; 9442
Bacteria; Gemmatimonadetes; Unclassified; Unclassified; Unclassified; sf_6; 2554
Bacteria; Proteobacteria; Alphaproteobacteria; Bradyrhizobiales; Unclassified; sf_1; 7255
Bacteria; Bacteroidetes; Bacteroidetes; Bacteroidales; Rikenellaceae; sf_5; 6317
Bacteria; Actinobacteria; Actinobacteria; Actinomycetales; Micrococcaceae; sf_1; 1266
Bacteria; Proteobacteria; Alphaproteobacteria; Rhodobacterales; Rhodobacteraceae; sf_1; 7049
Bacteria; Proteobacteria; Epsilonproteobacteria; Campylobacterales; Helicobacteraceae; sf_3; 10534
Bacteria; Proteobacteria; Alphaproteobacteria; Rhodobacterales; Rhodobacteraceae; sf_1; 7362
Bacteria; Bacteroidetes; Flavobacteria; Flavobacteriales; Flavobacteriaceae; sf_1; 5955
Bacteria; Proteobacteria; Unclassified; Unclassified; Unclassified; sf_21; 8509
Bacteria; Proteobacteria; Alphaproteobacteria; Rhodobacterales; Rhodobacteraceae; sf_1; 7373
Bacteria; Proteobacteria; Gammaproteobacteria; GAO cluster; Unclassified; sf_1; 9008
Bacteria; Proteobacteria; Alphaproteobacteria; Rhodobacterales; Rhodobacteraceae; sf_1; 7032
Bacteria; Proteobacteria; Alphaproteobacteria; Rhodobacterales; Rhodobacteraceae; sf_1; 6661
Bacteria; Bacteroidetes; Unclassified; Unclassified; Unclassified; sf_4; 5637
Bacteria; Proteobacteria; Gammaproteobacteria; Enterobacteriales; Enterobacteriaceae; sf_1; 9309
Bacteria; Proteobacteria; Alphaproteobacteria; Rhodobacterales; Rhodobacteraceae; sf_1; 6979
Bacteria; Proteobacteria; Gammaproteobacteria; Alteromonadales; Alteromonadaceae; sf_1; 9236
Bacteria; Proteobacteria; Alphaproteobacteria; Rhizobiales; Phyllobacteriaceae; sf_1; 7009
Bacteria; Proteobacteria; Gammaproteobacteria; Alteromonadales; Alteromonadaceae; sf_1; 9486
Bacteria; Cyanobacteria; Cyanobacteria; Nostocales; Unclassified; sf_1; 5174
Bacteria; Cyanobacteria; Cyanobacteria; Nostocales; Unclassified; sf_1; 5028
Bacteria; Proteobacteria; Gammaproteobacteria; Unclassified; Unclassified; sf_3; 8883
Bacteria; Chloroflexi; Anaerolineae; Unclassified; Unclassified; sf_9; 94
Bacteria; Proteobacteria; Alphaproteobacteria; Rhodobacterales; Rhodobacteraceae; sf_1; 7523
Bacteria; Bacteroidetes; Flavobacteria; Flavobacteriales; Flavobacteriaceae; sf_1; 5490
Bacteria; Cyanobacteria; Cyanobacteria; Nostocales; Unclassified; sf_1; 5175
Bacteria; Verrucomicrobia; Verrucomicrobiae; Verrucomicrobiales; Verrucomicrobia subdivision 7; sf_1; 760
Bacteria; Proteobacteria; Alphaproteobacteria; Consistiales; SAR11; sf_2; 7043
Bacteria; Chloroflexi; Anaerolineae; Chloroflexi-1f; Unclassified; sf_1; 765
Bacteria; Proteobacteria; Unclassified; Unclassified; Unclassified; sf_28; 10091
Bacteria; Proteobacteria; Gammaproteobacteria; GAO cluster; Unclassified; sf_1; 8980
Bacteria; Aquificae; Aquificae; Aquificales; Hydrogenothermaceae; sf_1; 220
Bacteria; Bacteroidetes; Sphingobacteria; Sphingobacteriales; Sphingobacteriaceae; sf_1; 5492
Bacteria; Proteobacteria; Gammaproteobacteria; Alteromonadales; Alteromonadaceae; sf_1; 8863
Bacteria; Cyanobacteria; Cyanobacteria; Spirulina; Unclassified; sf_1; 5034
Bacteria; Bacteroidetes; Flavobacteria; Flavobacteriales; Flavobacteriaceae; sf_1; 5499
Bacteria; Gemmatimonadetes; Unclassified; Unclassified; Unclassified; sf_5; 227
Bacteria; Proteobacteria; Alphaproteobacteria; Sphingomonadales; Sphingomonadaceae; sf_1; 7110
Bacteria; Proteobacteria; Alphaproteobacteria; Rhodobacterales; Rhodobacteraceae; sf_1; 7125
Bacteria; Cyanobacteria; Cyanobacteria; Chloroplasts; Chloroplasts; sf_5; 5130
Bacteria; Proteobacteria; Alphaproteobacteria; Rhodobacterales; Rhodobacteraceae; sf_1; 7536
Bacteria; Unclassified; Unclassified; Unclassified; Unclassified; sf_92; 9999
Bacteria; Proteobacteria; Deltaproteobacteria; Unclassified; Unclassified; sf_9; 9993
Bacteria; Proteobacteria; Alphaproteobacteria; Rhodobacterales; Rhodobacteraceae; sf_1; 6805
Bacteria; Unclassified; Unclassified; Unclassified; Unclassified; sf_148; 5022
Bacteria; Bacteroidetes; Bacteroidetes; Bacteroidales; Bacteroidaceae; sf_12; 5950
Bacteria; Proteobacteria; Alphaproteobacteria; Rhodobacterales; Rhodobacteraceae; sf_1; 7493
Bacteria; Verrucomicrobia; Verrucomicrobiae; Verrucomicrobiales; Verrucomicrobia subdivision 5; sf_1; 533
Bacteria; Proteobacteria; Deltaproteobacteria; Desulfobacterales; Desulfobacteraceae; sf_5; 9777
Bacteria; Proteobacteria; Alphaproteobacteria; Unclassified; Unclassified; sf_6; 6986
Bacteria; Proteobacteria; Alphaproteobacteria; Rhodobacterales; Rhodobacteraceae; sf_1; 6679
Bacteria; Cyanobacteria; Cyanobacteria; Nostocales; Unclassified; sf_1; 5072
Bacteria; Cyanobacteria; Cyanobacteria; Nostocales; Unclassified; sf_1; 5199
Bacteria; Cyanobacteria; Cyanobacteria; Nostocales; Unclassified; sf_1; 5191
Bacteria; Cyanobacteria; Cyanobacteria; Nostocales; Unclassified; sf_1; 5047
Bacteria; Bacteroidetes; Flavobacteria; Flavobacteriales; Flavobacteriaceae; sf_1; 5509
Bacteria; Bacteroidetes; Flavobacteria; Flavobacteriales; Cryomorphaceae; sf_1; 5400
Bacteria; Bacteroidetes; Flavobacteria; Flavobacteriales; Flavobacteriaceae; sf_1; 5301
Bacteria; Proteobacteria; Alphaproteobacteria; Fulvimarina; Unclassified; sf_1; 7281
Bacteria; Proteobacteria; Epsilonproteobacteria; Campylobacterales; Helicobacteraceae; sf_3; 10614
Bacteria; Firmicutes; Mollicutes; Mycoplasmatales; Mycoplasmataceae; sf_1; 4102
Bacteria; Dictyoglomi; Dictyoglomi; Dictyoglomales; Dictyoglomaceae; sf_9; 7579
Bacteria; Proteobacteria; Gammaproteobacteria; Alteromonadales; Alteromonadaceae; sf_1; 9586
Bacteria; Cyanobacteria; Cyanobacteria; Nostocales; Unclassified; sf_1; 5004
Bacteria; Proteobacteria; Alphaproteobacteria; Rhodobacterales; Rhodobacteraceae; sf_1; 7383
Bacteria; Proteobacteria; Gammaproteobacteria; Alteromonadales; Alteromonadaceae; sf_1; 8533
Bacteria; Proteobacteria; Gammaproteobacteria; Alteromonadales; Alteromonadaceae; sf_1; 9247
Bacteria; Proteobacteria; Gammaproteobacteria; Alteromonadales; Alteromonadaceae; sf_1; 8600
Bacteria; Proteobacteria; Alphaproteobacteria; Rhodobacterales; Rhodobacteraceae; sf_1; 312
Bacteria; Verrucomicrobia; Verrucomicrobiae; Verrucomicrobiales; Verrucomicrobiaceae; sf_6; 203
Bacteria; Actinobacteria; Actinobacteria; Actinomycetales; Microbacteriaceae; sf_1; 1135
Bacteria; Firmicutes; Clostridia; Clostridiales; Eubacteriaceae; sf_1; 28
Bacteria; Cyanobacteria; Cyanobacteria; Pseudanabaena; Unclassified; sf_1; 5008
Bacteria; Proteobacteria; Alphaproteobacteria; Rhodobacterales; Rhodobacteraceae; sf_1; 6955
Bacteria; Proteobacteria; Alphaproteobacteria; Rhodobacterales; Rhodobacteraceae; sf_1; 7084
Bacteria; Bacteroidetes; Sphingobacteria; Sphingobacteriales; Sphingobacteriaceae; sf_1; 6250
Bacteria; Proteobacteria; Alphaproteobacteria; Rhodobacterales; Rhodobacteraceae; sf_1; 7560
Bacteria; Proteobacteria; Alphaproteobacteria; Rhodobacterales; Rhodobacteraceae; sf_1; 7211
Bacteria; Proteobacteria; Alphaproteobacteria; Rhodobacterales; Rhodobacteraceae; sf_1; 6784
Bacteria; Bacteroidetes; Sphingobacteria; Sphingobacteriales; Flexibacteraceae; sf_19; 6261
Bacteria; Proteobacteria; Alphaproteobacteria; Rhodobacterales; Rhodobacteraceae; sf_1; 6827
Bacteria; Cyanobacteria; Cyanobacteria; Chloroplasts; Chloroplasts; sf_5; 5060
Bacteria; OD1; OP11-5; Unclassified; Unclassified; sf_1; 515
Bacteria; Proteobacteria; Alphaproteobacteria; Unclassified; Unclassified; sf_6; 7107
Bacteria; Proteobacteria; Deltaproteobacteria; Myxococcales; Polyangiaceae; sf_3; 10298
Bacteria; Actinobacteria; Actinobacteria; Unclassified; Unclassified; sf_1; 1370
Bacteria; Chloroflexi; Thermomicrobia; Unclassified; Unclassified; sf_2; 652
Bacteria; Bacteroidetes; Bacteroidetes; Bacteroidales; Prevotellaceae; sf_1; 6152
Bacteria; Spirochaetes; Spirochaetes; Spirochaetales; Spirochaetaceae; sf_1; 6458
Bacteria; Proteobacteria; Alphaproteobacteria; Rhodobacterales; Rhodobacteraceae; sf_1; 7262
Bacteria; Verrucomicrobia; Verrucomicrobiae; Verrucomicrobiales; Verrucomicrobiaceae; sf_6; 871
Bacteria; Proteobacteria; Gammaproteobacteria; Alteromonadales; Alteromonadaceae; sf_1; 9491
Bacteria; Bacteroidetes; Sphingobacteria; Sphingobacteriales; Flexibacteraceae; sf_19; 5728
Bacteria; Proteobacteria; Alphaproteobacteria; Sphingomonadales; Sphingomonadaceae; sf_1; 7576
Bacteria; Verrucomicrobia; Verrucomicrobiae; Verrucomicrobiales; Verrucomicrobiaceae; sf_7; 29
Bacteria; Chlorobi; Unclassified; Unclassified; Unclassified; sf_6; 5294
Bacteria; Cyanobacteria; Cyanobacteria; Chloroplasts; Chloroplasts; sf_5; 5039
Bacteria; Bacteroidetes; Flavobacteria; Flavobacteriales; Flavobacteriaceae; sf_1; 5758
Bacteria; Proteobacteria; Gammaproteobacteria; Oceanospirillales; Halomonadaceae; sf_1; 9446
Bacteria; Gemmatimonadetes; Unclassified; Unclassified; Unclassified; sf_5; 1127
Bacteria; Firmicutes; Clostridia; Clostridiales; Clostridiaceae; sf_12; 4156
Bacteria; Bacteroidetes; Sphingobacteria; Sphingobacteriales; Crenotrichaceae; sf_11; 5463
Bacteria; Cyanobacteria; Cyanobacteria; Plectonema; Unclassified; sf_1; 5010
Bacteria; Bacteroidetes; Sphingobacteria; Sphingobacteriales; Flexibacteraceae; sf_19; 5994
Bacteria; Proteobacteria; Gammaproteobacteria; Enterobacteriales; Enterobacteriaceae; sf_1; 8173
Bacteria; TM7; Unclassified; Unclassified; Unclassified; sf_1; 3025
Bacteria; Proteobacteria; Alphaproteobacteria; Rhizobiales; Unclassified; sf_1; 7339
Bacteria; Proteobacteria; Gammaproteobacteria; Oceanospirillales; Halomonadaceae; sf_1; 8598
Bacteria; Proteobacteria; Alphaproteobacteria; Rhizobiales; Bradyrhizobiaceae; sf_1; 7096
Bacteria; Cyanobacteria; Cyanobacteria; Chloroplasts; Chloroplasts; sf_5; 5006
Bacteria; Unclassified; Unclassified; Unclassified; Unclassified; sf_132; 9820
Bacteria; Proteobacteria; Alphaproteobacteria; Rhodobacterales; Rhodobacteraceae; sf_1; 6862
Bacteria; Cyanobacteria; Cyanobacteria; Plectonema; Unclassified; sf_1; 5210
Bacteria; Gemmatimonadetes; Unclassified; Unclassified; Unclassified; sf_5; 2047
Bacteria; Actinobacteria; Actinobacteria; Actinomycetales; Microbacteriaceae; sf_1; 1186
Bacteria; Proteobacteria; Alphaproteobacteria; Rhodobacterales; Rhodobacteraceae; sf_1; 7364
Bacteria; Proteobacteria; Alphaproteobacteria; Rhodobacterales; Rhodobacteraceae; sf_1; 7453
Bacteria; Proteobacteria; Alphaproteobacteria; Rhizobiales; Brucellaceae; sf_1; 6757
Bacteria; Proteobacteria; Deltaproteobacteria; Desulfuromonadales; Geobacteraceae; sf_1; 482
Bacteria; Proteobacteria; Deltaproteobacteria; Desulfobacterales; Desulfobacteraceae; sf_5; 10136
Bacteria; Cyanobacteria; Cyanobacteria; Chroococcales; Unclassified; sf_1; 5219
Bacteria; Chlorobi; Chlorobia; Chlorobiales; Chlorobiaceae; sf_1; 995
Bacteria; Acidobacteria; Acidobacteria; Acidobacteriales; Acidobacteriaceae; sf_16; 6414
Bacteria; Verrucomicrobia; Verrucomicrobiae; Verrucomicrobiales; Verrucomicrobiaceae; sf_6; 613
Bacteria; Proteobacteria; Alphaproteobacteria; Consistiales; Unclassified; sf_5; 7592
Bacteria; Proteobacteria; Alphaproteobacteria; Rhizobiales; Unclassified; sf_1; 6726
Bacteria; Cyanobacteria; Cyanobacteria; Chloroplasts; Chloroplasts; sf_5; 5182
Bacteria; Actinobacteria; Actinobacteria; Actinomycetales; Kineosporiaceae; sf_1; 1598

### Example 11: Clean Room Quality Testing

Traditional clean room testing relies on a wipe method and observing any spore growth in a petri dish. Comparison of the microbial communities detected using the wipe method with those detected by the PhyloChip of Example 7 are shown.

A total of 125 wipes that were applied to various clean rooms and satellite or spacecraft surfaces and their samples were compared. Each sample was about ˜250 mL each and hence concentrating samples are difficult. The samples were filtered using 0.45 μm filter followed by 0.2 μm filter. The resulting 10 mL fluid was concentrated using Amicon filters. DNA was extracted using a Maxwell extractor.

TABLE 12
Fourteen pooled samples according to spore count

| | Number of Samples pooled and |
|---|---|
| Spore Count | ID no. |
| Sample sets without spores: 7 sets | GI-15, 16, 17, 25, 26, 27, 28 |
| Spore count: 1: | GI-18 (10 samples pooled) |
| Spore count 2 to 4: | GI-19 (15 samples pooled) |
| Spore count 5 to 9: | GI-20 (5 samples pooled) |
| Spore count 10 to 11: | GI-21 (4 samples pooled) |
| Spore count 32: | GI-22 (1 sample) |
| Spore count 59: | GI-23 (1 sample) |
| Spore count 151: | GI-24 (1 sample) |

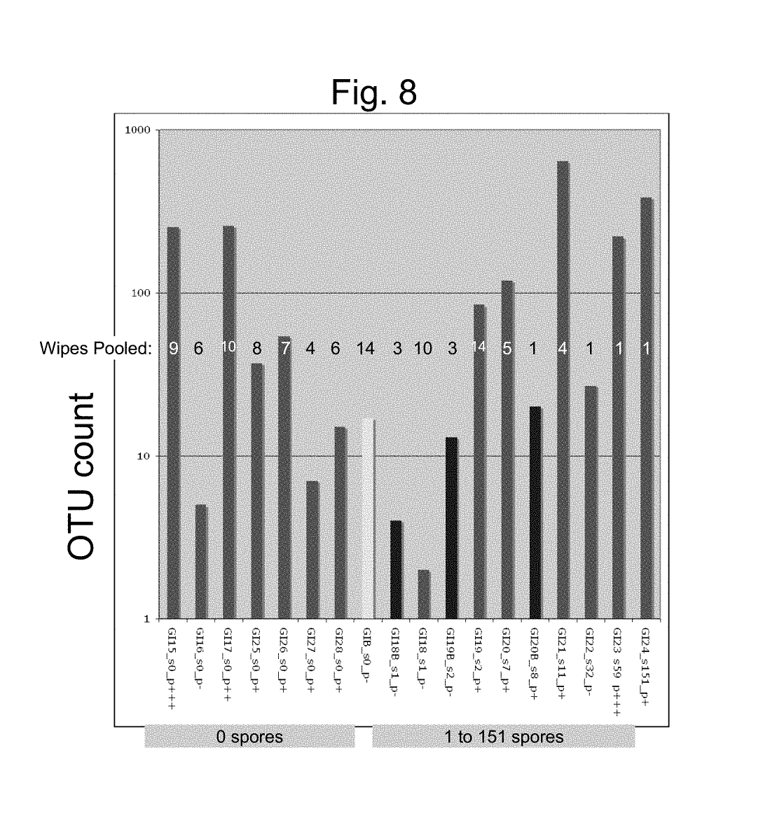

Referring now to FIG. 8, the petri dish method does not predict diversity of the microbial communities found by the PhyloChip. The PhyloChip detects OTUs when even zero spores were detected by the spore count method. As shown, up to 650 OTUs were detected using the methods of testing and analysis described in Examples 1 and 2. No relationship between spore count and PhyloChip OTU counts is observed.

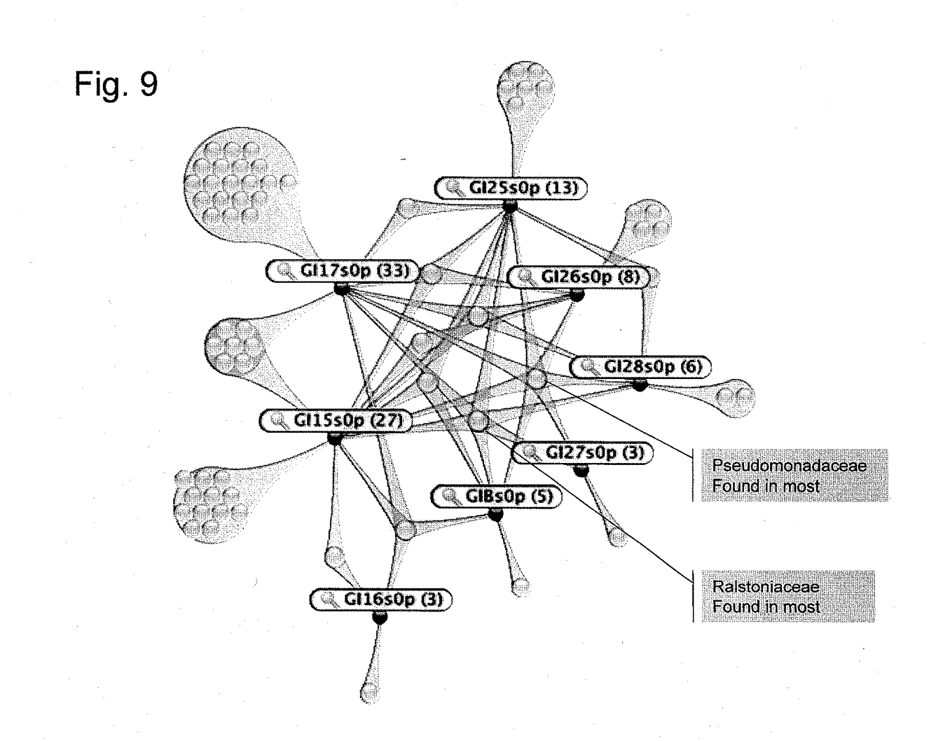

Referring to FIG. 9, the PhyloChip is able to detect what microbial families the samples have in common or which are unique. FIG. 9 shows a graphical network of the samples to show common or unique families. The dark dots are samples and the lighter dots are the family detected. Two families Pseudomonadaceae and Ralstoniaceae were found in most samples Families connected to single samples are unique, while families connected to many samples indicate families which are likely cosmopolitan among other similar environments where the sample was found.

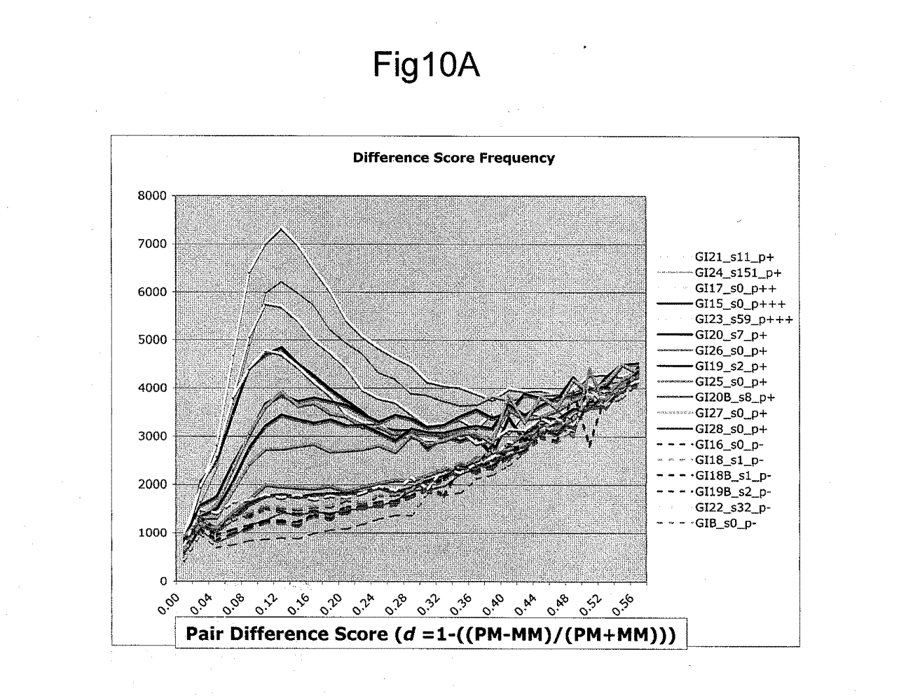

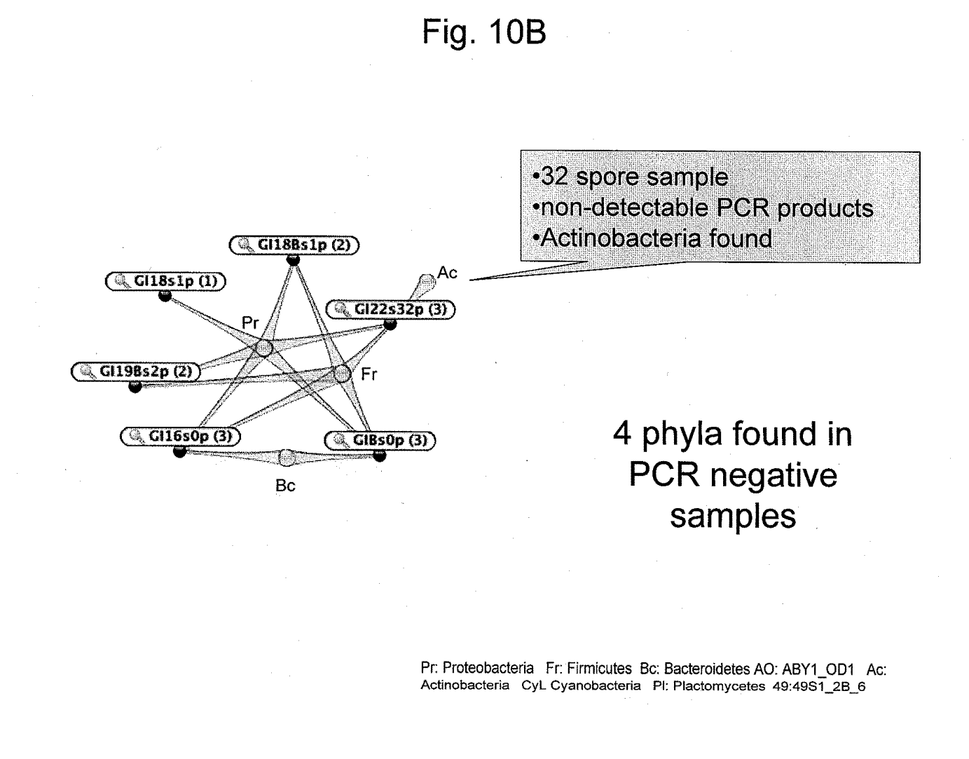

Referring now to FIGS. 10A and 10B, the pair difference score responses on the PhyloChip of Example 7 show that the PhyloChip is more sensitive to 16S amplicons and more sensitive than PCR methods. In FIG. 10A, the paired difference score responses are sensitive to 16S PCR products. Frequency of all probe pairs are shown. As shown, the closer the score to zero, the more positive the probe is determined to be. A sample that was not able to be PCR amplified correlated well with our PhyloChip detection results, showing very few responsive probe pairs. Inversely, if the PCR sample was positive, then a greater number of probe pairs responded positively. In FIG. 10B, four phyla were detected by the PhyloChip. Proteobacteria, Firmicutes, Bacteroidetes, and Actinobacteria, were detected even when no PCR products were detected.

### Example 12: Microbial Community Dynamics at the Rifle IFRC:Influence of Acetate Additions in the Field

Microbial community characterization of the Rifle, Colo. Integrated Field Research Challenge site began nearly10 years ago. Early methodologies involved analysis of groundwater and sediments using clonal library approaches and demonstrated enrichment of Geobacter-like sequences. Recent research efforts at Rifle have focused on three subsequent field-scale acetate amendment experiments (Winchester [2007], Big Rusty [2008], and Buckskin [2009]) and on characterizing a naturally bioreduced area—La Quinta (2009). All of these field-amendments replicated results from earlier experiments, with uranium reduction in groundwater during biostimulation. However, additional molecular approaches have been employed to characterize the bacterial communities including PLFA, qPCR, TRFLP, and microarray analyses (Akonni and Affymetrix-based LBNL G3 PhyloChip). Quantitative PCR demonstrated significant shifts in Geobacter species during field amendment. TRFLP profiling also indicated Geobacter-like sequences represented nearly 50% of the bacterial community in groundwater at early stages of acetate amendment, with replacement by bacteria distantly related to Acinetobacteria and Desulfobacter with time. The Akonni microarray detected signals for Geobacter, Pelobacter, and Geothrix, in addition to Dechloromonas and Dechlorosoma for Winchester (2007). Furthermore, the 2007 profiles differed from 2008, which is supported by PLFA and qPCR data, indicating a residual biomass/stimulated community going into the Big Rusty experiment. The G3 PhyloChip documented how acetate-stimulated groundwater samples differed from background sediment samples by high amounts of Geobacter species and, to a lesser extent, Desulfobacteraceae. Both arrays showed a decrease in Geobacter species during the amendment as predominantly iron-reducing conditions transitioned to predominantly sulfate-reducing conditions.

Later samples probed by the G3 PhyloChip contained high amounts of sulfate-reducing taxa bacteria, including Desulfobacteraceae Desulfovibrionales, Desulfitobacterium, and Desulfotomaculum. To ascertain the active bacteria at the Rifle IFRC, stable isotope probing methods were employed in groundwater and sediments during the Winchester experiment. Specifically, ¹³C acetate was used to assess the active microbes on three size fractions of sediments (coarse sand, fines [8-approximately 150 micron], groundwater [0.2-8 micron]) over a 24-day time frame. Results indicated differences between active bacteria in the planktonic and particle associated phases. with a Geobacter-like group (187, 210, 212 bp) active in the groundwater phase, an alpha Proteobacterium (166 bp) growing on the fines/sands, and an Acinetobacter sp. (277 bp) utilized much of the ¹³C acetate in both groundwater and particle-associated phases. Analysis of the microbial community in the naturally reduced sediment (La Quinta) indicated Geobacteraceae comprised 20% of the natural background community, 4 times greater than more oxidized sediment collected from the Rifle IFRC site. When La Quinta sediment was incubated with acetate, Geobacteraceae never became predominant, suggesting that the Geobacteraceae found in La Quinta may function differently from other organisms belonging to this family

### Example 13: Complexity and Heterogeneity in Biostimulated Sediment and Groundwater Communities During Iron, Sulfate, and Uranium Reduction

A phylogenetic microarray investigation into biostimulated iron- and sulfate-reducing bacterial (SRB) communities revealed unexpected similarity between sediment and groundwater fractions, variability in key functional groups, and an insight into potentially important low-abundance organisms. Bacterial communities from a range of acetate-amended and unstimulated samples associated with a U(VI) bioremediation experiment in Rifle, Colo., were compared using a newly developed LBNL PhyloChip, which is able to detect DNA from tens of thousands of organisms of even extremely low abundance. In contrast, more traditional techniques (e.g. clone libraries) tend to under represent low-abundance community members.

Addition of acetate to Rifle groundwater stimulated the indigenous microbial community to reduce Fe(III) and sulfate consecutively, and U(VI) concomitantly. It is likely that abundant Geobacter spp. were responsible for Fe(III) and U(VI) reduction during early stage biostimulation, while sulfate was primarily reduced by Desulfobacteraceae. Data also suggest that minor enrichments of non-acetate-oxidizing SRB groups—Peptococcaceae and previously undetected Desulfovibrio (See Anderson R T et al. (2003) Stimulating the in situ activity of Geobacter species to remove uranium from the groundwater of a uranium-contaminated aquifer. AEM 69: 5884-5891)—included potential competitors to residual Geobacter spp. for enzymatic U(VI) reduction during sulfate reduction. Communities were highly similar within specific sample treatments (acetate amended: [a, b] subsurface groundwater/sediment, and [c] laboratory or [d] in-well field column sediment/quartz; [e] naturally reduced subsurface sediment), with the exception that Geobacter demonstrated a strong preference for attachment to the Fe(III)-bearing Rifle sediment over quartz sand in column experiments (c). Curiously, a subset of sulfate-reducing sediments (d) displayed greater similarity to Fe(III)- and sulfate-reducing groundwater communities than to other sulfate-reducing sediments (b-e). This is likely due in part to the broad overlap in elevated Geobacter with Desulfobacteraceae and Desulfovibrionales, and to differential increases in Peptococcaceae, which were limited to selective sediments (c, e).

### Example 14: Uranium Biomineralization by Natural Microbial Phosphatase Activities in the Subsurface

The goal of this example is to examine the role of microbial phosphohydrolases in naturally occurring subsurface microorganisms for the purpose of promoting the immobilization uranium through the production of insoluble uranium phosphate minerals. The results of our prior NABIR-ERSP (SBR) project demonstrate that subsurface microorganisms isolated from radionuclide- and metal-contaminated soils at the DOE Oak Ridge Field Research Center (ORFRC) are acid-tolerant and resistant to numerous toxic heavy metals, including lead. In addition, many of these lead-resistant isolates exhibit phosphatase phenotypes (i.e., in particular those surmised to be phosphate-irrepressible) capable of ameliorating metal toxicity by the liberation of inorganic phosphate during growth on organophosphorus compounds, with the concomitant production of a metal-phosphate precipitate. Liberated phosphate from glycerol-3-phosphate was sufficient to precipitate as much as 95% of U(VI) as low-solubility uranium-phosphate minerals in synthetic groundwater containing either dissolved oxygen or nitrate as terminal electron acceptor in the pH range 5 to 7. In this example, we have developed an experimental approach to determine whether the activities of naturally occurring microbial phosphatases in subsurface microbial communities result in the immobilization of uranium via the formation of phosphate minerals in contaminated soils.

Characterization is being carried out of the subsurface microbial community responses of U(VI) and NO₃ contaminated ORFRC Area 2 and Area 3 soils, as well as the microbial population responses to exogenous organophosphate additions under oxic and anoxic growth conditions, soil slurry, and flow-through reactor experiments conducted at pH 5.5 and 7.0. Soil slurry and flow-through reactor experiments were conducted for 36 days and 80 days at 25° C. with 10 mM G2P and 15 mM NO₃⁻ as the sole C, P, and N sources, respectively. Under oxic growth conditions, greater than 4 mM soluble PO₄³⁻ was measured at the end of the slurry incubations, and NO₂⁻ was not detected. Preliminary data obtained for anoxic soil slurry incubations indicated an accumulation of greater than 1 mM PO₄³⁻, as well as the accumulation and subsequent removal of NO₂⁻. Following triplicate incubations, 16S rDNA diversity of slurries were analyzed via high-density 16S oligonucleotide microarrays (PhyloChip). Preliminary results suggest that under oxic conditions, the microbial community structure is enriched in proteobacterial taxa at low pH as compared to the diversity of unamended soils. Analyses of slurries incubated under anoxic conditions are under way to identify bacterial taxa capable of organophosphate hydrolysis under both oxic and anoxic environments. Flowthrough reactor studies of soils with an initial pH of 3.7 demonstrated robust microbial activities once a pore water pH of 5.5 was achieved. Both denitrification and organophosphate hydrolysis were measured within 2 days of pH adjustment. Our soil slurry and column studies demonstrate the potential efficacy of organophosphate-mediated sequestration of U(VI) by the microbial community residing in ORFRC contaminated subsurface soils.

### Example 15: Microbial Community Trajectories in Response to Accelerated Remediation of Subsurface Metal Contaminants

Remediation of subsurface metal contaminants at DOE sites involves microbial mechanisms of oxidation/reduction or complexation, which are controlled in large part by the ecology of the microbial community. Recognizing and quantifying the relationships between community structure, function, and key environmental factors may yield quantitative understanding that can inform future decisions on remediation strategies. We have previously found that U bioreduction and maintenance of low aqueous U concentrations is strongly dependent on the organic carbon (OC) supply rate. Our results also showed that OC supply rate had a significant effect on microbial community structure, while the effect of two different OC types was secondary over the duration of the experiment. The differences between communities attributable to different rates of OC supply diminished through time, despite the fact that different rates of OC supply resulted in different environmental conditions within the columns. Together, these data indicate that microbial communities stimulated for bioremediation may follow predictable trajectories.

Based on our prior work, and operating under the premise that microbial communities can be controlled and predicted, as well as the resulting remediation capacity, the objectives of our current project are to: (1) determine if the trajectories of microbial community structure, composition and function following OC amendment can be related to, and predicted by, key environmental determinants; and (2) assess the relative importance of the characteristics of the indigenous microbial community, sediment, groundwater, and OC supply rate as the major determinants of microbial community functional response and bioremediation capacity. We are analyzing three sediments (Oak Ridge, Tenn.; Rifle, Colo.; Hanford, Wash.) and their microbial communities using a reciprocal transplant experimental design. Initial characterization of the three sediments show that they vary in mineralogy; particle size distribution; bulk density; base cations; CEC; SAR; iron, manganese, phosphate, and sulfate concentrations; organic and inorganic carbon concentrations; pore-water chemistry; and microbial community size and composition. Flow-through reactors, receiving simulated groundwater at two OC supply rates, are being destructively sampled over a period of 18 months. Microbial community trajectories are being followed using: 16S PhyloChip analysis of community DNA (overall structure) and RNA (active members); GeoChip functional analysis of community DNA (functional potential) and community RNA (active functions); and meta-transcriptome analyses to explore functional capacities not included on extant arrays. Geochemical characteristics of reactor effluents and sediments are being used to model factors influencing microbial community structural and functional trajectories. These analyses will provide a framework for the microbial community ecology underlying subsurface metal remediation at DOE sites.

### Example 16: Quantitative Analysis Aids in Ordination

Subsurface sediments were collected from metal-contaminated DOE sites at Oak Ridge, Tenn., Hanford, W A, and Rifle, Colo. Multiple (n=13-15) gDNA extractions using 1-3 g sediment were performed from each site. Extracts were quantified then 10 ng of gDNA was amplified by 8-temperature gradient 16S PCR. From the temperature pools, 500 ng were hybridized to the G3 PhyloChip. Hybridization intensity for each OTU was determined as the trimmed mean of PM-MM differences for each OTU's set of probe pairs. NMDS ordinations were made in R using Bray-Curtis distance for relative abundance and Sorensen for presence/absence data.

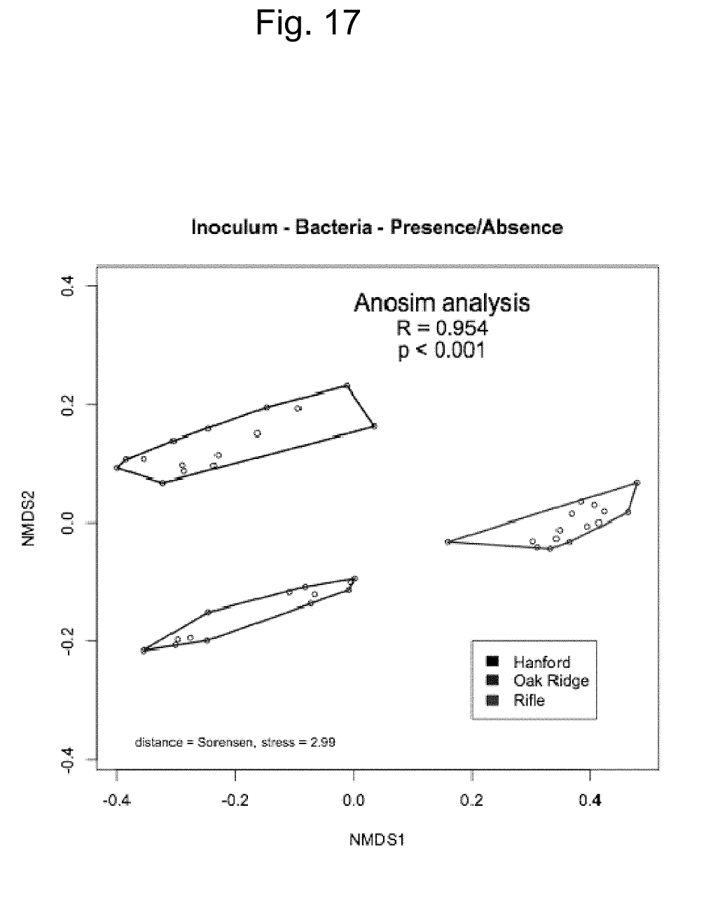

FIG. 17 is a chart showing PhyloChip results from similar biological communities form ordination clusters. OTUs were called present or absent from samples taken from subsurface sediments from three different locations. A distance matrix between the samples was created based on the Sorrensen distance. The distance matrix was ordinated using NMDS and colored by sample location. Anosim analysis revels that samples within groups are more similar in composition than samples from different groups.

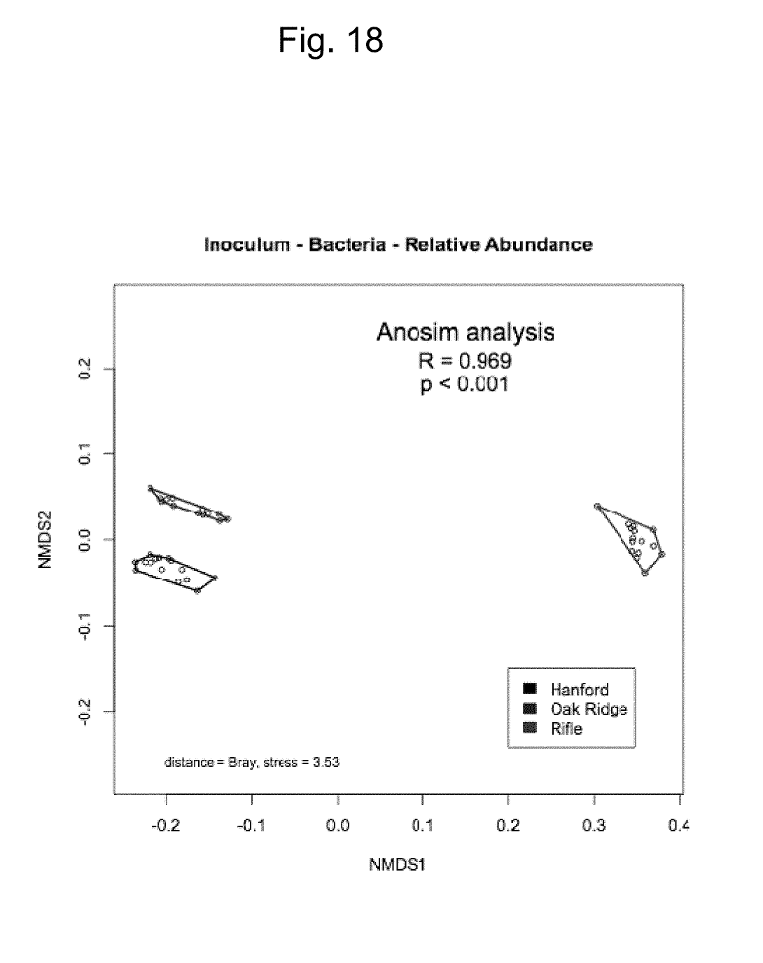

FIG. 18 is a chart showing PhyloChip results from similar biological communities form ordination clustersOTUs were quantified from samples taken from subsurface sediments from three different locations. A distance matrix between the samples was created based on the Bray-Curtis distance. The distance matrix was ordinated using NMDS and colored by sample location. Anosim analysis revels that samples within groups are more similar in composition than samples from different groups. The R value is greater compared to previous plot indicating that relationships among similar sample types are closer when utilizing the quantitative PhyloChip data.

### Example 17: Quantitative Analysis in Sludge Bioreactors

Activated sludge bioreactors are widely used to remove organics and nutrients from wastewater. However, the role of immigration in structuring activated sludge microbial communities is little understood. Converging lines of evidence from a year-long series of weekly samples at a full-scale wastewater treatment plant indicated a strong link between aeration basin influent NO₂ and shifts in activated sludge microbial community structure. To further investigate this association, we sampled four locations along a transect within this plant: 1) plant influent; 2) trickling filter biofilm; 3) trickling filter effluent; and 4) the activated sludge bioreactor. Here, we show via a polyphasic approach that influent NO₂ is a signature of microbial immigration from the upstream biofilm-based trickling filter to the activated sludge bioreactor. High-density phylogenetic microarray (PhyloChip) analyses revealed an overabundance of methanogens and sulfate-reducing bacteria in the trickling filter and suggested microbial transport to the activated sludge via the trickling filter effluent. Furthermore, ammonia-oxidizing bacterial (AOB) amoA copy number increased by an order of magnitude between plant influent and trickling filter effluent, indicating accumulation of AOB in the trickling filter and significant immigration to the activated sludge unit. Molecular fingerprinting (T-RFLP) analyses corroborated by clone libraries showed that Nitrosomonas europaea dominated the trickling filter, while a Witrosomonas-like′ lineage dominated in activated sludge. N. europaea was previously shown to dominate in activated sludge during elevated influent NO₂ events, suggesting that activated sludge AOB community dynamics are driven in part by immigration via sloughing from the upstream trickling filter.

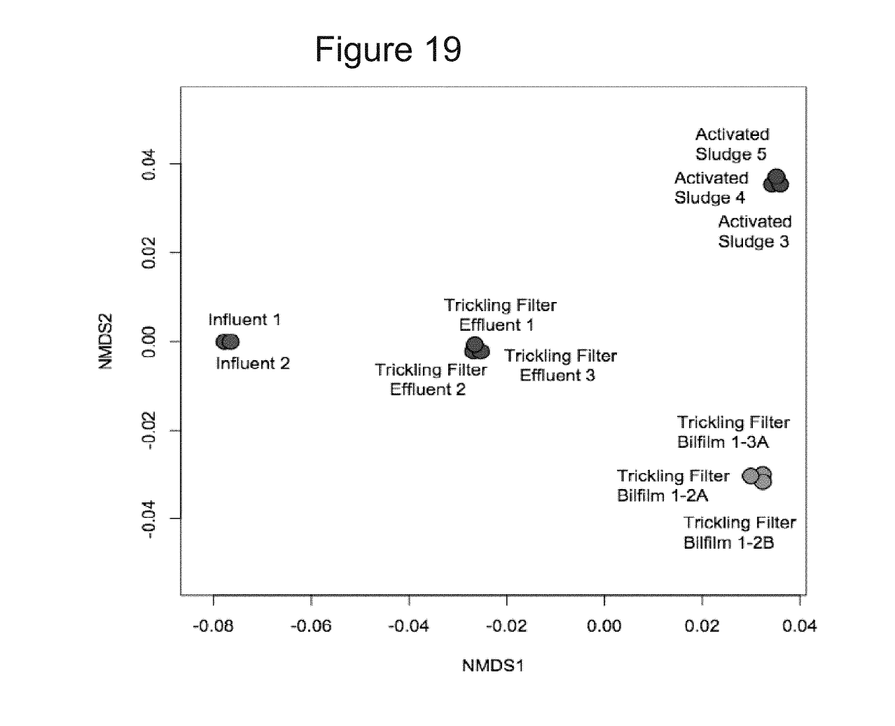

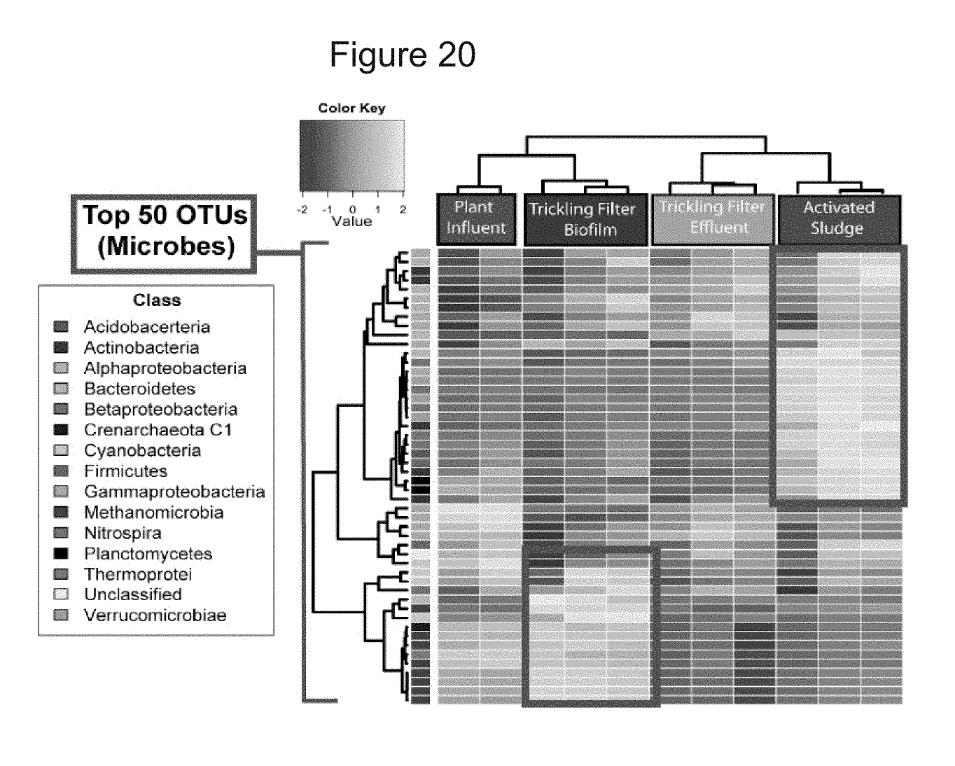

FIGS. 19 and 20 illustrate the analysis that was performed using the PhyloChip G3 array. FIG. 19 shows an NMS analysis demonstrating that the four sampling sites are quite distinct, and that the biological replicates show quite high levels of similarity. FIG. 20 is a heatplot summary of an analysis called the Method of Shrunken Centroids. The basic idea of this analysis is to identify the ˜50 or so microbial OTUs that most significantly define the observed differences in overall community structure between sampling locations. As we hypothesized, anaerobes (particularly methanogens) are well represented in this set of 50 microbial types, and we see evidence of transport between sampling locations (namely the trickling filter and the activated sludge aeration basin) of these microbes. In addition, Nitrospira (nitrite-oxidizing bacteria) are also fairly well represented in this “minimal” dataset. Notably, we see small levels of nitrite accumulation in one of the sampling locations—the trickling filter biofilm—in which the PhyloChip results indicate essentially an absence of Nitrospira, and essentially no nitrite accumulation in the downstream activated sludge unit, where Nitrospira are much more abundant.

Taken together, our results provide compelling evidence that immigration between coupled process units can significantly influence activated sludge microbial community structure.

### Example 18: PhyloChip G3 Analysis on the Impact of Climate Change on Redwood Forests

This project examined the potential impacts of climate change on the composition of soil microbial communities in coastal redwood forests. Understanding their response to climate change is important for predicting changes in ecosystem services and of interest to ecosystem stewards.

A 3-way reciprocal transplant experiment was conducted across the latitudinal gradient of coastal redwood forests. Samples were collected 1 year and 3 years after transplanting. Bacterial community composition was analyzed using a high-density 16S rDNA microarray (PhyloChip). Climatic variables and soil variables (rainfall, soil moisture, soil temperature, soil C and N availability, pH, soil texture) were measured. Changes in community composition were assessed with non-metric multidimensional scaling (for the entire community) and ANOVA (for individual taxa). The relationships between bacterial community composition and climatic and edaphic variables were examined with Mantel tests.

The change in climate had an intermediate to strong influence on bacterial community composition. The amount of rainfall and its impact on soil moisture were the strongest and most significant correlates with community composition. In addition, the number of bacterial species that responded to the change in climate increased from year 1 to year 3.

The results indicate that climate change has an intermediate to strong influence on bacterial community composition at a regional scale. The amount of rainfall had the most significant correlation with bacterial community composition. While other factors, such as species interactions or other stochastic processes, may also greatly influence changes in community composition over time, it appears that the number of species that respond to the impact of climate change increases with time and 3 years may not be long enough to assess the long-term impact of climate change on microbial community composition.

Table 13 shows significant standardized Mantel statistics (r) for the relationships between the bacterial community composition of transplanted samples and controls and environmental variables, for both one and three years after samples were transplanted.

TABLE 13

| | Axis 1 Mantel | Mantel test |
|---|---|---|
| Environmental variable | test r | p-value |
| 1 year after transplanted | | |
| Annual rainfall | 0.19 | 0.013 |
| Late spring rain | 0.19 | 0.019 |
| All env. variables | 0.19 | 0.015 |
| 3 years after transplanted | | |
| Annual rainfall | 0.17 | 0.009 |
| Summer rain | 0.19 | 0.007 |
| Gravimetric water content | 0.18 | 0.040 |
| Temperature | 0.13 | 0.047 |
| Maximum temperature | 0.12 | 0.034 |
| Annual rainfall + temperature | 0.17 | 0.011 |

TABLE 14
Bacteria (OTUs) that respond to transplanting
after 1 year and 3 years

| | | After 1 year | After 3 years |
|---|---|---|---|
| Phylum/Division | Class | (no.*) | (no.*) |
| Acidobacteria | | 1 | 17 |
| Actinobacteria | | 0 | 38 |
| Bacteroidetes | | 1 | 8 |
| Chlorflexi | | 0 | 14 |
| Firmicutes | | 16 | 39 |
| Planctomycetlaes | | 0 | 9 |
| Proteobacteria | Alpha- | 11 | 104 |
| | Beta- | 21 | 15 |
| | Delta- | 0 | 11 |
| | Gamma- | 20 | 13 |
| Spirochaetes | | 1 | 18 |
| Other | | 3 (from 2 | 39 (across |
| | | divisions) | 18 divisions) |
| TOTAL | | 74 | 325 |

The number of OTUs that have a difference in relative abundance (OTU intensity) between treatments (origin-incubation combinations) by ANOVA at p<0.10.

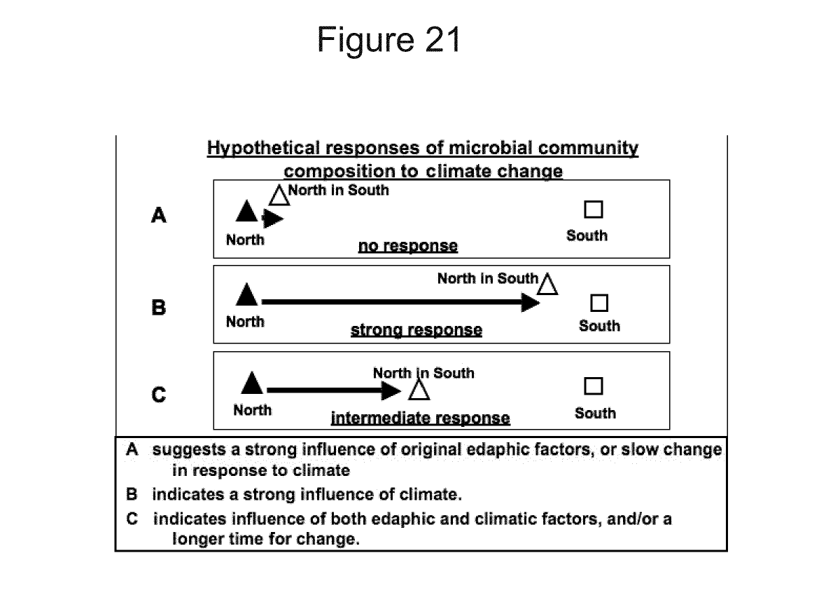

FIG. 21 is a representation of differing degrees of change in community composition in response to a change in climate. The open squares represent the position of a Southern-lat. site in an ordination, and the black squares represent the position of a Northen-lat.site. The open triangles represent the community of a Northan-lat. site that experienced the Southern-lat. climate. The length of the arrow shows the degree of change.

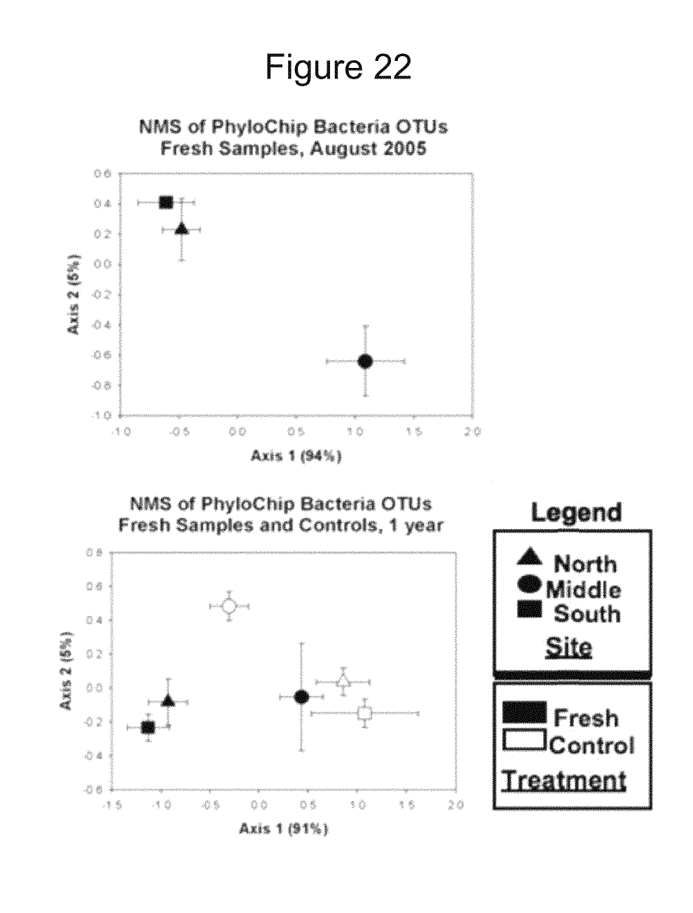

FIG. 22 is two charts showing NMS ordinations of: a) Fresh samples collected from the North, Mid and South-lat. sites in August 2005 and b) fresh samples and transplant-control samples from the same sited at the same time (1 year after transplanting). The fresh samples depicted in both graphs are the same samples. The bars represent 1 s.d. of 3 replicates.

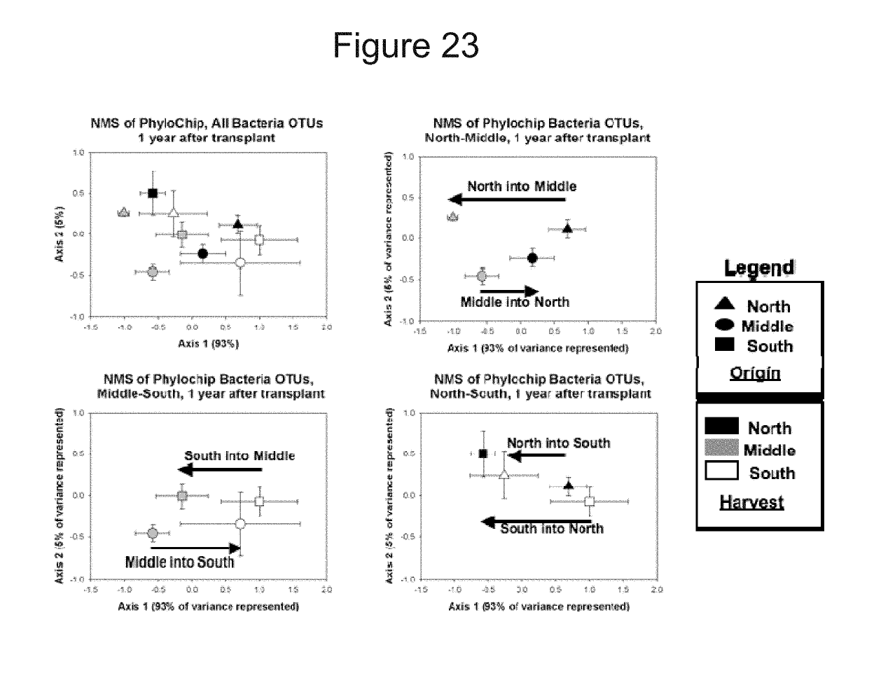

FIG. 23 is four charts showing NMS ordinations of reciprocally transplanted samples and transplanted controls collected 1 year after they were transplanted. Arrows show the trajectory of the change in composition of transplanted samples away from that of their site-of-origin controls.

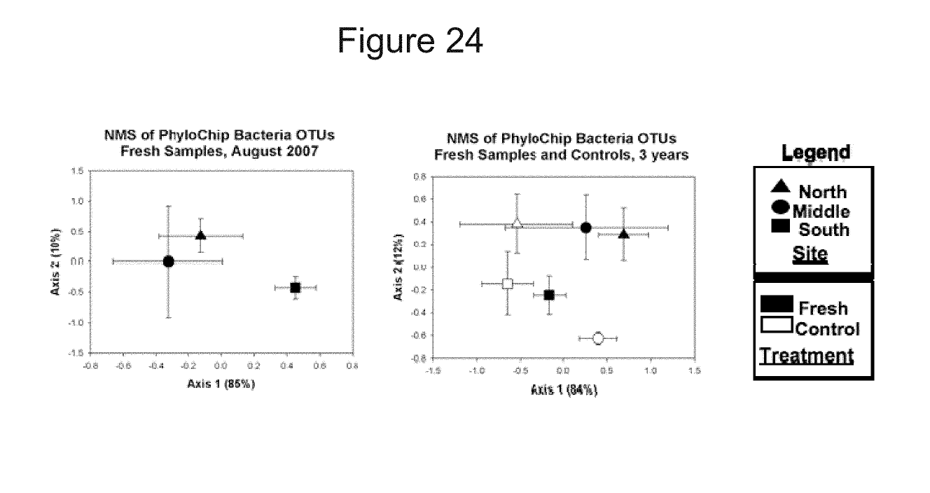

FIG. 24 shows 2 charts showing the NMS ordinations of: a) Fresh samples collected from the North, Mid and South-lat. sites in September 2007 and b) fresh samples and transplant-control samples from the same sites at the same time (3 years after transplanting). The fresh samples depicted in both graphs are the same samples. The bars represent 1 s.d. of 3 replicates.

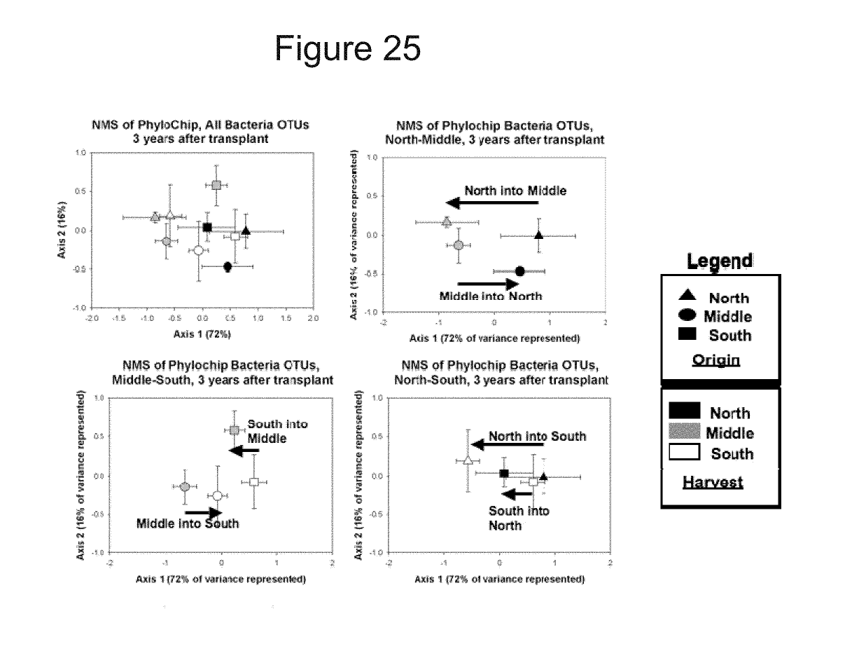

FIG. 25 is four charts showing NMS ordinations of reciprocally transplanted samples and transplanted controls collected 3 years after they were transplanted. Arrows show the trajectory of the change in composition of transplanted samples away from that of their site-of-origin controls.

### Example 19: Microbial Community Analysis of Mammalian and Avian Sources of Fecal Contamination in Coastal California

Wild and domestic animals that inhabit coastal areas deposit fecal microorganisms that impact water quality. The extent to which coastal waters are impaired by various human and animal sources of fecal pollution is hard to determine with single biomarkers and low-resolution profiling methods. High-throughput sequence analysis of gut microbial communities has potential to reliably identify fecal sources and resolve contentious water quality issues. In this study we characterized bacterial communities from a variety animal feces and human wastes to identify taxa that distinguish contamination sources. We then tested the utility of these findings during water pollution events.

Fresh fecal samples were collected from at least four geographically-distinct populations each of gulls, geese, pinnipeds (seals and sea lions), cows, horses and elk. Human sewage and septic waste were gathered from multiple locations. We analyzed bacterial 16S rRNA gene composition using the PhyloChip microrray, which is capable of quantifying differences in the relative abundance of both rare and abundant bacterial taxa by detecting the entire targeted pool of 16S rRNA gene copies in each sample.

Ambient water samples were collected weekly over two years at nine recreational beaches in N. California and during a major sewage spill in San Francisco Bay. Water samples were measured using common fecal indicator tests and analyzed using the PhyloChip for source identification.

Fecal bacterial communities strongly clustered by animal species/type. We identified thousands of bacterial taxa that distinguished human wastes from animal feces, and different animals from each other. Human waste samples clustered together despite differences in the scale and type of processing. Bacterial communities in cows and elk were nearly indistinguishable, and there was little variation among different populations of these ruminants. In contrast, bacterial communities in birds were much more variable among populations, even within the same species. Horse populations clustered with other grazers but were distinct in composition from the ruminants. Analysis of water samples during pollution events demonstrated that libraries of distinctive taxa developed from our source characterization could successfully identify or exclude causes of contamination.

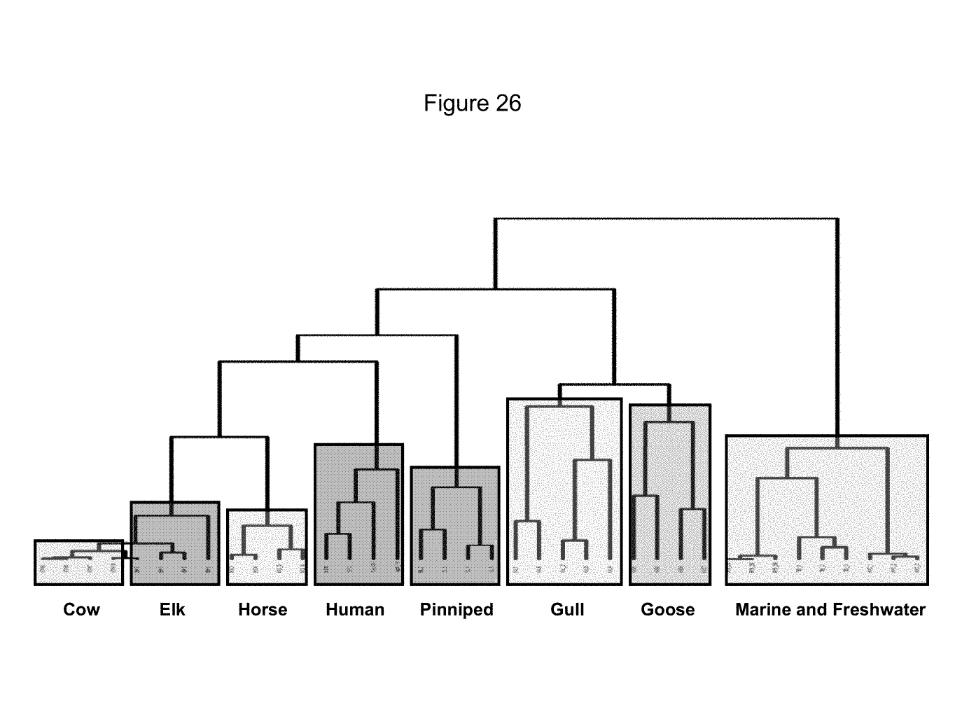

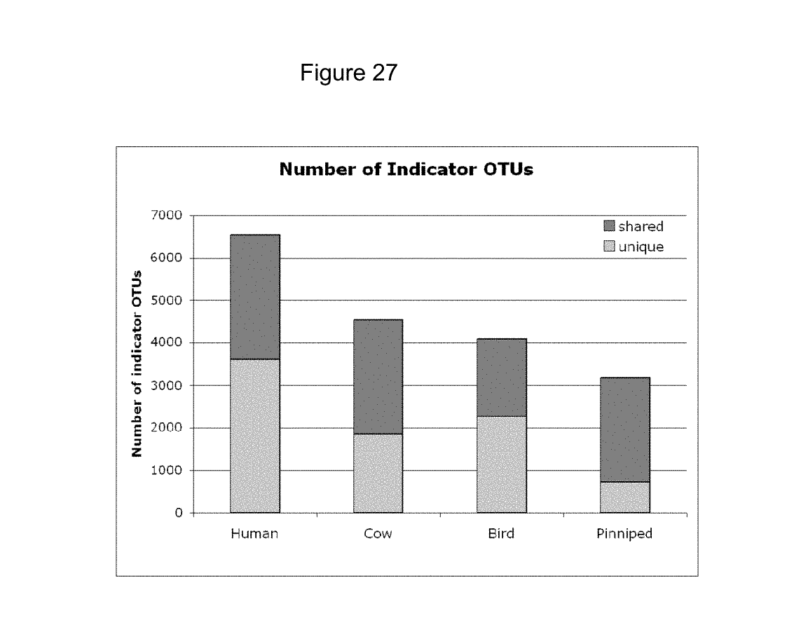

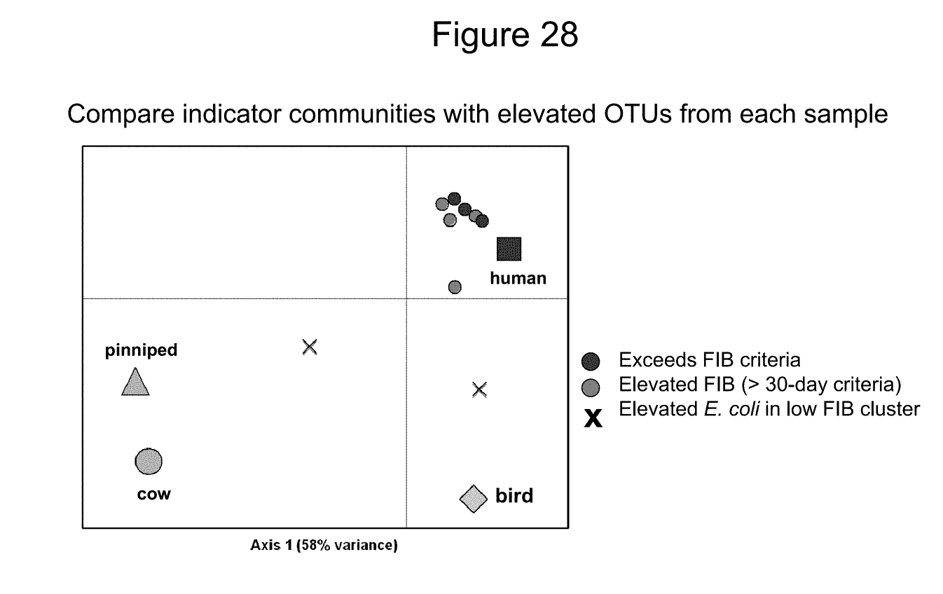

Cluster analysis of detected bacterial taxa in fecal samples and clean water samples was performed and showed that the PhyloChip G3 array detected 3513 different bacterial subfamilies in fecal samples (passed stage 1 analysis). Strong clustering by species and type of animal (ruminants and grazers, pinnipeds, birds) was shown and displayed in FIG. 26. Using the PhyloChip G3 array, human sources (septic tanks, sewage) are distinct from animals and wildlife, and background waters. Source identifier communities were defined for each source. Detected OTUs (pass stage 1) had significantly higher array intensity than background waters (t-test and difference in avg. array intensity >2000) (FIG. 27). In FIG. 28, indicator communities were compared to polluted water samples for source identification.

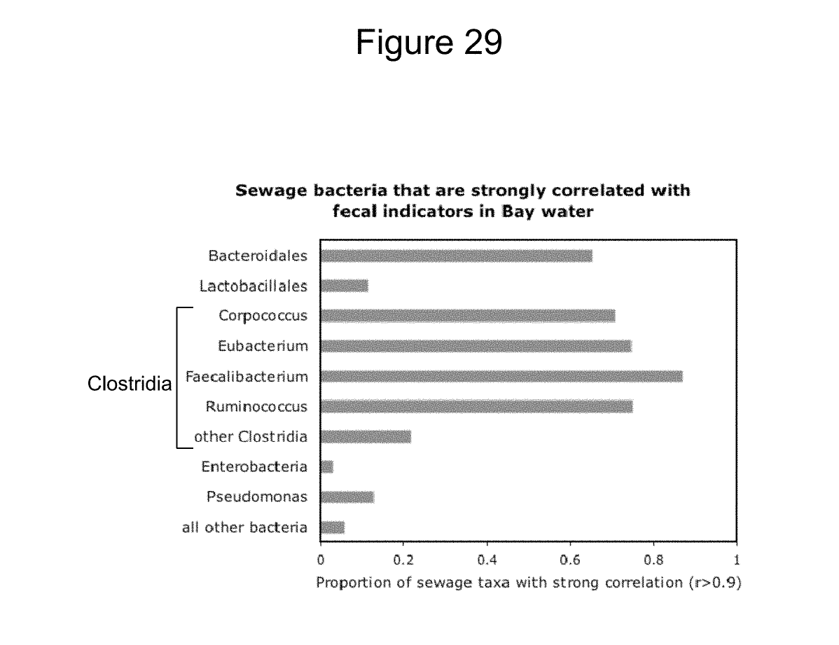

Sewage taxa with strong correlations to FIB are shown in FIG. 29. Abundances of 4,625 different taxa found in sewage were strongly correlated (r>0.9) with fecal indicators. The most correlated taxa were Bacteroidales and Clostridia.

Not shown is a phylogenetic tree of potential indicator taxa identified in Tomales Bay diffusion chamber experiment. Potential indicator taxa are OTUs that are unique to a particular waste and absent in the receiving waters. There were 165 potential indicator taxa identified for dairy farm waste and 119 indicator taxa identified for septic tank waste. A total of 13,341 different taxa were detected in waste and receiving water samples with the G3 chip.

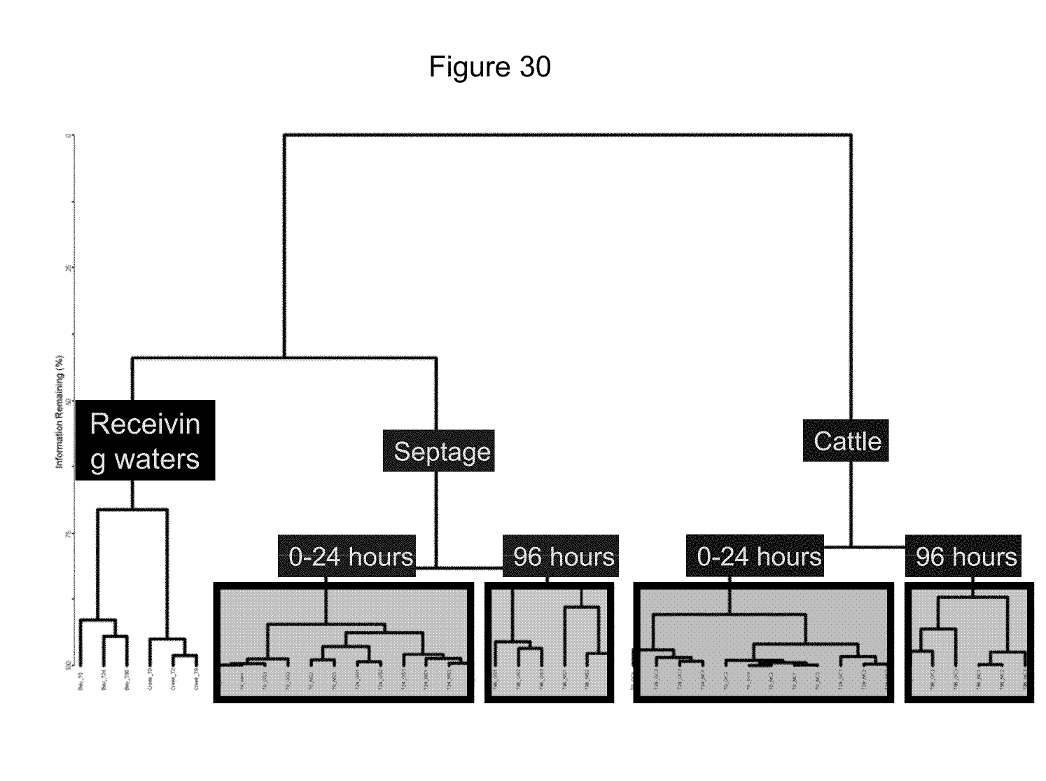

FIG. 30 shows results of cluster analysis which showed the comparison of community composition. Communities can be clustered according to the time in the receiving waters, source, and type of receiving waters.

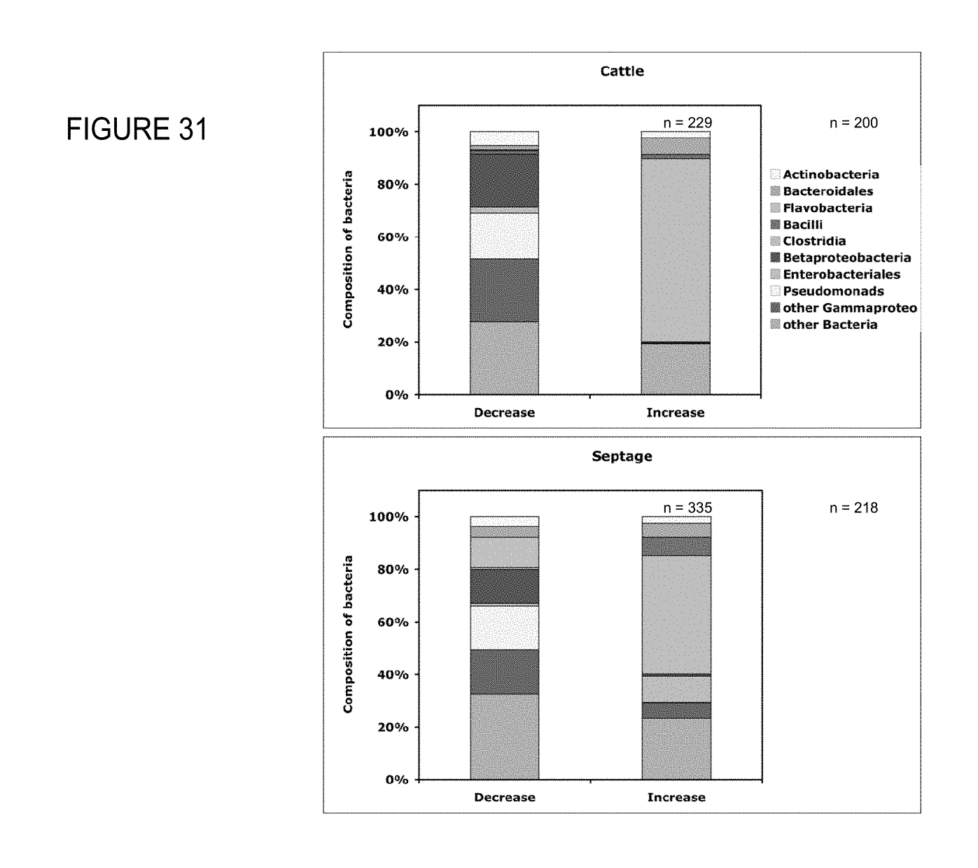

FIG. 31 is a bar chart showing the effect of time in receiving waters on fecal microbial communities. A four day immersion shows differences in persistence among taxonomic groups with similar shifts in cattle and septic communities. Most proteobacteria decrease in relative abundance over time. Clostridia increase in relative abundance over time.

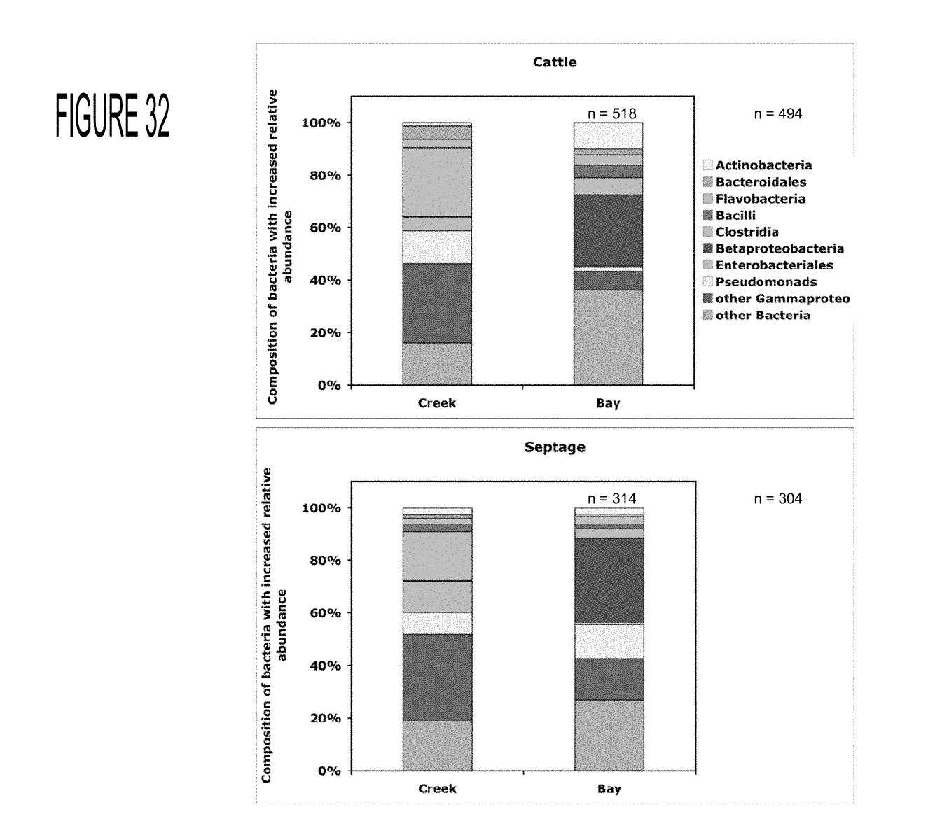

FIG. 32 is a bar chart showing the effect of creek versus bay water on waste microbial communities. Similar response of cattle and septage communities to different water types is illustrated. Clostridia, γ-proteobacteria, coliforms favored in creek while β-proteobacteria is favored in Bay. Selection of molecular indicators for monitoring should consider persistence of taxa under relevant conditions

Thus, different animals harbor distinct fecal microbial communities that can be exploited for source tracking in spite of intra-source variability due to diet, location or processing

### Example 20: Evaluation of Oil Spill Effects and Clean-Up on Ocean Microbiome

The methods, compositions, and systems of the invention can be applied to evaluate the effects of changes in an environment on the microbiome supporting and supported by that environment. In this example, an array of the invention is used to establish a baseline for the microbiome of healthy ocean environments, and this baseline is then used to assess the effects of an oil spill on the microbiome, as well as to assess the progress of recovery efforts.

Microbial DNA is isolated from ˜150 samples representing the diverse ecosystems affected by the oil spill, as well as ˜100 samples from similar, unaffected ecosystems. Samples are collected from a representative range of ocean depths, commercial and recreational fishing areas and coastal areas, e.g., beach and marsh surface water, inlets, and lagoons. Ideally, multiple samples (5-10) are collected per site initially and at each quarterly re-sampling. DNA is extracted from the sample, amplified, processed, and analyzed as in Example 2. Analysis by probe hybridization is conducted using an array, such as described in Examples 2 and 7. The presence, absence, and/or level is scored for each probe evaluated, and/or for each OTU represented by the probes evaluated. The result is a biosignature for unaffected ocean environments and a biosignature for ocean environments affected by the oil spill. Analysis and bioinformatic data mining of the results produces reports on the status of the microbial populations at each site, as well as an interpretative report indicating the scope of damage to the microbial ecosystem services as compared to undamaged, similar marine ecosystems.

Thereafter, samples are collected from each monitoring site on a quarterly basis, and changes from the initial biosignature of oil spill affected areas as well as continuing ecosystem damages relative to unaffected, similar ecosystem sampling sites, are assessed. The relative success of restoration efforts, measured in terms of degree of improvement in similarity between spill-affected biosignatures and unaffected biosignatures, can be used to inform the most appropriate actions for containment or dispersal of future oil spill disasters. Profiles for each healthy marine microbial ecosystem evaluated are established between 3-5 quarters of sampling and take into account normal seasonal fluctuations in the relative abundance and diversity of particular microbial species. By comparing microbial biosignatures from remediated sites with unaffected sites, including confidence and probability information, site specific restoration is tracked. Once these parameters are established, progress towards remediation from the oil spill damage and restoration of healthy, functioning marine ecosystems is projected and qualified. Degree of restoration is assigned a restoration score, which represents a percentage of similarity between the biosignatures of unaffected and affected ocean environments. High similarity of affected treated areas to unaffected area microbial populations provides evidence that spill areas have recovered and are capable of supporting healthy marine life. Tracking increases in similarity between the biosignatures of unaffected and affected ocean environment provides a projection of time to restoration to the unaffected state, as well as defining an endpoint for remediation efforts, wherein remediation efforts are halted once a threshold of similarity is reached. Thresholds can be higher than about 80%, 85%, 90%, 95%, 97.5%, 98%, 95.5%, 99%, 99.1%, 99.2%, 99.3%, 99.4%, 99.5%, 99.6%, 99.7%, 99.8%, 99.9%, 99.95%, or higher similarity.

### Example 21: Effects of Deep Water Oil Plume on Bacterial Community

The oil from the Deepwater Horizon spill in the Gulf of Mexico represents an enormous carbon input to this ecosystem, and hydrocarbon components in the oil could potentially serve as a carbon substrate for the microorganisms present in the water. The impact of the plume on the microbial community and its potential for hydrocarbon degradation was evaluated. This study covers 19 sampling sites on the cruises for two ships from May 25 to Jun. 2, 2010.

### Sample Collection

A colored dissolved organic matter (CDOM) WETstar fluorometer (WET Labs, Philomath, Oreg.) was attached to a CTD sampling rosette (Sea-Bird Electronics Inc., Bellevue, Wash.) and used to detect the presence of oil along depth profiles between the surface and seafloor. Fluorometer results were subsequently confirmed with laboratory hydrocarbon analysis. A total of seventeen samples were analyzed from ten locations.

Niskin bottles attached to the CTD rosette were used to capture water samples at various depths inside and outside waters with detected hydrocarbons. From each sample 800-2000 mL of water were filtered through sterile filter units containing 47 mm diameter polyethylsulfone membranes with 0.22 μm pore size (MO BIO Laboratories, Inc., Carlsbad, Calif.) and then immediately frozen and stored at −20° C. Filters were shipped on dry ice and stored at −80° C. until DNA and phospholipid fatty acid (PLFA) extraction.

100 mL of water was syringe-filtered and injected into pre-evacuated 25 mL serum bottles capped with thick butyl rubber stoppers. 100 mL of water was frozen in 125 mL HDPE bottles for nutrient analyses. For AODC 36 mL water was preserved in 4% formaldehyde (final concentration).

### DNA Extraction

One quarter of each filter was cut into small pieces and placed in a Lysing Marix E tube (MP Biomedicals, Solon, Ohio). 300 μL of Miller phosphate buffer and 300 μL of Miller SDS lysis buffer were added and mixed. 600 μL phenol:chloroform:isoamyl alcohol (25:24:1) was then added, and the tubes were bead-beat at 5.5 m/s for 45 sec in a FastPrep instrument. The tubes were spun at 16,000×g for 5 min at 4° C. 540 μL of supernatant was transferred to a 2 mL tube and an equal volume of chloroform was added. Tubes were mixed and then spun at 10,000×g for 5 min 400 μL aqueous phase was transferred to another tube and 2 volumes of Solution S3 (MoBio, Carlsbad, Calif.) was added and mixed by inversion. The rest of the clean-up procedures followed the instructions in the MoBio Soil DNA extraction kit. Samples were recovered in 60 μL Solution S5 and stored at −20° C.

### PCR Amplification

The 16S rRNA gene was amplified using PCR with primers 27F and 1492R for bacteria, and 4Fa and 1492R for archaea. Each PCR reaction contained 1×Ex Taq buffer (Takara Bio Inc., Japan), 0.025 units/μl Ex Taq polymerase, 0.8 mM dNTP mixture, 1.0 μg/μl BSA, and 200 pM each primer and 0.15-0.5 ng genomic DNA as template. For the PhyloChip assay (PhyloTech Inc., San Francisco, Calif.) analysis each sample was amplified in 4 replicate 25 μl reactions spanning a range of annealing temperatures. PCR conditions were 95° C. (3 min), followed by 30 cycles 95° C. (30 s), 46-56° C. (25 s), 72° C. (2 min), followed by a final extension 72° C. (10 min). Amplicons from each reaction were pooled for each sample, purified with the QIAquick PCR purification kit (Qiagen, Valencia, Calif.), and eluted in 20 μL elution buffer.

### Phylochip Assay Design

The PhyloChip microarray probe design was applied to all known high-quality 16S rRNA gene sequences containing at least 1,300 nucleotides. Sequences (Escherichia coli base pair positions 47 to 1473) were extracted from the NAST multiple sequence alignment available from the16S rRNA gene database, greengenes.lbl.gov. This region was selected because it is flanked by universally conserved segments that can be used as PCR priming sites to amplify bacterial or archaeal genomic material using only 2 to 4 primers. Putative chimeric sequences were identified and removed where Bellerophon divergence ratios >=1.1 with >=90% lane-masked identity to one or both putative parents were encountered. Sequences containing three or greater homo-octomers or longer, or those with >=0.3% ambiguous base calls, were also omitted. From the sub-alignment, putative 25-mer targets were selected with G+C content of 35-75%, secondary structure free energy (ΔG)>=−4 kcal/mol as calculated by RNAfold (17), complimentary melting temperature of 61° C.-80° C., and self-dimerazation melting temperature <35° C. as calculated by Thermalign.

Filtered rRNA gene sequences were clustered to enable selection of perfectly complementary probes representing each sequence of a cluster. Putative amplicons containing 17-mers with sequence identity to a cluster were included in that cluster. The resulting 59,959 clusters, each encapsulating an average of 0.5% sequence divergence were considered operational taxonomic units (OTUs). The OTUs represented 2 domains, 147 phyla, 1,123 classes, and 1, 219 orders demarcated within the archaea and bacteria. Each OTU was assigned to one of 1,464 families according to the placement of its member organisms in the taxonomic outline as maintained by Philip Hugenholtz (Hugenholtz 2002, Genome Biol. 3(2): 1-8). The OTUs comprising each family were clustered into sub-families by transitive (single linkage) sequence identity of 72% common heptamers. Altogether, 10,993 sub-families were found. The taxonomic position of each OTU as well as the accompanying NCBI accession numbers of the sequences composing each OTU are available in the files sequences_by_OTU_G3.gz, taxonomy_by_OTU_G3.gz.

For each OTU, multiple specific 25-mer targets were sought for prevalence in members of a given OTU but dissimilar from sequences outside the given OTU. In the first step of probe selection for a particular OTU, each of the sequences in the OTU was separated into overlapping 25-mers, the potential targets. Then each potential target was matched to as many sequences of the OTU as possible. The multiple sequence alignment provided by Greengenes was used to provide a discrete measurement of group size at each potential probe site. For example, if an OTU containing seven sequences possessed a probe site where one member was missing data, then the site-specific OTU size was only six. In ranking the possible targets, those having data for all members of that OTU were preferred over those found only in a fraction of the OTU members. In the second step, a subset of the prevalent targets was selected and the probe orientation was flipped to the reverse complement to minimize hybridization to unintended amplicons. Probes presumed to be potentially problematic were 25-mers containing a central 17-mer matching sequences in more than one OTU. Thus, probes that were unique to an OTU solely due to a distinctive base in one of four flanking bases were avoided. Also, probes having a common tree node near the root were favored over those with a common node near the terminal branch. Probes complementary to target sequences that were selected for fabrication are termed perfectly matching (PM) probes. As each PM probe was chosen, it was paired with a control 25-mer (mismatching probe, MM), identical in all positions except the thirteenth base. The MM probe did not contain a central 17-mer complimentary to sequences in any OTU. The PM and MM probes constitute a probe pair analyzed together. The average number of probe pairs assigned to each OTU was 37 (s.d. 9.6).

The chosen oligonucleotides were synthesized by a photolithographic method at Affymetrix Inc. (Santa Clara, Calif.) directly onto a glass surface at an approximate density of 10,000 molecules per μm² and placed into “midi 100 format” hybridization cartridges. The entire array of 1,016,064 probe features was arranged as a grid of 1,008 rows and columns. Additional probes for quality management, processing controls, image orientation, normalization controls, hierarchical taxonomic identification, for pathogen-specific signature detection and some implement additional targeted regions of the chromosome. Furthermore, probes complementary to lower confidence 16S sequences were included to enable broadening the phylogenetic scope of the analysis, when those sequences are validated with unambiguous entries into public repositories. The PhyloChip assay design includes control probes for preanalytic, processing, prelabeled hybridization controls, and negative controls. Preanalytic and hybridization controls can also be used in interpretation of background signal intensity and to support normalization of overall fluorescent intensity for sample to sample comparisons.

### Sample Preparation for PhyloChip Assay

From Deep Horizon nucleic acids, 500 ng of bacterial PCR product and 25 ng of archaeal PCR product were prepared for hybridization. PCR products were fragmented to a range of 50-200 bp as verified by agarose gels. Commercial kits were utilized for DNA preparation: Affymetrix (Santa Clara, Calif.) WT Double Stranded DNA Terminal Labeling, and Affymetrix GeneChip Hybridization, Wash, and Stain kits were used for analysis. Briefly, fragmented 16S amplicons and non-16S quantitative amplicon reference controls were labeled with biotin in 40 uL reactions containing: 8 μL of 5×TDF buffer, 40 units of TDF, 3.32 nanomoles of GeneChip labeling reagent. After incubating at 37° C. for 60 min, 2 uL of 0.5M EDTA was added to terminate the reaction. Labeled DNA was combined with 65 μL of 2×MES hybridization buffer, 20.4 μL of DMSO, 2 μL of Affymetrix control oligo B2, and 0.4 μL nuclease free water. Each reaction mixture was injected into the hybridization chamber of an array cartridge and incubated for 16 hours in an Affymetrix hybridization oven at 48° C. and 60 RPM. Hybridization solution was removed and the microarrays were stained and scanned according to the manufacturer's instructions.

### PhyloChip Assay Analysis

Fluorescent images were captured with the GeneChip Scanner 3000 7G (Affymetrix, Sanat Clara, Calif.). An individual array feature occupied approximately 8×8 pixels in the image file corresponding to a single probe 25mer on the surface. The central 9 pixels were ranked by intensity and the 75% percentile was used as the summary intensity for the feature. Probe intensities were background-subtracted and scaled to the Quantitative Standards (non-16S spike-ins) and outliers were identified as previously described(DeSantis et al. 2007, Microb. Ecol. 53: 371). The hybridization score (HybScore) for an OTU was calculated as the mean intensity of the perfectly matching probes exclusive of the maximum and minimum.

Comparison of the PM and corresponding MM intensities is summarized as the pair difference score, d, described above. The d scores are standardized to enable comparison of probe pairs with various nucleotide compositions. The goal in this transformation is determining if a pair's d value is more similar to d values derived from negative controls (NC, probe pairs without potential cross-hybridization to any 16S rRNA sequence nor Quantitative Standards) or to d values from positive controls, the Quantitative Standards (QS, probe pairs with PM's matching the non-16S rRNA genes which are spiked into the experiment). Because the d_{QS} values are dependent on their target's A+T count and T count, the QS pairs are grouped by these attributes into classes and a separate distribution of d_{QS} values are found for each. The dNC values are grouped in the same way. A distribution is estimated for each class from the observations. Each d value from an OTU probe set is compared to the distributions of d_{QS} and d_{NC} from the same class to produce a pair response score, r (described above). The r scores for a set of probe pairs complimentary to an OTU are considered collectively in Stage 1 probe set Presence/Absence scoring. At minimum, 18 probe pairs are considered. The r scores are ranked and the quartiles, rQ₁, rQ₂ and rQ₃ are found. For an OTU to pass Stage 1, all three of the following criteria must be met: rQ₁≥0.70, rQ₂>0.95, and rQ₃>0.98. OTUs which pass Stage 1 are considered in Stage 2 scoring for subfamily detection. In this stage, a cross-hybridization adjusted response score, rₓ, is calculated for all responsive probes (r>0.5), described above. After all penalties are considered, the rₓ values are ranked and quartiles found as above (rₓQ₁, rₓQ₂, rₓQ₃). Subfamilies having a rₓQ₃ values >=0.48 were considered present.

Significantly enriched OTUs within the plume were defined as those achieving a p-value <0.05 with Student's t-test upon log₂(HybScores), Stagel present call in >=4 of 9 plume samples, and an increase in mean HybScores compared to background (outside of plume samples) of >1000 units and >35%.

### PhyloChip Assay Performance

Twenty-six 16S rDNA mixtures from different species were prepared as mock communities using a semi-randomized Latin square structure described by Jacobson and Mathews (Jacobson et al. 1995, Journal of Combinatorial Designs 4: 405). A stepwise function was used so that each successive organism was added at a final concentration 37% greater than the previous organism. Each test organism was represented in all mixtures at each possible concentration step. The 26 DNA mixtures were hybridized in triplicate on different days. Also as a control, one hybridization was carried out using only the quantitative reference controls. All 16S probe pairs producing a response score, r, above 0.5 for the reference controls were masked from subsequent analysis.

Background-subtracted probe intensities from 12,202 replicate probes representing 3,548 different 25-mer combinations were used to determine the coefficient of variation (CV) for each assay. Overall, the variations were minor producing a mean CV=0.097. Additionally, a significant correlation was found between the concentrations of each gene in the Latin Square and the corresponding HybScore generating and average correlation coefficient, r=0.941).

The ability to detect and classify amplicons within the hybridization mix was evaluated using receiver operating characteristic (ROC) curves. The rQ₁, rQ₂ and rQ₃ probe set summarizations were collected from each of the possible OTUs from all Latin Square results. ROC curves were plotted to evaluate the effect of choosing a single threshold to determine presence. The y-axis, Expected Positive Rate, is the fraction of OTUs expected to be present that were called present. The x-axis, Unexpected Positive Rate, is the fraction of OTUs not-expected to present that were called present. Presence/Absence thresholds for each quartile were varied from 0, least stringent to 1, most stringent. For example, in the rQ₁ plot, a threshold of 0.5 allows 97.5% of the expected detection events to pass. Instead of relying on a single threshold to determine presence, all three quartiles of a probe set are examined to ensure the distribution of response scores are skewed toward 1. Collectively, rQ₁>0.70, rQ₂>0.95, and rQ₃>0.98 was required to achieve a 0.961 Expected Positive OTU Rate for amplicons >2 and <348 pM with a 0.020 Unexpected Positive OTU Rate. In Stage 2 rₓQ₃ subfamily thresholds set at 0.48 allowed a 0.969 Expected Positive Subfamily Rate with a corresponding 0.019 Unexpected Positive Subfamily Rate when applied to the Latin Square data over the same concentration range.

Hybridization results were reduced to a community profile from each PhyloChip assay in a format useful for multivariate statistics. OTUs passing Stage 1 within subfamilies passing Stage 2 constituted the community profile. Replicate community profiles of the Latin Square mock communities were compared by ordination. Inter-profile distance was calculated with either the Bray-Curtis or weighted Unifrac method and resulting distance matrices were ordinated with non-metric multidimentional scaling(NMDS). Profiles from each of the 26 mock communities were clearly distinguishable using either distance method. Analysis of variance using either distance matrix (Adonis) concluded a significant difference among mock-communities (p<0.005).

### Results

The plume significantly altered microbial community phylogenetic composition and structure. Using a phylogenetic microarray (PhyloChip assay), a 40% decline in detectable bacterial richness was found and a significant shift in microbial community composition. Ordination of community composition determined by phylogenetic microarray analysis revealed two distinct clusters of samples: one composed entirely of samples with detected oil and the other with samples that had no oil detected. No other physical or chemical factors other than hydrocarbons were significantly different between these groups, indicating that microorganisms are responding directly to the presence of dispersed oil.

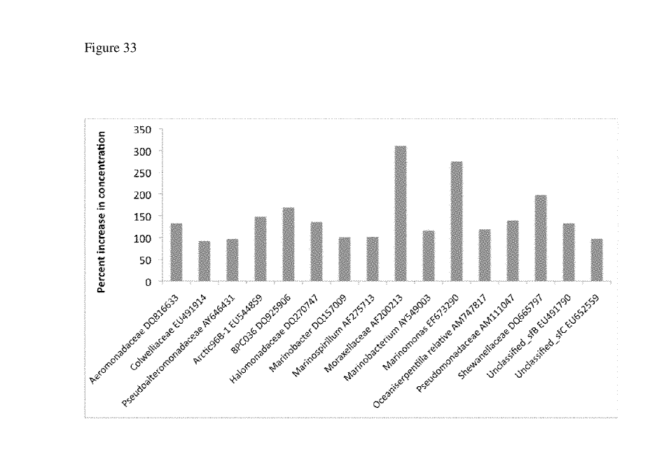

Only bacteria in the class γ-proteobacteria were significantly enriched in plume samples (Table 15). In plume samples 951 distinct bacterial taxa in 62 phyla were detected, but only sixteen distinct taxa that were all classified as g-proteobacteria were significantly enriched by the plume relative to deep waters outside the plume (Table 15, FIG. 33). Nearly all of enriched taxa are known to degrade hydrocarbons or are stimulated by the presence of oil in cold environments (Table 15). Plume-enriched bacteria include many psychrophilic and psychrotolerant species that are known from cold ocean waters, sea ice and circum-polar habitats. The results indicate that these γ-proteobacteria dominate the microbial community in the deep-sea plume. While cell densities are higher, taxonomic richness is lower and the diversity of enriched bacteria is restricted to these few γ-proteobacteria. Oceanospirillales in the γ-proteobacteria was detected in all 9 oil plume samples analyzed by the PhyloChip assay, and was significantly enriched relative to background deep seawater with no oil.

TABLE 15
γ-proteobacteria taxa enriched by the oil plume. Taxa that include
known hydrocarbon degraders or previously shown in cold waters
to become enriched in response to hydrocarbons are indicated.

| | | Hydrocarbon | Enriched by | Representative |
|---|---|---|---|---|
| Class | Family | degraders* | crude oil* | sequence |
| Aeromonadaceae | Aeromonadaceae | + | + | DQ816633.1 |
| | | | | Zebrafish gut clone |
| Alteromonadales | Colwelliaceae | ND | + | EU491914.1 East |
| | | | | Pacific Rise |
| | | | | deepwater clone |
| Alteromonadales | Pseudoalteromonadaceae | + | + | AY646431.1 |
| | | | | Pseudoalteromonas |
| | | | | sp. |
| Arctic96B-1 | Unclassified | ND | + | EU544859.1 Arctic |
| | | | | seawater clone |
| BPC036 | Unclassified | ND | + | DQ925906.1 |
| | | | | Guaymas Basin clone |
| Halomonadaceae | Halomonadaceae | + | + | DQ270747.1 |
| | | | | Halomonas sp. |
| Marinobacter | Marinobacter | + | + | DQ157009.2 |
| | | | | Marinobacter |
| | | | | haloterrigenus |
| Marinospirillum | Marinospirillum | + | + | AF275713.1 |
| | | | | Marinospirillum |
| | | | | alkaliphilum |
| Moraxellaceae | Moraxellaceae | + | + | AF200213.1 |
| | | | | Psychrophilic marine |
| | | | | isolate |
| Oceanospirillales | Marinobacterium | + | + | AY549003.2 Marine |
| | | | | bone clone |
| Oceanospirillales | Marinomonas | + | + | EF673290.1 |
| | | | | Marinomonas sp. |
| Oceanospirillales | Unclassified | + | + | AM747817.1 |
| | | | | Oceaniserpentilla |
| | | | | haliotidis |
| Pseudomonadaceae | Pseudomonadaceae | + | + | AM111047.1 |
| | | | | Pseudomonas sp. |
| Shewanellaceae | Shewanellaceae | + | + | DQ665797.1 |
| | | | | Shewanella |
| | | | | frigidimarina |
| Unclassified | Unclassified_sfB | ND | ND | EU491790.1 East |
| | | | | Pacific Rise seafloor |
| | | | | clone |
| Unclassified | Unclassified_sfC | ND | ND | EU652559.1 Yel Sea |
| | | | | sediment clone |

ND = No Data

FIG. 33 provides an illustration of enrichment of select bacterial taxa by the oil plume. Phylogenetic microarray analysis was used to calculate average difference in estimated concentration between plume and non-plume samples. Average difference is shown as a percentage of non-plume concentration for representative OTUs in enriched taxonomic subfamilies (Table 15).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-110. (canceled)

111. A system for detecting the presence of an operation taxon unit (OTU) in a sample, said system comprising:
(a) an array comprising nucleic acid probe pairs, wherein each probe pair consists of a perfect match (PM) probe and a mismatch (MM) probe that differs from the PM probe of the pair by at least one nucleotide, and wherein the array has at least three sets of probe pairs, comprising:
(i) an OTU detection set wherein the PM probes are complementary to nucleic acids from the OTU;
(ii) a positive control set wherein the PM probes are complementary to positive control nucleic acids added to a labeled nucleic acid sample; and
(iii) a negative control set wherein the PM probes and the MM probes are not complementary to the nucleic acids in the labeled nucleic acid sample; and
(b) computer executable instructions for detecting the presence of the OTU when the array is contacted with the labeled nucleic acid sample and hybridization signal intensities of the three sets of nucleic acid probe pairs are measured, said instructions comprising:
(i) comparing signal intensity of a first PM probe from the OTU detection set of probe pairs with signal intensity of its corresponding MM probe;
(ii) measuring a distribution of signal intensities for the positive control set of probe pairs;
(iii) measuring a distribution of signal intensities for the negative control set of probe pairs;
(iv) using the compared signal intensities and the distributions of signal intensities to calculate a probability that the first PM probe is positive for the OTU;
(v) reducing the probability based on a potential for the first PM probe to cross-hybridize with nucleic acids from other OTUs; and
(vi) determining the presence of the OTU based on the reduced probability.

112. The system of claim 111, wherein said system detects presence, absence, relative abundance, and/or quantity of more than 10,000 different OTUs of a single domain in a single assay with confidence greater than 95%.

113. The system of claim 111, wherein said labeled nucleic acid sample comprises a plurality of first nucleic acid sequences that comprise less than 0.01 percent of the total nucleic acids in a population of nucleic acid sequences from the sample, wherein each of the plurality of first nucleic acid sequences is at least 95% homologous to all of the other first nucleic acid sequences in the population of nucleic acid sequences, and wherein said first nucleic acid sequences are selected from the group consisting of 16S rRNA gene, 23S rRNA gene, 5S rRNA gene, 5.85 rRNA gene, 12S rRNA gene, 18S rRNA gene, 28S rRNA gene, gyrB gene, rpoB gene, fusA gene, recA gene, cox1 gene, nif13 gene, RNA molecules derived therefrom, and a combination thereof.

114. The system of claim 111, wherein said system comprises at least 20,000 OTU detection PM probes, and a plurality of MM probes for each PM probe, wherein each PM probe detects a different first nucleic acid sequence, and each MM probe sequence differs from the PM probe to which it corresponds by at least one nucleotide.

115. The system of claim 114, comprising one or more of the following characteristics:
(a) an MM probe is positioned on the array adjacent or close to its corresponding PM probe on the array; and
(b) no MM probe comprises a central 15-mer that is identical to the complement of any sequence to which any PM probe specifically hybridizes.

116. The system of claim 111, wherein said system is configured to produce a biosignature that is indicative of fecal contamination.

117. The system of claim 111, wherein said OTU detection PM probes selectively hybridize to one or more highly conserved polynucleotides.

118. The system of claim 117, wherein one or more of said highly conserved polynucleotides are 16S rRNA gene, 23S rRNA gene, 5S rRNA gene, 5.85 rRNA gene, 12S rRNA gene, 18S rRNA gene, 28S rRNA gene, gyrB gene, rpoB gene, fusA gene, recA gene, cox1 gene, nif13 gene, RNA molecules derived therefrom, or a combination thereof.

119. The system of claim 117, wherein said negative control probes comprise sequences that are not complementary to sequences found in the highly conserved polynucleotides.

120. The system of claim 117, wherein said highly conserved polynucleotides are amplicons.

121. The system of claim 111, wherein said nucleic acid probe pairs are attached to a substrate.

122. The system of claim 121, wherein said substrate comprises a bead or microsphere.

123. The system of claim 122, wherein said substrate comprises glass, plastic, or silicon.

124. The system of claim 111, wherein said OTU is bacterial, archaeal, or fungal.

125. The system of claim 111, wherein said OTU detection set comprises a plurality of probes capable of determining the presence, absence, relative abundance, and/or quantity of at least 10,000 different OTUs in a single assay.

126. The system of claim 125, wherein said system removes data from at least a subset of said OTU detection probes before making a final call on the presence, absence, relative abundance, and/or quantity of said OTUs.

127. The system of claim 126, wherein said data is removed based on OTU detection probe cross-hybridization potential.

128. The system of claim 125, wherein said system is capable of performing sequencing reactions on the same highly conserved region of each of said OTUs.

129. The system of claim 125, comprising one or more species-specific probes.

130. The system of claim 111, configured to detect OTU detection probe signal intensities as a measure of OTU abundance.

131. The system of claim 111, wherein said determining comprises a normalization to enable comparison of probe pairs with various nucleotide compositions.

132. The system of claim 131, wherein said normalization is performed by considering either or both of the thymine content and the adenine plus thymine content of the target nucleic acid.

133. The system of claim 111, wherein said determining comprises calculating a pair difference score, d.

134. The system of claim 111, wherein said probability is a response score, r.

135. The system of claim 111, wherein said reducing is performed at each level of a phylogenetic tree, starting with the lowest level.

136. The system of claim 111, wherein after determining that an OTU is present, the determination is propagated upward through a taxonomic hierarchy by considering any OTU as present if at least one of its subordinate OTUs is present.

137. The system of claim 136, wherein said OTU that is present is selected from a domain, sub-domain, kingdom, sub-kingdom, phylum, sub-phylum, class, sub-class, order, sub-order, family, subfamily, genus, subgenus, species, and a combination thereof.
